(19) United States
(12) Patent Application Publication
(10) Pub. No.: US 2024/0175877 A1
(43) Pub. Date: May 30, 2024

(54) ANTI-ARA H 2 ANTIBODIES AND USES THEREOF

(71) Applicant: The General Hospital Corporation, Boston, MA (US)

(72) Inventor: Sarita U. PATIL, Newton, MA (US)

(21) Appl. No.: 18/285,065

(22) PCT Filed: Mar. 31, 2022
(86) PCT No.: PCT/US2022/022888

§ 371 Date: Sep. 29, 2023

Related U.S. Application Data
(60) Provisional application No. 63/169,034, filed on Mar. 31, 2021.

(51) Int. Cl. G01N 33/68; C07K 16/16

(52) U.S. Cl. CPC G01N 33/6854; C07K 16/16; G01N 2800/24; G01N 2800/52

(57) ABSTRACT

The invention provides anti-Ara h 2 antibodies (e.g., an anti-Ara h 2 neutralizing antibody) and methods of using the same, e.g., for treating and/or preventing peanut allergy or sensitivity. Also provided herein are anti-Ara h 2 antibodies and methods of using the same, e.g., for diagnostics and methods of monitoring peanut oral immunotherapy. Also provided are combinations of anti-Ara h 2 antibodies for use in determining a treatment response of an individual with a peanut allergy to peanut exposure. Also provided are methods for assessing a treatment response of an individual with a peanut allergy to peanut exposure.

### CROSS-REFERENCE TO RELATED APPLICATIONS

This application claims priority to U.S. Provisional Application No. 63/169,034 filed Mar. 31, 2021, which is incorporated by reference in its entirety.

### FIELD OF THE INVENTION

The present invention is related to human antibodies and antigen-binding fragments of human antibodies that specifically bind to a peanut allergen, therapeutic compositions comprising the antibodies and methods of using those antibodies including diagnostic uses.

### BACKGROUND OF THE INVENTION

Peanut allergy is one of the most common causes of severe food allergy attacks. Peanut allergy symptoms can be life-threatening (anaphylaxis). For some people with peanut allergy, even miniscule amounts of peanuts can cause a serious reaction.

Rates of food allergy have nearly tripled in the past two decades, now affecting about 8% of children in the US, and incurring $25 billion in costs annually. There are over 3 million people in the US. More than 1% of children in the United States and Europe have peanut allergy caused by IgE, or allergy antibodies. They can have severe, even life-threatening allergic responses, known as anaphylaxis, to even tiny exposures to peanut. Peanut allergy tends to be persistent even after childhood in about 80% of affected patients. Currently, the only available treatment is avoidance and emergent use of epinephrine, which places our most vulnerable population at significant risk of mortality and morbidity. Recently, Palforzia, an oral immunotherapy (OIT) has been approved by the FDA as a treatment for food allergy.

OIT clinical trials conducted at Massachusetts General Hospital, like others, have consistently shown that less than one-third of patients develop lasting tolerance after cessation of peanut OIT, and the rate of reactions and anaphylaxis requiring epinephrine during OIT poses a serious and significant burden. Therefore, even with potential future implementation of OIT, a significant portion of patients with IgE-mediated food allergy will require treatment to prevent anaphylactic reactions on accidental encounters with peanut allergen.

A need therefore exists for compositions and methods for treating peanut allergy.

### SUMMARY OF THE INVENTION

The invention provides antibodies and antigen-binding fragments thereof that bind specifically to the peanut allergen, Ara h 2. Such antibodies may be useful to bind the Ara h 2 allergen in vivo following exposure of a sensitized patient to a peanut allergen, and as such, may act to either promote clearance of Ara h 2, neutralization of the effects of Ara h 2 or to block the binding of the allergen to pre-formed IgE on the surface of mast cells or basophils. By doing so, the antibodies disclosed herein may prevent the release of histamine or other inflammatory mediators from mast cells or basophils, thereby preventing or diminishing the untoward effects observed in patients sensitized to a peanut allergen such as Ara h 2.

Peanut allergy occurs when an individual's immune system mistakenly identifies peanut proteins such as Ara h 2 as something harmful. Direct or indirect contact with peanuts causes the individual's immune system to release symptom-causing chemicals such as histamines into their bloodstream. Exposure to peanuts can trigger an allergic reaction and can occur in various ways including, without limitation, direct contact (e.g., eating peanuts or peanut-containing foods or through direct skin contact with peanuts); cross-contact (e.g., an unintended introduction of peanuts into a product) or inhalation (e.g., an allergic reaction may occur if an individual inhales dust or aerosols containing peanuts, from a source such as peanut flour or peanut oil cooking spray).

In certain applications, the antibodies described herein may reduce, minimize, or prevent at least one symptom in a patient sensitive to the Ara h 2 peanut allergen, such as skin reactions such as hives, redness or swelling, itching or tingling in or around the mouth or throat, digestive problems such as diarrhea, stomach cramps, nausea, or vomiting, tightening of the throat, sneezing, congestion, nasal blockage, coughing, wheezing, bronchoconstriction, rhinitis, or shortness of breath. In other applications, the antibodies may be capable of preventing even more serious in vivo complications associated with exposure to the peanut allergen in sensitized individuals, such as asthmatic responses, swelling of the throat that makes it difficult to breath, a severe drop in blood pressure (shock), rapid pulse, dizziness, lightheadedness or loss of consciousness, anaphylaxis, or even death. Children and adults who have a severe peanut allergy are especially at risk of having this life-threatening reaction.

The antibody compositions and methods disclosed herein address an unmet need in the art.

Expert guidelines for treatment of peanut allergy recommend that, for example, patients are counseled to avoid the allergen and to use injectable epinephrine for treatment of anaphylaxis should exposure occur. However, up to 25% of pediatric visits to an emergency department are for treatment of IgE-mediated hypersensitivity, and there is growing recognition of the burden of the costs of this strategy, both in direct medical costs as well as indirect costs. The financial, social, and psychological burden of food allergy is considerable, and widely evident in our daily lives.

Passive protection using peanut-specific protective antibodies disclosed herein provide an extra margin of safety for patients with IgE-mediated food allergies, for example, by preventing allergic reactions to accidental ingestion or exposure. One of the most common reasons that patients and their families seek further therapy for food allergy is for the prevention of IgE-mediated food allergic reactions. Passive protection allows clinicians to provide a therapy with low risk for allergic reactions and which can provide long-lasting protection against reactions to accidental exposures.

Furthermore, patients who are not eligible for other treatments such as OIT, due to underlying eosinophilic esophagitis or extreme sensitivity to the allergen, would still be able to tolerate a strategy of passive protection. In clinical trials, the majority of patients experience side effects and even those who undergo OIT can still have episodes of treatment-related anaphylaxis. Passive protection offers the possibility of treatment without the side effects of allergen exposure. Lastly, passive immunization with peanut-specific antibodies may be an effective adjunctive therapy to immunotherapy as a strategy to decrease the high rate of adverse events known to occur with immunotherapy.

The development of peanut-specific antibodies that effectively neutralize the allergen further provide a useful diagnostic tool for monitoring clinical tolerance development during allergen-specific immunotherapy. In most forms of immunotherapy, both oral or sublingual, patients are subjected to repeated oral challenges to evaluate the development of tolerance. Suppression of effector cells and basophils has been shown to be effective biomarkers of tolerance in OIT, and that this suppression is mediated by antibodies in the serum.

Furthermore, a competitive assay using the antibodies described herein would be highly effective for monitoring tolerance during immunotherapy. In this assay, a comparison of individuals' OIT-induced allergen-specific antibodies with the pre-defined protective antibodies is utilized to determine whether their post-OIT antibody repertoire will effectively protect them against peanut exposures, without the need for repeated financially and medically intensive oral food challenges. As OIT is increasingly adopted outside of clinical trials, a robust methodology of monitoring tolerance is needed. The diagnostic test disclosed herein that relies on patient serum is superior as opposed to the methodology employed in previous clinical trials, which relies of a technically challenging and biologically variable assay that activates short-lived effector cells.

In one aspect, the invention features a combination of anti-Ara h 2 antibodies for use in determining a treatment response of an individual with a peanut allergy to peanut exposure, wherein the combination comprises one or more anti-Ara h 2 antibodies that bind the following Ara h 2 epitope bin: (a) a first epitope bin comprising epitopes of one or more of anti-Ara h 2 antibodies P34, T1, T3, P33, P17, P21, P3, P13, P10, P11, P14, P19, P28, P30, P39, U1, 13FU1P1A4, 13FU1P1B4, 14FU2P1A11, 15FU1P1A3, 13FU1P2B12, 11FUP1A2, 18FU1P1A7, 23FUP1A8, 23FUP1B8, 23FUP1C4, 23FUP1D6, 23FUP1 D12, 24BU7P1A10, 24BU7P1B6, 24BU7P1 D1, 24BU7P1C10, 24BU7P1D9, 24BU7P1C2, 105BU7P1A11, 105BU7P1C3, 105BU7P1D7, 105BU7P1D12, and 33BU7P1 D11, wherein P34 comprises the following complementarity determining regions (CDRs): a CDR-H1 comprising the amino acid sequence of GFTFDDYT (SEQ ID NO:341), a CDR-H2 comprising the amino acid sequence of IRWDGSRT (SEQ ID NO:342), a CDR-H3 comprising the amino acid sequence of VKDGGLRYFDS (SEQ ID NO:343), a CDR-L1 comprising the amino acid sequence of QSLLHSNGIHY (SEQ ID NO:344), a CDR-L2 comprising the amino acid sequence of LGS (SEQ ID NO:345), and a CDR-L3 comprising the amino acid sequence of MQSLQTFT (SEQ ID NO:346).

In another aspect, the invention features a combination of anti-Ara h 2 antibodies for use in determining a treatment response of an individual with a peanut allergy to peanut exposure, wherein the combination comprises two or more anti-Ara h 2 antibodies that bind the following Ara h 2 epitope bins: (a) a first epitope bin comprising epitopes of one or more of anti-Ara h 2 antibodies P34, T1, T3, P33, P17, P21, P3, P13, P10, P11, P14, P19, P28, P30, P39, U1, 13FU1P1A4, 13FU1P1B4, 14FU2P1A11, 15FU1P1A3, 13FU1P2B12, 11FUP1A2, 18FU1P1A7, 23FUP1A8, 23FUP1B8, 23FUP1C4, 23FUP1D6, 23FUP1 D12, 24BU7P1A10, 24BU7P1B6, 24BU7P1 D1, 24BU7P1C10, 24BU7P1D9, 24BU7P1C2, 105BU7P1A11, 105BU7P1C3, 105BU7P1D7, 105BU7P1D12, and 33BU7P1 D11, wherein P34 comprises the following complementarity determining regions (CDRs): a CDR-H1 comprising the amino acid sequence of GFTFDDYT (SEQ ID NO:341), a CDR-H2 comprising the amino acid sequence of IRWDGSRT (SEQ ID NO:342), a CDR-H3 comprising the amino acid sequence of VKDGGLRYFDS (SEQ ID NO:343), a CDR-L1 comprising the amino acid sequence of QSLLHSNGIHY (SEQ ID NO:344), a CDR-L2 comprising the amino acid sequence of LGS (SEQ ID NO:345), and a CDR-L3 comprising the amino acid sequence of MQSLQTFT (SEQ ID NO:346); and (b) a second epitope bin comprising epitopes of one or more of anti-Ara h 2 antibodies P31, T4, T5, S4, 14FU2P1 D6, 15FU1P3A6, 13FU1P2B10, and 27FU1P3A10, wherein P31 comprises the following CDRs: a CDR-H1 comprising the amino acid sequence of GDPFTSYY (SEQ ID NO:301), a CDR-H2 comprising the amino acid sequence of IFTTGST (SEQ ID NO:302), a CDR-H3 comprising the amino acid sequence of ARVRRYCSGGRCYPYFYMDV (SEQ ID NO:303), a CDR-L1 comprising the amino acid sequence of ESISSW (SEQ ID NO:304), a CDR-L2 comprising the amino acid sequence of EAS (SEQ ID NO:305), and a CDR-L3 comprising the amino acid sequence of QHYNSDSLT (SEQ ID NO:306).

In another aspect, the invention features a combination of anti-Ara h 2 antibodies for use in determining a treatment response of an individual with a peanut allergy to peanut exposure, wherein the combination comprises two or more anti-Ara h 2 antibodies that bind the following Ara h 2 epitope bins: (a) a first epitope bin comprising epitopes of one or more of anti-Ara h 2 antibodies P34, T1, T3, P33, P17, P21, P3, P13, P10, P11, P14, P19, P28, P30, P39, U1, 13FU1P1A4, 13FU1P1B4, 14FU2P1A11, 15FU1P1A3, 13FU1P2B12, 11FUP1A2, 18FU1P1A7, 23FUP1A8, 23FUP1B8, 23FUP1C4, 23FUP1D6, 23FUP1D12, 24BU7P1A10, 24BU7P1B6, 24BU7P1D1, 24BU7P1C10, 24BU7P1D9, 24BU7P1C2, 105BU7P1A11, 105BU7P1C3, 105BU7P1D7, 105BU7P1D12, and 33BU7P1D11, wherein P34 comprises the following complementarity determining regions (CDRs): a CDR-H1 comprising the amino acid sequence of GFTFDDYT (SEQ ID NO:341), a CDR-H2 comprising the amino acid sequence of IRWDGSRT (SEQ ID NO:342), a CDR-H3 comprising the amino acid sequence of VKDGGLRYFDS (SEQ ID NO:343), a CDR-L1 comprising the amino acid sequence of QSLLHSNGIHY (SEQ ID NO:344), a CDR-L2 comprising the amino acid sequence of LGS (SEQ ID NO:345), and a CDR-L3 comprising the amino acid sequence of MQSLQTFT (SEQ ID NO:346); and (c) a third epitope bin comprising an epitope of anti-Ara h 2 antibody S1, 27FU1P3A4, 6BU4P2B1, and 89BU7P1B10, wherein S1 comprises the following CDRs: a CDR-H1 comprising the amino acid sequence of GFSFSDSY (SEQ ID NO:381), a CDR-H2 comprising the amino acid sequence of ISGSGEII (SEQ ID NO:382), a CDR-H3 comprising the amino acid sequence of ARPSDYFETSEELD (SEQ ID NO:383), a CDR-L1 comprising the amino acid sequence of QSISTY (SEQ ID NO:384), a CDR-L2 comprising the amino acid sequence of AAS (SEQ ID NO:385), and a CDR-L3 comprising the amino acid sequence of HQSYSAPRT (SEQ ID NO:386).

In another aspect, the invention features a combination of anti-Ara h 2 antibodies for use in determining a treatment response of an individual with a peanut allergy to peanut exposure, wherein the combination comprises two or more anti-Ara h 2 antibodies that bind the following Ara h 2 epitope bins: (b) a second epitope bin comprising epitopes of one or more of anti-Ara h 2 antibodies P31, T4, T5, S4, 14FU2P1D6, 15FU1P3A6, 13FU1P2B10, and 27FU1P3A10, wherein P31 comprises the following CDRs: a CDR-H1 comprising the amino acid sequence of GDPFTSYY (SEQ ID NO:301), a CDR-H2 comprising the amino acid sequence of IFTTGST (SEQ ID NO:302), a CDR-H3 comprising the amino acid sequence of ARVRRYCSGGRCYPYFYMDV (SEQ ID NO:303), a CDR-L1 comprising the amino acid sequence of ESISSW (SEQ ID NO:304), a CDR-L2 comprising the amino acid sequence of EAS (SEQ ID NO:305), and a CDR-L3 comprising the amino acid sequence of QHYNSDSLT (SEQ ID NO:306); and (c) a third epitope bin comprising an epitope of anti-Ara h 2 antibody S1, 27FU1P3A4, 6BU4P2B1, and 89BU7P1B10, wherein S1 comprises the following CDRs: a CDR-H1 comprising the amino acid sequence of GFSFSDSY (SEQ ID NO:381), a CDR-H2 comprising the amino acid sequence of ISGSGEII (SEQ ID NO:382), a CDR-H3 comprising the amino acid sequence of ARPSDYFETSEELD (SEQ ID NO:383), a CDR-L1 comprising the amino acid sequence of QSISTY (SEQ ID NO:384), a CDR-L2 comprising the amino acid sequence of AAS (SEQ ID NO:385), and a CDR-L3 comprising the amino acid sequence of HQSYSAPRT (SEQ ID NO:386).

In another aspect, the invention features a combination of anti-Ara h 2 antibodies for use in determining a treatment response of an individual with a peanut allergy to peanut exposure, wherein the combination comprises three or more anti-Ara h 2 antibodies that bind the following Ara h 2 epitope bins: (a) a first epitope bin comprising epitopes of one or more of anti-Ara h 2 antibodies P34, T1, T3, P33, P17, P21, P3, P13, P10, P11, P14, P19, P28, P30, P39, U1, 13FU1P1A4, 13FU1P1B4, 14FU2P1A11, 15FU1P1A3, 13FU1P2B12, 11FUP1A2, 18FU1P1A7, 23FUP1A8, 23FUP1B8, 23FUP1C4, 23FUP1D6, 23FUP1D12, 24BU7P1A10, 24BU7P1B6, 24BU7P1D1, 24BU7P1C10, 24BU7P1D9, 24BU7P1C2, 105BU7P1A11, 105BU7P1C3, 105BU7P1D7, 105BU7P1D12, and 33BU7P1D11, wherein P34 comprises the following complementarity determining regions (CDRs): a CDR-H1 comprising the amino acid sequence of GFTFDDYT (SEQ ID NO:341), a CDR-H2 comprising the amino acid sequence of IRWDGSRT (SEQ ID NO:342), a CDR-H3 comprising the amino acid sequence of VKDGGLRYFDS (SEQ ID NO:343), a CDR-L1 comprising the amino acid sequence of QSLLHSNGIHY (SEQ ID NO:344), a CDR-L2 comprising the amino acid sequence of LGS (SEQ ID NO:345), and a CDR-L3 comprising the amino acid sequence of MQSLQTFT (SEQ ID NO:346); (b) a second epitope bin comprising epitopes of one or more of anti-Ara h 2 antibodies P31, T4, T5, S4, 14FU2P1D6, 15FU1P3A6, 13FU1P2B10, and 27FU1P3A10, wherein P31 comprises the following CDRs: a CDR-H1 comprising the amino acid sequence of GDPFTSYY (SEQ ID NO:301), a CDR-H2 comprising the amino acid sequence of IFTTGST (SEQ ID NO:302), a CDR-H3 comprising the amino acid sequence of ARVRRYCSGGRCYPYFYMDV (SEQ ID NO:303), a CDR-L1 comprising the amino acid sequence of ESISSW (SEQ ID NO:304), a CDR-L2 comprising the amino acid sequence of EAS (SEQ ID NO:305), and a CDR-L3 comprising the amino acid sequence of QHYNSDSLT (SEQ ID NO:306); (c) a third epitope bin comprising an epitope of anti-Ara h 2 antibody S1, 27FU1P3A4, 6BU4P2B1, and 89BU7P1B10, wherein S1 comprises the following CDRs: a CDR-H1 comprising the amino acid sequence of GFSFSDSY (SEQ ID NO:381), a CDR-H2 comprising the amino acid sequence of ISGSGEII (SEQ ID NO:382), a CDR-H3 comprising the amino acid sequence of ARPSDYFETSEELD (SEQ ID NO:383), a CDR-L1 comprising the amino acid sequence of QSISTY (SEQ ID NO:384), a CDR-L2 comprising the amino acid sequence of AAS (SEQ ID NO:385), and a CDR-L3 comprising the amino acid sequence of HQSYSAPRT (SEQ ID NO:386); and, optionally, (d) a fourth epitope bin comprising epitopes of one or more of anti-Ara h 2 antibodies P7, P6, 111BU7P1A12, 111BU7P1D2, 111BU7P1D5, 24BU7P1D3, and 24BU7P1B1, wherein P7 comprises the following CDRs: a CDR-H1 comprising the amino acid sequence of GFTFTRYA (SEQ ID NO:41), a CDR-H2 comprising the amino acid sequence of ISHDGGTK (SEQ ID NO:42), a CDR-H3 comprising the amino acid sequence of AKTCSSPSCYDTAYYFDY (SEQ ID NO:43), a CDR-L1 comprising the amino acid sequence of QSLGNY (SEQ ID NO:44), a CDR-L2 comprising the amino acid sequence of DAS (SEQ ID NO:45), and a CDR-L3 comprising the amino acid sequence of QQRSQFMWT (SEQ ID NO:46).

In some embodiments of any of the preceding aspects, the epitopes of the one or more anti-Ara h 2 antibodies of the fourth epitope bin comprise the amino acid sequence of DPYS (SEQ ID NO:1880) or DPYSZS (SEQ ID NO:1881).

In some embodiments of any of the preceding aspects, the combination further comprises one or more anti-Ara h 2 antibodies that bind: (e) a fifth epitope bin comprising epitopes of one or more anti-Ara h 2 antibodies 24B7D4, T6, 15FU1P3A1, 23FUP1C10, 23FUP1D8, and 24BU7P1 D4.

In some embodiments of any of the preceding aspects, the epitope of the one or more anti-Ara h 2 antibodies of the fifth epitope bin comprises the amino acid sequence of QSQLER (SEQ ID NO:1882).

In some embodiments of any of the preceding aspects, the combination further comprises one or more anti-Ara h 2 antibodies that bind: (f) a sixth epitope bin comprising epitopes of one or more of anti-Ara h 2 antibodies P8, P16, and P22.

In some embodiments of any of the preceding aspects, the epitopes of the one or more anti-Ara h 2 antibodies of the sixth epitope bin comprise the amino acid sequence of KRELRNL (SEQ ID NO:1883).

In some embodiments of any of the preceding aspects, the combination further comprises one or more anti-Ara h 2 antibodies that bind: (g) a seventh epitope bin comprising epitopes of one or more anti-Ara h 2 antibodies 105BU7P1D6 and 105BU7P1D8.

In some embodiments of any of the preceding aspects, the epitopes of the one or more anti-Ara h 2 antibodies of the seventh epitope bin comprise the amino acid sequence of RQQEQQ (SEQ ID NO:1885).

In some embodiments of any of the preceding aspects, the combination further comprises one or more anti-Ara h 2 antibodies that bind: (h) an eighth epitope bin comprising an epitope of anti-Ara h 2 antibody 29BU7P1D1.

In some embodiments of any of the preceding aspects, the epitopes of the one or more anti-Ara h 2 antibodies of the seventh epitope bin comprise the amino acid sequence of CEALQQ (SEQ ID NO:1887).

In another aspect, the invention features a combination of anti-Ara h 2 antibodies for use in determining a treatment response of an individual with a peanut allergy to peanut exposure, wherein the combination comprises anti-Ara h 2 antibodies: (a) P34, P33, or P17; (b) P31; (c) S1; and (d) P7.

In some embodiments of any of the preceding aspect, the combination comprises anti-Ara h 2 antibodies: (a) P34; (b) P31; (c) S1; and (d) P7.

In another aspect, the invention features a method for assessing a treatment response of an individual with a peanut allergy to peanut exposure, the method comprising measuring anti-Ara h 2 antibodies in a sample from a subject using a competitive assay comprising one or more anti-Ara h 2 antibodies (e.g., one or more of the anti-Ara h 2 antibodies disclosure herein).

In some embodiments, the competitive assay includes one or more anti-Ara h 2 antibodies from one or more of the epitope bins disclosed herein, e.g., any of the combinations of antibodies disclosed herein. For example, in some embodiments, the competitive assay comprises one or more anti-Ara h 2 antibodies that are included in one or more of the following conformational epitope bins: Bin 1, Bin 2, Bin 3, Bin 1 and Bin 2, Bin 1 and Bin 3, Bin 2 and Bin 3, or Bin 1, Bin 2, and Bin 3. In some embodiments, the competitive assay comprises one or more anti-Ara h 2 antibodies that are included in any of the linear epitope bins described herein.

In some embodiments, the one or more anti-Ara h 2 antibodies comprise a combination of one or more anti-Ara h 2 antibodies that bind the following Ara h 2 epitope bins: (a) a first epitope bin comprising epitopes of one or more of anti-Ara h 2 antibodies P34, T1, T3, P33, P17, P21, P3, P13, P10, P11, P14, P19, P28, P30, P39, U1, 13FU1P1A4, 13FU1P1B4, 14FU2P1A11, 15FU1P1A3, 13FU1P2B12, 11FUP1A2, 18FU1P1A7, 23FUP1A8, 23FUP1B8, 23FUP1C4, 23FUP1D6, 23FUP1D12, 24BU7P1A10, 24BU7P1B6, 24BU7P1D1, 24BU7P1C10, 24BU7P1D9, 24BU7P1C2, 105BU7P1A11, 105BU7P1C3, 105BU7P1D7, 105BU7P1D12, and 33BU7P1D11, wherein P34 comprises the following CDRs: a CDR-H1 comprising the amino acid sequence of GFTFDDYT (SEQ ID NO:341), a CDR-H2 comprising the amino acid sequence of IRWDGSRT (SEQ ID NO:342), a CDR-H3 comprising the amino acid sequence of VKDGGLRYFDS (SEQ ID NO:343), a CDR-L1 comprising the amino acid sequence of QSLLHSNGIHY (SEQ ID NO:344), a CDR-L2 comprising the amino acid sequence of LGS (SEQ ID NO:345), and a CDR-L3 comprising the amino acid sequence of MQSLQTFT (SEQ ID NO:346).

In some embodiments, the one or more anti-Ara h 2 antibodies comprise a combination of two or more anti-Ara h 2 antibodies that bind the following Ara h 2 epitope bins: (a) a first epitope bin comprising epitopes of one or more of anti-Ara h 2 antibodies P34, T1, T3, P33, P17, P21, P3, P13, P10, P11, P14, P19, P28, P30, P39, U1, 13FU1P1A4, 13FU1P1B4, 14FU2P1A11, 15FU1P1A3, 13FU1P2B12, 11FUP1A2, 18FU1P1A7, 23FUP1A8, 23FUP1B8, 23FUP1C4, 23FUP1D6, 23FUP1D12, 24BU7P1A10, 24BU7P1B6, 24BU7P1D1, 24BU7P1C10, 24BU7P1D9, 24BU7P1C2, 105BU7P1A11, 105BU7P1C3, 105BU7P1D7, 105BU7P1D12, and 33BU7P1D11, wherein P34 comprises the following CDRs: a CDR-H1 comprising the amino acid sequence of GFTFDDYT (SEQ ID NO:341), a CDR-H2 comprising the amino acid sequence of IRWDGSRT (SEQ ID NO:342), a CDR-H3 comprising the amino acid sequence of VKDGGLRYFDS (SEQ ID NO:343), a CDR-L1 comprising the amino acid sequence of QSLLHSNGIHY (SEQ ID NO:344), a CDR-L2 comprising the amino acid sequence of LGS (SEQ ID NO:345), and a CDR-L3 comprising the amino acid sequence of MQSLQTFT (SEQ ID NO:346); and (b) a second epitope bin comprising epitopes of one or more of anti-Ara h 2 antibodies P31, T4, T5, S4, 14FU2P1 D6, 15FU1P3A6, 13FU1P2B10, and 27FU1P3A10, wherein P31 comprises the following CDRs: a CDR-H1 comprising the amino acid sequence of GDPFTSYY (SEQ ID NO:301), a CDR-H2 comprising the amino acid sequence of IFTTGST (SEQ ID NO:302), a CDR-H3 comprising the amino acid sequence of ARVRRYCSGGRCYPYFYMDV (SEQ ID NO:303), a CDR-L1 comprising the amino acid sequence of ESISSW (SEQ ID NO:304), a CDR-L2 comprising the amino acid sequence of EAS (SEQ ID NO:305), and a CDR-L3 comprising the amino acid sequence of QHYNSDSLT (SEQ ID NO:306).

In some embodiments, the one or more anti-Ara h 2 antibodies comprise a combination of two or more anti-Ara h 2 antibodies that bind the following Ara h 2 epitope bins: (a) a first epitope bin comprising epitopes of one or more of anti-Ara h 2 antibodies P34, T1, T3, P33, P17, P21, P3, P13, P10, P11, P14, P19, P28, P30, P39, U1, 13FU1P1A4, 13FU1P1B4, 14FU2P1A11, 15FU1P1A3, 13FU1P2B12, 11FUP1A2, 18FU1P1A7, 23FUP1A8, 23FUP1B8, 23FUP1C4, 23FUP1D6, 23FUP1D12, 24BU7P1A10, 24BU7P1B6, 24BU7P1D1, 24BU7P1C10, 24BU7P1D9, 24BU7P1C2, 105BU7P1A11, 105BU7P1C3, 105BU7P1D7, 105BU7P1D12, and 33BU7P1D11, wherein P34 comprises the following CDRs: a CDR-H1 comprising the amino acid sequence of GFTFDDYT (SEQ ID NO:341), a CDR-H2 comprising the amino acid sequence of IRWDGSRT (SEQ ID NO:342), a CDR-H3 comprising the amino acid sequence of VKDGGLRYFDS (SEQ ID NO:343), a CDR-L1 comprising the amino acid sequence of QSLLHSNGIHY (SEQ ID NO:344), a CDR-L2 comprising the amino acid sequence of LGS (SEQ ID NO:345), and a CDR-L3 comprising the amino acid sequence of MQSLQTFT (SEQ ID NO:346); and (c) a third epitope bin comprising an epitope of anti-Ara h 2 antibody S1, 27FU1P3A4, 6BU4P2B1, and 89BU7P1B10, wherein S1 comprises the following CDRs: a CDR-H1 comprising the amino acid sequence of GFSFSDSY (SEQ ID NO:381), a CDR-H2 comprising the amino acid sequence of ISGSGEII (SEQ ID NO:382), a CDR-H3 comprising the amino acid sequence of ARPSDYFETSEELD (SEQ ID NO:383), a CDR-L1 comprising the amino acid sequence of QSISTY (SEQ ID NO:384), a CDR-L2 comprising the amino acid sequence of AAS (SEQ ID NO:385), and a CDR-L3 comprising the amino acid sequence of HQSYSAPRT (SEQ ID NO:386).

In some embodiments, the one or more anti-Ara h 2 antibodies comprise a combination of two or more anti-Ara h 2 antibodies that bind the following Ara h 2 epitope bins: (b) a second epitope bin comprising epitopes of one or more of anti-Ara h 2 antibodies P31, T4, T5, S4, 14FU2P1 D6, 15FU1P3A6, 13FU1P2B10, and 27FU1P3A10, wherein P31 comprises the following CDRs: a CDR-H1 comprising the amino acid sequence of GDPFTSYY (SEQ ID NO:301), a CDR-H2 comprising the amino acid sequence of IFTTGST (SEQ ID NO:302), a CDR-H3 comprising the amino acid sequence of ARVRRYCSGGRCYPYFYMDV (SEQ ID NO:303), a CDR-L1 comprising the amino acid sequence of ESISSW (SEQ ID NO:304), a CDR-L2 comprising the amino acid sequence of EAS (SEQ ID NO:305), and a CDR-L3 comprising the amino acid sequence of QHYNSDSLT (SEQ ID NO:306); and (c) a third epitope bin comprising an epitope of anti-Ara h 2 antibody S1, 27FU1P3A4, 6BU4P2B1, and 89BU7P1B10, wherein S1 comprises the following CDRs: a CDR-H1 comprising the amino acid sequence of GFSFSDSY (SEQ ID NO:381), a CDR-H2 comprising the amino acid sequence of ISGSGEII (SEQ ID NO:382), a CDR-H3 comprising the amino acid sequence of ARPSDYFETSEELD (SEQ ID NO:383), a CDR-L1 comprising the amino acid sequence of QSISTY (SEQ ID NO:384), a CDR-L2 comprising the amino acid sequence of AAS (SEQ ID NO:385), and a CDR-L3 comprising the amino acid sequence of HQSYSAPRT (SEQ ID NO:386).

In some embodiments, the one or more anti-Ara h 2 antibodies comprise a combination of three or more anti-Ara h 2 antibodies that bind the following Ara h 2 epitope bins: (a) a first epitope bin comprising epitopes of one or more of anti-Ara h 2 antibodies P34, T1, T3, P33, P17, P21, P3, P13, P10, P11, P14, P19, P28, P30, P39, U1, 13FU1P1A4, 13FU1P1B4, 14FU2P1A11, 15FU1P1A3, 13FU1P2B12, 11FUP1A2, 18FU1P1A7, 23FUP1A8, 23FUP1B8, 23FUP1C4, 23FUP1D6, 23FUP1D12, 24BU7P1A10, 24BU7P1B6, 24BU7P1D1, 24BU7P1C10, 24BU7P1D9, 24BU7P1C2, 105BU7P1A11, 105BU7P1C3, 105BU7P1 D7, 105BU7P1 D12, and 33BU7P1D11, wherein P34 comprises the following CDRs: a CDR-H1 comprising the amino acid sequence of GFTFDDYT (SEQ ID NO:341), a CDR-H2 comprising the amino acid sequence of IRWDGSRT (SEQ ID NO:342), a CDR-H3 comprising the amino acid sequence of VKDGGLRYFDS (SEQ ID NO:343), a CDR-L1 comprising the amino acid sequence of QSLLHSNGIHY (SEQ ID NO:344), a CDR-L2 comprising the amino acid sequence of LGS (SEQ ID NO:345), and a CDR-L3 comprising the amino acid sequence of MQSLQTFT (SEQ ID NO:346); (b) a second epitope bin comprising epitopes of one or more of anti-Ara h 2 antibodies P31, T4, T5, S4, 14FU2P1 D6, 15FU1P3A6, 13FU1P2B10, and 27FU1P3A10, wherein P31 comprises the following CDRs: a CDR-H1 comprising the amino acid sequence of GDPFTSYY (SEQ ID NO:301), a CDR-H2 comprising the amino acid sequence of IFTTGST (SEQ ID NO:302), a CDR-H3 comprising the amino acid sequence of ARVRRYCSGGRCYPYFYMDV (SEQ ID NO:303), a CDR-L1 comprising the amino acid sequence of ESISSW (SEQ ID NO:304), a CDR-L2 comprising the amino acid sequence of EAS (SEQ ID NO:305), and a CDR-L3 comprising the amino acid sequence of QHYNSDSLT (SEQ ID NO:306); (c) a third epitope bin comprising an epitope of anti-Ara h 2 antibody S1, 27FU1P3A4, 6BU4P2B1, and 89BU7P1B10, wherein S1 comprises the following CDRs: a CDR-H1 comprising the amino acid sequence of GFSFSDSY (SEQ ID NO:381), a CDR-H2 comprising the amino acid sequence of ISGSGEII (SEQ ID NO:382), a CDR-H3 comprising the amino acid sequence of ARPSDYFETSEELD (SEQ ID NO:383), a CDR-L1 comprising the amino acid sequence of QSISTY (SEQ ID NO:384), a CDR-L2 comprising the amino acid sequence of AAS (SEQ ID NO:385), and a CDR-L3 comprising the amino acid sequence of HQSYSAPRT (SEQ ID NO:386); and, optionally, (d) a fourth epitope bin comprising epitopes of one or more of anti-Ara h 2 antibodies P7, P6, 111BU7P1A12, 111BU7P1D2, 111BU7P1D5, 24BU7P1D3, and 24BU7P1B1.

In some embodiments, the epitopes of the one or more anti-Ara h 2 antibodies of the fourth epitope bin comprise the amino acid sequence of DPYS (SEQ ID NO:1880) or DPYSZS (SEQ ID NO:1881).

In some embodiments, the combination further comprises one or more anti-Ara h 2 antibodies that bind: (e) a fifth epitope bin comprising epitopes of one or more anti-Ara h 2 antibodies 24B7D4, T6, 15FU1P3A1, 23FUP1C10, 23FUP1D8, and 24BU7P1D4.

In some embodiments, the epitope of the one or more anti-Ara h 2 antibodies of the fifth epitope bin comprises the amino acid sequence of QSQLER (SEQ ID NO:1882).

In some embodiments, the combination further comprises one or more anti-Ara h 2 antibodies that bind: (f) a sixth epitope bin comprising epitopes of one or more of anti-Ara h 2 antibodies P8, P16, and P22.

In some embodiments, the epitopes of the one or more anti-Ara h 2 antibodies of the sixth epitope bin comprise the amino acid sequence of KRELRNL (SEQ ID NO:1883).

In some embodiments, the combination further comprises one or more anti-Ara h 2 antibodies that bind: (g) a seventh epitope bin comprising epitopes of one or more anti-Ara h 2 antibodies 105BU7P1D6, and 105BU7P1D8.

In some embodiments, the epitopes of the one or more anti-Ara h 2 antibodies of the seventh epitope bin comprise the amino acid sequence of RQQEQQ (SEQ ID NO:1885).

In some embodiments, the combination further comprises one or more anti-Ara h 2 antibodies that bind: (h) an eighth epitope bin comprising an epitope of anti-Ara h 2 antibody 29BU7P1D1.

In some embodiments, the epitopes of the one or more anti-Ara h 2 antibodies of the seventh epitope bin comprise the amino acid sequence of CEALQQ (SEQ ID NO:1887).

In some embodiments, the combination comprises anti-Ara h 2 antibodies: (a) P34, P33, or P17; (b) P31; (c) S1; and (d) P7.

In some embodiments, the combination comprises anti-Ara h 2 antibodies: (a) P34; (b) P31; (c) S1; and (d) P7.

In some embodiments, the competitive assay comprises bio-layer interferometry (BLI).

In some embodiments, the sample is a plasma sample.

In another aspect, the invention features a kit comprising one or more anti-Ara h 2 antibodies and instructions for use in determining the presence or level of Ara h 2 antibodies in a sample.

In some embodiments, the kit includes one or more anti-Ara h 2 antibodies from one or more of the epitope bins disclosed herein, e.g., any of the combinations of antibodies disclosed herein. For example, in some embodiments, the competitive assay comprises one or more anti-Ara h 2 antibodies that are included in one or more of the following conformational epitope bins: Bin 1, Bin 2, Bin 3, Bin 1 and Bin 2, Bin 1 and Bin 3, Bin 2 and Bin 3, or Bin 1, Bin 2, and Bin 3. In some embodiments, the competitive assay comprises one or more anti-Ara h 2 antibodies that are included in any of the linear epitope bins described herein.

In some embodiments, the one or more anti-Ara h 2 antibodies comprise a combination of one or more anti-Ara h 2 antibodies that bind the following Ara h 2 epitope bin: (a) a first epitope bin comprising epitopes of one or more of anti-Ara h 2 antibodies P34, T1, T3, P33, P17, P21, P3, P13, P10, P11, P14, P19, P28, P30, P39, U1, 13FU1P1A4, 13FU1P1B4, 14FU2P1A11, 15FU1P1A3, 13FU1P2B12, 11FUP1A2, 18FU1P1A7, 23FUP1A8, 23FUP1B8, 23FUP1C4, 23FUP1D6, 23FUP1D12, 24BU7P1A10, 24BU7P1B6, 24BU7P1D1, 24BU7P1C10, 24BU7P1D9, 24BU7P1C2, 105BU7P1A11, 105BU7P1C3, 105BU7P1D7, 105BU7P1D12, and 33BU7P1D11, wherein P34 comprises the following CDRs: a CDR-H1 comprising the amino acid sequence of GFTFDDYT (SEQ ID NO:341), a CDR-H2 comprising the amino acid sequence of IRWDGSRT (SEQ ID NO:342), a CDR-H3 comprising the amino acid sequence of VKDGGLRYFDS (SEQ ID NO:343), a CDR-L1 comprising the amino acid sequence of QSLLHSNGIHY (SEQ ID NO:344), a CDR-L2 comprising the amino acid sequence of LGS (SEQ ID NO:345), and a CDR-L3 comprising the amino acid sequence of MQSLQTFT (SEQ ID NO:346).

In some embodiments, the one or more anti-Ara h 2 antibodies comprise a combination of two or more anti-Ara h 2 antibodies that bind the following Ara h 2 epitope bins: (a) a first epitope bin comprising epitopes of one or more of anti-Ara h 2 antibodies P34, T1, T3, P33, P17, P21, P3, P13, P10, P11, P14, P19, P28, P30, P39, U1, 13FU1P1A4, 13FU1P1B4, 14FU2P1A11, 15FU1P1A3, 13FU1P2B12, 11FUP1A2, 18FU1P1A7, 23FUP1A8, 23FUP1B8, 23FUP1C4, 23FUP1D6, 23FUP1D12, 24BU7P1A10, 24BU7P1B6, 24BU7P1D1, 24BU7P1C10, 24BU7P1D9, 24BU7P1C2, 105BU7P1A11, 105BU7P1C3, 105BU7P1D7, 105BU7P1D12, and 33BU7P1D11, wherein P34 comprises the following CDRs: a CDR-H1 comprising the amino acid sequence of GFTFDDYT (SEQ ID NO:341), a CDR-H2 comprising the amino acid sequence of IRWDGSRT (SEQ ID NO:342), a CDR-H3 comprising the amino acid sequence of VKDGGLRYFDS (SEQ ID NO:343), a CDR-L1 comprising the amino acid sequence of QSLLHSNGIHY (SEQ ID NO:344), a CDR-L2 comprising the amino acid sequence of LGS (SEQ ID NO:345), and a CDR-L3 comprising the amino acid sequence of MQSLQTFT (SEQ ID NO:346); and (b) a second epitope bin comprising epitopes of one or more of anti-Ara h 2 antibodies P31, T4, T5, S4, 14FU2P1 D6, 15FU1P3A6, 13FU1P2B10, and 27FU1P3A10, wherein P31 comprises the following CDRs: a CDR-H1 comprising the amino acid sequence of GDPFTSYY (SEQ ID NO:301), a CDR-H2 comprising the amino acid sequence of IFTTGST (SEQ ID NO:302), a CDR-H3 comprising the amino acid sequence of ARVRRYCSGGRCYPYFYMDV (SEQ ID NO:303), a CDR-L1 comprising the amino acid sequence of ESISSW (SEQ ID NO:304), a CDR-L2 comprising the amino acid sequence of EAS (SEQ ID NO:305), and a CDR-L3 comprising the amino acid sequence of QHYNSDSLT (SEQ ID NO:306).

In some embodiments, the one or more anti-Ara h 2 antibodies comprise a combination of two or more anti-Ara h 2 antibodies that bind the following Ara h 2 epitope bins: (a) a first epitope bin comprising epitopes of one or more of anti-Ara h 2 antibodies P34, T1, T3, P33, P17, P21, P3, P13, P10, P11, P14, P19, P28, P30, P39, U1, 13FU1P1A4, 13FU1P1B4, 14FU2P1A11, 15FU1P1A3, 13FU1P2B12, 11FUP1A2, 18FU1P1A7, 23FUP1A8, 23FUP1B8, 23FUP1C4, 23FUP1D6, 23FUP1D12, 24BU7P1A10, 24BU7P1B6, 24BU7P1D1, 24BU7P1C10, 24BU7P1D9, 24BU7P1C2, 105BU7P1A11, 105BU7P1C3, 105BU7P1D7, 105BU7P1D12, and 33BU7P1D11, wherein P34 comprises the following CDRs: a CDR-H1 comprising the amino acid sequence of GFTFDDYT (SEQ ID NO:341), a CDR-H2 comprising the amino acid sequence of IRWDGSRT (SEQ ID NO:342), a CDR-H3 comprising the amino acid sequence of VKDGGLRYFDS (SEQ ID NO:343), a CDR-L1 comprising the amino acid sequence of QSLLHSNGIHY (SEQ ID NO:344), a CDR-L2 comprising the amino acid sequence of LGS (SEQ ID NO:345), and a CDR-L3 comprising the amino acid sequence of MQSLQTFT (SEQ ID NO:346); and (c) a third epitope bin comprising an epitope of anti-Ara h 2 antibody S1, 27FU1P3A4, 6BU4P2B1, and 89BU7P1B10, wherein S1 comprises the following CDRs: a CDR-H1 comprising the amino acid sequence of GFSFSDSY (SEQ ID NO:381), a CDR-H2 comprising the amino acid sequence of ISGSGEII (SEQ ID NO:382), a CDR-H3 comprising the amino acid sequence of ARPSDYFETSEELD (SEQ ID NO:383), a CDR-L1 comprising the amino acid sequence of QSISTY (SEQ ID NO:384), a CDR-L2 comprising the amino acid sequence of AAS (SEQ ID NO:385), and a CDR-L3 comprising the amino acid sequence of HQSYSAPRT (SEQ ID NO:386).

In some embodiments, the one or more anti-Ara h 2 antibodies comprise a combination of two or more anti-Ara h 2 antibodies that bind the following Ara h 2 epitope bins: (b) a second epitope bin comprising epitopes of one or more of anti-Ara h 2 antibodies P31, T4, T5, S4, 14FU2P1 D6, 15FU1P3A6, 13FU1P2B10, and 27FU1P3A10, wherein P31 comprises the following CDRs: a CDR-H1 comprising the amino acid sequence of GDPFTSYY (SEQ ID NO:301), a CDR-H2 comprising the amino acid sequence of IFTTGST (SEQ ID NO:302), a CDR-H3 comprising the amino acid sequence of ARVRRYCSGGRCYPYFYMDV (SEQ ID NO:303), a CDR-L1 comprising the amino acid sequence of ESISSW (SEQ ID NO:304), a CDR-L2 comprising the amino acid sequence of EAS (SEQ ID NO:305), and a CDR-L3 comprising the amino acid sequence of QHYNSDSLT (SEQ ID NO:306); and (c) a third epitope bin comprising an epitope of anti-Ara h 2 antibody S1, 27FU1P3A4, 6BU4P2B1, and 89BU7P1B10, wherein S1 comprises the following CDRs: a CDR-H1 comprising the amino acid sequence of GFSFSDSY (SEQ ID NO:381), a CDR-H2 comprising the amino acid sequence of ISGSGEII (SEQ ID NO:382), a CDR-H3 comprising the amino acid sequence of ARPSDYFETSEELD (SEQ ID NO:383), a CDR-L1 comprising the amino acid sequence of QSISTY (SEQ ID NO:384), a CDR-L2 comprising the amino acid sequence of AAS (SEQ ID NO:385), and a CDR-L3 comprising the amino acid sequence of HQSYSAPRT (SEQ ID NO:386).

In some embodiments, the one or more anti-Ara h 2 antibodies comprise a combination of three or more anti-Ara h 2 antibodies that bind the following Ara h 2 epitope bins: (a) a first epitope bin comprising epitopes of one or more of anti-Ara h 2 antibodies P34, T1, T3, P33, P17, P21, P3, P13, P10, P11, P14, P19, P28, P30, P39, U1, 13FU1P1A4, 13FU1P1B4, 14FU2P1A11, 15FU1P1A3, 13FU1P2B12, 11FUP1A2, 18FU1P1A7, 23FUP1A8, 23FUP1B8, 23FUP1C4, 23FUP1D6, 23FUP1D12, 24BU7P1A10, 24BU7P1B6, 24BU7P1D1, 24BU7P1C10, 24BU7P1D9, 24BU7P1C2, 105BU7P1A11, 105BU7P1C3, 105BU7P1D7, 105BU7P1D12, and 33BU7P1D11, wherein P34 comprises the following CDRs: a CDR-H1 comprising the amino acid sequence of GFTFDDYT (SEQ ID NO:341), a CDR-H2 comprising the amino acid sequence of IRWDGSRT (SEQ ID NO:342), a CDR-H3 comprising the amino acid sequence of VKDGGLRYFDS (SEQ ID NO:343), a CDR-L1 comprising the amino acid sequence of QSLLHSNGIHY (SEQ ID NO:344), a CDR-L2 comprising the amino acid sequence of LGS (SEQ ID NO:345), and a CDR-L3 comprising the amino acid sequence of MQSLQTFT (SEQ ID NO:346); (b) a second epitope bin comprising epitopes of one or more of anti-Ara h 2 antibodies P31, T4, T5, S4, 14FU2P1 D6, 15FU1P3A6, 13FU1P2B10, and 27FU1P3A10, wherein P31 comprises the following CDRs: a CDR-H1 comprising the amino acid sequence of GDPFTSYY (SEQ ID NO:301), a CDR-H2 comprising the amino acid sequence of IFTTGST (SEQ ID NO:302), a CDR-H3 comprising the amino acid sequence of ARVRRYCSGGRCYPYFYMDV (SEQ ID NO:303), a CDR-L1 comprising the amino acid sequence of ESISSW (SEQ ID NO:304), a CDR-L2 comprising the amino acid sequence of EAS (SEQ ID NO:305), and a CDR-L3 comprising the amino acid sequence of QHYNSDSLT (SEQ ID NO:306); (c) a third epitope bin comprising an epitope of anti-Ara h 2 antibody S1, 27FU1P3A4, 6BU4P2B1, and 89BU7P1B10, wherein S1 comprises the following CDRs: a CDR-H1 comprising the amino acid sequence of GFSFSDSY (SEQ ID NO:381), a CDR-H2 comprising the amino acid sequence of ISGSGEII (SEQ ID NO:382), a CDR-H3 comprising the amino acid sequence of ARPSDYFETSEELD (SEQ ID NO:383), a CDR-L1 comprising the amino acid sequence of QSISTY (SEQ ID NO:384), a CDR-L2 comprising the amino acid sequence of AAS (SEQ ID NO:385), and a CDR-L3 comprising the amino acid sequence of HQSYSAPRT (SEQ ID NO:386); and (d) a fourth epitope bin comprising epitopes of one or more of anti-Ara h 2 antibodies P7, P6, 111BU7P1A12, 111BU7P1D2, 111BU7P1D5, 24BU7P1D3, and 24BU7P1B1, wherein P7 comprises the following CDRs: a CDR-H1 comprising the amino acid sequence of GFTFTRYA (SEQ ID NO:41), a CDR-H2 comprising the amino acid sequence of ISHDGGTK (SEQ ID NO:42), a CDR-H3 comprising the amino acid sequence of AKTCSSPSCYDTAYYFDY (SEQ ID NO:43), a CDR-L1 comprising the amino acid sequence of QSLGNY (SEQ ID NO:44), a CDR-L2 comprising the amino acid sequence of DAS (SEQ ID NO:45), and a CDR-L3 comprising the amino acid sequence of QQRSQFMWT (SEQ ID NO:46).

In some embodiments, the epitopes of the one or more anti-Ara h 2 antibodies of the fourth epitope bin comprise the amino acid sequence of DPYS (SEQ ID NO:1880) or DPYSZS (SEQ ID NO:1881).

In some embodiments, the combination further comprises one or more anti-Ara h 2 antibodies that bind: (e) a fifth epitope bin comprising epitopes of one or more anti-Ara h 2 antibodies 24B7D4, T6, 15FU1P3A1, 23FUP1C10, 23FUP1D8, and 24BU7P1D4.

In some embodiments, the epitope of the one or more anti-Ara h 2 antibodies of the fifth epitope bin comprises the amino acid sequence of QSQLER (SEQ ID NO:1882).

In some embodiments, the combination further comprises one or more anti Ara h 2 antibodies that bind: (f) a sixth epitope bin comprising epitopes of one or more of anti-Ara h 2 antibodies P8, P16, and P22.

In some embodiments, the epitopes of the one or more anti-Ara h 2 antibodies of the sixth epitope bin comprise the amino acid sequence of KRELRNL (SEQ ID NO:1883).

In some embodiments, the combination further comprises one or more anti-Ara h 2 antibodies that bind: (g) a seventh epitope bin comprising epitopes of one or more anti-Ara h 2 antibodies 105BU7P1D6, and 105BU7P1D8.

In some embodiments, the epitopes of the one or more anti-Ara h 2 antibodies of the seventh epitope bin comprise the amino acid sequence of RQQEQQ (SEQ ID NO:1885).

In some embodiments, the combination further comprises one or more anti-Ara h 2 antibodies that bind: (h) an eighth epitope bin comprising an epitope of anti-Ara h 2 antibody 29BU7P1D1.

In some embodiments, the epitopes of the one or more anti-Ara h 2 antibodies of the seventh epitope bin comprise the amino acid sequence of CEALQQ (SEQ ID NO:1887).

In some embodiments, the combination comprises anti-Ara h 2 antibodies: (a) P34, P33, or P17; (b) P31; (c) S1; and (d) P7.

In some embodiments, the combination comprises anti-Ara h 2 antibodies: (a) P34; (b) P31; (c) S1; and (d) P7.

Other features and advantages of the invention will be apparent from the following Detailed Description and the Claims.

### BRIEF DESCRIPTION OF THE DRAWINGS

The application file contains at least one drawing executed in color. Copies of this patent or patent application with color drawings will be provided by the Office upon request and payment of the necessary fee.

FIG. 1 is a graph showing results of an exemplary assay of the present disclosure.

FIGS. 2A and 2B are a series of graphs showing that two antibodies from a non-tolerant subject bind the same epitope.

FIG. 3 is a graph showing results from an experiment performing linear epitope mapping of the DPYSZS epitope.

FIG. 4 is a schematic diagram of the commercial peptide microarray assay used for mapping of linear epitopes of anti-Ara h 2 antibodies. Each spot in the microarray represents a single individual peptide. After incubation of the peptide microarray with serum or antibody samples, bound antibodies or proteins can be detected using fluorescently labeled secondary antibodies.

FIG. 5 is a fluorescence readout image of a mini-array incubated with the antibody sample 111BU7P1D5 (here: sample dilution 1:1000). Colors: black—no signal, shades of red—increasing intensity of detected signal, and white—detector saturation. Individual subarrays are framed green. Human IgG control spots are located beneath the subarrays.

FIG. 6 is a heatmap showing results from the peptide microarray assay showing incubations of the antibody samples, controls and all probed peptides; the y-axis represents peptide sequences in the library, and x-axis specifies samples applied. The MMC2 values are shown as color coded ranging from white (0 or low intensity) over yellow (middle intensity) to red (high intensity).

FIGS. 7A and 7B show sequences of peptides bound by antibodies as described in Example 6.

FIGS. 8A-8C show sequences of peptides bound by antibodies as described in Example 6.

FIG. 9A shows a representative grid of conformational Ara h 2 epitopes as determined by biolayer interferometry (BLI). Each box in the grid represents one experiment in which a primary Ara h 2 specific monoclonal antibody (left) is introduced to a sensor covered with biotinylated Ara h 2 followed by a secondary Ara h 2 specific monoclonal antibody (top).

FIG. 9B shows an inset of experimental results from FIG. 9A showing an increase in nanomolar (nm) change of secondary antibodies in different bins and no significant change in antibodies within the same bin.

FIG. 9C shows a map of cloned monoclonal antibodies (sustained unresponsiveness (SU) antibodies=36; transient desensitization (TD) antibodies=44) from both SU and TD patients (n=9 and n=10 respectively).

FIG. 9D shows BLI results showing simultaneous binding of three monoclonal antibodies, one from each conformational bin and the linear DPYSP^{OH}S epitope.

### DETAILED DESCRIPTION OF THE INVENTION

Peanut-specific IgG blocking antibodies as well as combinations of such antibodies are disclosed to prevent IgE-mediated allergic reactions in affected patients. Administered subcutaneously, these antibodies provide protection to peanut allergic patients (or patients having peanut hypersensitivity) as a preventive measure. While this therapy would not induce long-term tolerance, as OIT does, it would prevent allergic reactions, which carry mortality and morbidity, without the risks inherent to OIT. Moreover, the approach of using passive protection is particularly important in our most vulnerable population, young children, for whom food allergies can carry additional social and psychological burdens.

The use of peanut-specific protective antibodies would provide passive protection to pediatric or adult patients with IgE-mediated peanut hypersensitivity, diagnosed by a combination of skin prick testing, specific IgE measurement, component IgE measurement, or oral food challenge. The medication may be administered as a subcutaneous injection, able to be administered at home or in an outpatient office setting by an Allergist. The antibodies described herein may prevent allergic reactions to accidental peanut exposures, direct or indirect, by providing clinical tolerance to an exposure of about 50-300 mg of peanut protein, which is roughly equivalent to 1 peanut. In some instances, the antibodies described herein may provide a clinical tolerance to an exposure to at least about 50-300 mg (e.g., about 50 mg, about 60 mg, about 70 mg, about 80 mg, about 90 mg, about 100 mg, about 120 mg, about 140 mg, about 160 mg, about 180 mg, about 200 mg, about 220 mg, about 240 mg, about 260 mg, about 280 mg, about 300 mg, about 350 mg, about 400 mg, about 450 mg, about 500 mg, about 600 mg, about 700 mg, about 800 mg, about 900 mg, about 1 g, about 3 g, about 5 g, about 10 g, about 30 g, or more) of peanut protein.

The peanut-specific protective antibodies directed towards the immunodominant peanut antigen Ara h 2 as is disclosed herein were cloned from the circulation of pediatric patients who had sustained tolerance to peanut after OIT. From the same peanut OIT study, the suppression of basophil activation as a good biomarker of future tolerance is known. Furthermore, the suppression of basophils is mediated by IgG in the sera of tolerant patients, from whom the Ara h 2-specific antibodies described herein have been cloned. The protective antibodies were identified as those able to outcompete non-tolerant antibodies using biolayer light interferometry, as those have better antigen blocking capabilities.

Below we describe the invention as follows:
I. Definitions for understanding the specification;
II. Compositions and Methods including Therapy and Diagnostics; and
III. Examples.

### I. DEFINITIONS

The term “about” as used herein refers to the usual error range for the respective value readily known to the skilled person in this technical field. Reference to “about” a value or parameter herein includes (and describes) embodiments that are directed to that value or parameter per se.

The term “peanut allergen” refers to Ara h 2, and isoforms thereof or a protein with an amino acid sequence of at least 90%, preferably of at least 92%, further preferably of at least 95%, and again further preferably of at least 98% amino acid sequence identity with such a peanut allergen and isoform thereof.

Preferably, the peanut allergen is Ara h 2 with an amino acid sequence of at least, 90%, 92%, 93%, 94%, 95%, 96%, 97%, 98% or even 99% identity with Ara h 2:

MAKLTILVALALFLLAAHASARQQWELQGDRRCQSQLERANLRPCEQHLMQKIQRDEDSYGRD PYSPSQDPYSPSQDPDRRDPYSPSPYDRRGAGSSQHQERCCNELNEFENNQRCMCEALQQIMENQSD RLQGRQQEQQFKRELRNLPQQCGLRAPQRCDLEVESGGRDRY (SEQ ID NO: 441)
*Signal sequence is amino acids 1-22, 4-hydroxyproline is located at positions 67, 74, and 86.

Allergenic fragments of Ara h 2 are also considered peanut allergens and may be identified according to standard methods.

The terms “anti-Ara h 2 antibody,” “an antibody that binds to Ara h 2,” and “an antibody that specifically binds to Ara h 2” refer to an antibody that is capable of binding Ara h 2 with sufficient affinity such that the antibody is useful as a preventative, diagnostic, and/or therapeutic agent in targeting Ara h 2. In one embodiment, the extent of binding of an anti-Ara h 2 antibody to an unrelated, non-Ara h 2 protein is less than about 10% of the binding of the antibody to Ara h 2 as measured, e.g., by a radioimmunoassay (RIA). In certain embodiments, an antibody that binds to Ara h 2 has a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g., from 10⁻⁸ M to 10⁻¹³ M, e.g., from 10⁻⁹ M to 10⁻¹³ M). In certain embodiments, an antibody that binds to Ara h 2 has a K_{D} of between about 0.0001 nM and about 100 nM. In certain embodiments, an anti-Ara h 2 antibody binds to an epitope of Ara h 2 that is conserved among Ara h 2 from different peanut species.

The term “antibody” as used herein in the broadest sense encompasses various antibody structures, including but not limited to monoclonal antibodies, polyclonal antibodies, multispecific antibodies (e.g., bispecific antibodies), and antibody fragments so long as they exhibit the desired antigen-binding activity. An “antibody” can refer, for example, to a glycoprotein comprising at least two heavy chains (HCs) and two light chains (LCs) inter-connected by disulfide bonds, or an antigen binding portion thereof. Each heavy chain is comprised of a heavy chain variable region (VH) and a heavy chain constant region (CH). The heavy chain constant region may be comprised of three domains, CH1, CH2, and/or CH3. Each light chain is comprised of a light chain variable region (VL) and a light chain constant region (CL). The VH and VL regions can be further subdivided into regions of hypervariability, termed “complementarity determining regions” (CDRs), interspersed with regions that are more conserved, termed “framework regions” (FRs). Each VH and VL may be composed, for example, of three CDRs and four FRs, arranged from amino-terminus to carboxyl-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The terms “full-length antibody,” “intact antibody,” and “whole antibody” are used herein interchangeably to refer to an antibody having a structure substantially similar to a native antibody structure or having heavy chains that contain an Fc region as defined herein.

The term “human antibody” includes antibodies having variable and constant regions (if present) of human germline immunoglobulin sequences. Human antibodies of the invention can include amino acid residues not encoded by human germline immunoglobulin sequences (e.g., mutations introduced by random or site-specific mutagenesis in vitro or by somatic mutation in vivo) (see, Lonberg, N. et al. (1994) Nature 368(6474): 856-859); Lonberg, N. (1994) Handbook of Experimental Pharmacology 113:49-101; Lonberg, N. and Huszar, D. (1995) Intern. Rev. Immunol. Vol. 13: 65-93, and Harding, F. and Lonberg, N. (1995) Ann. N.Y. Acad. Sci 764:536-546). However, the term “human antibody” does not include antibodies in which CDR sequences derived from the germline of another mammalian species, such as a mouse, have been grafted onto human framework sequences (i.e., humanized antibodies).

The term “monoclonal antibody,” as used herein, refers to an antibody which displays a single binding specificity and affinity for a particular epitope. Accordingly, the term “human monoclonal antibody,” or “HuMab,” refers to an antibody which displays a single binding specificity, and which has variable and constant regions derived from human germline immunoglobulin sequences. In one embodiment, human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

An “antibody fragment” refers to a molecule other than an intact antibody that comprises a portion of an intact antibody that specifically binds to the antigen (e.g., an Ara h 2 protein described above to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. These antibody fragments are obtained using conventional techniques, and the fragments are screened for utility in the same manner as are intact antibodies. Antibody fragments can be produced by recombinant DNA techniques, or by enzymatic or chemical cleavage of intact immunoglobulins.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K)). Affinity can be measured by common methods known in the art, including those described herein. Specific illustrative and exemplary embodiments for measuring binding affinity are described below.

The term “K_{D} is measured by a surface plasmon resonance assay,” when used in the context of the claims, means that the K_{D} is measured according the method described in Example 2 which measures kinetic parameters for binding of anti-Ara h 2 antibodies to Ara h 2 described in Patil et al., J. Allergy Clin Immunol. 136(1):125-134 (2015). Typically, the antibodies disclosed herein bind to Ara h 2 with a dissociation equilibrium constant (K_{D}) of less than about 10⁻⁶ M, such as less than approximately 10⁻⁷ M, 10⁻⁸ M, 10⁻⁹ M, or 10⁻¹⁰ M or even lower when determined by surface plasmon resonance (SPR) technology in a BIACORE 3000 instrument using recombinant Ara h 2 as the analyte and the antibody as the ligand.

The term “EC50,” as used herein, refers to the concentration of an antibody or an antigen-binding portion thereof, which induces a response, either in an in vivo or an in vitro assay, such as neutralization of Ara h 2 (e.g., blocking Ara h 2 binding with a binding partner (e.g., an IgE antibody)) as is described herein, which is 50% of the maximal response (i.e., halfway between the maximal response and the baseline).

The terms “effective amount,” “effective dose,” and “effective dosage” as used herein are defined as an amount sufficient to achieve, or at least partially achieve, the desired effect. The term “therapeutically effective dose” or “therapeutically effective amount” is defined as an amount sufficient to prevent, cure, or at least partially arrest, the allergic reaction and its complications in a patient already suffering from an allergic reaction to peanut exposure or at risk to being exposed to Ara h 2 or an allergenic fragment thereof. Amounts effective for this use will depend upon the severity of the allergic reaction being treated and the general state of the patient's own immune system.

The term “epitope” or “antigenic determinant” refers to a site on an antigen to which an immunoglobulin or antibody specifically binds on Ara h 2. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods of determining spatial conformation of epitopes include techniques in the art and those described herein, for example, x-ray crystallography, cryo-electron microscopy, and 2-dimensional nuclear magnetic resonance. See, for example, Epitope Mapping Protocols in Methods in Molecular Biology, Vol. 66, G. E. Morris, Ed. (1996). Epitopes can also be defined by point mutations in the target protein (e.g., Ara h 2 or an allergic-inducing fragment thereof), which affect the binding of the antibody (e.g., monoclonal antibody).

The term “host cell,” as used herein, is intended to refer to a cell into which an expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

An “isolated antibody” is one which has been identified and separated and/or recovered from a component of its natural environment and/or is substantially free of other antibodies having different antigenic specificities (e.g., an isolated antibody that specifically binds to Ara h 2 is substantially free of antibodies that specifically bind antigens other than Ara h 2). Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using CoomassieM blue or, preferably, silver stain. Isolated antibody includes the antibody in situ within recombinant cells since at least one component of the antibody's natural environment will not be present. Similarly, isolated antibody includes the antibody in medium around recombinant cells. Ordinarily, however, isolated antibody will be prepared by at least one purification step.

The term “nucleic acid molecule,” as used herein, is intended to include DNA molecules and RNA molecules. A nucleic acid molecule may be single-stranded or double-stranded, but preferably is double-stranded DNA.

The term “isolated nucleic acid,” as used herein in reference to nucleic acids molecules encoding antibodies or antibody portions (e.g., VH, VL, CDRs) that bind to Ara h 2, is intended to refer to a nucleic acid molecule in which the nucleotide sequences encoding the antibody or antibody portion are free of other nucleotide sequences encoding antibodies that bind antigens other than Ara h 2, which other sequences may naturally flank the nucleic acid in human genomic DNA.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program is registered under U.S. Copyright Registration No. TXU510087.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, the terms “specific binding,” “selective binding,” “selectively binds,” and “specifically binds,” refer to antibody binding to an epitope on a predetermined antigen. Typically, the antibody binds with an affinity (Kn) of approximately less than 10⁻⁷ M, such as approximately less than 10⁻⁸ M, 10⁻⁹ M or 10⁻¹⁰ M or even lower when determined by surface plasmon resonance (SPR) technology in a BIACORE 3000 instrument, which can be performed, for example, using recombinant Ara h 2 as the analyte and the antibody as the ligand. In some embodiments, binding by the antibody to the predetermined antigen is with an affinity that is at least two-fold greater than its affinity for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen.”

A “subject,” a “patient,” or an “individual” is typically a human such as an adult, a child, or an infant.

As used herein, “administering” is meant a method of giving a dosage of a compound (e.g., an anti-Ara h 2 antibody or a nucleic acid encoding an anti-Ara h 2 antibody) or a composition (e.g., a pharmaceutical composition, e.g., a pharmaceutical composition including an anti-Ara h 2 antibody) to a subject. Preferably, the antibodies described herein are administered subcutaneously. The compositions utilized in the methods described herein can be administered, for example, intramuscularly, intravenously, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. The method of administration can vary depending on various factors (e.g., the compound or composition being administered, and the severity of the peanut allergy being treated). Preferably, the antibody or a combination of antibodies (for example, a combination of the T3 and T4 antibodies disclosed herein) are administered as a subcutaneous injection.

As used herein, the term “vector” is meant to include, but is not limited to, a nucleic acid molecule (e.g., a nucleic acid molecule that is capable of transporting another nucleic acid to which it has been linked), a virus (e.g., a lentivirus or an adenovirus, e.g., a recombinant adeno-associated virus (rAAV)), cationic lipid (e.g., liposome), cationic polymer (e.g., polysome), virosome, nanoparticle, or dentrimer. Accordingly, one type of vector is a viral vector, wherein additional DNA segments (e.g., transgenes, e.g., transgenes encoding the heavy and/or light chain genes of an anti-Ara h 2 antibody) may be ligated into the viral genome, and the viral vector may then be administered (e.g., by electroporation, e.g., electroporation into muscle tissue) to the subject in order to allow for transgene expression in a manner analogous to gene therapy. Another type of vector is a “plasmid,” which refers to a circular double stranded DNA loop into which additional DNA segments may be ligated. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids.

### II. COMPOSITIONS AND METHODS

In one aspect, the invention is based, in part, on anti-Ara h 2 antibodies. Such antibodies are useful, for example, for treating a subject having, or at risk of developing, a peanut allergy following exposure, direct or indirect, to a peanut allergen such as Ara h 2.

### A. Anti-Ara h 2 Antibodies

The invention provides isolated antibodies (e.g., any of the antibodies described herein) that bind to Ara h 2.

Accordingly, in one aspect, the invention provides isolated antibody that specifically binds to Ara h 2.

Exemplary antibodies include those described in the Appendix.

Antibodies of the invention may, for example, be monoclonal, human, humanized, or chimeric.

The antibodies can be full-length antibodies or antibody fragments thereof (e.g., an antibody fragment that binds Ara h 2). The antibody fragment may be selected from the group consisting of Fab, Fab′-SH, Fv, scFv, and (Fab′)₂ fragments. In some instances, the antibody is an IgG antibody (e.g., an IgG1 antibody). An antibody of the invention may have a half-life of ≥3 days (e.g., ≥1 week, e.g., ≥2 weeks, e.g., ≥1 month, e.g., ≥2 months, e.g., ≥3 months, e.g., ≥4 months, e.g., ≥5 months, e.g., ≥6 months).

In a further aspect, an anti-Ara h 2 antibody according to any of the above embodiments may incorporate any of the features, singly or in combination, as described in Sections 1-5 below.

### 1. Antibody Affinity

In certain embodiments, an antibody provided herein may have a dissociation constant (K)) of ≤10 μM, ≤1 μM, ≤100 nM, ≤10 nM, ≤1 nM, ≤0.1 nM, or ≤0.01 nM.

In one embodiment, K_{D} is measured by a radiolabeled antigen binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, K_{D} is measured using a BIACORE® surface plasmon resonance assay. For example, an assay using a BIACORE®-3000 (BIAcore, Inc., Piscataway, NJ) is performed at 25° C. with immobilized antigen CM5 chips at ˜10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{D}) is calculated as the ratio kₒₙ/k_{off}. See, for example, Chen et al., J. Mol. Biol. 293:865-881 (1999). If the on-rate exceeds 10⁶M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

### 2. Antibody Fragments

In certain embodiments, an antibody provided herein is an antibody fragment. Antibody fragments include, but are not limited to, Fab, Fab′, Fab′-SH, F(ab′)₂, Fv, and scFv fragments, which are known in the art. Also included are diabodies, which have two antigen-binding sites that may be bivalent or bispecific, as is known in the art. Triabodies and tetrabodies are also known. Single-domain antibodies are also antibody fragments comprising all or a portion of the heavy chain variable domain or all or a portion of the light chain variable domain of an antibody. In certain embodiments, a single-domain antibody is a human single-domain antibody.

Antibody fragments can be made by various techniques, including but not limited to proteolytic digestion of an intact antibody as well as production by recombinant host cells (e.g., E. coli or phage), as described herein.

### 3. Chimeric and Humanized Antibodies

In certain embodiments, an antibody provided herein is a chimeric antibody. In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 4. Human Antibodies

In certain embodiments, an antibody provided herein is a human antibody (e.g., a human monoclonal antibody (HuMab), e.g., an anti-Ara h 2 HuMab). Human antibodies can be produced using various techniques known in the art.

In some instances, human antibodies are obtained by cloning the heavy and light chain genes directly from human B cells obtained from a human subject as is described herein in Example 1. The B cells are separated from peripheral blood (e.g., by flow cytometry, e.g., FACS), stained for B cell marker(s), and assessed for antigen binding. The RNA encoding the heavy and light chain variable regions (or the entire heavy and light chains) is extracted and reverse transcribed into DNA, from which the antibody genes are amplified (e.g., by PCR) and sequenced. The known antibody sequences can then be used to express recombinant human antibodies against a known target antigen (e.g., Ara h 2).

In some instances, human antibodies may be prepared by administering an immunogen (e.g., Ara h 2) to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. Human variable regions from intact antibodies generated by such animals may be further modified, for example, by combining with a different human constant region.

In some instances, human antibodies can also be made by hybridoma-based methods, as described in further detail below. Human myeloma and mouse-human heteromyeloma cell lines for the production of human monoclonal antibodies have been described.

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### 5. Antibody Variants

In certain embodiments, amino acid sequence variants of the anti-Ara h 2 antibodies are contemplated.

For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen-binding.

In certain embodiments, antibody variants having one or more amino acid substitutions are provided. Sites of interest for substitutional mutagenesis include the CDRs and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, for example, retained/improved antigen binding, decreased immunogenicity, or improved ADCC or CDC.

TABLE 1
Exemplary and Preferred Amino Acid Substitutions

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated and the variant antibodies displayed on phage and screened for a particular biological activity (e.g., binding affinity).

Alterations (e.g., substitutions) may be made in CDRs, for example, to improve antibody affinity. Such alterations may be made in CDR “hotspots,” i.e., residues encoded by codons that undergo mutation at high frequency during the somatic maturation process, and/or residues that contact antigen, with the resulting variant VH or VL being tested for binding affinity. Affinity maturation by constructing and reselecting from secondary libraries is known in the art. In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. Such alterations may, for example, be outside of antigen contacting residues in the CDRs. In certain embodiments of the variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

In certain embodiments, alterations may be made to the Fc region of an antibody. These alterations can be made alone, or in addition to, alterations to one or more of the antibody variable domains (i.e., VH or VL regions) or regions thereof (e.g., one or more CDRs or FRs). The alterations to the Fc region may result in reduced antibody effector functions (e.g., complement-dependent cytotoxicity (CDC))

In certain instances, the invention contemplates an antibody, e.g., antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CYTOTOX 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al. J. Immunol. Methods 202:163 (1996); Cragg, M. S. et al. Blood. 101:1045-1052 (2003); and Cragg, M. S. and M. J. Glennie Blood. 103:2738-2743 (2004)). FcRn binding and in vivo clearance/half-life determinations can also be performed using methods known in the art (see, e.g., Petkova, S. B. et al. Int'l. Immunol. 18(12):1759-1769 (2006)).

Antibodies with reduced effector function include those with substitution of one or more of Fc region residues 238, 265, 269, 270, 297, 327 and 329 (U.S. Pat. Nos. 6,737,056 and 8,219,149). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. Nos. 7,332,581 and 8,219,149).

In certain instances, the proline at position 329 of a wild-type human Fc region in the antibody is substituted with glycine or arginine or an amino acid residue large enough to destroy the proline sandwich within the Fc/Fc.gamma receptor interface that is formed between the proline 329 of the Fc and tryptophan residues Trp 87 and Trp 110 of FcgRIII (Sondermann et al.: Nature 406, 267-273 (20 Jul. 2000)). In certain instances, the antibody comprises at least one further amino acid substitution. In one instance, the further amino acid substitution is S228P, E233P, L234A, L235A, L235E, N297A, N297D, or P331S, and still in another instance the at least one further amino acid substitution is L234A and L235A of the human IgG1 Fc region or S228P and L235E of the human IgG4 Fc region (see e.g., US 2012/0251531), and still in another instance the at least one further amino acid substitution is L234A and L235A and P329G of the human IgG1 Fc region.

In certain embodiments, alterations of the amino acid sequences of the Fc region of the antibody may alter the half-life of the antibody in the host. Certain mutations that alter binding to the neonatal Fc receptor (FcRn) may extend half-life of antibodies in serum. For example, antibodies that have tyrosine in heavy chain position 252, threonine in position 254, and glutamic acid in position 256 of the heavy chain can have dramatically extended half-life in serum (see, e.g., U.S. Pat. No. 7,083,784).

In other embodiments, Fc modifications are introduced to maximize dosing levels and to prevent anaphylaxis of peanut allergy.

In certain instances, antibodies of the invention can be altered to increase or decrease the extent to which the antibody is glycosylated. Addition or deletion of glycosylation sites to an antibody of the invention may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GIcNAc), galactose, and sialic acid, as well as a fucose attached to a GIcNAc in the “stem” of the biantennary oligosaccharide structure. In some instances, modifications of the oligosaccharide in an antibody of the invention are made in order to create antibody variants with certain improved properties.

In certain instances, it is desirable to create cysteine engineered anti-Ara h 2 antibodies, e.g., “thioMAbs,” in which one or more residues of an antibody are substituted with cysteine residues. In particular instances, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain instances, any one or more of the following residues are substituted with cysteine: V205 (Kabat numbering) of the light chain; A118 (EU numbering) of the heavy chain; and S400 (EU numbering) of the heavy chain Fc region. Cysteine engineered antibodies may be generated as described, for example, in U.S. Pat. No. 7,521,541.

In certain instances, an antibody of the invention provided herein are further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### B. Isolation and Characterization of Human Antibodies to Ara h 2

An exemplary method for identifying anti-Ara h 2 antibodies is described in the Examples.

Sequence information for antibodies described herein can be ascertained using sequencing techniques which are well known in the art.

Similarly, affinity of the antibodies for anti-Ara h 2 antibodies can also be assessed using standard techniques. For example, Biacore 3000 can be used to determine the affinity of such antibodies. Antibodies are captured on the surface of a Biacore chip (GE healthcare), for example, via amine coupling (Sensor Chip CM5). The captured antibodies can be exposed to various concentrations of Ara h 2 in solution, and the Kₒₙ and K_{off} for an affinity (K_{D}) can be calculated, for example, by BIAevaluation software.

Antibodies can also be characterized for binding to Ara h 2 using a variety of known techniques, such as ELISA, Western blot, biolayer interferometry (BLI), etc. Generally, the antibodies are initially characterized by ELISA. Briefly, microtiter plates can be coated with purified Ara h 2 in PBS, and then blocked with irrelevant proteins such as bovine serum albumin (BSA) diluted in PBS. Dilutions of plasma are added to each well and incubated for 1-2 hours at 37° C. The plates are washed with PBS/Tween 20 and then incubated with a goat-anti-human IgG Fc-specific polyclonal reagent conjugated to alkaline phosphatase for 1 hour at 37° C. After washing, the plates are developed with ABTS substrate, and analyzed at OD of 405. In some examples, the ELISA may be an ImmunoCAP™ ELISA assay.

In other instances, competition assays may be used to identify an antibody that competes with an anti-Ara h 2 antibody for binding to Ara h 2. In certain embodiments, such a competing antibody binds to the same epitope (e.g., a linear or a conformational epitope) that is bound by an anti-Ara h 2 antibody of the invention. Detailed exemplary methods for mapping an epitope to which an antibody binds are provided in Morris (1996) “Epitope Mapping Protocols,” in Methods in Molecular Biology vol. 66 (Humana Press, Totowa, NJ).

In an exemplary competition assay, immobilized Ara h 2 is incubated in a solution comprising a first labeled antibody that binds to Ara h 2 and a second unlabeled antibody that is being tested for its ability to compete with the first antibody for binding to Ara h 2. The second antibody may be present in a hybridoma supernatant. As a control, immobilized Ara h 2 is incubated in a solution comprising the first labeled antibody but not the second unlabeled antibody. After incubation under conditions permissive for binding of the first antibody to Ara h 2, excess unbound antibody is removed, and the amount of label associated with immobilized Ara h 2 is measured. If the amount of label associated with immobilized Ara h 2 is substantially reduced in the test sample relative to the control sample, then that indicates that the second antibody is competing with the first antibody for binding to Ara h 2.

### C. Compositions

In another aspect, the invention features a pharmaceutical composition comprising a therapeutically effective amount of one or more isolated human antibodies or antigen-binding fragments thereof that specifically bind Ara h 2, together with one or more pharmaceutically acceptable excipients.

In one embodiment, the pharmaceutical composition comprises a therapeutically effective amount of two or more isolated human antibodies or antigen-binding fragments thereof that specifically bind Ara h 2 together with one or more pharmaceutically acceptable excipients.

In one embodiment, the invention features a composition, which is a combination of a therapeutically effective amount of one or more anti-Ara h 2 antibodies or antigen-binding fragments thereof of the invention, and a therapeutically effective amount of a second therapeutic agent. The second therapeutic agent may be a small molecule drug, a protein/polypeptide, an antibody, a nucleic acid molecule, such as an anti-sense molecule, or a siRNA. The second therapeutic agent may be synthetic or naturally derived.

The second therapeutic agent may be any agent that is advantageously combined with an antibody or fragment thereof of the invention, for example, a second antibody other than those described herein that is capable of blocking the binding of Ara h 2 to IgE present on mast cells or basophils. A second therapeutic agent may also be any agent that is used as standard of care in treating an allergic response to any allergen. Such second therapeutic agent may be an antihistamine, epinephrine, a decongestant, a corticosteroid, or a biologic (e.g., an anti-IgE antibody such as omalizumab (XOLAIRO)).

In certain embodiments, the second therapeutic agent may be an agent that helps to counteract or reduce any possible side effect(s) associated with the antibody or antigen-binding fragment of an antibody of the invention, if such side effect(s) should occur.

It will also be appreciated that the antibodies and pharmaceutically acceptable compositions of the present invention can be employed in combination therapies, that is, the antibodies and pharmaceutically acceptable compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an antibody may be administered concurrently with another agent used to treat the same disorder), or they may achieve different effects (e.g., control of any adverse effects). As used herein, additional therapeutic agents that are normally administered to treat or prevent a particular disease, or condition, are appropriate for the disease, or condition, being treated.

When multiple therapeutics are co-administered, dosages may be adjusted accordingly, as is recognized in the pertinent art.

Accordingly, the present invention provides a composition, e.g., a pharmaceutical composition, containing one or more (e.g., 1, 2, 3, or 4 or more) of the anti-Ara h 2 antibodies, or antibody fragments thereof, disclosed herein (e.g., in the Appendix). The antibodies, if desired, may be modified according to any of the modifications outlined above. The pharmaceutical compositions may be formulated together with a pharmaceutically acceptable carrier, excipient, or diluent. In some instances, the pharmaceutical compositions include two or more of the anti-Ara h 2 antibodies. Preferably, each of the antibodies of the composition binds to a distinct epitope of Ara h 2.

A pharmaceutical composition described herein can be administered by a variety of methods known in the art. As will be appreciated by the skilled artisan, the route and/or mode of administration will vary depending upon the desired results. The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art.

To administer a compound of the invention by certain routes of administration, it may be necessary to coat the compound with, or co-administer the compound with, a material to prevent its inactivation. For example, the compound may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Active ingredients of the pharmaceutical composition may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions.

Sustained-release preparations may be prepared. Suitable examples of sustained-release preparations include semipermeable matrices of solid hydrophobic polymers containing the antibody, which matrices are in the form of shaped articles, for example, films, or microcapsules.

The formulations to be used for in vivo administration are generally sterile. Sterility may be readily accomplished, e.g., by filtration through sterile filtration membranes. Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof. Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, such as TWEEN® 80. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Alternatively, genes encoding the anti-Ara h 2 antibodies may be delivered directly into the subject for expression rather than administering purified antibodies for prevention or therapy. For example, viral vectors, such as recombinant viruses, can be used to deliver the heavy and light chain genes. In one example, rAAV virus particles can be used to deliver anti-HIV monoclonal antibodies (Balazs et al. Nature. 481: 81, 2012). Antibody genes could also be effectively delivered by electroporation of muscle cells with plasmid DNA containing heavy and/or light chain genes (e.g., VH and/or VL genes) (Muthumani et al. Hum Vaccin Immunother. 10: 2253, 2013). Lentivirus vectors or other nucleic acids (e.g., RNA) capable of delivering transgenes could also be used to delivery antibody genes to establish serum antibody levels capable of prevention.

Also contemplated are kits including human anti-Ara h 2 antibodies and, optionally, instructions for use. The kits can further contain one or more additional reagents, such as a second, different anti-Ara h 2 antibody having a complementary activity that binds to an epitope on Ara h 2 that is distinct from the epitope to which the first anti-Ara h 2 antibody binds.

### D. Therapy

Any of the anti-Ara h 2 antibodies described herein (e.g., in the Appendix) and compositions containing the antibodies can be used in a variety of in vitro and in vivo therapeutic applications. In some embodiments, an anti-Ara h 2 antibody may be used as a monotherapy. In some embodiments, an anti-Ara h 2 antibody may be used as a combination therapy.

The invention provides an anti-Ara h 2 antibody for use as a medicament. In further aspects, an anti-Ara h 2 antibody for use in treating a peanut allergy is provided. In certain embodiments, an anti-Ara h 2 antibody for use in a method of treatment is provided. In certain embodiments, the invention provides an anti-Ara h 2 antibody for use in a method of treating an individual having a peanut allergy comprising administering to the individual an effective amount of the anti-Ara h 2 antibody. In one such embodiment, the method further comprises administering to the individual an effective amount of at least one additional therapeutic agent, for example, as described below.

The invention provides an anti-Ara h 2 antibody in the manufacture or preparation of a medicament. In a further aspect, the invention provides for the use of an anti-Ara h 2 antibody in the manufacture or preparation of a medicament. In one embodiment, the medicament is for treatment of a peanut allergy. In a further embodiment, the medicament is for use in a method of treating a peanut allergy, e.g., comprising administering to an individual having a peanut allergy an effective amount of the medicament. In one such embodiment, the method further comprises administering to the individual an effective amount of at least one additional therapeutic agent, e.g., as described below.

In a further aspect, the invention provides a method for treating a peanut allergy. In some instances, the method comprises administering the individual having such a peanut allergy an effective amount of an anti-Ara h 2 antibody. In one embodiment, the method comprises administering to an individual having such peanut allergy an effective amount of an anti-Ara h 2 antibody (e.g., any anti-Ara h 2 antibody disclosed herein). In one such embodiment, the method further comprises administering to the individual an effective amount of at least one additional therapeutic agent, as described below.

In a further aspect, the invention provides pharmaceutical formulations comprising any of the anti-Ara h 2 antibodies provided herein, e.g., for use in any of the above therapeutic methods. In one embodiment, a pharmaceutical formulation comprises any of the anti-Ara h 2 antibodies provided herein and a pharmaceutically acceptable carrier.

In a further aspect, the invention features a method of treating a subject having a peanut allergy or who is sensitized to peanuts comprising administering a therapeutically effective amount of an antibody (e.g., a human monoclonal antibody) that specifically binds to Ara h 2 or a pharmaceutical composition thereof, thereby treating the subject.

Accordingly, the invention features method for treating a patient who demonstrates a sensitivity to a peanut allergen (e.g., Ara h 2), an allergic reaction against a peanut allergen, or to Ara h 2 protein, or for treating at least one symptom or complication associated with a sensitivity to, or allergic reaction against a peanut allergen, an allergic reaction against a peanut allergen, or to Ara h 2 protein, comprising administering an effective amount of one or more isolated human monoclonal antibodies or antigen-binding fragments thereof that bind specifically to Ara h 2, e.g., in the Appendix, according to claims described herein to a patient in need thereof, wherein the sensitivity to, or an allergic reaction against, to a peanut allergen, an allergic reaction against a peanut allergen, or to Ara h 2 protein, is lessened in severity and/or duration, or at least one symptom or complication associated with the sensitivity to, or allergic reaction against, to a peanut allergen, an allergic reaction against a peanut allergen, or to Ara h 2 protein is ameliorated, or that the frequency and/or duration of, or the severity of the sensitivity to or allergic reaction against, to a peanut allergen, an allergic reaction against a peanut allergen, or to Ara h 2 protein, is reduced following administration of one or more of the isolated human monoclonal antibodies or fragments thereof that bind specifically to Ara h 2.

Preferably, the method of treatment further comprises administering an effective amount of a second therapeutic agent useful for diminishing an allergic reaction to a peanut allergen, an allergic reaction against a peanut allergen, or to Ara h 2 protein. In some instances, the second therapeutic agent is selected from the group consisting of a corticosteroid, a bronchial dilator, an antihistamine, epinephrine, or a decongestant. Typically, treatment results in a reduction in allergic rhinitis, allergic conjunctivitis, allergic asthma, or an anaphylactic response following exposure, direct or indirect, of the patient to a peanut allergen, an allergic reaction against a peanut allergen, or to Ara h 2 protein.

Furthermore, as is discussed herein, antibodies of the invention can be used either alone or in combination with other agents in a therapy. For instance, an antibody of the invention may be co-administered with at least one additional therapeutic agent (e.g., a corticosteroid, a bronchial dilator, an antihistamine, epinephrine, and/or a decongestant). Such combination therapies encompass combined administration (where two or more therapeutic agents are included in the same or separate formulations), and separate administration, in which case, administration of the antibody of the invention can occur prior to, simultaneously, and/or following, administration of the additional therapeutic agent or agents. In one embodiment, administration of the anti-Ara h 2 antibody and administration of an additional therapeutic agent occur within about one month, or within about one, two or three weeks, or within about one, two, three, four, five, or six days, of each other.

Anti-Ara h 2 antibodies described herein may also be used in combination.

An antibody, e.g., as described in the Appendix or in the claims, can be administered by any suitable means, including parenteral, intrapulmonary, intranasal, oral, mucosal, intravenous, and, if desired for local treatment, intralesional administration. Parenteral infusions include intramuscular, intravenous, intraarterial, intraperitoneal, or subcutaneous administration. In some instances, an anti-Ara h 2 antibody (e.g., any anti-Ara h 2 antibody disclosed herein) may be administered orally, intrarectally, mucosally, intravenously, intramuscularly, intradermally, transdermally, subcutaneously, percutaneously, intraarterially, intraperitoneally, intravitreally, topically, intralesionally, intraarticularly, intraprostatically, intrapleurally, intratracheally, intrathecally, intranasally, intravaginally, intratumorally, intraperitoneally, peritoneally, intraventricularly, intracranially, subconjunctivally, intravesicularly, intrapericardially, intraumbilically, intraorbitally, ocularly, intraocularly, juxtasclerally, subtenonly, superchoroidally, by inhalation, by injection, by eye drop, by implantation, by infusion, by continuous infusion, by localized perfusion bathing target cells directly, by catheter, by lavage, in cremes, or in lipid compositions. In certain instances, antibody genes (e.g., genes encoding any one or more of the anti-Ara h 2 antibodies of the invention could be administered as a gene therapy to produce the one or more anti-Ara h 2 antibodies in the subject using either DNA vectors or viral vectors (e.g., rAAV vectors). Dosing can be by any suitable route, for example, by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of a peanut allergy or a subject who is hypersensitive to peanuts, the appropriate dosage of an antibody of the invention (when used alone or in combination with one or more other additional therapeutic agents) will depend on the severity of the allergic reaction to be prevented/treated, the duration of effective antibody concentration required, the type of antibody, the severity and course of the disease, whether the antibody is administered for preventive or therapeutic purposes, previous therapy, the patient's clinical history and response to the antibody, and the discretion of the attending allergist. The antibody is suitably administered to the patient at one time or over a series of treatments. For repeated administrations over several days or longer, depending on the condition, the treatment would generally be sustained until a desired suppression of disease symptoms occurs. Doses may be administered intermittently, e.g. every week, every two weeks, every three weeks, every four weeks, every five weeks, every six weeks, every seven weeks, every eight weeks, every nine weeks, every ten weeks, every eleven weeks, or every twelve weeks (e.g., such that the patient receives from about two to about twenty, or e.g., about six doses of the antibody). For example, a dose may be administered once per month, once every two months, or once every three months (e.g., by subcutaneous injection) as an initial higher loading dose, followed by one or more lower doses may be administered. However, other dosage regimens may be useful. The progress of this therapy is easily monitored by conventional techniques and assays.

Actual dosage levels of the active ingredients in the pharmaceutical compositions of the present invention may be varied so as to obtain an amount of the active ingredient which is effective to achieve the desired therapeutic response and duration for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. An allergist having ordinary skill in the art can readily determine and prescribe the effective amount of the pharmaceutical composition required. For example, the allergist can start doses of the compounds of the invention employed in the pharmaceutical composition at levels lower than that required in order to achieve the desired therapeutic effect and gradually increase the dosage until the desired effect is achieved. In general, a suitable daily dose of compositions of the invention will be that amount of the compound which is the lowest dose effective to produce a therapeutic effect. Such an effective dose will generally depend upon the factors described above. It is preferred that administration be intravenous, intramuscular, intraperitoneal, or subcutaneous, preferably administered proximal to the site of the target. If desired, the effective daily dose of therapeutic compositions may be administered as two, three, four, five, six or more sub-doses administered separately at appropriate intervals throughout the day, optionally, in unit dosage forms. While it is possible for a compound of the present invention to be administered alone, it is preferable to administer the compound as a pharmaceutical formulation (composition).

Therapeutic compositions can be administered with medical devices known in the art. For example, in a preferred embodiment, a therapeutic composition of the invention can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

In some instances, the antibody-based therapy may be combined with an additional therapy for more efficacious treatment (e.g., additive or synergistic treatment) of the subject. Accordingly, subjects treated with antibodies of the invention can be additionally administered (prior to, simultaneously with, or following administration of a human antibody of the invention) with another therapeutic agent which enhances or augments the therapeutic effect of the human antibodies.

### E. Diagnostics

In certain embodiments, any of the anti-Ara h 2 antibodies of the invention are useful for in vitro or in vivo detection of peanut tolerance in allergic individuals. The term “detecting” as used herein encompasses quantitative or qualitative detection. In certain embodiments, a biological sample comprises a cell or tissue.

Current data suggests that there are three dominant conformational epitope bins where IgE binds to the Ara h 2 antigen and the recombinant IgG neutralizing anti Ara h 2 antibodies will compete for these sites and inhibit or displace IgE binding. With these neutralizing antibodies, the current tandem epitope binning by biolayer light interferometry assays can be used to assay whether a sample (e.g. a serum sample from a patient believed to have been exposed to a peanut allergen or a patient undergoing an OIT) contains antibodies that bind the same epitope bin, and whether those antibodies can effectively compete with the neutralizing antibodies. This test is useful to track the progress of therapy or to predict whether a patient will obtain the outcome of sustained unresponsiveness after OIT by assaying that patient's early OIT serum sample.

Accordingly, prior to the initiation of OIT, IgG antibodies isolated from patient serum will lack significant Ara h 2 neutralizing capacity and will be unable to compete with antibodies disclosed herein. During the course of treatment, patients that are responding to OIT will begin to produce IgG antibodies that are capable of competing with the anti-Ara h 2 antibodies while patients that are not responding to the therapy will maintain low levels of Ara h 2 neutralizing antibodies. The results of the competition assay can be used to predict whether an individual patient will attain a desired outcome of the treatment or can be used to adjust the duration of treatment to improve the likelihood of a desired outcome.

### F. Articles of Manufacture

In another aspect of the invention, an article of manufacture containing materials useful for the treatment, prevention and/or diagnosis of the disorders described above is provided. The article of manufacture comprises a container and a label or package insert on or associated with the container.

Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes. In some embodiments, the article of manufacture comprises an additional therapeutic agent (e.g., a corticosteroid, a bronchial dilator, an antihistamine, epinephrine, and/or a decongestant).

Other embodiments of the present invention are described in the following Examples. The present invention is further illustrated by the following examples which should not be construed as further limiting. The contents of the appendix, and all references, patents and published patent applications cited throughout this application are expressly incorporated herein by reference.

### III. EXAMPLES

### Example 1—Isolation of Genes Encoding Anti-Ara h 2 Antibodies from Patients Having Sustained Unresponsiveness to Peanut Allergen after One Month of Peanut Avoidance

The peanut-specific protective antibodies directed towards the immunodominant peanut antigen Ara h 2 were cloned from the circulation of pediatric patients who had sustained tolerance to peanut after OIT. From the same peanut OIT study, suppression of basophil activation was identified as a good biomarker of future tolerance. Furthermore, the suppression of basophils is mediated by IgG in the sera of tolerant patients, from whom Ara h 2-specific antibodies were cloned. The protective antibodies were identified as those able to outcompete non-tolerant antibodies using biolayer light interferometry, as those have better antigen blocking capabilities.

### Patient Selection

Peripheral blood was obtained after IRB-approved consent from peanut allergic participants of an open-label randomized trial of peanut OIT (NCT01324401). Study inclusion criteria included participants aged 7-21 years old with a diagnosis of peanut allergy based on a clinical history of reaction to peanut within one hour of ingestion and either an elevated skin prick test (>8 mm wheal) or elevated peanut-specific IgE (CAP FEIA>10 kU/L).

A total of 30 patients were enrolled, including 4 in an observational control arm, who were monitored for 1 year, after which they also received active OIT with premeasured peanut flour (Medium Roast, Golden Peanut Co., Alpharetta, GA). The protocol began with a 1 day modified rush before the build up phase to a 12 week maintenance phase with 4 grams of peanut protein, at the end of which desensitization was evaluated with an oral food challenge. After 4 weeks of peanut avoidance, subjects underwent a double blind oral food challenge to peanut.

Of the 22 subjects who were effectively desensitized, 9 subjects continued to have sustained unresponsiveness (SU) after a month of peanut avoidance and 13 subjects were found to have only transient desensitization (TD).

### Specific Immunoglobulin E Measurement

Measurement of antigen-specific immunoglobulin levels in the serum or plasma from peripheral blood of subjects undergoing OIT was determined using the ImmunoCAP 1000 instrument (Phadia AB) according to the manufacturer's instructions.

### Ara h 2 Specific B Cells Identification

Ara h 2 specific B cells from peripheral blood were identified by flow cytometry using a fluorescent multimer created as described by Patil et al., J. Allergy Clin Immunol. 136(1):125-134 (2015). Briefly, peripheral blood mononuclear cells (PBMCs) isolated by density gradient centrifugation (Ficoll-Paque Plus; GE Healthcare) from peripheral blood of patients undergoing peanut OIT were then stained using CD3-APC (eBioscience clone OKT3), CD14-APC (eBioscience, clone 61D3), CD16-APC (eBioscience clone CB16), CD19-APC-Cy7 (BD Biosciences clone SJ25C1), CD27-PE (BD Pharmingen clone M-T271), CD38-Violet 421 (BD Biosciences clone HIT2), IgM-PE-Cy5 (BD Pharmingen clone G20-127), and AF488-Ara h 2 multimer. Data were analyzed using FlowJo 8.8.7 software (TreeStar).

Ara h 2 specific B cells, identified as multimer⁺CD19⁺ cells were isolated 1-3 months after the start of OIT or 3-6 months after post-OIT peanut avoidance and re-ingestion for single-cell indexed fluorescence-activated cell sorting (BD Aria II, BD Fusion SORP) into 96-well plates (Eppendorf) for further single cell nested RT-PCR BCR amplification. Briefly, cells sorted into RNAsin containing single strand buffer were frozen at −80 C and subsequently underwent heat lysis with NP-40 and random hexamers. The RT reaction was followed by 2 nested PCR reactions for both heavy and light chains, as described by Patil et al., J. Allergy Clin Immunol. 136(1):125-134 (2015).

Successfully amplified products were Sanger sequenced (Genewiz Inc.) using second PCR primers. Consensus sequences combining both the forward and reverse sequences were determined using pairwise alignment in Geneious (Biomatters Ltd). These sequences were then analyzed using IMGTN-BLAST to identify germline V, D, and J sequences with the highest identity, CDR3 regions, and nucleotide and amino acid changes from germline sequence.

Purified (Qiagen PCR purification kit) paired heavy and light chains subsequently underwent a third PCR reaction for the addition of restriction enzyme sites, followed by repeat PCR purification. For expression as recombinant antibodies on an IgG1 backbone, digested heavy and light PCR amplicons were ligated into heavy and light chain vectors, respectively, as described by Patil et al., J. Allergy Clin Immunol. 136(1):125-134 (2015), and transformed in competent E. coli NEB5a bacteria (New England Biolabs) for ampicillin selection of plasmids with Sanger-sequence verified PCR amplicons. Plasmid DNA (25 ng) from selected heavy and light chains were transfected into HEK293 T cells using GenJet™ In Vitro DNA Transfection Reagent (SignaGen). Supernatants were harvested from cells after three days of culture at 37° C. with 5% CO2 in serum free HL-1 media (Lonza) supplemented with Pen-Strep and 8 mM Glutamax (Gibco). Antibodies were purified from supernatants using Protein G beads (ThermoFischer).

Recombinant Ara h 2 antibody specificity was verified by ImmunoCAP measurement for each antibody. Epitope binning was performed using SA-sensors (ForteBio) loaded with 0.5 uM concentration of biotinylated Ara h 2 (Indoor Technologies) by biolayer light interferometry (ForteBio Octet Red96). Epitope binning was performed using Octet Data Analysis Software.

In order to be expressed as monoclonal antibodies, restriction sites were added during cloning through the use of highly permissive, degenerate primers situated in the FR1 and FR4 regions, for subsequent cloning into a vector with an IgG1 constant region. As a result of both PCR error introduction rates as well as the primers required to add the requisite restriction enzyme cleavage sites, the original VDJ sequence cloned from RNA, rather than DNA, is now altered. The altered proteins are expressed as recombinant antibodies for further characterization. Furthermore, the original antibodies may have been an isotype other than IgG1 but are then artificially made into IgG1 (or any other) constant region containing antibody.

### Example 2—Antibody Binding to Ara h 2 as Determined by Surface Plasmon Resonance

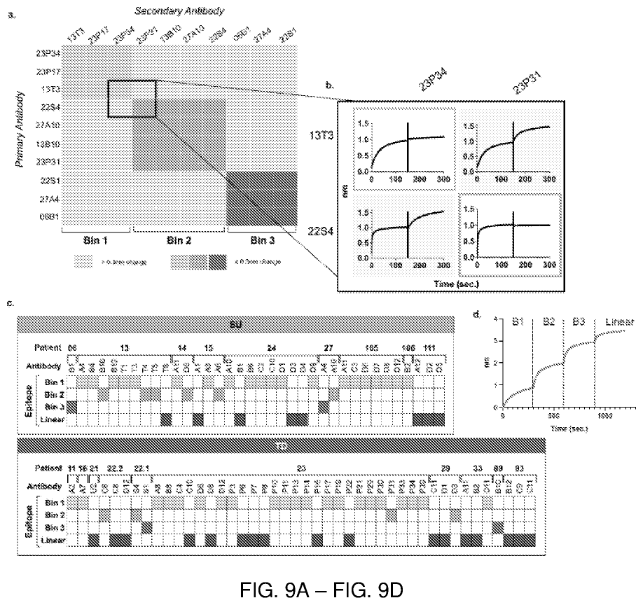

Calculated equilibrium dissociative constants (K)) for antigen binding to anti-Ara h 2 antibodies was determined using a real-time surface plasmon resonance biosensor (Biacore 3000) assay. Exemplary results are shown in Table 2. BLI data are shown in FIGS. 9A-9D.

TABLE 2

| ab | KD |
|---|---|
| T1 | 4.85E−09 |
| T3 | 1.93E−10 |
| T4 | 1.43E−12 |
| T5 | 1.12E−10 |

### Example 3—Anti-Ara h 2 Antibody Epitope Characterization

The epitopes of antibodies T1, T3, T4, and T5 were characterized using biotinylated Ara h 2 by epitope binning by biolayer light interferometry (ForteBio, Octet). Experiments showed that antibodies T1 and T3 share the same epitope bin (Bin 1) and antibodies T4 and T5 share a different epitope bin (Bin 2).

### Example 4—Ara h 2 Tolerization Assay

An assay was developed to detect tolerization to the Ara h 2 antigen. The assay can be used to assess treatment response of an individual with a peanut allergy to peanut exposure. The assay may include measuring anti-Ara h 2 antibodies in a sample from a subject (e.g., a plasma sample) using a competitive assay that includes one or more anti-Ara h 2 antibodies, including any anti-Ara h 2 antibody disclosed herein (see, e.g., Example 7).

In some examples, a combination of anti-Ara h 2 antibodies is used. In some examples, the combination includes one or more anti-Ara h 2 antibodies that bind one or more of the epitope bins or linear epitopes described in Example 7 (see Table 5).

### Results

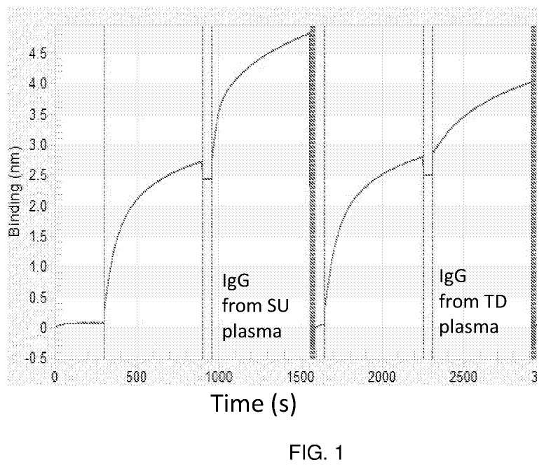

Using a bio-layer interferometry (BLI) streptavidin-sensor (Octet K2), biotinylated native Ara h 2 was loaded. The sensor was then used to bind a mix of P7, P31, P34, and S1 antibodies (each at a concentration of 1 ng/μL), followed by buffer, then tested with IgG purified from post-oral immunotherapy (OIT) patient plasma. As is shown in FIG. 1, IgG from a patient with sustained unresponsiveness (SU) or tolerance bound better than from a patient with transient desensitization (TD), or who lost tolerance after oral immunotherapy.

### Example 5—Conformational Bins of Antibodies that Bind Ara h 2 Antigen

Epitope mapping for antibodies that bind Ara h 2 antigen were performed using competitive assays, epitope mapping using chimeric antigens, and epitope mapping using biotinylated peptides.

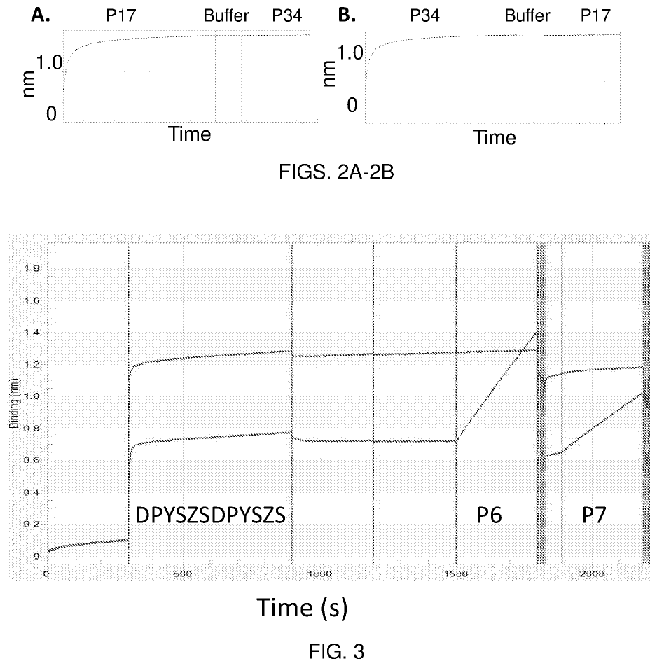

Two antibodies from a non-tolerant subject bind the same epitope, assayed by second antibodies P34 (FIG. 2A) and P27 (FIG. 2B) against the saturating antibodies P17 (FIG. 2A) and P34 (FIG. 2B) in a tandem BLI experiment performed after loading a streptavidin sensor with biotinylated native Ara h2 (Octet K2).

A summary of the conformational bins is described in Example 7.

### Example 6—Mapping of Linear Epitopes of Antibodies that Bind Ara h 2 Antigen

Three linear epitopes of antibodies that bind Ara 2 h antigen were mapped (see also Example 7 and Table 5). FIG. 3 shows exemplary results from linear epitope mapping of the DPYSZS epitope. Using a BLI streptavidin-sensor (Octet K2), biotinylated peptide DPYSZSDPYSZS (red line) was loaded). The sensor was then used to bind a mix of P6, then P7, as labeled.

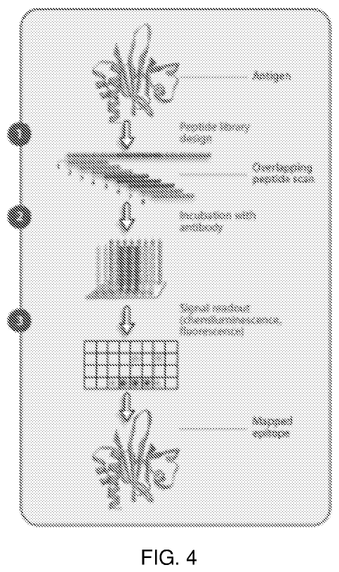

Additional data on the mapping of the three linear epitopes described in Example 7 was obtained using peptide mapping using microarrays performed by a commercial service. PT Peptide Technologies' PepStar™ peptide microarrays comprise purified synthetic peptides derived from antigens (for principle of epitope detection see FIG. 4) or other sources that are chemoselectively and covalently immobilized on the glass surface. An optimized hydrophilic linker moiety is inserted between the glass surface and the antigen derived peptide sequence to avoid false negatives caused by sterical hindrances.

### Methods and Materials

### Sequences

For the peptide mapping using microarrays experiment, the following library of 65 15-meric peptides was synthesized and immobilized on microarray slides:

TABLE 3
Peptide library

| SEQ ID NO. | Index | Peptide Sequence | Annotation |
|---|---|---|---|
| 522. | 1 | MAKLTILVALALELL | Peptide_001 |
| 523. | 2 | LTILVALALFLLAAH | Peptide_002 |
| 524. | 3 | LVALALFLLAAHASA | Peptide_003 |
| 525. | 4 | LALFLLAAHASARQQ | Peptide_004 |
| 526. | 5 | FLLAAHASARQQWEL | Peptide_005 |
| 527. | 6 | AAHASARQQWELQGD | Peptide_006 |
| 528. | 7 | ASARQQWELQGDRRC | Peptide_007 |
| 529. | 8 | RQQWELQGDRRCQSQ | Peptide_008 |
| 530. | 9 | WELQGDRRCQSQLER | Peptide_009 |
| 531. | 10 | QGDRRCQSQLERANL | Peptide_010 |
| 532. | 11 | RRCQSQLERANLRPC | Peptide_011 |
| 533. | 12 | QSQLERANLRPCEQH | Peptide_012 |
| 534. | 13 | LERANLRPCEQHLMQ | Peptide_013 |
| 535. | 14 | ANLRPCEQHLMQKIQ | Peptide_014 |
| 536. | 15 | RPCEQHLMQKIQRDE | Peptide_015 |
| 537. | 16 | EQHLMQKIQRDEDSY | Peptide_016 |
| 538. | 17 | LMQKIQRDEDSYGRD | Peptide_017 |
| 539. | 18 | KIQRDEDSYGRDPYS | Peptide_018 |
| 540. | 19 | RDEDSYGRDPYSZSQ | Peptide_019 |
| 541. | 20 | DSYGRDPYSZSQDPY | Peptide_020 |
| 542. | 21 | GRDPYSZSQDPYSZS | Peptide_021 |
| 543. | 22 | PYSZSQDPYSZSQDP | Peptide_022 |
| 544. | 23 | SQDPYSZSQDPDRR | Peptide_023 |
| 545. | 24 | DPYSZSQDPDRRDPY | Peptide_024 |
| 546. | 25 | SZSQDPDRRDPYSZS | Peptide_025 |
| 547. | 26 | QDPDRRDPYSZSPYD | Peptide_026 |
| 548. | 27 | DRRDPYSZSPYDRRG | Peptide_027 |
| 549. | 28 | DPYSZSPYDRRGAGS | Peptide_028 |
| 550. | 29 | SZSPYDRRGAGSSQH | Peptide_029 |
| 551. | 30 | PYDRRGAGSSQHQER | Peptide_030 |
| 552. | 31 | RRGAGSSQHQERCCN | Peptide_031 |
| 553. | 32 | AGSSQHQERCCNELN | Peptide_032 |
| 554. | 33 | SQHQERCCNELNEFE | Peptide_033 |
| 555. | Index | Peptide Sequence | Annotation |
| 556. | 34 | QERCCNELNEFENNQ | Peptide_034 |
| 557. | 35 | CCNELNEFENNQRCM | Peptide_035 |
| 558. | 36 | ELNEFENNQRCMCEA | Peptide_036 |
| 559. | 37 | EFENNQRCMCEALQQ | Peptide_037 |
| 560. | 38 | NNQRCMCEALQQIME | Peptide_038 |
| 561. | 39 | RCMCEALQQIMENQS | Peptide_039 |
| 562. | 40 | CEALQQIMENQSDRL | Peptide_040 |
| 563. | 41 | LQQIMENQSDRLQGR | Peptide_041 |
| 564. | 42 | IMENQSDRLQGRQQE | Peptide_042 |
| 565. | 43 | NQSDRLQGRQQEQQF | Peptide_043 |
| 566. | 44 | DRLQGROQEQQFKRE | Peptide_044 |
| 567. | 45 | QGRQQEQQFKRELRN | Peptide_045 |
| 568. | 46 | QQEQQFKRELRNLPQ | Peptide_046 |
| 569. | 47 | QQFKRELRNLPQQCG | Peptide_047 |
| 570. | 48 | KRELRNLPQQCGLRA | Peptide_048 |
| 571. | 49 | LRNLPQQCGLRAPQR | Peptide_049 |
| 572. | 50 | LPQQCGLRAPQRCDL | Peptide_050 |
| 573. | 51 | QCGLRAPQRCDLEVE | Peptide_051 |
| 574. | 52 | LRAPQRCDLEVESGG | Peptide_052 |
| 575. | 53 | PQRCDLEVESGGRDR | Peptide_053 |
| 576. | 54 | QRCDLEVESGGRDRY | Peptide_054 |
| 577. | 55 | RDEDSYGRDPYSPSQ | Peptide_055 |
| 578. | 56 | DSYGRDPYSPSQDPY | Peptide_056 |
| 579. | 57 | GRDPYSPSQDPYSPS | Peptide_057 |
| 580. | 58 | PYSPSQDPYSPSQDP | Peptide_058 |
| 581. | 59 | PSQDPYSPSQDPDRR | Peptide_059 |
| 582. | 60 | DPYSPSQDPDRRDPY | Peptide_060 |
| 583. | 61 | SPSQDPDRRDPYSPS | Peptide_061 |
| 584. | 62 | QDPDRRDPYSPSPYD | Peptide_062 |
| 585. | 63 | DRRDPYSPSPYDRRG | Peptide_063 |
| 586. | 64 | DPYSPSPYDRRGAGS | Peptide_064 |
| 587. | 65 | SPSPYDRRGAGSSQH | Peptide_065 |

Full-length human IgG, human IgE and mouse IgG were co-immobilized on microarray slides as assay controls.

### Assay Conditions

The peptide mapping using microarrays was performed with 40 antibody samples diluted 1:1000 in blocking buffer (see below). Sample dilutions were incubated for 1 hour at 30° C. on multiwell microarray slides. The slides contained 21 individual mini-arrays (1 mini-array per sample dilution).

Subsequent to sample incubation, secondary fluorescently labeled anti-human-IgG antibody at 1 μg/ml was added into the corresponding wells and left to react for 1 hour.

Additional control incubations applying the secondary antibody only was performed in parallel on the microarray slides to assess false-positive binding to peptides.

After washing and drying, the slides were scanned with a high-resolution laser scanner at 635 nm to obtain fluorescence intensity profiles. Resulting images were quantified to yield a mean pixel value for each peptide.

### Samples

The following samples in Table 4 were used for antibody profiling:

TABLE 4

| | Applied |
|---|---|
| | sample |
| Samples: | dilution |

| | | |
|---|---|---|
| 1 | P6 | 1:1000 |
| 2 | P7 | 1:1000 |
| 3 | 111BU7P1A12 | 1:1000 |
| 4 | 111BU7P1D2 | 1:1000 |
| 5 | T6 | 1:1000 |
| 6 | 24BU7P1D4 | 1:1000 |
| 7 | 105BU7P1D6 | 1:1000 |
| 8 | 105BU7P1D8 | 1:1000 |
| 9 | 111BU7P1D5 | 1:1000 |
| 10 | 23FU1P1D8 | Sample not |
| | | available |
| 11 | 13FU1P1A4 | 1:1000 |
| 12 | 13FU1P2B12 | 1:1000 |
| 13 | P34 | 1:1000 |
| 14 | T1 | 1:1000 |
| 15 | T3 | 1:1000 |
| 16 | T4 | 1:1000 |
| 17 | T5 | 1:1000 |
| 18 | P31 | 1:1000 |
| 19 | 13FU1P2B10 | 1:1000 |
| 20 | 27FU1P3A10 | 1:1000 |
| 21 | 14FU1P1D6 | 1:1000 |
| 22 | S4 | 1:1000 |
| 23 | S1 | 1:1000 |
| 24 | 27FU1P3A4 | 1:1000 |
| 25 | P22 | 1:1000 |
| 26 | P8 | 1:1000 |
| 27 | 24BU7P1D3 | 1:1000 |
| 28 | 6BU4P2B1 | 1:1000 |
| 29 | 23FU1P1C10 | 1:1000 |
| 30 | 15FU1P3A1 | 1:1000 |
| 31 | 15FU1P3A6 | 1:1000 |
| 32 | 24BU7P1B1 | 1:1000 |
| 33 | P16 | 1:1000 |

The secondary antibody 1 was anti human IgG (H+L) (Jackson Immunoresearch 109-605-098). The label was ALEXA® Fluor 647. The applied concentration of secondary antibody was 1 μg/mL.

### Buffers and Solutions

50 mM TBS-buffer including 0.1% TWEEN® 20 (JPT), pH 7.2
Blocking buffer (Pierce International, Superblock TBS T20)

### Hardware and Software

Peptide microarrays (JPT Peptide Technologies GmbH, Berlin, Germany; batch #3399)
Axon Genepix Scanner 4300 SL50
Spot-recognition software GenePix
Microsoft Excel, R

### Assay

The assay was performed using a Multiwell incubation chamber.
Samples were diluted in blocking buffer
Diluted samples were applied to microarrays for 1 h at 30° C.
Microarrays were incubated with secondary antibody diluted in blocking buffer for 1 h at 30° C.
Microarrays were dried.

Before each step, microarrays were washed with washing buffer.

### Quantification

Microarrays were scanned using a high resolution fluorescence scanner. Laser settings and applied resolution were identical for all performed measurements. The resulting images were analyzed und quantified using spot-recognition software GenePix (Molecular Devices). For each spot, the mean signal intensity was extracted (between 0 and 65535 arbitrary units).

For further data evaluation, the MMC2 values were determined. The MMC2 equals the mean value of all three instances on the microarray except when the coefficient of variation (CV)—standard-deviation divided by the mean value—is larger 0.5. In this case the mean of the two closest values (MC2) is assigned to MMC2.

### Results and Data Evaluation

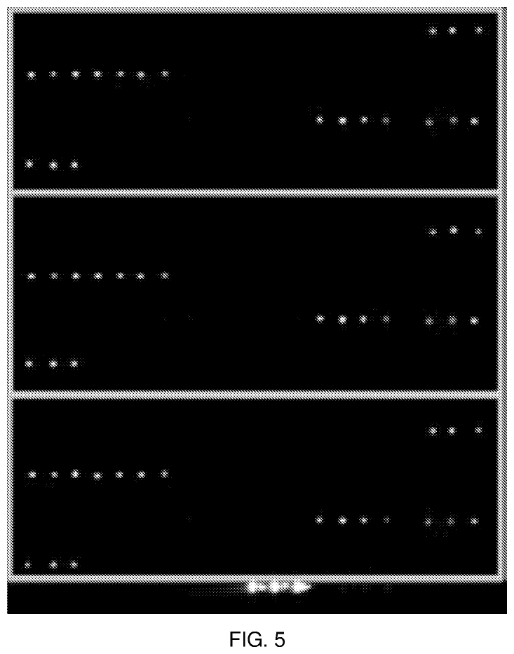

An example of a fluorescence readout image of one mini-array reflecting typical microarray incubation is shown in FIG. 5. Only low background levels were observed for all sample dilutions.

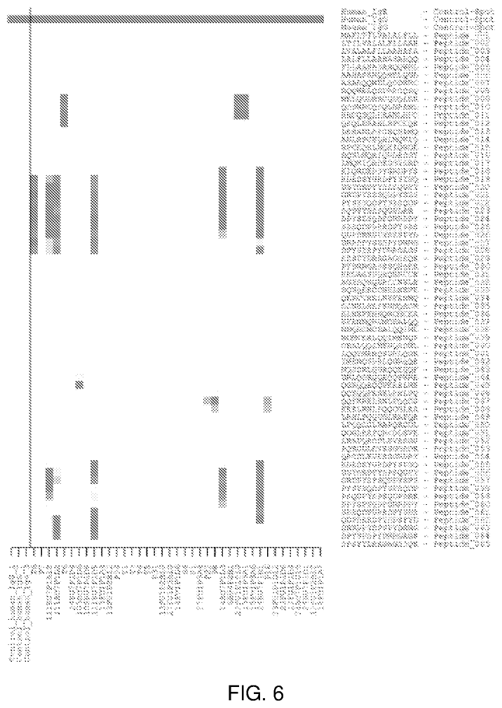

To visualize obtained results and to compare binding regions across the individual incubations, a heatmap diagram (FIG. 6) was computed showing fluorescence intensities in a color-coded manner from white (no binding) to red (strong binding). For all evaluations the MMC2-value of the mean pixel intensity for each peptide was calculated (please refer to paragraph 6.5 for details of calculation). The thick black line on the heatmap in FIG. 6 separates control incubations applying anti-human-IgG secondary antibody only.

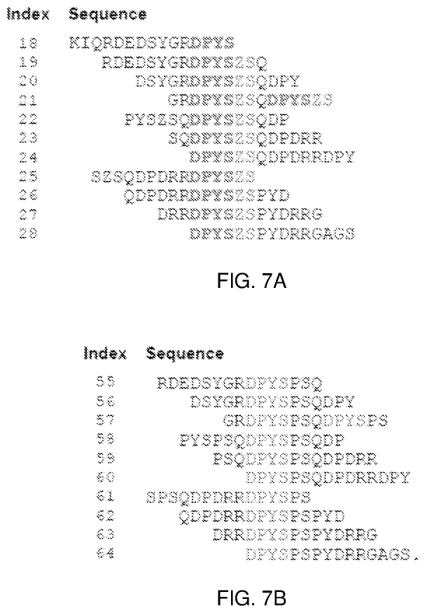

The obtained heatmap revealed a highly significant binding of P6, 111BU7P1A12, 111BU7P1D2, 111BU7P1D5, 24BU7P1D3, 24BU7P1B1 and a weak binding of P7 to the peptides shown in FIG. 7A. Overlapping peptides 19-28 contain a common motif “DPYSZS” which may represent a specific minimal epitope of the antibodies. Interestingly, in addition to peptides 19-28, samples 24BU7P1D3 and 24BU7P1B1 demonstrated a strong binding also to peptide 18. This suggests that both antibodies recognize even a shorter epitope “DPYS.” Motif “DPYS” is also present in peptides 55-64 as shown in FIG. 7B.

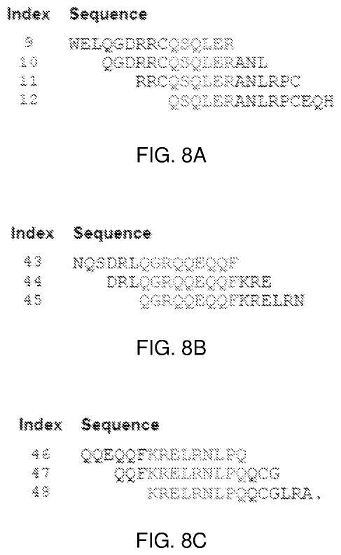

All antibodies described above bind to these peptides though to different extents. Antibodies T6, 24BU7P1D4, 23FU1P1C10 and 15FU1P3A1 demonstrated another common interaction profile comprising peptides 9-12, as shown in FIG. 8A. These peptides share the common binding motif “QSQLER”. Besides, signals of different intensity were measured for antibodies 105BU7P1D6 and 105BU7P1D8 with peptides 44-45, as shown in FIG. 8B, and or antibodies P22, P8 and P16 with peptides 46-48, as shown in FIG. 8C.

All remaining antibody samples showed no significant binding to the peptide library immobilized on the microarray.

Highly significant signals were obtained at control spots containing full-length human IgG demonstrating a good assay performance.

All control incubations containing detection antibody alone revealed no unspecific interactions with peptides.

### Summary

Antibodies P6, 111BU7P1A12, 111BU7P1D2, 111BU7P1D5 showed a highly significant interaction with overlapping peptides 19-28 representing the epitope “DPYSZS.”

Samples 24BU7P1D3 and 24BU7P1B1 showed a strong interaction with the common motif “DPYS' present in peptides 18-28 and 55-64.

Antibodies T6, 24BU7P1D4, 23FU1P1C10 and 15FU1P3A1 demonstrated a strong binding to peptides 9-12 which comprised a common motif “QSQLER.”

Only weak interactions were detected in the control incubation containing secondary antibody only.

Highly significant signals were obtained at control spots containing full-length human IgG demonstrating a good assay performance.

### Example 7—Summary of Arm h 2 Conformational Epitope Bins and Linear Epitopes Bound by Selected Anti-Arm h 2 Antibodies

Table 5 shows a summary of Aa h 2 epitope bins and linear epitopes bound by selected anti-Ara h 2 antibodies. The numbering of Ara h 2 residues is relative to the numbering in SEQ ID NO: 441:

MAKLTILVALALFLLAAHASARQQWELQGDRRCQSQLERANLRPCEQHLM
QKIQRDEDSYGRDPYSPSQDPYSPSQDPDRRDPYSPSPYDRRGAGSSQHQ
ERCCNELNEFENNQRCMCEALQQIMENQSDRLQGRQQEQQFKRELRNLPQ
QCGLRAPQRCDLEVESGGRDRY.

TABLE 5

| Antibody | Bin/Linear Epitope | Ara h 2 residues |
|---|---|---|
| P3 | 1 | 33-56, 114-132 |
| P6 | DPYSZS | 63-75 |
| P7 | DPYSZS | 63-75 |
| P8 | KRELRNLPQ | 142-150 |
| P10 | 1 | 33-56, 114-132 |
| P11 | 1 | 33-56, 114-132 |
| P13 | 1 | 33-56, 114-132 |
| P14 | 1 | 33-56, 114-132 |
| P16 | KRELRNLPQ | 142-150 |
| P17 | 1 | 33-56, 114-132 |
| P19 | 1 | 33-56, 114-132 |
| P22 | KRELRNLPQ | 142-150 |
| P21 | 1 | 33-56, 114-132 |
| P28 | 1 | 33-56, 114-132 |
| P30 | 1 | 33-56, 114-132 |
| P31 | 2 | 57-111 |
| P33 | 1 | 33-56, 114-132 |
| P34 | 1 | 33-56, 114-132 |
| P39 | 1 | 33-56, 114-132 |
| S1 | 3 | 34, 37-46, 119, 123-128 |
| S4 | 2 | 57-111 |
| 24B7D4 | QSQLER | 34-39 |

### Example 8—Ara h 2 Epitope Recognition by High Affinity Antibodies

Additional studies to evaluate Ara h 2 epitope recognition by high affinity antibodies were performed.

FIG. 9A shows a representative grid of conformational Ara h 2 epitopes as determined by BLI. Average nm increase of binding antibodies was 0.77 nm (standard deviation (SD)=0.25) and non-binding antibodies was 0.04 (SD 0.05). FIG. 9B shows an inset of experimental results from the experiments summarized in FIG. 9A. Secondary antibodies were grouped into a separate epitope if binding was ≥0.3 nm (≥6× the SD of non-binding antibodies). Three distinct conformational epitopes, Bin 1, 2, and 3, were identified using this method.

FIG. 9C shows a map of cloned monoclonal antibodies (SU Ab=36; TD Ab=44) from both SU and TD patients (n=9 and n=10 respectively). The majority (SU=56%; TD=48%) of antibodies bound to the Bin 1 epitope and antibodies from all three conformational bins were found in both groups.

FIG. 9D shows BLI results showing simultaneous binding of three monoclonal antibodies, one from each conformational bin and the linear DPYSP^{OH}S epitope. The DPYSP^{OH}S epitope is the same as DPYSZS. The “—OH” refers to a hydroxylproline important in binding.

### Example 9—Additional Summary of Ara h 2 Conformational Epitope Bins and Linear Epitopes Bound by Selected Anti-Ara h 2 Antibodies

Table 6 shows a summary of Ara h 2 epitope bins and linear epitopes bound by selected anti-Ara h 2 antibodies. Table 6 shows the clone name, epitope bin, chain, and patient outcome (i.e., non-tolerant or tolerant). For Bin 1 antibodies, Table 6 also shows whether the antibody belongs in a special sub-bin of Bin 1 which is only found in tolerant individuals. In general, antibodies that are indicated as being non-tolerant are particularly useful for diagnostic assays, while antibodies indicated as being tolerant are particularly useful as therapeutic antibodies. The right-hand column of Table 6 identifies antibodies that are expected to be especially advantageous as therapeutic antibodies.

It is to be understood that the antibodies disclosed herein can be reformatted into other antibody chains, e.g., IgG (e.g., IgG1, IgG2, IgG3, or IgG4), IgD, IgE, IgA, or IgM.

TABLE 6

| | | | | | Espe- |
|---|---|---|---|---|---|
| | | | | | cially |
| | | | | | Advan- |
| | | | | | tageous |
| Clone | Epitope | | | Bin1 | Thera- |
| Name | Bin | Chain | Outcome | SubBin | peutic |
| 22BU2S1 | Bin3 | IgH | non-tolerant | | |
| 22BU2S4 | Bin2 | IgH | non-tolerant | | |
| 21BU2U1 | Bin1 | IgH | non-tolerant | | |
| 23BU2P3 | Bin1 | IgH | non-tolerant | | |
| 23BU2P6 | DPYSZS | IgH | non-tolerant | | Yes |
| 23BU2P7 | DPYSZS | IgH | non-tolerant | | Yes |
| 23BU2P8 | KRELRNLPQ | IgH | non-tolerant | | Yes |
| 23BU2P10 | Bin1 | IgH | non-tolerant | | |
| 23BU2P11 | Bin1 | IgH | non-tolerant | | |
| 23BU2P13 | Bin1 | IgH | non-tolerant | | |
| 23BU2P14 | Bin1 | IgH | non-tolerant | | |
| 23BU2P16 | KRELRNLPQ | IgH | non-tolerant | | Yes |
| 23BU2P17 | Bin1 | IgH | non-tolerant | | |
| 23BU2P19 | Bin1 | IgH | non-tolerant | | |
| 23BU2P21 | Bin1 | IgH | non-tolerant | | |
| 23BU2P22 | KRELRNLPQ | IgH | non-tolerant | | Yes |
| 23BU2P30 | Bin1 | IgH | non-tolerant | | |
| 23BU2P31 | Bin2 | IgH | non-tolerant | | |
| 23BU2P33 | Bin1 | IgH | non-tolerant | | |
| 23BU2P34 | Bin1 | IgH | non-tolerant | | |
| 23BU2P39 | Bin1 | IgH | non-tolerant | | |
| 13BU2T1 | Bin1 | IgH | tolerant | Yes | Yes |
| 13BU2T3 | Bin1 | IgH | tolerant | | Yes |
| 13BU2T4 | Bin2 | IgH | tolerant | | Yes |
| 13BU2T5 | Bin2 | IgH | tolerant | | Yes |
| 13BU2T6 | QSQLER | IgH | tolerant | | |
| 13FU1P1A4 | Bin1 | IgH | tolerant | Yes | Yes |
| 13FU1P1B4 | Bin1 | IgH | tolerant | Yes | Yes |
| 14FU2P1A11 | Bin1 | IgH | tolerant | | |
| 14FU2P1D6 | Bin2 | IgH | tolerant | | |
| 15FU1P1A3 | Bin1 | IgH | tolerant | | |
| 15FU1P3A1 | QSQLER | IgH | tolerant | | Yes |
| 15FU1P3A6 | Bin2 | IgH | tolerant | | |
| 13FU1P2B10 | Bin2 | IgH | tolerant | | Yes |
| 13FU1P2B12 | Bin1 | IgH | tolerant | | Yes |
| 27FU1P3A4 | Bin3 | IgH | tolerant | | |
| 27FU1P3A10 | Bin2 | IgH | tolerant | | |
| 6BU4P2B1 | Bin3 | IgH | tolerant | | Yes |
| 11FUP1A2 | Bin1 | IgH | non-tolerant | | |
| 18FU1P1A7 | Bin1 | IgH | non-tolerant | | |
| 23FUP1A8 | Bin1 | IgH | non-tolerant | | |
| 23FUP1B8 | Bin1 | IgH | non-tolerant | | |
| 23FUP1C4 | Bin1 | IgH | non-tolerant | | |
| 23FUP1C10 | QSQLER | IgH | non-tolerant | | |
| 23FUP1D6 | Bin1 | IgH | non-tolerant | | |
| 23FUP1D8 | QSQLER | IgH | non-tolerant | | |
| 23FUP1D12 | Bin1 | IgH | non-tolerant | | |
| 24BU7P1A10 | Bin1 | IgH | tolerant | | |
| 24BU7P1B6 | Bin1 | IgH | tolerant | | |
| 24BU7P1D1 | Bin1 | IgH | tolerant | | |
| 24BU7P1D4 | QSQLER | IgH | tolerant | | Yes |
| 24BU7P1C10 | Bin1 | IgH | tolerant | | |
| 24BU7P1D3 | DPYS | IgH | tolerant | | |
| 24BU7P1D9 | Bin1 | IgH | tolerant | | |
| 24BU7P1B1 | DPYS | IgH | tolerant | | Yes |
| 24BU7P1C2 | Bin1 | IgH | tolerant | | |
| 105BU7P1A11 | Bin1 | IgH | tolerant | | |
| 105BU7P1C3 | Bin1 | IgH | tolerant | Yes | |
| 105BU7P1D6 | QGRQQEQQF | IgH | tolerant | | Yes |
| 105BU7P1D7 | Bin1 | IgH | tolerant | | |
| 105BU7P1D8 | QGRQQEQQF | IgH | tolerant | | Yes |
| 105BU7P1D12 | Bin1 | IgH | tolerant | Yes | |
| 111BU7P1A12 | DPYSZS | IgH | tolerant | | Yes |
| 111BU7P1D2 | DPYSZS | IgH | tolerant | | Yes |
| 111BU7P1D5 | DPYSZS | IgH | tolerant | | Yes |
| 33BU7P1D11 | Bin1 | IgH | non-tolerant | | |
| 89BU7P1B10 | Bin3 | IgH | non-tolerant | | |
| 29BU7P1D1 | CEALQQ | IgH | non-tolerant | | Yes |

Table 7 shows a table listing alternative nomenclature for certain antibodies disclosed herein. The “alternative clone names” represent alternative names for the same antibody. For example, “P3” is the same antibody as “23BU2P3.”

TABLE 7

| Clone | Alternative |
|---|---|
| Name | Clone Name |
| P3 | 23BU2P3 |
| P6 | 23BU2P6 |
| P7 | 23BU2P7 |
| P8 | 23BU2P8 |
| P10 | 23BU2P10 |
| P11 | 23BU2P11 |
| P13 | 23BU2P13 |
| P14 | 23BU2P14 |
| P16 | 23BU2P16 |
| P17 | 23BU2P17 |
| P19 | 23BU2P19 |
| P21 | 23BU2P21 |
| P22 | 23BU2P22 |
| P28 | 23BU2P28 |
| P30 | 23BU2P30 |
| P31 | 23BU2P31 |
| P33 | 23BU2P33 |
| P34 | 23BU2P34 |
| P39 | 23BU2P39 |
| S1 | 22BU2S1 |
| S4 | 22BU2S4 |

Therefore, the present disclosure provides anti-Ara h 2 antibodies and combinations thereof useful for diagnostic and therapeutic purposes, e.g., for diagnosis and treatment of peanut allergy.

Table 8 below provides the sequences of the above-mentioned antibodies, peptides, and Ara h 2. For polypeptide sequences, X is any natural occurring amino acid. For nucleotide sequences n is A, T, G, C or U.

TABLE 8

| SEQ ID NO | | P3 |
|---|---|---|
| 1. | CDR-H1 | GFIFADYT |
| 2. | CDR-H2 | ISWNSGGV |
| 3. | CDR-H3 | VKDNGYRAFDL |
| 4. | CDR-L1 | QSLVHSNGYNY |
| 5. | CDR-L2 | MGS |
| 6. | CDR-L3 | MQSLQTWT |
| 7. | VH | QVQLVESGGGLVQPGRSLRLSCAASGFIFADYTMHWVRQSPG |
| | | KDLEWVSRISWNSGGVEYADSVKGRFTISRDNAKNSLYLQMNS |
| | | LRVEDTALYYCVKDNGYRAFDLWGLGTMVTVSS |
| 8. | VL | DVVMTQSPVSLPVTPGEPASISCRSSQSLVHSNGYNYLDWYLQ |
| | | KPGQSPQLLIYMGSNRASGVPDRFSGSGSGTDFTLKISRVEAED |
| | | VGVYYCMQSLQTWTFGQGTKVEIK |
| 9. | FR-H1 | QVQLVESGGGLVQPGRSLRLSCAAS |
| 10. | FR-H2 | MHWVRQSPGKDLEWVSR |
| 11. | FR-H3 | EYADSVKGRFTISRDNAKNSLYLQMNSLRVEDTALYYC |
| 12. | FR-H4 | WGLGTMVTVSS |
| 13. | FR-L1 | DVVMTQSPVSLPVTPGEPASISCRSS |
| 14. | FR-L2 | LDWYLQKPGQSPQLLIY |
| 15. | FR-L3 | NRASGVPDRFSGSGSGTDFTLKISRVEAEDVGVYYC |
| 16. | FR-L4 | FGQGTKVEIK |
| 17. | heavychain | QVQLVESGGGLVQPGRSLRLSCAASGFIFADYTMHWVRQSPG |
| | | KDLEWVSRISWNSGGVEYADSVKGRFTISRDNAKNSLYLQMNS |
| | | LRVEDTALYYCVKDNGYRAFDLWGLGTMVTVSSKGPSVFPLAP |
| | | SSKSTSGGTAALGXLVKDYFPEPVTVSWNSGALTSGVHTFPAVL |
| | | QSSGLYSLSSVVTVPSSSLGTQTYICNVNHKPSNTKVDKXVEPK |
| | | SCDKTHTCPPCPAPELLGGPSVFLFPPKPKDTLMISRTPEVTCV |
| | | VVDVSHEDPEVKFNWYVDGVEVHNAKTKPREEQYNSTYRVVS |
| | | VLTVLHQDWLNGKEYKCKVSNKALPAPIEKTISKAKGQPREPQV |
| | | YTLPPSRDEXTKNQVSLTCLVKGFYPSDIAVEWESNGQPENNY |
| | | KTTPPVLDSDGSFFLYSKLTVDKSRWQQGNVFSCSVMHEALHN |
| | | HYTQKSLSLSPGK |
| 18. | lightchain | DVVMTQSPVSLPVTPGEPASISCRSSQSLVHSNGYNYLDWYLQ |
| | | KPGQSPQLLIYMGSNRASGVPDRFSGSGSGTDFTLKISRVEAED |
| | | VGVYYCMQSLQTWTFGQGTKVEIKAPSVFIFPPSDEQLKSGTAS |
| | | VVCLLNNFYPREAKVQWKVDNALQSGNSQESVTEQDSKDSTYS |
| | | LSSTLTLSKADYEKHKVYACEVTHQGLSSPVTKSFNRGEC*K |
| 19. | VH_nuc | caggtgcagctggtggagtctgggggaggcttggtacaacctggcaggtccctgagactc |
| | | tcctgtgcagcctctggattcatttttgccgattataccatgcactgggtccggcaaagtccag |
| | | ggaaggacctggagtgggtctctagaattagttggaatagtgggggcgtagagtatgcgg |
| | | actctgtgaagggccgattcaccatctccagagacaacgccaagaactccctctatcttca |
| | | aatgaacagtctgagagttgaagacacggccttatattactgtgtaaaagataatggttatc |
| | | gtgcatttgatctttggggcctagggacaatggtcaccgtctcttcag |
| 20. | VL_nuc | gatgttgtgatgactcagtctccagtctccctgcccgtcacccctggagagccggcctccatc |
| | | tcctgcaggtctagtcagagcctcgtgcatagtaatggatacaactatttggattggtacctgc |
| | | agaagccagggcagtctccacagctcctgatctatatgggttcaaatcgggcctccggggt |
| | | ccctgacaggttcagtggcagtggatcaggcacagattttacactgaaaatcagcagagt |
| | | ggaggctgaggatgttggagtttattactgcatgcaaagtctacaaacgtggacgttcggcc |
| | | aagggaccaaggtggaaatcaaac |
| SEQ ID NO | | P6 |
| 21. | CDR-H1 | GFSFEDYG |
| 22. | CDR-H2 | INWNGQST |
| 23. | CDR-H3 | ARVGRGVTGGGIRAFDI |
| 24. | CDR-L1 | QSISSDY |
| 25. | CDR-L2 | GAS |
| 26. | CDR-L3 | QKYSNSPVIT |
| 27. | VH | XVQLVESGGSLVRPGGSVRLSCTASGFSFEDYGMTWVRQGPG |
| | | MGLEWVSGINWNGQSTGYTDSVKGRFTISRDDAKNSLHLQMN |
| | | NLRVEDTALYYCARVGRGVTGGGIRAFDIWGQGTMVTVSP |
| 28. | VL | EIXXTQSPGTLSMSPGERATLSCRASQSISSDYLAWYQHKPGQ |
| | | APRLLIYGASSRATGIPDRFSGSGSGTDFTLTISRLEPEDFAVYY |
| | | CQKYSNSPVITFGQGTRLEIK |
| 29. | FR-H1 | XVQLVESGGSLVRPGGSVRLSCTAS |
| 30. | FR-H2 | MTWVRQGPGMGLEWVSG |
| 31. | FR-H3 | GYTDSVKGRFTISRDDAKNSLHLQMNNLRVEDTALYYC |
| 32. | FR-H4 | WGQGTMVTVSP |
| 33. | FR-L1 | EIXXTQSPGTLSMSPGERATLSCRAS |
| 34. | FR-L2 | LAWYQHKPGQAPRLLIY |
| 35. | FR-L3 | SRATGIPDRFSGSGSGTDFTLTISRLEPEDFAVYYC |
| 36. | FR-L4 | FGQGTRLEIK |
| 37. | heavychain | XVQLVESGGSLVRPGGSVRLSCTASGFSFEDYGMTWVRQGPG |
| | | MGLEWVSGINWNGQSTGYTDSVKGRFTISRDDAKNSLHLQMN |
| | | NLRVEDTALYYCARVGRGVTGGGIRAFDIWGQGTMVTVSPKGP |
| | | SVFPLAPSSKSTSGGTAALGXLVKDYFPEPVTVSWNSGALTSGV |
| | | HTFPAVLQSSGLYSLSSVVTVPSSSLGTQTYICNVNHKPSNTKV |
| | | DKXVEPKSCDKTHTCPPCPAPELLGGPSVFLFPPKPKDTLMISR |
| | | TPEVTCVVVDVSHEDPEVKFNWYVDGVEVHNAKTKPREEQYNS |
| | | TYRVVSVLTVLHQDWLNGKEYKCKVSNKALPAPIEKTISKAKGQ |
| | | PREPQVYTLPPSRDEXTKNQVSLTCLVKGFYPSDIAVEWESNG |
| | | QPENNYKTTPPVLDSDGSFFLYSKLTVDKSRWQQGNVFSCSVM |
| | | HEALHNHYTQKSLSLSPGK |
| 38. | lightchain | EIXLTQSPATLSLSPGERATLSCRASQSLGNYLAWYQQKPGQAP |
| | | RLLIYDASDRATGIPARFSGSGSGTDFTLTISSLEPEDFAVYYCQ |
| | | QRSQFMWTFGQGTKVEIKAPSVFIFPPSDEQLKSGTASVVCLLN |
| | | NFYPREAKVQWKVDNALQSGNSQESVTEQDSKDSTYSLSSTLT |
| | | LSKADYEKHKVYACEVTHQGLSSPVTKSFNRGEC*K |
| 39. | VH_nuc | nnngtgcagctggtggagtcggggggaagtttggtacggccgggggggtccgtgagact |
| | | ctcctgtacagcctctggatttagttttgaagactatggcatgacctgggtccgccaaggtcc |
| | | agggatggggctggagtgggtctccggtattaattggaatggtcagagtacaggttacaca |
| | | gactctgtgaagggccgattcaccatctccagagacgacgccaagaactccctgcatcta |
| | | caaatgaacaatctgagagtcgaggatacggccctatattattgtgcgagagtagggagg |
| | | ggagttactggcgggggaatcagggcttttgacatctggggccaagggacaatggtcacc |
| | | gtctctccag |
| 40. | VL_nuc | gaaattgngttgacgcagtctccagccaccctgtctttgtctccaggggaaagagccactct |
| | | ctcctgcagggccagtcagagtcttggcaactacttagcctggtaccaacagaaacctggc |
| | | caggctcccaggctcctcatctatgatgcatccgaccgggccactggcatcccagccaggt |
| | | tcagtggcagtgggtctgggacagacttcactctcaccatcagcagccttgagcctgaaga |
| | | ttttgcagtttattactgtcagcagcgtagccaatttatgtggacgttcggccaagggaccaa |
| | | ggtggaaatcaaac |
| SEQ ID NO | | P7 |
| 41. | CDR-H1 | GFTFTRYA |
| 42. | CDR-H2 | ISHDGGTK |
| 43. | CDR-H3 | AKTCSSPSCYDTAYYFDY |
| 44. | CDR-L1 | QSLGNY |
| 45. | CDR-L2 | DAS |
| 46. | CDR-L3 | QQRSQFMWT |
| 47. | VH | QVQLVESGGGVVQPGRSLRLSCVVSGFTFTRYAFHWVRQAPG |
| | | KGLEWVAVISHDGGTKNYADSVEGRFTISRDNSESALYLQMNSL |
| | | RPEDTAIYYCAKTCSSPSCYDTAYYFDYWGQGTPVTVSS |
| 48. | VL | EIXLTQSPATLSLSPGERATLSCRASQSLGNYLAWYQQKPGQAP |
| | | RLLIYDASDRATGIPARFSGSGSGTDFTLTISSLEPEDFAVYYCQ |
| | | QRSQFMWTFGQGTKVEIK |
| 49. | FR-H1 | QVQLVESGGGVVQPGRSLRLSCVVS |
| 50. | FR-H2 | FHWVRQAPGKGLEWVAV |
| 51. | FR-H3 | NYADSVEGRFTISRDNSESALYLQMNSLRPEDTAIYYC |
| 52. | FR-H4 | WGQGTPVTVSS |
| 53. | FR-L1 | EIXLTQSPATLSLSPGERATLSCRAS |
| 54. | FR-L2 | LAWYQQKPGQAPRLLIY |
| 55. | FR-L3 | DRATGIPARFSGSGSGTDFTLTISSLEPEDFAVYYC |
| 56. | FR-L4 | FGQGTKVEIK |
| 57. | heavychain | QVQLVESGGGVVQPGRSLRLSCVVSGFTFTRYAFHWVRQAPG |
| | | KGLEWVAVISHDGGTKNYADSVEGRFTISRDNSESALYLQMNSL |
| | | RPEDTAIYYCAKTCSSPSCYDTAYYFDYWGQGTPVTVSSKGPS |
| | | VFPLAPSSKSTSGGTAALGXLVKDYFPEPVTVSWNSGALTSGV |
| | | HTFPAVLQSSGLYSLSSVVTVPSSSLGTQTYICNVNHKPSNTKV |
| | | DKXVEPKSCDKTHTCPPCPAPELLGGPSVFLFPPKPKDTLMISR |
| | | TPEVTCVVVDVSHEDPEVKFNWYVDGVEVHNAKTKPREEQYNS |
| | | TYRVVSVLTVLHQDWLNGKEYKCKVSNKALPAPIEKTISKAKGQ |
| | | PREPQVYTLPPSRDEXTKNQVSLTCLVKGFYPSDIAVEWESNG |
| | | QPENNYKTTPPVLDSDGSFFLYSKLTVDKSRWQQGNVFSCSVM |
| | | HEALHNHYTQKSLSLSPGK |
| 58. | lightchain | EIXXTQSPGTLSLSPGERATLSCTASQRVNSDSVAWYQQRPGL |
| | | APRLLIYDASHRATGIPDRFSGGRGGTGFTLTIRALEPEDFAVYY |
| | | CQQYGESPLTFGQGTKVEIKAPSVFIFPPSDEQLKSGTASVVCLL |
| | | NNFYPREAKVQWKVDNALQSGNSQESVTEQDSKDSTYSLSSTL |
| | | TLSKADYEKHKVYACEVTHQGLSSPVTKSFNRGEC*K |
| 59. | VH_nuc | caggtgcagctggtggagtctgggggaggcgtggtccagcctgggaggtccctgagact |
| | | ctcctgtgtagtctctggattcaccttcactaggtatgcttttcactgggtccgccaggctccag |
| | | gcaaggggctggagtgggtggcagttatatcacatgatggaggcactaaaaactacgca |
| | | gactccgtggagggccgattcaccatctccagagacaattccgagagcgcactctatctgc |
| | | aaatgaacagcctgagacctgaggacacggctatatattactgtgcgaaaacttgtagtag |
| | | tcccagttgttatgatacggcatactactttgactactggggccagggaaccccggtcaccg |
| | | tctcctcag |
| 60. | VL_nuc | gaaatagngtngacgcagtctccaggcaccctgtctttgtctccaggcgagagggccacc |
| | | ctctcctgcacggccagtcagagagtgaatagcgactccgtagcctggtatcagcagaga |
| | | cctggcctggcgcccaggctcctcatctatgatgcatcccacagggccactggcatcccag |
| | | acaggttcagtggcggtaggggtgggacaggcttcactctcaccatcagggcactggagc |
| | | ctgaagattttgcagtatattactgtcaacagtatggtgagtcacctctaacgttcggccaag |
| | | ggaccaaggtggaaatcaaac |
| SEQ ID NO | | P8 |
| 61. | CDR-H1 | GFSFTGSA |
| 62. | CDR-H2 | VQSYSHSFAT |
| 63. | CDR-H3 | TRPFSGYDLMSDFFPN |
| 64. | CDR-L1 | QRVNSDS |
| 65. | CDR-L2 | DAS |
| 66. | CDR-L3 | QQYGESPLT |
| 67. | VH | XCXLVESGGDLVQPGGSLKLSCATSGFSFTGSAIHWVRQSSGK |
| | | GLEWLGRVQSYSHSFATAYSASLEGRFTISRDESENTAYLQMN |
| | | SLKPEDTAVYYCTRPFSGYDLMSDFFPNWGQGTLVTVSS |
| 68. | VL | EIXXTQSPGTLSLSPGERATLSCTASQRVNSDSVAWYQQRPGL |
| | | APRLLIYDASHRATGIPDRFSGGRGGTGFTLTIRALEPEDFAVYY |
| | | CQQYGESPLTFGQGTKVEIK |
| 69. | FR-H1 | XCXLVESGGDLVQPGGSLKLSCATS |
| 70. | FR-H2 | IHWVRQSSGKGLEWLGR |
| 71. | FR-H3 | AYSASLEGRFTISRDESENTAYLQMNSLKPEDTAVYYC |
| 72. | FR-H4 | WGQGTLVTVSS |
| 73. | FR-L1 | EIXXTQSPGTLSLSPGERATLSCTAS |
| 74. | FR-L2 | VAWYQQRPGLAPRLLIY |
| 75. | FR-L3 | HRATGIPDRFSGGRGGTGFTLTIRALEPEDFAVYYC |
| 76. | FR-L4 | FGQGTKVEIK |
| 77. | heavychain | XCXLVESGGDLVQPGGSLKLSCATSGFSFTGSAIHWVRQSSGK |
| | | GLEWLGRVQSYSHSFATAYSASLEGRFTISRDESENTAYLQMN |
| | | SLKPEDTAVYYCTRPFSGYDLMSDFFPNWGQGTLVTVSSKGPS |
| | | VFPLAPSSKSTSGGTAALGXLVKDYFPEPVTVSWNSGALTSGV |
| | | HTFPAVLQSSGLYSLSSVVTVPSSSLGTQTYICNVNHKPSNTKV |
| | | DKXVEPKSCDKTHTCPPCPAPELLGGPSVFLFPPKPKDTLMISR |
| | | TPEVTCVVVDVSHEDPEVKFNWYVDGVEVHNAKTKPREEQYNS |
| | | TYRVVSVLTVLHQDWLNGKEYKCKVSNKALPAPIEKTISKAKGQ |
| | | PREPQVYTLPPSRDEXTKNQVSLTCLVKGFYPSDIAVEWESNG |
| | | QPENNYKTTPPVLDSDGSFFLYSKLTVDKSRWQQGNVFSCSVM |
| | | HEALHNHYTQKSLSLSPGK |
| 78. | lightchain | GCXVTQSPISLPVTPGEPASISCRSSQSLIHSNGYNYLDWYLQK |
| | | PGQSPQLLISLGSKRASGVPERFSGSGSGTDFTLKISRVEAEDV |
| | | GIYYCMQALQTPWTFGQGTKVEIKAPSVFIFPPSDEQLKSGTAS |
| | | VVCLLNNFYPREAKVQWKVDNALQSGNSQESVTEQDSKDSTYS |
| | | LSSTLTLSKADYEKHKVYACEVTHQGLSSPVTKSFNRGEC*K |
| 79. | VH_nuc | nngtgcannctggtggagtctgggggagacttggtccagcctggggggtccctaaaactc |
| | | tcctgtgcaacctctggtttttccttcaccggctctgccatccactgggtccgccagtcttccgg |
| | | gaaaggcctggaatggcttggccgagttcaaagttattctcacagtttcgcgacagcttattct |
| | | gcgtcgctggaaggcaggttcaccatctccagagatgagtcagagaacacggcgtatctc |
| | | caaatgaatagcctgaaaccggaggacacggccgtctattattgtacaagacctttctcag |
| | | gttacgatttgatgagtgatttttttcccaactggggccagggaaccctggtcaccgtctcctc |
| | | ag |
| 80. | VL_nuc | ggatgttnngtgactcagtctccaatatccctgcccgtcacccctggagagccggcctccat |
| | | ctcttgcaggtctagtcagagcctcatacatagtaatggatacaattacttggattggtacctg |
| | | cagaagccagggcagtctccacagctcctgatctctttgggctctaagcgggcctccgggg |
| | | tccctgagaggttcagtggcagtggatcaggcacagattttacactgaaaatcagcagagt |
| | | ggaggctgaggatgttgggatttattactgcatgcaagctctacaaactccgtggacgttcg |
| | | gccaagggaccaaggtggaaatcaaac |
| SEQ ID NO | | P10 |
| 81. | CDR-H1 | GFTFEDYT |
| 82. | CDR-H2 | ISWKGGAI |
| 83. | CDR-H3 | VKDNGFRSFDS |
| 84. | CDR-L1 | QSLIHSNGYNY |
| 85. | CDR-L2 | LGS |
| 86. | CDR-L3 | MQALQTPWT |
| 87. | VH | XVXXVESGGDLVQPGRSLRLSCVISGFTFEDYTMHWVRLVPGK |
| | | GLEWVSGISWKGGAIGYADSVKGRFTISRDNGKNSLHLQMNSL |
| | | RPEDTALYHCVKDNGFRSFDSWGRGTLVAVSS |
| 88. | VL | GCXVTQSPISLPVTPGEPASISCRSSQSLIHSNGYNYLDWYLQK |
| | | PGQSPQLLISLGSKRASGVPERFSGSGSGTDFTLKISRVEAEDV |
| | | GIYYCMQALQTPWTFGQGTKVEIK |
| 89 | FR-H1 | XVXXVESGGDLVQPGRSLRLSCVIS |
| 90. | FR-H2 | MHWVRLVPGKGLEWVSG |
| 91. | FR-H3 | GYADSVKGRFTISRDNGKNSLHLQMNSLRPEDTALYHC |
| 92. | FR-H4 | WGRGTLVAVSS |
| 93. | FR-L1 | GCXVTQSPISLPVTPGEPASISCRSS |
| 94. | FR-L2 | LDWYLQKPGQSPQLLIS |
| 95. | FR-L3 | KRASGVPERFSGSGSGTDFTLKISRVEAEDVGIYYC |
| 96. | FR-L4 | FGQGTKVEIK |
| 97. | heavychain | XVXXVESGGDLVQPGRSLRLSCVISGFTFEDYTMHWVRLVPGK |
| | | GLEWVSGISWKGGAIGYADSVKGRFTISRDNGKNSLHLQMNSL |
| | | RPEDTALYHCVKDNGFRSFDSWGRGTLVAVSSKGPSVFPLAPS |
| | | SKSTSGGTAALGXLVKDYFPEPVTVSWNSGALTSGVHTFPAVL |
| | | QSSGLYSLSSVVTVPSSSLGTQTYICNVNHKPSNTKVDKXVEPK |
| | | SCDKTHTCPPCPAPELLGGPSVFLFPPKPKDTLMISRTPEVTCV |
| | | VVDVSHEDPEVKFNWYVDGVEVHNAKTKPREEQYNSTYRVVS |
| | | VLTVLHQDWLNGKEYKCKVSNKALPAPIEKTISKAKGQPREPQV |
| | | YTLPPSRDEXTKNQVSLTCLVKGFYPSDIAVEWESNGQPENNY |
| | | KTTPPVLDSDGSFFLYSKLTVDKSRWQQGNVFSCSVMHEALHN |
| | | HYTQKSLSLSPGK |
| 98. | lightchain | RCXXTQSPLSLPVTPGEPASISCRSSQSLLHSNGYNYLDWYLQK |
| | | PGQSPQLLIYFGSNRASGVPDRFSGSGSGTDFTLNITRVEAEDV |
| | | GVYYCMQALQSWTFGQGTKVEIKAPSVFIFPPSDEQLKSGTASV |
| | | VCLLNNFYPREAKVQWKVDNALQSGNSQESVTEQDSKDSTYSL |
| | | SSTLTLSKADYEKHKVYACEVTHQGLSSPVTKSFNRGEC*K |
| 99. | VH_nuc | nnngtgcnnnnggtggagtctgggggagatctggtacagcctggcaggtccctgagact |
| | | ctcttgtgtaatctctggattcacttttgaagattacaccatgcactgggtccggctagttccag |
| | | ggaagggcctggagtgggtctcaggtataagttggaaaggtggtgccataggctatgcgg |
| | | actctgtgaagggccggttcaccatctccagagacaacggcaagaactccctgcatctgc |
| | | aaatgaacagtctgagacctgaggacacggccttatatcactgtgtgaaagataatggtttt |
| | | cggtcctttgattcctggggccggggaaccctggtcgccgtctcctcag |
| 100. | VL_nuc | cgatgttnngngactcagtctccactctccctgcccgtcacccctggagagccggcctccat |
| | | ctcctgcaggtctagtcagagcctcctgcatagtaatggatacaactatttggattggtacctg |
| | | cagaagccagggcagtctccacaactcctgatctatttcggttctaatcgggcctccggggt |
| | | ccctgacaggttcagtggcagtggatcaggcacagattttacactgaacatcaccagagtg |
| | | gaggctgaggatgttggggtttattactgcatgcaagctctacaaagttggacgttcggcca |
| | | agggaccaaggtggaaatcaaac |
| SEQ ID NO | | P11 |
| 101. | CDR-H1 | GFTFRDYG |
| 102. | CDR-H2 | IRYDERNK |
| 103. | CDR-H3 | VKDSGLRYFNL |
| 104. | CDR-L1 | QSLLHSNGYNY |
| 105. | CDR-L2 | FGS |
| 106. | CDR-L3 | MQALQSWT |
| 107. | VH | XVQLVESGGGVVQPGGSLRLSCAASGFTFRDYGMHWVRQAPG |
| | | KGLEWVAFIRYDERNKYYVDSVKGRFTVSRDNSKSTLYLQMNS |
| | | LRAEDTAVYYCVKDSGLRYFNLWGRGTLVTVSS |
| 108. | VL | RCXXTQSPLSLPVTPGEPASISCRSSQSLLHSNGYNYLDWYLQK |
| | | PGQSPQLLIYFGSNRASGVPDRFSGSGSGTDFTLNITRVEAEDV |
| | | GVYYCMQALQSWTFGQGTKVEIK |
| 109. | FR-H1 | XVQLVESGGGVVQPGGSLRLSCAAS |
| 110. | FR-H2 | MHWVRQAPGKGLEWVAF |
| 111. | FR-H3 | YYVDSVKGRFTVSRDNSKSTLYLQMNSLRAEDTAVYYC |
| 112. | FR-H4 | WGRGTLVTVSS |
| 113. | FR-L1 | RCXXTQSPLSLPVTPGEPASISCRSS |
| 114. | FR-L2 | LDWYLQKPGQSPQLLIY |
| 115. | FR-L3 | NRASGVPDRFSGSGSGTDFTLNITRVEAEDVGVYYC |
| 116. | FR-L4 | FGQGTKVEIK |
| 117. | heavychain | XVQLVESGGGVVQPGGSLRLSCAASGFTFRDYGMHWVRQAPG |
| | | KGLEWVAFIRYDERNKYYVDSVKGRFTVSRDNSKSTLYLQMNS |
| | | LRAEDTAVYYCVKDSGLRYFNLWGRGTLVTVSSKGPSVFPLAP |
| | | SSKSTSGGTAALGXLVKDYFPEPVTVSWNSGALTSGVHTFPAVL |
| | | QSSGLYSLSSVVTVPSSSLGTQTYICNVNHKPSNTKVDKXVEPK |
| | | SCDKTHTCPPCPAPELLGGPSVFLFPPKPKDTLMISRTPEVTCV |
| | | VVDVSHEDPEVKFNWYVDGVEVHNAKTKPREEQYNSTYRVVS |
| | | VLTVLHQDWLNGKEYKCKVSNKALPAPIEKTISKAKGQPREPQV |
| | | YTLPPSRDEXTKNQVSLTCLVKGFYPSDIAVEWESNGQPENNY |
| | | KTTPPVLDSDGSFFLYSKLTVDKSRWQQGNVFSCSVMHEALHN |
| | | HYTQKSLSLSPGK |
| 118. | lightchain | DVXMTQSPLSLPVTPGEPASISCRSSQSLLHSNGYNYLDWYLQ |
| | | KPGQSPQLLIYFGSNRASGVPDRFSGSGSGTDFTLKISRVEAED |
| | | VGVYYCMQAQQTPWTFGQGTKVEIKAPSVFIFPPSDEQLKSGT |
| | | ASVVCLLNNFYPREAKVQWKVDNALQSGNSQESVTEQDSKDST |
| | | YSLSSTLTLSKADYEKHKVYACEVTHQGLSSPVTKSFNRGEC*K |
| 119. | VH_nuc | nnngtgcagctggtggagtctgggggaggcgtggtccagcctggggggtccctgagact |
| | | ctcctgtgcagcgtctggattcaccttcagagactatggcatgcactgggtccgccaggctc |
| | | caggcaaggggctggagtgggtggcatttatacgatatgatgagagaaataaatattatgt |
| | | agactccgtgaagggccgattcaccgtctccagagacaactccaagagcacactgtatct |
| | | ccaaatgaacagcctcagagctgaggacacggctgtatattactgtgtgaaagattccgg |
| | | gttgaggtacttcaatctctggggccgtggcaccctggtcaccgtctcctcag |
| 120. | VL_nuc | gatgttgngatgactcagtctccactctccctgcccgtcacccctggagagccggcctccat |
| | | ctcctgcaggtctagtcagagcctcctccatagtaatggatacaactatttggattggtacctg |
| | | cagaagccagggcagtctccacagctcctgatctatttcggttctaatcgggcctccggggt |
| | | ccctgacaggttcagtggcagtggatcaggcacagattttacactgaaaatcagcagagt |
| | | ggaggctgaggatgttggggtttattactgcatgcaagctcaacaaactccgtggacgttcg |
| | | gccaagggaccaaggtggaaatcaaac |
| SEQ ID NO | | P13 |
| 121. | CDR-H1 | GFTFSDYA |
| 122. | CDR-H2 | IRFDGTKI |
| 123. | CDR-H3 | AKDSGFRSFEV |
| 124. | CDR-L1 | QSLLHSNGYNY |
| 125. | CDR-L2 | FGS |
| 126. | CDR-L3 | MQAQQTPWT |
| 127. | VH | XCTLVQSGGGVVPPGGSLRLSCAASGFTFSDYAMHWVRQAPG |
| | | KGLEWVTFIRFDGTKIDYKDSVKGRFTISRDDSKNTLYLEMNTLS |
| | | TEDTAVYFCAKDSGFRSFEVWGRGTLVTVSS |
| 128. | VL | DVXMTQSPLSLPVTPGEPASISCRSSQSLLHSNGYNYLDWYLQ |
| | | KPGQSPQLLIYFGSNRASGVPDRFSGSGSGTDFTLKISRVEAED |
| | | VGVYYCMQAQQTPWTFGQGTKVEIK |
| 129. | FR-H1 | XCTLVQSGGGVVPPGGSLRLSCAAS |
| 130. | FR-H2 | MHWVRQAPGKGLEWVTF |
| 131. | FR-H3 | DYKDSVKGRFTISRDDSKNTLYLEMNTLSTEDTAVYFC |
| 132. | FR-H4 | WGRGTLVTVSS |
| 133. | FR-L1 | DVXMTQSPLSLPVTPGEPASISCRSS |
| 134. | FR-L2 | LDWYLQKPGQSPQLLIY |
| 135. | FR-L3 | NRASGVPDRFSGSGSGTDFTLKISRVEAEDVGVYYC |
| 136. | FR-L4 | FGQGTKVEIK |
| 137. | heavychain | XCTLVQSGGGVVPPGGSLRLSCAASGFTFSDYAMHWVRQAPG |
| | | KGLEWVTFIRFDGTKIDYKDSVKGRFTISRDDSKNTLYLEMNTLS |
| | | TEDTAVYFCAKDSGFRSFEVWGRGTLVTVSSKGPSVFPLAPSS |
| | | KSTSGGTAALGXLVKDYFPEPVTVSWNSGALTSGVHTFPAVLQ |
| | | SSGLYSLSSVVTVPSSSLGTQTYICNVNHKPSNTKVDKXVEPKS |
| | | CDKTHTCPPCPAPELLGGPSVFLFPPKPKDTLMISRTPEVTCVV |
| | | VDVSHEDPEVKFNWYVDGVEVHNAKTKPREEQYNSTYRVVSVL |
| | | TVLHQDWLNGKEYKCKVSNKALPAPIEKTISKAKGQPREPQVYT |
| | | LPPSRDEXTKNQVSLTCLVKGFYPSDIAVEWESNGQPENNYKTT |
| | | PPVLDSDGSFFLYSKLTVDKSRWQQGNVFSCSVMHEALHNHYT |
| | | QKSLSLSPGK |
| 138. | lightchain | DIXXTQSPLSLPVTPGEPASISCRSSQSLLHSNGYNYLDWYLQK |
| | | PGQSPQLLISLGSNRASGVPDRFSGSGSGTDFTLKISRVEAEDV |
| | | GVYYCMQALQTWTFGQGTKVEIKAPSVFIFPPSDEQLKSGTASV |
| | | VCLLNNFYPREAKVQWKVDNALQSGNSQESVTEQDSKDSTYSL |
| | | SSTLTLSKADYEKHKVYACEVTHQGLSSPVTKSFNRGEC*K |
| 139. | VH_nuc | nggtgcacgctggtgcagtctgggggaggcgtagtcccgcccggggggtccctgagact |
| | | ctcctgtgcagcgtctggattcaccttcagtgactatgccatgcactgggtccgccaggctcc |
| | | aggcaaggggctggagtgggtgacatttatacgatttgatggaactaaaatagactataaa |
| | | gactccgtgaagggccgcttcaccatctccagagacgattccaagaacaccctttatctgg |
| | | agatgaacaccctgagtactgaagacacggctgtgtatttctgtgcgaaagattcaggttttc |
| | | ggtccttcgaggtctggggccgtggcaccctggtcactgtctcctcag |
| 140. | VL_nuc | gatattgnngngactcagtctccactctccctgcccgtcacccctggagagccggcctccat |
| | | ctcctgcaggtctagccagagcctcctgcatagtaatggatacaactatttggattggtacct |
| | | gcagaagccagggcagtctccacagctcctgatctctttgggttctaatcgggcctccgggg |
| | | tccctgacaggttcagtggcagtggatcaggcacagattttacactgaaaatcagcagagt |
| | | ggaggctgaggatgttggggtttattactgcatgcaagctctacaaacttggacgttcggcc |
| | | aagggaccaaggtggaaatcaaac |
| SEQ ID NO | | P14 |
| 141. | CDR-H1 | GFTFDDYT |
| 142. | CDR-H2 | IKWNSRAI |
| 143. | CDR-H3 | VKDTGLRSFHS |
| 144. | CDR-L1 | QSLLHSNGYNY |
| 145. | CDR-L2 | LGS |
| 146. | CDR-L3 | MQALQTWT |
| 147. | VH | QVXXVESGGGLVQPGGSLRLSCAASGFTFDDYTMHWVRQPPG |
| | | KGLEWVSSIKWNSRAIDYADSVKGRFTISRDNAKNSLFLQMNTL |
| | | RTEDTAFYYCVKDTGLRSFHSWGQGTLLTVSS |
| 148. | VL | DIXXTQSPLSLPVTPGEPASISCRSSQSLLHSNGYNYLDWYLQK |
| | | PGQSPQLLISLGSNRASGVPDRFSGSGSGTDFTLKISRVEAEDV |
| | | GVYYCMQALQTWTFGQGTKVEIK |
| 149. | FR-H1 | QVXXVESGGGLVQPGGSLRLSCAAS |
| 150. | FR-H2 | MHWVRQPPGKGLEWVSS |
| 151. | FR-H3 | DYADSVKGRFTISRDNAKNSLFLQMNTLRTEDTAFYYC |
| 152. | FR-H4 | WGQGTLLTVSS |
| 153. | FR-L1 | DIXXTQSPLSLPVTPGEPASISCRSS |
| 154. | FR-L2 | LDWYLQKPGQSPQLLIS |
| 155. | FR-L3 | NRASGVPDRFSGSGSGTDFTLKISRVEAEDVGVYYC |
| 156. | FR-L4 | FGQGTKVEIK |
| 157. | heavychain | QVXXVESGGGLVQPGGSLRLSCAASGFTFDDYTMHWVRQPPG |
| | | KGLEWVSSIKWNSRAIDYADSVKGRFTISRDNAKNSLFLQMNTL |
| | | RTEDTAFYYCVKDTGLRSFHSWGQGTLLTVSSKGPSVFPLAPS |
| | | SKSTSGGTAALGXLVKDYFPEPVTVSWNSGALTSGVHTFPAVL |
| | | QSSGLYSLSSVVTVPSSSLGTQTYICNVNHKPSNTKVDKXVEPK |
| | | SCDKTHTCPPCPAPELLGGPSVFLFPPKPKDTLMISRTPEVTCV |
| | | VVDVSHEDPEVKFNWYVDGVEVHNAKTKPREEQYNSTYRVVS |
| | | VLTVLHQDWLNGKEYKCKVSNKALPAPIEKTISKAKGQPREPQV |
| | | YTLPPSRDEXTKNQVSLTCLVKGFYPSDIAVEWESNGQPENNY |
| | | KTTPPVLDSDGSFFLYSKLTVDKSRWQQGNVFSCSVMHEALHN |
| | | HYTQKSLSLSPGK |
| 158. | lightchain | RNXETQSPGTLSLSPGERATLSCTASQRVNSDSVAWYQQRPGL |
| | | APRLLIYDASHRATGIPDRFSGGRGGTGFTLTIRALEPEDFAVYY |
| | | CQQYGESPLTFGQGTKVEIKAPSVFIFPPSDEQLKSGTASVVCLL |
| | | NNFYPREAKVQWKVDNALQSGNSQESVTEQDSKDSTYSLSSTL |
| | | TLSKADYEKHKVYACEVTHQGLSSPVTKSFNRGEC*K |
| 159. | VH_nuc | caggtgncnncggtggagtcggggggaggcttggtacagcctgggggtccctgagact |
| | | ctcctgtgcagcctctggattcacctttgatgattacaccatgcattgggtccggcaacctcca |
| | | gggaagggcctggagtgggtctcaagtatcaagtggaacagtcgtgccatagactatgcg |
| | | gactctgtgaagggccgattcaccatctccagagacaacgccaagaactccctgtttctgc |
| | | aaatgaatactctgagaactgaggacacggccttctattactgtgtgaaggatacgggact |
| | | acggtcctttcactcctggggccagggaaccctgctcaccgtctcctcag |
| 160. | VL_nuc | cgaaatanngagacgcagtctccaggcaccctgtctttgtctccaggcgagagggccacc |
| | | ctctcctgcacggccagtcagagagtgaatagcgactccgtagcctggtatcagcagaga |
| | | cctggcctggcgcccaggctcctcatctatgatgcatcccacagggccactggcatcccag |
| | | acaggttcagtggcggtaggggtgggacaggcttcactctcaccatcagggcactggagc |
| | | ctgaagattttgcagtatattactgtcaacagtatggtgagtcacctctaacgttcggccaag |
| | | ggaccaaggtggaaatcaaac |
| SEQ ID NO | | P16 |
| 161. | CDR-H1 | GFSFTGSA |
| 162. | CDR-H2 | VQSYSHSFAT |
| 163. | CDR-H3 | TRPFSGYDLMSDFFPN |
| 164. | CDR-L1 | QRVNSDS |
| 165. | CDR-L2 | DAS |
| 166. | CDR-L3 | QQYGESPLT |
| 167. | VH | XVXXVESGGDLVQPGGSLKLSCATSGFSFTGSAIHWVRQSSGK |
| | | GLEWLGRVQSYSHSFATAYSASLEGRFTISRDESENTAYLQMNS |
| | | LKPEDTAVYYCTRPFSGYDLMSDFFPNWGQGTLVTVSS |
| 168. | VL | RNXETQSPGTLSLSPGERATLSCTASQRVNSDSVAWYQQRPGL |
| | | APRLLIYDASHRATGIPDRFSGGRGGTGFTLTIRALEPEDFAVYYC |
| | | QQYGESPLTFGQGTKVEIK |
| 169. | FR-H1 | XVXXVESGGDLVQPGGSLKLSCATS |
| 170. | FR-H2 | IHWVRQSSGKGLEWLGR |
| 171. | FR-H3 | AYSASLEGRFTISRDESENTAYLQMNSLKPEDTAVYYC |
| 172. | FR-H4 | WGQGTLVTVSS |
| 173. | FR-L1 | RNXETQSPGTLSLSPGERATLSCTAS |
| 174. | FR-L2 | VAWYQQRPGLAPRLLIY |
| 175. | FR-L3 | HRATGIPDRFSGGRGGTGFTLTIRALEPEDFAVYYC |
| 176. | FR-L4 | FGQGTKVEIK |
| 177. | heavychain | XVXXVESGGDLVQPGGSLKLSCATSGFSFTGSAIHWVRQSSGK |
| | | GLEWLGRVQSYSHSFATAYSASLEGRFTISRDESENTAYLQMNS |
| | | LKPEDTAVYYCTRPFSGYDLMSDFFPNWGQGTLVTVSSKGPSVF |
| | | PLAPSSKSTSGGTAALGXLVKDYFPEPVTVSWNSGALTSGVHTF |
| | | PAVLQSSGLYSLSSVVTVPSSSLGTQTYICNVNHKPSNTKVDKXV |
| | | EPKSCDKTHTCPPCPAPELLGGPSVFLFPPKPKDTLMISRTPEVT |
| | | CVVVDVSHEDPEVKFNWYVDGVEVHNAKTKPREEQYNSTYRVV |
| | | SVLTVLHQDWLNGKEYKCKVSNKALPAPIEKTISKAKGQPREPQV |
| | | YTLPPSRDEXTKNQVSLTCLVKGFYPSDIAVEWESNGQPENNYK |
| | | TTPPVLDSDGSFFLYSKLTVDKSRWQQGNVFSCSVMHEALHNH |
| | | YTQKSLSLSPGK |
| 178. | lightchain | DIXXTQSPLSLPVTPGEPASISCRSSQSLLHSNGIHYLDWYLQKP |
| | | GQSPQLLIYLGSKRASGVPDRFSGSGSGTDFTLKISRVEAEDVGV |
| | | YYCMQSLQTFTFGPGTKVDIKAPSVFIFPPSDEQLKSGTASVVCL |
| | | LNNFYPREAKVQWKVDNALQSGNSQESVTEQDSKDSTYSLSSTL |
| | | TLSKADYEKHKVYACEVTHQGLSSPVTKSFNRGEC*K |
| 179. | VH_nuc | nnngtgcnnnnngtggagtctgggggagacttggtccagcctggggggtccctaaaactct |
| | | cctgtgcaacctctggtttttccttcaccggctctgccatccactgggtccgccagtcttccggg |
| | | aaaggcctggaatggcttggccgagttcaaagttattctcacagtttcgcgacagcttattctg |
| | | cgtcgctggaaggcaggttcaccatctccagagatgagtcagagaacacggcgtatctcca |
| | | aatgaatagcctgaaaccggaggacacggccgtctattattgtacaagacctttctcaggtta |
| | | cgatttgatgagtgatttttttcccaactggggccagggaaccctggtcaccgtctcctcag |
| 180. | VL_nuc | gatattgnngngactcagtctccactctccctgcccgtcacccctggagagccggcctccat |
| | | ctcctgcaggtcaagtcagagcctcctgcacagtaatggaatccactatttggattggtatctg |
| | | cagaagccagggcagtctccacagctcctgatctatttgggttctaaacgggcctccggggt |
| | | ccctgacaggttcagtggcagtggatcaggcacagattttacacttaaaatcagcagagtgg |
| | | aggctgaggatgttggggtttattactgcatgcaatctctacaaaccttcactttcggccctggg |
| | | accaaagtggatatcaaac |
| SEQ ID NO | | P17 |
| 181. | CDR-H1 | GFTFDDYT |
| 182. | CDR-H2 | IRYDGTRA |
| 183. | CDR-H3 | VKDGGLRYFDY |
| 184. | CDR-L1 | QSLLHSNGIHY |
| 185. | CDR-L2 | LGS |
| 186 | CDR-L3 | MQSLQTFT |
| 187. | VH | XCRLVESGGDVVQPGGSLRLSCEASGFTFDDYTMHWVRQVPGK |
| | | SLEWLSLIRYDGTRAEYADSVKGRFTISRDNSKNSLFLQMNSLKT |
| | | DDSAFYYCVKDGGLRYFDYWGQGTLVTVSS |
| 188. | VL | DIXXTQSPLSLPVTPGEPASISCRSSQSLLHSNGIHYLDWYLQKP |
| | | GQSPQLLIYLGSKRASGVPDRFSGSGSGTDFTLKISRVEAEDVGV |
| | | YYCMQSLQTFTFGPGTKVDIK |
| 189. | FR-H1 | XCRLVESGGDVVQPGGSLRLSCEAS |
| 190. | FR-H2 | MHWVRQVPGKSLEWLSL |
| 191. | FR-H3 | EYADSVKGRFTISRDNSKNSLFLQMNSLKTDDSAFYYC |
| 192. | FR-H4 | WGQGTLVTVSS |
| 193. | FR-L1 | DIXXTQSPLSLPVTPGEPASISCRSS |
| 194. | FR-L2 | LDWYLQKPGQSPQLLIY |
| 195 | FR-L3 | KRASGVPDRFSGSGSGTDFTLKISRVEAEDVGVYYC |
| 196. | FR-L4 | FGPGTKVDIK |
| 197. | heavychain | XCRLVESGGDVVQPGGSLRLSCEASGFTFDDYTMHWVRQVPGK |
| | | SLEWLSLIRYDGTRAEYADSVKGRFTISRDNSKNSLFLQMNSLKT |
| | | DDSAFYYCVKDGGLRYFDYWGQGTLVTVSSKGPSVFPLAPSSK |
| | | STSGGTAALGXLVKDYFPEPVTVSWNSGALTSGVHTFPAVLQSS |
| | | GLYSLSSVVTVPSSSLGTQTYICNVNHKPSNTKVDKXVEPKSCDK |
| | | THTCPPCPAPELLGGPSVFLFPPKPKDTLMISRTPEVTCVVVDVS |
| | | HEDPEVKFNWYVDGVEVHNAKTKPREEQYNSTYRVVSVLTVLH |
| | | QDWLNGKEYKCKVSNKALPAPIEKTISKAKGQPREPQVYTLPPSR |
| | | DEXTKNQVSLTCLVKGFYPSDIAVEWESNGQPENNYKTTPPVLD |
| | | SDGSFFLYSKLTVDKSRWQQGNVFSCSVMHEALHNHYTQKSLS |
| | | LSPGK |
| 198. | lightchain | DVGXTQSPVSLPVTPGEPASISCRSSQSLXHSNGYNYLDWYLQK |
| | | PGQSPQLLIYMGSIRASGVPDRFSGSGSGTDFTLKISRVEAEDVG |
| | | VYYCMQSLQTWTFGQGTKVEIKAPSVFIFPPSDEQLKSGTASVV |
| | | CLLNNFYPREAKVQWKVDNALQSGNSQESVTEQDSKDSTYSLS |
| | | STLTLSKADYEKHKVYACEVTHQGLSSPVTKSFNRGEC*K |
| 199. | VH_nuc | nngtgcaggctggtggagtctgggggagacgtggtgcagcctggggggtccctaagactct |
| | | cctgtgaagcctctggattcacctttgatgattatactatgcactgggtccgtcaagttccgggg |
| | | aagagtctggagtggctctctcttattcgttacgacgggactagggcagaatatgcagactcc |
| | | gtgaagggtcgattcaccatctccagagacaacagcaaaaactccctttttctgcaaatgaa |
| | | cagtctgaaaactgacgactccgccttctattattgtgtaaaagatggtggattacgatactttg |
| | | actactggggccagggcactctggtcaccgtctcctcag |
| 200. | VL_nuc | gatgttggngngactcagtctccagtctccctgcccgtcacccctggagagccggcctccat |
| | | ctcctgcaggtctagtcagagcctcntacatagtaatggatacaactatttggattggtacctg |
| | | cagaagccagggcagtctccacagctcctgatctatatgggttcaattcgggcctccggggt |
| | | ccctgacaggttcagtggcagtggatcaggcacagattttacactgaaaatcagcagagtg |
| | | gaggctgaggatgttggggtttattactgcatgcaaagtctacaaacgtggacgttcggccaa |
| | | gggaccaaggtggaaatcaaac |
| SEQ ID NO | | P19 |
| 201. | CDR-H1 | GFIFGDYT |
| 202. | CDR-H2 | ISWNSGSM |
| 203. | CDR-H3 | VKDNGYRAFDF |
| 204. | CDR-L1 | QSLXHSNGYNY |
| 205. | CDR-L2 | MGS |
| 206. | CDR-L3 | MQSLQTWT |
| 207. | VH | XVXXVESGGGLVQPGRSLRLSCAASGFIFGDYTMHWVRQTPGK |
| | | GLEWVSRISWNSGSMEYADSVKGRLTISRDNAKNSLHLQMNSLR |
| | | VEDTALYYCVKDNGYRAFDFWGQGTMVTVSS |
| 208. | VL | DVGXTQSPVSLPVTPGEPASISCRSSQSLXHSNGYNYLDWYLQK |
| | | PGQSPQLLIYMGSIRASGVPDRFSGSGSGTDFTLKISRVEAEDVG |
| | | VYYCMQSLQTWTFGQGTKVEIK |
| 209. | FR-H1 | XVXXVESGGGLVQPGRSLRLSCAAS |
| 210. | FR-H2 | MHWVRQTPGKGLEWVSR |
| 211. | FR-H3 | EYADSVKGRLTISRDNAKNSLHLQMNSLRVEDTALYYC |
| 212. | FR-H4 | WGQGTMVTVSS |
| 213. | FR-L1 | DVGXTQSPVSLPVTPGEPASISCRSS |
| 214. | FR-L2 | LDWYLQKPGQSPQLLIY |
| 215. | FR-L3 | IRASGVPDRFSGSGSGTDFTLKISRVEAEDVGVYYC |
| 216. | FR-L4 | FGQGTKVEIK |
| 217. | heavychain | XVXXVESGGGLVQPGRSLRLSCAASGFIFGDYTMHWVRQTPGK |
| | | GLEWVSRISWNSGSMEYADSVKGRLTISRDNAKNSLHLQMNSLR |
| | | VEDTALYYCVKDNGYRAFDFWGQGTMVTVSSKGPSVFPLAPSS |
| | | KSTSGGTAALGXLVKDYFPEPVTVSWNSGALTSGVHTFPAVLQS |
| | | SGLYSLSSVVTVPSSSLGTQTYICNVNHKPSNTKVDKXVEPKSCD |
| | | KTHTCPPCPAPELLGGPSVFLFPPKPKDTLMISRTPEVTCVVVDV |
| | | SHEDPEVKFNWYVDGVEVHNAKTKPREEQYNSTYRVVSVLTVLH |
| | | QDWLNGKEYKCKVSNKALPAPIEKTISKAKGQPREPQVYTLPPSR |
| | | DEXTKNQVSLTCLVKGFYPSDIAVEWESNGQPENNYKTTPPVLD |
| | | SDGSFFLYSKLTVDKSRWQQGNVFSCSVMHEALHNHYTQKSLS |
| | | LSPGK |
| 218. | lightchain | RCXXTQSPLSLPVTPGEPASISCRSSQSLLHSNGYNYLDWYLQK |
| | | PGQSPQLLIYLGSNRASGVPDRFSGSGSGTDFTLKISRVEAEDVG |
| | | VYYCMQALQRWTFGQGTKVEIKAPSVFIFPPSDEQLKSGTASVV |
| | | CLLNNFYPREAKVQWKVDNALQSGNSQESVTEQDSKDSTYSLS |
| | | STLTLSKADYEKHKVYACEVTHQGLSSPVTKSFNRGEC*K |
| 219. | VH_nuc | naggtgcangnggtggagtctgggggaggcttggtacagcctggcaggtccctgagactct |
| | | cctgtgcagcctctggattcatttttggcgattataccatgcactgggtccggcaaactccagg |
| | | gaagggcctggagtgggtctctagaattagttggaatagtggaagtatggaatatgcggact |
| | | ctgtgaagggccgactcaccatctccagagacaacgccaagaactccctccatcttcaaat |
| | | gaacagtctgagagttgaggacacggccttatattactgtgtaaaagataatggttatcgtgct |
| | | tttgatttttggggccaagggacaatggtcaccgtctcttcag |
| 220. | VL_nuc | cgatgttgngngactcagtctccactctccctgcccgtcacccctggagagccggcctccatc |
| | | tcctgcaggtctagtcagagcctcctgcatagtaatggatacaactatttggattggtacctgc |
| | | agaagccagggcagtctccacagctcctgatctatttgggttctaatcgggcctccggggtcc |
| | | ctgacaggttcagtggcagtggatcaggcacagattttacactgaaaatcagcagagtgga |
| | | ggctgaggatgttggggtttattactgcatgcaagctctacaaaggtggacgttcggccaagg |
| | | gaccaaggtggaaatcaaac |
| SEQ ID NO | | P21 |
| 221. | CDR-H1 | GFDISGSA |
| 222. | CDR-H2 | IRSRSHAFAT |
| 223. | CDR-H3 | TRPFRGYDLSSDFYPN |
| 224. | CDR-L1 | QTITSGS |
| 225. | CDR-L2 | DAS |
| 226. | CDR-L3 | QQYGETPQT |
| 227. | VH | GAXLVQSGGGLVQPGGSLKVSCAVSGFDISGSAIHWVRQTSGK |
| | | GLEWLGRIRSRSHAFATAYAPSVRGRFTISTDESKNTAFLMMNS |
| | | LNSDDTAVYYCTRPFRGYDLSSDFYPNWGQGTLVTVSS |
| 228. | VL | EIGETQSPGTLSLSPGEGATLSCRASQTITSGSLAWYQQRPGLA |
| | | PRLLIYDASTRATGIPKRFSGSGSGTDFTLTISRLEPEDFAVYYC |
| | | QQYGETPQTFGQGTKVEIK |
| 229. | FR-H1 | GAXLVQSGGGLVQPGGSLKVSCAVS |
| 230. | FR-H2 | IHWVRQTSGKGLEWLGR |
| 231. | FR-H3 | AYAPSVRGRFTISTDESKNTAFLMMNSLNSDDTAVYYC |
| 232. | FR-H4 | WGQGTLVTVSS |
| 233. | FR-L1 | EIGETQSPGTLSLSPGEGATLSCRAS |
| 234. | FR-L2 | LAWYQQRPGLAPRLLIY |
| 235. | FR-L3 | TRATGIPKRFSGSGSGTDFTLTISRLEPEDFAVYYC |
| 236. | FR-L4 | FGQGTKVEIK |
| 237. | heavychain | GAXLVQSGGGLVQPGGSLKVSCAVSGFDISGSAIHWVRQTSGK |
| | | GLEWLGRIRSRSHAFATAYAPSVRGRFTISTDESKNTAFLMMNS |
| | | LNSDDTAVYYCTRPFRGYDLSSDFYPNWGQGTLVTVSSKGPSV |
| | | FPLAPSSKSTSGGTAALGXLVKDYFPEPVTVSWNSGALTSGVHT |
| | | FPAVLQSSGLYSLSSVVTVPSSSLGTQTYICNVNHKPSNTKVDK |
| | | XVEPKSCDKTHTCPPCPAPELLGGPSVFLFPPKPKDTLMISRTP |
| | | EVTCVVVDVSHEDPEVKFNWYVDGVEVHNAKTKPREEQYNSTY |
| | | RVVSVLTVLHQDWLNGKEYKCKVSNKALPAPIEKTISKAKGQPR |
| | | EPQVYTLPPSRDEXTKNQVSLTCLVKGFYPSDIAVEWESNGQP |
| | | ENNYKTTPPVLDSDGSFFLYSKLTVDKSRWQQGNVFSCSVMHE |
| | | ALHNHYTQKSLSLSPGK |
| 238. | lightchain | DVVMTQSPLSLPVTPGEPASISCRSSQSLLHSNGYNYLDWYLQ |
| | | KPGQSPQLLIYFGSKRASGVPDRFSGSGSGTDFTLRISRVEAEDI |
| | | GVYYCMQAQQTPWTFGQGTKVEIKAPSVFIFPPSDEQLKSGTA |
| | | SVVCLLNNFYPREAKVQWKVDNALQSGNSQESVTEQDSKDSTY |
| | | SLSSTLTLSKADYEKHKVYACEVTHQGLSSPVTKSFNRGEC*K |
| 239. | VH_nuc | ggtgccnnnctggtgcagtctggcggaggactggtccagcctgggggatccctgaaagtc |
| | | tcctgtgcagtctctgggttcgacatcagcggctctgccatacactgggtccgccagacctc |
| | | cgggaaagggctggagtggcttggccgaattagaagcagatctcacgcttttgcgacggc |
| | | ctatgctccgtcggtgagaggcaggttcaccatttccacagatgagtcaaagaacacggc |
| | | attcttgatgatgaacagcctgaatagcgacgacacggccgtttactactgtactcgaccatt |
| | | tcgaggttatgatttgtcgagtgacttttatcccaactggggccagggaaccctggtcaccgt |
| | | ctcctcag |
| 240. | VL_nuc | gatgttgtgatgactcagtctccactctccctgcccgtcacccctggagagccggcctccatc |
| | | tcctgcaggtctagtcagagcctcttgcatagtaatggatacaactatttggattggtacctgc |
| | | agaagccagggcagtctccacaactcctgatctatttcggttctaaacgggcctccggggtc |
| | | cctgacaggttcagtggcagtggctcaggcacagattttacactgagaatcagcagagtg |
| | | gaggctgaggatattggggtttattactgcatgcaagctcaacagactccgtggacgttcgg |
| | | ccaagggaccaaggtggaaatcaaac |
| SEQ ID NO | | P22 |
| 241. | CDR-H1 | GFTFSDYS |
| 242. | CDR-H2 | IRYDGSNK |
| 243. | CDR-H3 | VKDSGLRAFEI |
| 244. | CDR-L1 | QSLLHSNGYNY |
| 245. | CDR-L2 | LGS |
| 246. | CDR-L3 | MQALQRWT |
| 247. | VH | QVXLVESGGGVVQPGGSLRLSCAASGFTFSDYSIHWVRQAPGK |
| | | GLEWVAFIRYDGSNKDYADSVKGRITISRDNSKNTLYLQMNSLR |
| | | AEDTAVYYCVKDSGLRAFEIWGPGTMVTVSS |
| 248. | VL | RCXXTQSPLSLPVTPGEPASISCRSSQSLLHSNGYNYLDWYLQK |
| | | PGQSPQLLIYLGSNRASGVPDRFSGSGSGTDFTLKISRVEAEDV |
| | | GVYYCMQALQRWTFGQGTKVEIK |
| 249. | FR-H1 | QVXLVESGGGVVQPGGSLRLSCAAS |
| 250. | FR-H2 | IHWVRQAPGKGLEWVAF |
| 251. | FR-H3 | DYADSVKGRITISRDNSKNTLYLQMNSLRAEDTAVYYC |
| 252. | FR-H4 | WGPGTMVTVSS |
| 253. | FR-L1 | RCXXTQSPLSLPVTPGEPASISCRSS |
| 254. | FR-L2 | LDWYLQKPGQSPQLLIY |
| 255. | FR-L3 | NRASGVPDRFSGSGSGTDFTLKISRVEAEDVGVYYC |
| 256. | FR-L4 | FGQGTKVEIK |
| 257. | heavychain | QVXLVESGGGVVQPGGSLRLSCAASGFTFSDYSIHWVRQAPGK |
| | | GLEWVAFIRYDGSNKDYADSVKGRITISRDNSKNTLYLQMNSLR |
| | | AEDTAVYYCVKDSGLRAFEIWGPGTMVTVSSKGPSVFPLAPSS |
| | | KSTSGGTAALGXLVKDYFPEPVTVSWNSGALTSGVHTFPAVLQ |
| | | SSGLYSLSSVVTVPSSSLGTQTYICNVNHKPSNTKVDKXVEPKS |
| | | CDKTHTCPPCPAPELLGGPSVFLFPPKPKDTLMISRTPEVTCVV |
| | | VDVSHEDPEVKFNWYVDGVEVHNAKTKPREEQYNSTYRVVSVL |
| | | TVLHQDWLNGKEYKCKVSNKALPAPIEKTISKAKGQPREPQVYT |
| | | LPPSRDEXTKNQVSLTCLVKGFYPSDIAVEWESNGQPENNYKTT |
| | | PPVLDSDGSFFLYSKLTVDKSRWQQGNVFSCSVMHEALHNHYT |
| | | QKSLSLSPGK |
| 258. | lightchain | EIGETQSPGTLSLSPGEGATLSCRASQTITSGSLAWYQQRPGLA |
| | | PRLLIYDASTRATGIPKRFSGSGSGTDFTLTISRLEPEDFAVYYC |
| | | QQYGETPQTFGQGTKVEIKAPSVFIFPPSDEQLKSGTASVVCLL |
| | | NNFYPREAKVQWKVDNALQSGNSQESVTEQDSKDSTYSLSSTL |
| | | TLSKADYEKHKVYACEVTHQGLSSPVTKSFNRGEC*K |
| 259. | VH_nuc | caggtgcanctggtggagtctgggggaggcgtggtccagcctggggggtccctgagact |
| | | ctcctgtgcagcgtctggattcaccttcagtgactatagcattcactgggtccgccaggctcc |
| | | aggcaaggggctggaatgggtggcatttataaggtatgatggaagtaataaagactatgc |
| | | agactccgtgaagggccgaataaccatctccagagacaattccaagaacaccctgtatct |
| | | gcaaatgaacagtctgagagctgaggacacggctgtgtattactgtgtgaaagattccgga |
| | | ctacgtgcttttgagatctggggcccagggacaatggtcaccgtctcttcag |
| 260. | VL_nuc | gaaataggngagacgcagtctccaggcaccctgtctttgtctccaggggaaggcgccac |
| | | cctctcctgtagggccagtcagactattaccagcggctctttagcctggtatcagcagagac |
| | | ctggcctggcgcccaggctcctcatctatgatgcgtccaccagggccactggcatcccaaa |
| | | gaggttcagtggcagtgggtctgggacagacttcactctcacaattagcagactggagcct |
| | | gaagattttgcagtatattactgtcagcaatatggtgaaacacctcaaacgttcggccaagg |
| | | gaccaaggtggagatcaaac |
| SEQ ID NO | | P28 |
| 261. | CDR-H1 | GFTFDDYT |
| 262. | CDR-H2 | ISWKSGSL |
| 263. | CDR-H3 | VKDSGLRSFDV |
| 264. | CDR-L1 | QSLLHSNGYNY |
| 265. | CDR-L2 | FGS |
| 266. | CDR-L3 | MQAQQTPWT |
| 267. | VH | XVQLVESGGGLVQPGRSLRISCEASGFTFDDYTMHWVRQTPGK |
| | | GLEWVSGISWKSGSLGYADSVKGRFTISRDNAKNSLYLEVHSLR |
| | | PEDSAFYYCVKDSGLRSFDVWGRGTLLTVSS |
| 268. | VL | DVVMTQSPLSLPVTPGEPASISCRSSQSLLHSNGYNYLDWYLQK |
| | | PGQSPQLLIYFGSKRASGVPDRFSGSGSGTDFTLRISRVEAEDIG |
| | | VYYCMQAQQTPWTFGQGTKVEIK |
| 269. | FR-H1 | XVQLVESGGGLVQPGRSLRISCEAS |
| 270. | FR-H2 | MHWVRQTPGKGLEWVSG |
| 271. | FR-H3 | GYADSVKGRFTISRDNAKNSLYLEVHSLRPEDSAFYYC |
| 272. | FR-H4 | WGRGTLLTVSS |
| 273. | FR-L1 | DVVMTQSPLSLPVTPGEPASISCRSS |
| 274. | FR-L2 | LDWYLQKPGQSPQLLIY |
| 275. | FR-L3 | KRASGVPDRFSGSGSGTDFTLRISRVEAEDIGVYYC |
| 276. | FR-L4 | FGQGTKVEIK |
| 277. | heavychain | XVQLVESGGGLVQPGRSLRISCEASGFTFDDYTMHWVRQTPGK |
| | | GLEWVSGISWKSGSLGYADSVKGRFTISRDNAKNSLYLEVHSLR |
| | | PEDSAFYYCVKDSGLRSFDVWGRGTLLTVSSKGPSVFPLAPSSK |
| | | STSGGTAALGXLVKDYFPEPVTVSWNSGALTSGVHTFPAVLQSS |
| | | GLYSLSSVVTVPSSSLGTQTYICNVNHKPSNTKVDKXVEPKSCDK |
| | | THTCPPCPAPELLGGPSVFLFPPKPKDTLMISRTPEVTCVVVDVS |
| | | HEDPEVKFNWYVDGVEVHNAKTKPREEQYNSTYRVVSVLTVLH |
| | | QDWLNGKEYKCKVSNKALPAPIEKTISKAKGQPREPQVYTLPPSR |
| | | DEXTKNQVSLTCLVKGFYPSDIAVEWESNGQPENNYKTTPPVLD |
| | | SDGSFFLYSKLTVDKSRWQQGNVFSCSVMHEALHNHYTQKSLS |
| | | LSPGK |
| 278. | lightchain | DIXETQSPSTLSASVGDRVTITCRASQSISSWLAWYQQKPGKAPK |
| | | LLIYRASRLESGVPSRFSGSGSGTEFTLTISSLQPDDFATYYCQQ |
| | | YDTYLITFGQGTRLEIKAPSVFIFPPSDEQLKSGTASVVCLLNNFY |
| | | PREAKVQWKVDNALQSGNSQESVTEQDSKDSTYSLSSTLTLSKA |
| | | DYEKHKVYACEVTHQGLSSPVTKSFNRGEC*K |
| 279. | VH_nuc | nnngtgcagctggtggagtctgggggaggcttggtacagcctggcaggtccctgagaatct |
| | | cctgtgaagcctctggattcacctttgatgattataccatgcactgggtccggcaaactccagg |
| | | gaagggcctggagtgggtctctggcattagttggaaaagtggtagcctaggctatgcggact |
| | | ctgtgaagggccggttcaccatctccagagacaacgccaagaactccctctatttggaagtg |
| | | cacagtctgagacctgaggactcggccttctattactgtgtaaaagatagtggactacggag |
| | | cttcgatgtctggggccggggcaccctgctcactgtctcctcag |
| 280. | VL_nuc | gacatcnnngagacccagtctccttccaccctgtctgcatctgtaggggacagagtcaccat |
| | | cacttgccgggccagtcagagtattagtagctggttggcctggtatcagcagaaaccaggg |
| | | aaagcccctaaactcctgatctatcgggcgtctcgtttagaaagtggggtcccatcaaggttc |
| | | agcggcagtggatctgggacagaattcactctcaccatcagcagcctgcagcctgatgatttt |
| | | gcaacttattactgccaacaatatgatacttacctgatcaccttcggccaagggacacgactg |
| | | gagattaaac |
| SEQ ID NO | | P30 |
| 281. | CDR-H1 | GFTFSNYA |
| 282. | CDR-H2 | MSGRGGRT |
| 283. | CDR-H3 | AKDLPSDNSGLNSAEFFHV |
| 284. | CDR-L1 | QSISSW |
| 285. | CDR-L2 | RAS |
| 286. | CDR-L3 | QQYDTYLIT |
| 287. | VH | EVQLVESGGGLVQPGGSVRLSCAASGFTFSNYAMSWVRQTPGK |
| | | GLEWVSGMSGRGGRTDYADSVKGRFTISRDSSNSTLYLQMNSLR |
| | | AEDTALYYCAKDLPSDNSGLNSAEFFHVWGQGALVTVSS |
| 288. | VL | DIXETQSPSTLSASVGDRVTITCRASQSISSWLAWYQQKPGKAPK |
| | | LLIYRASRLESGVPSRFSGSGSGTEFTLTISSLQPDDFATYYCQQY |
| | | DTYLITFGQGTRLEIK |
| 289. | FR-H1 | EVQLVESGGGLVQPGGSVRLSCAAS |
| 290. | FR-H2 | MSWVRQTPGKGLEWVSG |
| 291. | FR-H3 | DYADSVKGRFTISRDSSNSTLYLQMNSLRAEDTALYYC |
| 292. | FR-H4 | WGQGALVTVSS |
| 293. | FR-L1 | DIXETQSPSTLSASVGDRVTITCRAS |
| 294. | FR-L2 | LAWYQQKPGKAPKLLIY |
| 295. | FR-L3 | RLESGVPSRFSGSGSGTEFTLTISSLQPDDFATYYC |
| 296. | FR-L4 | FGQGTRLEIK |
| 297. | heavychain | EVQLVESGGGLVQPGGSVRLSCAASGFTFSNYAMSWVRQTPGK |
| | | GLEWVSGMSGRGGRTDYADSVKGRFTISRDSSNSTLYLQMNSLR |
| | | AEDTALYYCAKDLPSDNSGLNSAEFFHVWGQGALVTVSSKGPSV |
| | | FPLAPSSKSTSGGTAALGXLVKDYFPEPVTVSWNSGALTSGVHTF |
| | | PAVLQSSGLYSLSSVVTVPSSSLGTQTYICNVNHKPSNTKVDKXVE |
| | | PKSCDKTHTCPPCPAPELLGGPSVFLFPPKPKDTLMISRTPEVTCV |
| | | VVDVSHEDPEVKFNWYVDGVEVHNAKTKPREEQYNSTYRVVSVL |
| | | TVLHQDWLNGKEYKCKVSNKALPAPIEKTISKAKGQPREPQVYTL |
| | | PPSRDEXTKNQVSLTCLVKGFYPSDIAVEWESNGQPENNYKTTPP |
| | | VLDSDGSFFLYSKLTVDKSRWQQGNVFSCSVMHEALHNHYTQKS |
| | | LSLSPGK |
| 298. | lightchain | DIRETQSPSTLSASVGDRVTITCRASESISSWLAWYQQKPGKAPKL |
| | | LIYEASTLETGVPSRFSGSGSGTEFTLTIRSLQPDDFATYYCQHYN |
| | | SDSLTFGGGTKVEIKAPSVFIFPPSDEQLKSGTASVVCLLNNFYPR |
| | | EAKVQWKVDNALQSGNSQESVTEQDSKDSTYSLSSTLTLSKADY |
| | | EKHKVYACEVTHQGLSSPVTKSFNRGEC*K |
| 299. | VH_nuc | gaggtgcagctggtggagtctgggggaggcttggtacagccgggggggtccgtgagactct |
| | | cctgtgcagcctctggattcacctttagtaattatgccatgagctgggtccgccagactccgggg |
| | | aaggggctggagtgggtctcaggtatgagtggtaggggtggtaggactgactacgcagactc |
| | | cgtgaagggccggttcaccatctccagagacagttccaacagcaccctctatctacaaatga |
| | | acagcctgagagccgaggacacggccttatattactgtgcgaaagatttaccctctgataata |
| | | gtggcctcaactccgctgaattcttccatgtctggggacagggcgccctggtcaccgtctcctca |
| | | g |
| 300. | VL_nuc | gacatccgngagacccagtctccatccaccctgtctgcatctgtaggggacagagtcaccat |
| | | cacttgccgggccagtgagagtattagtagctggttggcctggtatcagcagaaaccaggga |
| | | aagcccctaaactcctgatctatgaggcgtctactttagaaactggggtcccatcaagattcag |
| | | cggcagtggatctgggacagaattcactctcaccatcagaagcctgcagcctgatgattttgc |
| | | aacttattactgccaacactataatagtgactctctcactttcggcggcgggaccaaggtggag |
| | | atcaaac |
| SEQ ID NO | | P31 |
| 301. | CDR-H1 | GDPFTSYY |
| 302. | CDR-H2 | IFTTGST |
| 303. | CDR-H3 | ARVRRYCSGGRCYPYFYMDV |
| 304. | CDR-L1 | ESISSW |
| 305. | CDR-L2 | EAS |
| 306. | CDR-L3 | QHYNSDSLT |
| 307. | VH | QVQLQESGPGLVEPSETLSLTCTVSGDPFTSYYWTWIRQPAGKGL |
| | | EWLGRIFTTGSTSYNPSLKSRVTMSVDTSKSQFSLKLTAVTAADTA |
| | | VYYCARVRRYCSGGRCYPYFYMDVWGKGTTVIVSS |
| 308. | VL | DIRETQSPSTLSASVGDRVTITCRASESISSWLAWYQQKPGKAPKL |
| | | LIYEASTLETGVPSRFSGSGSGTEFTLTIRSLQPDDFATYYCQHYNS |
| | | DSLTFGGGTKVEIK |
| 309. | FR-H1 | QVQLQESGPGLVEPSETLSLTCTVS |
| 310. | FR-H2 | WTWIRQPAGKGLEWLGR |
| 311. | FR-H3 | SYNPSLKSRVTMSVDTSKSQFSLKLTAVTAADTAVYYC |
| 312. | FR-H4 | WGKGTTVIVSS |
| 313. | FR-L1 | DIRETQSPSTLSASVGDRVTITCRAS |
| 314. | FR-L2 | LAWYQQKPGKAPKLLIY |
| 315. | FR-L3 | TLETGVPSRFSGSGSGTEFTLTIRSLQPDDFATYYC |
| 316. | FR-L4 | FGGGTKVEIK |
| 317. | heavychain | QVQLQESGPGLVEPSETLSLTCTVSGDPFTSYYWTWIRQPAGKGL |
| | | EWLGRIFTTGSTSYNPSLKSRVTMSVDTSKSQFSLKLTAVTAADTA |
| | | VYYCARVRRYCSGGRCYPYFYMDVWGKGTTVIVSSKGPSVFPLAP |
| | | SSKSTSGGTAALGXLVKDYFPEPVTVSWNSGALTSGVHTFPAVLQ |
| | | SSGLYSLSSVVTVPSSSLGTQTYICNVNHKPSNTKVDKXVEPKSCD |
| | | KTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQD |
| | | WLNGKEYKCKVSNKALPAPIEKTISKAKGQPREPQVYTLPPSRDEX |
| | | TKNQVSLTCLVKGFYPSDIAVEWESNGQPENNYKTTPPVLDSDGS |
| | | FFLYSKLTVDKSRWQQGNVFSCSVMHEALHNHYTQKSLSLSPGK |
| 318. | lightchain | RCXXTQSPLYLPVTPGEPASISCRSSQSLLHSNGIHYLDWYLQKPG |
| | | QSPQLLIYLGSKRASGVPDRFSGSGSGTDFTLKISRVEAEDVGVYY |
| | | CMQSLQTFTFGPGTKVDIKAPSVFIFPPSDEQLKSGTASVVCLLNNF |
| | | YPREAKVQWKVDNALQSGNSQESVTEQDSKDSTYSLSSTLTLSKA |
| | | DYEKHKVYACEVTHQGLSSPVTKSFNRGEC*K |
| 319. | VH_nuc | caggtgcagctgcaggagtcgggcccaggcctggtggagccttcggagaccctgtccctcac |
| | | ctgcactgtctctggggaccccttcactagttactattggacatggatccggcagcccgccggga |
| | | agggactggagtggctagggcgtatctttaccactgggagcaccagctacaacccctccctca |
| | | agagtcgagtcaccatgtcagtggacacgtccaagagtcagttctccctgaaactgaccgctgt |
| | | gaccgccgcggacacggccgtctattactgtgcgagagtcagaagatattgtagtggtggaag |
| | | gtgctacccctacttctacatggacgtctggggcaaagggaccacggtcatcgtctcctca |
| 320. | VL_nuc | cgatgttgngngactcagtctccactctacctgcccgtcacccctggagagccggcctccatctc |
| | | ctgcaggtctagtcagagcctcctgcatagtaatggaatccactatttggattggtacctgcagaa |
| | | gccagggcagtctccacagctcctgatctatttgggttctaagcgggcctccggggtccctgaca |
| | | ggttcagtggcagtggatcaggcacagattttacactgaaaatcagcagagtggaggctgagg |
| | | atgttggagtttattactgcatgcaatctctacaaaccttcactttcggccctgggaccaaagtgga |
| | | tatcaaac |
| SEQ ID NO | | P33 |
| 321. | CDR-H1 | GFTFDDYT |
| 322. | CDR-H2 | IRWDGSRT |
| 323. | CDR-H3 | VKDGGLRYFDS |
| 324. | CDR-L1 | QSLLHSNGIHY |
| 325. | CDR-L2 | LGS |
| 326. | CDR-L3 | MQSLQTFT |
| 327. | VH | RCXLVESGGLVVQPGGSLRLSCEASGFTFDDYTMHWVRQSPQKG |
| | | LEWVSLIRWDGSRTEYADSVKGRFTISRDNSKNSLYLQMNTLRAD |
| | | DSAFYFCVKDGGLRYFDSWGQGTLVTVSS |
| 328. | VL | RCXXTQSPLYLPVTPGEPASISCRSSQSLLHSNGIHYLDWYLQKPG |
| | | QSPQLLIYLGSKRASGVPDRFSGSGSGTDFTLKISRVEAEDVGVYY |
| | | CMQSLQTFTFGPGTKVDIK |
| 329. | FR-H1 | RCXLVESGGLVVQPGGSLRLSCEAS |
| 330. | FR-H2 | MHWVRQSPQKGLEWVSL |
| 331. | FR-H3 | EYADSVKGRFTISRDNSKNSLYLQMNTLRADDSAFYFC |
| 332. | FR-H4 | WGQGTLVTVSS |
| 333. | FR-L1 | RCXXTQSPLYLPVTPGEPASISCRSS |
| 334. | FR-L2 | LDWYLQKPGQSPQLLIY |
| 335. | FR-L3 | KRASGVPDRFSGSGSGTDFTLKISRVEAEDVGVYYC |
| 336. | FR-L4 | FGPGTKVDIK |
| 337. | heavychain | RCXLVESGGLVVQPGGSLRLSCEASGFTFDDYTMHWVRQSPQKG |
| | | LEWVSLIRWDGSRTEYADSVKGRFTISRDNSKNSLYLQMNTLRAD |
| | | DSAFYFCVKDGGLRYFDSWGQGTLVTVSSKGPSVFPLAPSSKSTS |
| | | GGTAALGXLVKDYFPEPVTVSWNSGALTSGVHTFPAVLQSSGLYS |
| | | LSSVVTVPSSSLGTQTYICNVNHKPSNTKVDKXVEPKSCDKTHTCP |
| | | PCPAPELLGGPSVFLFPPKPKDTLMISRTPEVTCVVVDVSHEDPEV |
| | | KFNWYVDGVEVHNAKTKPREEQYNSTYRVVSVLTVLHQDWLNGK |
| | | EYKCKVSNKALPAPIEKTISKAKGQPREPQVYTLPPSRDEXTKNQV |
| | | SLTCLVKGFYPSDIAVEWESNGQPENNYKTTPPVLDSDGSFFLYSK |
| | | LTVDKSRWQQGNVFSCSVMHEALHNHYTQKSLSLSPGK |
| 338. | lightchain | EIVMTQSPLSLPVTPGEPASISCRSSQSLLHSNGIHYLDWYLQKPG |
| | | QSPQLLIYLGSKRASGVPDRFSGSGSGTDFTLKISRVEAEDVGVYY |
| | | CMQSLQTFTFGPGTKVDIKAPSVFIFPPSDEQLKSGTASVVCLLNNF |
| | | YPREAKVQWKVDNALQSGNSQESVTEQDSKDSTYSLSSTLTLSKA |
| | | DYEKHKVYACEVTHQGLSSPVTKSFNRGEC*K |
| 339. | VH_nuc | aggtgccnnctggtggagtctgggggactcgtggtacagcctggggggtccctgagactctcct |
| | | gtgaagcctctggattcacctttgatgattacaccatgcactgggtccgtcaatctccgcagaag |
| | | ggtctggagtgggtctctcttattcgttgggatgggagtaggacagagtatgcagactctgtgaa |
| | | gggtcgattcaccatctccagagacaacagcaagaactccctgtatctgcaaatgaacactctc |
| | | agagctgacgactccgccttctatttttgtgtaaaagatggtggcttacgctactttgactcctgggg |
| | | ccagggaactctggtcaccgtctcctcag |
| 340. | VL_nuc | gaaattgtgatgactcagtctccactctccctgcccgtcacccctggagagccggcctccatctc |
| | | ctgcaggtcaagtcagagcctcctgcacagtaatggaatccactatttggattggtacctgcaga |
| | | agccagggcagtctccacagctcctgatctatttgggttctaaacgggcctccggggtccctgac |
| | | aggttcagtggcagtggatcaggcacagattttacactgaaaatcagcagagtggaggctgag |
| | | gatgttggggtttattactgcatgcaatctctacaaaccttcactttcggccctgggaccaaagtgg |
| | | atatcaaac |
| SEQ ID NO | | P34 |
| 341. | CDR-H1 | GFTFDDYT |
| 342. | CDR-H2 | IRWDGSRT |
| 343. | CDR-H3 | VKDGGLRYFDS |
| 344. | CDR-L1 | QSLLHSNGIHY |
| 345. | CDR-L2 | LGS |
| 346. | CDR-L3 | MQSLQTFT |
| 347. | VH | QVQLVESGGLVVQPGGSLRLSCEASGFTFDDYTMHWVRQSPQKGL |
| | | EWVSLIRWDGSRTEYADSVKGRFTISRDNSKNSLYLQMNTLRADDS |
| | | AFYFCVKDGGLRYFDSWGQGTLVTVSS |
| 348. | VL | EIVMTQSPLSLPVTPGEPASISCRSSQSLLHSNGIHYLDWYLQKPGQ |
| | | SPQLLIYLGSKRASGVPDRFSGSGSGTDFTLKISRVEAEDVGVYYCM |
| | | QSLQTFTFGPGTKVDIK |
| 349. | FR-H1 | QVQLVESGGLVVQPGGSLRLSCEAS |
| 350. | FR-H2 | MHWVRQSPQKGLEWVSL |
| 351. | FR-H3 | EYADSVKGRFTISRDNSKNSLYLQMNTLRADDSAFYFC |
| 352. | FR-H4 | WGQGTLVTVSS |
| 353. | FR-L1 | EIVMTQSPLSLPVTPGEPASISCRSS |
| 354. | FR-L2 | LDWYLQKPGQSPQLLIY |
| 355. | FR-L3 | KRASGVPDRFSGSGSGTDFTLKISRVEAEDVGVYYC |
| 356. | FR-L4 | FGPGTKVDIK |
| 357. | heavychain | QVQLVESGGLVVQPGGSLRLSCEASGFTFDDYTMHWVRQSPQKGL |
| | | EWVSLIRWDGSRTEYADSVKGRFTISRDNSKNSLYLQMNTLRADDS |
| | | AFYFCVKDGGLRYFDSWGQGTLVTVSSKGPSVFPLAPSSKSTSGGT |
| | | AALGXLVKDYFPEPVTVSWNSGALTSGVHTFPAVLQSSGLYSLSSV |
| | | VTVPSSSLGTQTYICNVNHKPSNTKVDKXVEPKSCDKTHTCPPCPA |
| | | PELLGGPSVFLFPPKPKDTLMISRTPEVTCVVVDVSHEDPEVKFNWY |
| | | VDGVEVHNAKTKPREEQYNSTYRVVSVLTVLHQDWLNGKEYKCKV |
| | | SNKALPAPIEKTISKAKGQPREPQVYTLPPSRDEXTKNQVSLTCLVK |
| | | GFYPSDIAVEWESNGQPENNYKTTPPVLDSDGSFFLYSKLTVDKSR |
| | | WQQGNVFSCSVMHEALHNHYTQKSLSLSPGK |
| 358. | lightchain | RCXXTQSPLSLPVTPGEPASISCRSSQSLLHSNGYNYLDWYLQKPG |
| | | QSPQLLIYLGSNRASGVPDRFSGSGSGTDFTLKISRVEAEDVGVYYC |
| | | MQAQQSWTFGQGTKVEIKAPSVFIFPPSDEQLKSGTASVVCLLNNF |
| | | YPREAKVQWKVDNALQSGNSQESVTEQDSKDSTYSLSSTLTLSKAD |
| | | YEKHKVYACEVTHQGLSSPVTKSFNRGEC*K |
| 359. | VH_nuc | caggtgcagctggtggagtctgggggactcgtggtacagcctggggggtccctgagactctcct |
| | | gtgaagcctctggattcacctttgatgattacaccatgcactgggtccgtcaatctccgcagaagg |
| | | gtctggagtgggtctctcttattcgttgggatgggagtaggacagagtatgcagactctgtgaagg |
| | | gtcgattcaccatctccagagacaacagcaagaactccctgtatctgcaaatgaacactctcag |
| | | agctgacgactccgccttctatttttgtgtaaaagatggtggcttacgctactttgactcctggggcca |
| | | gggaactctggtcaccgtctcctcag |
| 360. | VL_nuc | cgatgttgngngactcagtctccactctccctgcccgtcacccctggagagccggcctccatctcc |
| | | tgcaggtctagtcagagcctcctgcatagtaatggatacaactatttggattggtacctgcagaag |
| | | ccagggcagtctccacaactcctgatctatttgggttctaatcgggcctccggggtccctgacagg |
| | | ttcagtggcagtggatcaggcacagattttacactgaaaatcagcagagtggaggctgaggatg |
| | | ttggggtttattactgcatgcaagctcaacaaagttggacgttcggccaagggaccaaggtggaa |
| | | atcaaac |
| SEQ ID NO | | P39 |
| 361. | CDR-H1 | GFTFEDYT |
| 362. | CDR-H2 | IHWNSEKR |
| 363. | CDR-H3 | VKDSGLRSLQY |
| 364. | CDR-L1 | QSLLHSNGYNY |
| 365. | CDR-L2 | LGS |
| 366. | CDR-L3 | MQAQQSWT |
| 367. | VH | EVXXVESGGGLVQPGGALRLSCSASGFTFEDYTMHWVRQFPGGGL |
| | | EWVSNIHWNSEKRDYADSVKGRFTISRDNAKNSLYLEMNNVRGEDT |
| | | AFYYCVKDSGLRSLQYWGQGTLVTVSS |
| 368. | VL | RCXXTQSPLSLPVTPGEPASISCRSSQSLLHSNGYNYLDWYLQKPGQ |
| | | SPQLLIYLGSNRASGVPDRFSGSGSGTDFTLKISRVEAEDVGVYYCM |
| | | QAQQSWTFGQGTKVEIK |
| 369. | FR-H1 | EVXXVESGGGLVQPGGALRLSCSAS |
| 370. | FR-H2 | MHWVRQFPGGGLEWVSN |
| 371. | FR-H3 | DYADSVKGRFTISRDNAKNSLYLEMNNVRGEDTAFYYC |
| 372. | FR-H4 | WGQGTLVTVSS |
| 373. | FR-L1 | RCXXTQSPLSLPVTPGEPASISCRSS |
| 374. | FR-L2 | LDWYLQKPGQSPQLLIY |
| 375. | FR-L3 | NRASGVPDRFSGSGSGTDFTLKISRVEAEDVGVYYC |
| 376. | FR-L4 | FGQGTKVEIK |
| 377. | heavychain | EVXXVESGGGLVQPGGALRLSCSASGFTFEDYTMHWVRQFPGGGL |
| | | EWVSNIHWNSEKRDYADSVKGRFTISRDNAKNSLYLEMNNVRGEDT |
| | | AFYYCVKDSGLRSLQYWGQGTLVTVSSKGPSVFPLAPSSKSTSGGT |
| | | AALGXLVKDYFPEPVTVSWNSGALTSGVHTFPAVLQSSGLYSLSSVV |
| | | TVPSSSLGTQTYICNVNHKPSNTKVDKXVEPKSCDKTHTCPPCPAPE |
| | | LLGGPSVFLFPPKPKDTLMISRTPEVTCVVVDVSHEDPEVKFNWYVD |
| | | GVEVHNAKTKPREEQYNSTYRVVSVLTVLHQDWLNGKEYKCKVSNK |
| | | ALPAPIEKTISKAKGQPREPQVYTLPPSRDEXTKNQVSLTCLVKGFYP |
| | | SDIAVEWESNGQPENNYKTTPPVLDSDGSFFLYSKLTVDKSRWQQG |
| | | NVFSCSVMHEALHNHYTQKSLSLSPGK |
| 378. | lightchain | XIXETQSPSSLSASVGDRVTITCRASQSISTYLNWYQQKPGKAPNLLIY |
| | | AASSLHSGVPSRFRGSGSGTDFTLTITSLQPDDFATYYCHQSYSAPR |
| | | TFGQGTTVEIKAPSVFIFPPSDEQLKSGTASVVCLLNNFYPREAKVQW |
| | | KVDNALQSGNSQESVTEQDSKDSTYSLSSTLTLSKADYEKHKVYACE |
| | | VTHQGLSSPVTKSFNRGEC*K |
| 379. | VH_nuc | gaggtgcnnnnggtggagtctgggggaggcctggtacagcctggcggggccctgagactctcct |
| | | gctcagcctctggattcacttttgaagattacacaatgcactgggtccggcaatttccagggggggg |
| | | cctggagtgggtctcaaatattcattggaatagtgaaaaaagagactatgcggactctgtgaaggg |
| | | ccggttcaccatctccagagacaacgccaagaactccctgtatttggaaatgaacaatgtgcgag |
| | | gtgaagacacggccttctattactgtgtaaaagattcggggctacggtcccttcagtactggggcca |
| | | gggaaccctggtcaccgtctcctcag |
| 380. | VL_nuc | nacatccnngagacccagtctccatcttccctgtctgcatctgtaggagacagagtcaccatcactt |
| | | gccgggcaagtcagagcattagcacctatttaaattggtatcaacaaaaaccagggaaagcccc |
| | | taacctcctgatctatgctgcatccagtttgcacagtggggtcccatcaaggttcagggggagtgga |
| | | tctgggacagatttcactctcaccatcaccagtctgcaacctgacgattttgccacttactactgtcac |
| | | cagagttacagtgcccctcgaacattcggccaagggaccacggtggaaatcaaac |
| SEQ ID NO | | S1 |
| 381. | CDR-H1 | GFSFSDSY |
| 382. | CDR-H2 | ISGSGEII |
| 383. | CDR-H3 | ARPSDYFETSEELD |
| 384. | CDR-L1 | QSISTY |
| 385. | CDR-L2 | AAS |
| 386. | CDR-L3 | HQSYSAPRT |
| 387. | VH | VXXVQSGGGLVKPGGSLRLSCTASGFSFSDSYMSWIRQAPGK |
| | | GLEWLTYISGSGEIISYADSVKGRFTISRDNAKKSVYLQMDSLR |
| | | AEDTAVYYCARPSDYFETSEELDWGQGTLVTVSS |
| 388. | VL | XIXETQSPSSLSASVGDRVTITCRASQSISTYLNWYQQKPGKAP |
| | | NLLIYAASSLHSGVPSRFRGSGSGTDFTLTITSLQPDDFATYYC |
| | | HQSYSAPRTFGQGTTVEIK |
| 389. | FR-H1 | VXXVQSGGGLVKPGGSLRLSCTAS |
| 390. | FR-H2 | MSWIRQAPGKGLEWLTY |
| 391. | FR-H3 | SYADSVKGRFTISRDNAKKSVYLQMDSLRAEDTAVYYC |
| 392. | FR-H4 | WGQGTLVTVSS |
| 393. | FR-L1 | XIXETQSPSSLSASVGDRVTITCRAS |
| 394. | FR-L2 | LNWYQQKPGKAPNLLIY |
| 395. | FR-L3 | SLHSGVPSRFRGSGSGTDFTLTITSLQPDDFATYYC |
| 396. | FR-L4 | FGQGTTVEIK |
| 397. | heavychain | VXXVQSGGGLVKPGGSLRLSCTASGFSFSDSYMSWIRQAPGK |
| | | GLEWLTYISGSGEIISYADSVKGRFTISRDNAKKSVYLQMDSLR |
| | | AEDTAVYYCARPSDYFETSEELDWGQGTLVTVSSKGPSVFPLA |
| | | PSSKSTSGGTAALGXLVKDYFPEPVTVSWNSGALTSGVHTFPA |
| | | VLQSSGLYSLSSVVTVPSSSLGTQTYICNVNHKPSNTKVDKXVE |
| | | PKSCDKTHTCPPCPAPELLGGPSVFLFPPKPKDTLMISRTPEVT |
| | | CVVVDVSHEDPEVKFNWYVDGVEVHNAKTKPREEQYNSTYRV |
| | | VSVLTVLHQDWLNGKEYKCKVSNKALPAPIEKTISKAKGQPREP |
| | | QVYTLPPSRDEXTKNQVSLTCLVKGFYPSDIAVEWESNGQPEN |
| | | NYKTTPPVLDSDGSFFLYSKLTVDKSRWQQGNVFSCSVMHEA |
| | | LHNHYTQKSLSLSPGK |
| 398. | lightchain | TQSPSSLSASVGDRVTITCQASQDISKSLNWYHQKPGKAPTVLI |
| | | YDASNLETGVPSRFSGSGSGTEFTFTISSLQSEDFGTYYCQQY |
| | | DNVPMYTFGQGTKVETKAPSVFIFPPSDEQLKSGTASVVCLLN |
| | | NFYPREAKVQWKVDNALQSGNSQESVTEQDSKDSTYSLSSTL |
| | | TLSKADYEKHKVYACEVTHQGLSSPVTKSFNRGEC*K |
| 399. | VH_nuc | aggtgcanncngtgcagtctgggggaggcttggtcaagcctggagggtccctcagactc |
| | | tcctgcacagcctctggattcagcttcagtgactcctacatgagctggatccgccaggctc |
| | | cagggaagggtctggagtggctgacatacatcagtggtagtggtgaaatcatttcctacg |
| | | cggactctgtgaagggccgattcaccatctccagggacaacgccaagaagtcagtgtat |
| | | ctgcaaatggacagcctgagagccgaggacacggccgtctattactgtgcgcggccctc |
| | | cgattattttgaaactagtgaagagctggactggggccagggaaccctggtcaccgtctc |
| | | ctcag |
| 400. | VL_nuc | gacccagtctccatcctccctgtctgcatctgtaggagacagagtcactatcacttgccag |
| | | gcgagtcaggacatcagtaaatctttaaattggtatcaccagaaaccagggaaagcccc |
| | | tacggtcctgatctacgatgcatccaatttggaaacaggggtcccatcaaggttcagtgga |
| | | agtggatctgggacagagtttactttcaccatcagcagcctgcagagtgaagattttggaa |
| | | catattactgtcaacagtatgataatgtccccatgtacacttttggtcaggggaccaaggtg |
| | | gagacgaaac |
| SEQ ID NO | | S4 |
| 401. | CDR-H1 | GMSISSYY |
| 402. | CDR-H2 | IFTTGST |
| 403. | CDR-H3 | ARLRRVVVPRVSWYFDL |
| 404. | CDR-L1 | QDISKS |
| 405. | CDR-L2 | DAS |
| 406. | CDR-L3 | QQYDNVPMYT |
| 407. | VH | RCKLQESGPGLVKPSESLSLTCNVSGMSISSYYWSWIRQPAGK |
| | | GLEWIGRIFTTGSTKDNPSLKSRVTMSVDTSRNQFSLTLTSVTAA |
| | | DTAVYYCARLRRVVVPRVSWYFDLWGHGTLVTVSS |
| 408. | VL | TQSPSSLSASVGDRVTITCQASQDISKSLNWYHQKPGKAPTVLI |
| | | YDASNLETGVPSRFSGSGSGTEFTFTISSLQSEDFGTYYCQQYD |
| | | NVPMYTFGQGTKVETK |
| 409. | FR-H1 | RCKLQESGPGLVKPSESLSLTCNVS |
| 410. | FR-H2 | WSWIRQPAGKGLEWIGR |
| 411. | FR-H3 | KDNPSLKSRVTMSVDTSRNQFSLTLTSVTAADTAVYYC |
| 412. | FR-H4 | WGHGTLVTVSS |
| 413. | FR-L1 | TQSPSSLSASVGDRVTITCQAS |
| 414. | FR-L2 | LNWYHQKPGKAPTVLIY |
| 415. | FR-L3 | NLETGVPSRFSGSGSGTEFTFTISSLQSEDFGTYYC |
| 416. | FR-L4 | FGQGTKVETK |
| 417. | heavychain | RCKLQESGPGLVKPSESLSLTCNVSGMSISSYYWSWIRQPAGK |
| | | GLEWIGRIFTTGSTKDNPSLKSRVTMSVDTSRNQFSLTLTSVTAA |
| | | DTAVYYCARLRRVVVPRVSWYFDLWGHGTLVTVSSKGPSVFPL |
| | | APSSKSTSGGTAALGXLVKDYFPEPVTVSWNSGALTSGVHTFP |
| | | AVLQSSGLYSLSSVVTVPSSSLGTQTYICNVNHKPSNTKVDKXV |
| | | EPKSCDKTHTCPPCPAPELLGGPSVFLFPPKPKDTLMISRTPEV |
| | | TCVVVDVSHEDPEVKFNWYVDGVEVHNAKTKPREEQYNSTYR |
| | | VVSVLTVLHQDWLNGKEYKCKVSNKALPAPIEKTISKAKGQPRE |
| | | PQVYTLPPSRDEXTKNQVSLTCLVKGFYPSDIAVEWESNGQPE |
| | | NNYKTTPPVLDSDGSFFLYSKLTVDKSRWQQGNVFSCSVMHEA |
| | | LHNHYTQKSLSLSPGK |
| 418. | lightchain | DIXXTQSPDSVAVSLGQRATINCESSQSVFDDSSNKNYLAWYQ |
| | | HKPGQPPKLLIYWASSRESGVPDRFIGSGSGTDFTLTISSLQAAD |
| | | VAVYYCLQYYSTPHSFGQGTKVAINAPSVFIFPPSDEQLKSGTA |
| | | SVVCLLNNFYPREAKVQWKVDNALQSGNSQESVTEQDSKDSTY |
| | | SLSSTLTLSKADYEKHKVYACEVTHQGLSSPVTKSFNRGEC*K |
| 419. | VH_nuc | aggtgcaagctgcaggagtcgggcccaggactggtgaagccttcggagagcctgtccct |
| | | cacctgcaatgtctctggtatgtccatcagtagttattactggagctggatccgacagcccgc |
| | | cgggaagggactggagtggattgggcgtatatttaccactgggagcaccaaggacaatc |
| | | cctccctcaagagtcgagtcaccatgtcagtagacacgtcgaggaaccagttctccctgac |
| | | cctgacatctgtgaccgccgcggacacggccgtatattactgtgcgagactgaggcgggtt |
| | | gttgttcctcgggtctcctggtacttcgatctctggggccatggcaccctggtcactgtctcctc |
| | | ag |
| 420. | VL_nuc | gacatcgnnnnnacccagtctccagactccgtggctgtgtctctgggccagagggccacc |
| | | atcaactgcgagtccagccagagtgttttcgatgactccagcaataagaactacttagcttg |
| | | gtatcaacacaaaccaggacagcctcctaaactactcatttactgggcatctagccggga |
| | | atccggggtccctgaccgattcattggcagcgggtctgggacagacttcactctcaccatca |
| | | gcagcctgcaggctgctgatgtggcagtttattactgtcttcaatattatagtactcctcactcttt |
| | | tggccaggggaccaaggtggcgattaacc |
| SEQ ID NO | | 24B7D4 |
| 421. | CDR-H1 | GYSFTRFD |
| 422. | CDR-H2 | MNPKSGHS |
| 423. | CDR-H3 | ARGVDNRX |
| 424. | CDR-L1 | QSVFDDSSNKNY |
| 425. | CDR-L2 | WAS |
| 426. | CDR-L3 | LQYYSTPHS |
| 427. | VH | SGAEVKKPGASVKVSCKTSGYSFTRFDINWVRQATGQGLEWM |
| | | GWMNPKSGHSGPAQKFQGRITMTVNTSISTAYMELSSLRFEDT |
| | | AVYYCARGVDNRXWGQGTLITVSS |
| 428. | VL | DIXXTQSPDSVAVSLGQRATINCESSQSVFDDSSNKNYLAWYQ |
| | | HKPGQPPKLLIYWASSRESGVPDRFIGSGSGTDFTLTISSLQAAD |
| | | VAVYYCLQYYSTPHSFGQGTKVAIN |
| 429. | FR-H1 | SGAEVKKPGASVKVSCKTS |
| 430. | FR-H2 | INWVRQATGQGLEWMGW |
| 431. | FR-H3 | GPAQKFQGRITMTVNTSISTAYMELSSLRFEDTAVYYC |
| 432. | FR-H4 | WGQGTLITVSS |
| 433. | FR-L1 | DIXXTQSPDSVAVSLGQRATINCESS |
| 434. | FR-L2 | LAWYQHKPGQPPKLLIY |
| 435. | FR-L3 | SRESGVPDRFIGSGSGTDFTLTISSLQAADVAVYYC |
| 436. | FR-L4 | FGQGTKVAIN |
| 437. | heavychain | SGAEVKKPGASVKVSCKTSGYSFTRFDINWVRQATGQGLEWM |
| | | GWMNPKSGHSGPAQKFQGRITMTVNTSISTAYMELSSLRFEDT |
| | | AVYYCARGVDNRXWGQGTLITVSSKGPSVFPLAPSSKSTSGGT |
| | | AALGXLVKDYFPEPVTVSWNSGALTSGVHTFPAVLQSSGLYSLS |
| | | SVVTVPSSSLGTQTYICNVNHKPSNTKVDKXVEPKSCDKTHTCP |
| | | PCPAPELLGGPSVFLFPPKPKDTLMISRTPEVTCVVVDVSHEDP |
| | | EVKFNWYVDGVEVHNAKTKPREEQYNSTYRVVSVLTVLHQDWL |
| | | NGKEYKCKVSNKALPAPIEKTISKAKGQPREPQVYTLPPSRDEX |
| | | TKNQVSLTCLVKGFYPSDIAVEWESNGQPENNYKTTPPVLDSD |
| | | GSFFLYSKLTVDKSRWQQGNVFSCSVMHEALHNHYTQKSLSLS |
| | | PGK |
| 438. | lightchain | DIXXTQSPDSVAVSLGQRATINCESSQSVFDDSSNKNYLAWYQ |
| | | HKPGQPPKLLIYWASSRESGVPDRFIGSGSGTDFTLTISSLQAAD |
| | | VAVYYCLQYYSTPHSFGQGTKVAINAPSVFIFPPSDEQLKSGTA |
| | | SVVCLLNNFYPREAKVQWKVDNALQSGNSQESVTEQDSKDSTY |
| | | SLSSTLTLSKADYEKHKVYACEVTHQGLSSPVTKSFNRGEC*K |
| 439. | VH_nuc | agtctggggctgaggtgaagaagcctggggcctcagtgaaggtctcctgcaagacttctg |
| | | gatacagcttcacccgttttgatatcaactgggtgcgacaggccactggacaagggcttga |
| | | gtggatgggatggatgaaccctaagagtggtcactcaggccctgcacagaagttccagg |
| | | gcagaatcaccatgaccgttaacacctccataagtacagcctacatggagctgagcagcc |
| | | tgagatttgaggacacggccgtttattattgtgcgcgaggcgtggataatcgtnnctggggc |
| | | cagggaaccctaatcaccgtctcctcag |
| 440. | VL_nuc | gacatcgnnnnnacccagtctccagactccgtggctgtgtctctgggccagagggccacc |
| | | atcaactgcgagtccagccagagtgttttcgatgactccagcaataagaactacttagcttg |
| | | gtatcaacacaaaccaggacagcctcctaaactactcatttactgggcatctagccggga |
| | | atccggggtccctgaccgattcattggcagcgggtctgggacagacttcactctcaccatca |
| | | gcagcctgcaggctgctgatgtggcagtttattactgtcttcaatattatagtactcctcactcttt |
| | | tggccaggggaccaaggtggcgattaacc |
| SEQ ID NO | | Ara h 2 |
| 441. | Ara h 2 | MAKLTILVALALFLLAAHASARQQWELQGDRRCQSQLERANLRP |
| | | CEQHLMQKIQRDEDSYGRDPYSPSQDPYSPSQDPDRRDPYSP |
| | | SPYDRRGAGSSQHQERCCNELNEFENNQRCMCEALQQIMENQ |
| | | SDRLQGRQQEQQFKRELRNLPQQCGLRAPQRCDLEVESGGRD |
| | | RY |
| SEQ ID NO | | T1 |
| 442. | CDR-H1 | GFKFQNYG |
| 443. | CDR-H2 | ISGSDEST |
| 444. | CDR-H3 | AKATAPAGKYYYYGMDV |
| 445. | CDR-L1 | QTVSSY |
| 446. | CDR-L2 | DAS |
| 447. | CDR-L3 | HQRSNWPPVHT |
| 448. | VH | XARLVESGGGVVQPGGSLRLSCVASGFKFQNYGMSWVR |
| | | QAPGKGLEWVADISGSDESTYYADSVKGRFTISRDTSKNT |
| | | LHLQMSSLRAEDTALYYCAKATAPAGKYYYYGMDVWGQG |
| | | TTVTVSS |
| 449. | VL | QSPATLSLSPGEIATLSCRASQTVSSYLAWYQLKPGQAPRL |
| | | LIYDASRRAAGIPARFSGSESGTDFTLTISSLEPEDSAVYYC |
| | | HQRSNWPPVHTFGQGTKLEIK |
| 450. | FR-H1 | XARLVESGGGVVQPGGSLRLSCVAS |
| 451. | FR-H2 | MSWVRQAPGKGLEWVAD |
| 452. | FR-H3 | YYADSVKGRFTISRDTSKNTLHLQMSSLRAEDTALYYC |
| 453. | FR-H4 | WGQGTTVTVSS |
| 454. | FR-L1 | QSPATLSLSPGEIATLSCRAS |
| 455. | FR-L2 | LAWYQLKPGQAPRLLIY |
| 456. | FR-L3 | RRAAGIPARFSGSESGTDFTLTISSLEPEDSAVYYC |
| 457. | FR-L4 | FGQGTKLEIK |
| 458. | heavychain | XARLVESGGGVVQPGGSLRLSCVASGFKFQNYGMSWVR |
| | | QAPGKGLEWVADISGSDESTYYADSVKGRFTISRDTSKNT |
| | | LHLQMSSLRAEDTALYYCAKATAPAGKYYYYGMDVWGQG |
| | | TTVTVSSKGPSVFPLAPSSKSTSGGTAALGXLVKDYFPEPV |
| | | TVSWNSGALTSGVHTFPAVLQSSGLYSLSSVVTVPSSSLG |
| | | TQTYICNVNHKPSNTKVDKXVEPKSCDKTHTCPPCPAPELL |
| | | GGPSVFLFPPKPKDTL |
| 459. | lightchain | QSPATLSLSPGEIATLSCRASQTVSSYLAWYQLKPGQAPRL |
| | | LIYDASRRAAGIPARFSGSESGTDFTLTISSLEPEDSAVYYC |
| | | HQRSNWPPVHTFGQGTKLEIKAPSVFIFPPSDEQLKSGTAS |
| | | VVCLLNNFYPREAKVQWKVDNALQSGNSQESVTEQDSKD |
| | | STYSLSSTLTLSKADYEKHKVYACEVTHQGLSSPVTKSFNR |
| | | GEC*K |
| 460. | VH_nuc | ngtgccaggctggtggagtctgggggaggcgtggttcagcctggggggtccctga |
| | | gactctcctgtgtagcctctggattcaaatttcagaactatggcatgagctgggtccgc |
| | | caggctccagggaaggggctggagtgggtcgcagatattagtggaagtgatgaa |
| | | agcacttactatgcagactccgtgaagggccggttcaccatctccagagacacttc |
| | | caagaacacactgcatctgcaaatgagcagt |
| 461. | VL_nuc | cagtctccagccaccctgtctctgtctccaggggaaatagccaccctctcctgcagg |
| | | gccagtcagactgttagcagctacttagcctggtaccaactcaaacctggccaggc |
| | | tcccaggctcctcatctatgatgcgtccaggagggccgctggcatcccagccagatt |
| | | cagtggcagtgagtctgggacagacttcactctcaccatcagcagcctagagcctg |
| | | aagattctgcagtctattactgtcaccag |
| SEQ ID NO | | T3 |
| 462. | CDR-H1 | GFTFRDYS |
| 463. | CDR-H2 | IRFDGTTK |
| 464. | CDR-H3 | AKDNGWRAFDH |
| 465. | CDR-L1 | QSLLHRNGYIY |
| 466. | CDR-L2 | FVS |
| 467. | CDR-L3 | MQALETPWT |
| 468. | VH | GGGVVQPGTSLRLSCVASGFTFRDYSMHWVRQAPGKGLE |
| | | WVSFIRFDGTTKDYRDSVKGRFIISRDDSKNTLYLQMTSLR |
| | | VEDTALYYCAKDNGWRAFDHWGQGALVTVSS |
| 469. | VL | VMTQSPLSLPVTPGEAASISCRSSQSLLHRNGYIYLDWYLQ |
| | | RPGQSPQLLISFVSKRASGAPDRFSGSGSGTDFTLTISRVE |
| | | AEDFGVYFCMQALETPWTFGPGTKLEIK |
| 470. | FR-H1 | GGGVVQPGTSLRLSCVAS |
| 471. | FR-H2 | MHWVRQAPGKGLEWVSF |
| 472. | FR-H3 | DYRDSVKGRFIISRDDSKNTLYLQMTSLRVEDTALYYC |
| 473. | FR-H4 | WGQGALVTVSS |
| 474. | FR-L1 | VMTQSPLSLPVTPGEAASISCRSS |
| 475. | FR-L2 | LDWYLQRPGQSPQLLIS |
| 476. | FR-L3 | KRASGAPDRFSGSGSGTDFTLTISRVEAEDFGVYFC |
| 477. | FR-L4 | FGPGTKLEIK |
| 478. | heavychain | GGGVVQPGTSLRLSCVASGFTFRDYSMHWVRQAPGKGLE |
| | | WVSFIRFDGTTKDYRDSVKGRFIISRDDSKNTLYLQMTSLR |
| | | VEDTALYYCAKDNGWRAFDHWGQGALVTVSSKGPSVFPL |
| | | APSSKSTSGGTAALGXLVKDYFPEPVTVSWNSGALTSGVH |
| | | TFPAVLQSSGLYSLSSVVTVPSSSLGTQTYICNVNHKPSNT |
| | | KVDKXVEPKSCDKTHTCPPCPAPELLGGPSVFLFPPKPKD |
| | | TLMISRTPEVTCVVVDVSHEDPEVKFNWYVDGVEVHNAKT |
| | | KPREEQYNSTYRVVSVLTVLHQDWLNGKEYKCKVSNKALP |
| | | APIEKTISKAKGQPREPQVYTLPPSRDEXTKNQVSLTCLVK |
| | | GFYPSDIAVEWESNGQPENNYKTTPPVLDSDGSFFLYSKL |
| | | TVDKSRWQQGNVFSCSVMHEALHNHYTQKSLSLSPGK |
| 479. | lightchain | VMTQSPLSLPVTPGEAASISCRSSQSLLHRNGYIYLDWYLQ |
| | | RPGQSPQLLISFVSKRASGAPDRFSGSGSGTDFTLTISRVE |
| | | AEDFGVYFCMQALETPWTFGPGTKLEIKAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC*K |
| 480. | VH_nuc | cggggggcggcgtggtccagcctgggacgtccctgagactctcctgtgtagcgtct |
| | | ggattcaccttccgtgactattccatgcactgggtccgccaggctccaggcaaggg |
| | | gctggagtgggtgtcatttatacggtttgatgggaccactaaggactatcgagactct |
| | | gtgaagggccgattcatcatctccagagacgactccaagaacacactgtatctgca |
| | | gatgacgagcctgagagttgaggacacggctttatattactgtgcgaaagacaatg |
| | | ggtggcgggcctttgaccactggggccagggagccctggtcaccgtctcctcag |
| 481. | VL_nuc | gtgatgactcagtctccactctccctgcccgtcacccctggagaggcggcctccatc |
| | | tcctgcaggtctagtcagagcctcctccatcgtaatggatacatctacttggattggta |
| | | cctgcagaggccagggcagtctccacaactcctgatctctttcgtttctaaacgggcc |
| | | tccggggcccctgacaggttcagtggctctggttcaggcacagattttacactgaca |
| | | atcagcagagtggaggctgaggattttggggtttatttctgcatgcaagcgctagaa |
| | | accccctggacgttcggcccagggaccaaactggagatcaaac |
| SEQ ID NO | | T4 |
| 482. | CDR-H1 | GDSISSYY |
| 483. | CDR-H2 | IFTSGST |
| 484. | CDR-H3 | ARDRRALSSDGNWYWYFDL |
| 485. | CDR-L1 | QTITRN |
| 486. | CDR-L2 | GAS |
| 487. | CDR-L3 | QQSDNTPRT |
| 488. | VH | ESGPRLVKPSETLSLTCIVSGDSISSYYWGWIRQPAGRGLE |
| | | WIGRIFTSGSTTYNPSLKSRVSMSVETSKNQFSLTLTSVTAA |
| | | DTAVYFCARDRRALSSDGNWYWYFDLWGRGTLVAVSS |
| 489. | VL | TQSPSSLSASVGDRVTITCRASQTITRNLNWYQQKSGEAPK |
| | | LLIYGASILQSGVPSRFTGSGSGTDFTLTISNLQPEDFASYSC |
| | | QQSDNTPRTFGQGTKVEIK |
| 490. | FR-H1 | ESGPRLVKPSETLSLTCIVS |
| 491. | FR-H2 | WGWIRQPAGRGLEWIGR |
| 492. | FR-H3 | TYNPSLKSRVSMSVETSKNQFSLTLTSVTAADTAVYFC |
| 493. | FR-H4 | WGRGTLVAVSS |
| 494. | FR-L1 | TQSPSSLSASVGDRVTITCRAS |
| 495. | FR-L2 | LNWYQQKSGEAPKLLIY |
| 496. | FR-L3 | ILQSGVPSRFTGSGSGTDFTLTISNLQPEDFASYSC |
| 497. | FR-L4 | FGQGTKVEIK |
| 498. | heavychain | ESGPRLVKPSETLSLTCIVSGDSISSYYWGWIRQPAGRGLE |
| | | WIGRIFTSGSTTYNPSLKSRVSMSVETSKNQFSLTLTSVTAA |
| | | DTAVYFCARDRRALSSDGNWYWYFDLWGRGTLVAVSSKG |
| | | PSVFPLAPSSKSTSGGTAALGXLVKDYFPEPVTVSWNSGAL |
| | | TSGVHTFPAVLQSSGLYSLSSVVTVPSSSLGTQTYICNVNHK |
| | | PSNTKVDKXVEPKSCDKTHTCPPCPAPELLGGPSVFLFPPK |
| | | PKDTLMISRTPEVTCVVVDVSHEDPEVKFNWYVDGVEVHN |
| | | AKTKPREEQYNSTYRVVSVLTVLHQDWLNGKEYKCKVSNK |
| | | ALPAPIEKTISKAKGQPREPQVYTLPPSRDEXTKNQVSLTCL |
| | | VKGFYPSDIAVEWESNGQPENNYKTTPPVLDSDGSFFLYSK |
| | | LTVDKSRWQQGNVFSCSVMHEALHNHYTQKSLSLSPGK |
| 499. | lightchain | TQSPSSLSASVGDRVTITCRASQTITRNLNWYQQKSGEAPK |
| | | LLIYGASILQSGVPSRFTGSGSGTDFTLTISNLQPEDFASYSC |
| | | QQSDNTPRTFGQGTKVEIKAPSVFIFPPSDEQLKSGTASVV |
| | | CLLNNFYPREAKVQWKVDNALQSGNSQESVTEQDSKDSTY |
| | | SLSSTLTLSKADYEKHKVYACEVTHQGLSSPVTKSFNRGEC |
| | | *K |
| 500. | VH_nuc | ggagtcgggcccacgactggtgaagccttcggagaccctgtccctcacctgcattgt |
| | | ctctggtgactccatcagtagttattattggggctggatccggcagcccgccgggagg |
| | | ggactggagtggattgggcggatctttaccagcgggagcaccacctataatccctcc |
| | | ctcaagagtcgagtctccatgtcagtagagacatccaagaaccagttctccctgaca |
| | | ctgacctctgtgaccgccgcggacacggccgtttatttctgtgcgagagatcgaagg |
| | | gcactttcgtctgacggcaactggtactggtacttcgatctctggggccgtggcaccct |
| | | ggtcgctgtctcctcgg |
| 501. | VL_nuc | gacccagtctccatcctccctgtctgcatctgtcggagacagagtcaccatcacttgcc |
| | | gggcaagtcagaccattactcggaatttaaattggtatcagcagaaatcaggggaa |
| | | gcccctaagctcctgatctatggtgcatccattttgcaaagtggggtcccatcaaggttc |
| | | actggcagtggatctgggacagatttcactctcaccatcagtaatctgcaacctgaag |
| | | attttgcaagttactcctgtcaacagagtgacaataccccgcggacgttcggccaag |
| | | ggaccaaggtggagatcaaac |
| SEQ ID NO | | T5 |
| 502. | CDR-H1 | GGSMSSYY |
| 503. | CDR-H2 | IFTTGST |
| 504. | CDR-H3 | VRDRRGRSHDSNWYWYFDL |
| 505. | CDR-L1 | QTLSRN |
| 506. | CDR-L2 | GAS |
| 507. | CDR-L3 | QQSDNTPRT |
| 508. | VH | QVQLQESGPGLVKPSETLSLTCTVSGGSMSSYYWGWIRQP |
| | | AGRGLEWIGRIFTTGSTIYNASLNSRVSMSVDTSKNQFSLKL |
| | | TSVTAADTALYFCVRDRRGRSHDSNWYWYFDLWGRGTLV |
| | | TVSS |
| 509. | VL | RVIITCRASQTLSRNLNWYQQKPGEAPKLLIYGASTLQSGVP |
| | | SRFTGSGSGTDFTLIISGLQPEDFATYYCQQSDNTPRTFGQ |
| | | GT |
| 510. | FR-H1 | QVQLQESGPGLVKPSETLSLTCTVS |
| 511. | FR-H2 | WGWIRQPAGRGLEWIGR |
| 512. | FR-H3 | IYNASLNSRVSMSVDTSKNQFSLKLTSVTAADTALYFC |
| 513. | FR-H4 | WGRGTLVTVSS |
| 514. | FR-L1 | RVIITCRAS |
| 515. | FR-L2 | LNWYQQKPGEAPKLLIY |
| 516. | FR-L3 | TLQSGVPSRFTGSGSGTDFTLIISGLQPEDFATYYC |
| 517. | FR-L4 | FGQGT |
| 518. | heavychain | QVQLQESGPGLVKPSETLSLTCTVSGGSMSSYYWGWIRQP |
| | | AGRGLEWIGRIFTTGSTIYNASLNSRVSMSVDTSKNQFSLKL |
| | | TSVTAADTALYFCVRDRRGRSHDSNWYWYFDLWGRGTLV |
| | | TVSSKGPSVFPLAPSSKSTSGGTAALGXLVKDYFPEPVTVS |
| | | WNSGALTSGVHTFPAVLQSSGLYSLSSVVTVPSSSLGTQTY |
| | | ICNVNHKPSNTKVDKXVEPKSCDKTHTCPPCPAPELLGGPS |
| | | VFLFPPKPKDTLMISRTPEVTCVVVDVSHEDPEVKFNWYVD |
| | | GVEVHNAKTKPREEQYNSTYRVVSVLTVLHQDWLNGKEYK |
| | | CKVSNKALPAPIEKTISKAKGQPREPQVYTLPPSRDEXTKNQ |
| | | VSLTCLVKGFYPSDIAVEWESNGQPENNYKTTPPVLDSDGS |
| | | FFLYSKLTVDKSRWQQGNVFSCSVMHEALHNHYTQKSLSL |
| | | SPGK |
| 519. | lightchain | RVIITCRASQTLSRNLNWYQQKPGEAPKLLIYGASTLQSGVP |
| | | SRFTGSGSGTDFTLIISGLQPEDFATYYCQQSDNTPRTFGQ |
| | | GTAPSVFIFPPSDEQLKSGTASVVCLLNNFYPREAKVQWKV |
| | | DNALQSGNSQESVTEQDSKDSTYSLSSTLTLSKADYEKHKV |
| | | YACEVTHQGLSSPVTKSFNRGEC*K |
| 520. | VH_nuc | caggtgcagctgcaggagtcgggcccaggactggtgaagccttcggagaccctgt |
| | | ccctcacctgcactgtctctggtggctccatgagtagttactactggggctggatccgg |
| | | cagcccgccgggaggggactggagtggattgggcgaatcttcaccactgggagca |
| | | ccatctacaacgcctccctcaacagtcgagtctccatgtcagtagacacgtccaaga |
| | | atcagttctccctgaaactgacctctgtgaccgccgcggacacggccttgtatttctgtg |
| | | tgagagatcgaagagggcgatcgcatgacagcaactggtactggtacttcgatctct |
| | | ggggccgtggcaccctggtcactgtctcctcgg |
| 521. | VL_nuc | cagagtcatcatcacttgccgggcaagtcagacccttagccgcaatttaaattggtat |
| | | cagcagaaaccaggggaagcccctaaactcctgatctatggtgcatccactttacaa |
| | | agtggggtcccatcaaggttcactggcagtgggtctgggacagatttcactctcatcat |
| | | tagtggtctgcaacctgaagattttgcaacttactactgtcagcagagtgacaataccc |
| | | cgcggacgttcggccaagggaccaa |
| SEQ ID NO | | 13BU2T6 |
| 600. | CDR-H1 | GYTFNRYD |
| 601. | CDR-H2 | MNPKTGNT |
| 602. | CDR-H3 | ARGVDATH |
| 603. | CDR-L1 | QSVFDSSSNKNY |
| 604. | CDR-L2 | WAS |
| 605. | CDR-L3 | QQYHSTPHT |
| 606. | VH | VKVSCKASGYTFNRYDINWVRQATGQGLEWVGWMNPKTG |
| | | NTGYAQKFQGRVTMTRDTSMSTAYMELNNLTSEDTAVYYC |
| | | ARGVDATHWGQGTRVTVSS |
| 607. | VL | TQSPDSLAVSLGERATINCKSSQSVFDSSSNKNYLGWYQQ |
| | | NPGQPPKLLIYWASNRESGVPDRFSGSGSGTDFTLTIDSLQ |
| | | AEDVAIYYCQQYHSTPHTFGQGTKLEIK |
| 608. | FR-H1 | VKVSCKAS |
| 609. | FR-H2 | INWVRQATGQGLEWVGW |
| 610. | FR-H3 | GYAQKFQGRVTMTRDTSMSTAYMELNNLTSEDTAVYYC |
| 611. | FR-H4 | WGQGTRVTVSS |
| 612. | FR-L1 | TQSPDSLAVSLGERATINCKSS |
| 613. | FR-L2 | LGWYQQNPGQPPKLLIY |
| 614. | FR-L3 | NRESGVPDRFSGSGSGTDFTLTIDSLQAEDVAIYYC |
| 615. | FR-L4 | FGQGTKLEIK |
| 616. | heavychain | VKVSCKASGYTFNRYDINWVRQATGQGLEWVGWMNPKTG |
| | | NTGYAQKFQGRVTMTRDTSMSTAYMELNNLTSEDTAVYYC |
| | | ARGVDATHWGQGTRVTVSSKGPSVFPLAPSSKSTSGGTAA |
| | | LGXLVKDYFPEPVTVSWNSGALTSGVHTFPAVLQSSGLYSL |
| | | SSVVTVPSSSLGTQTYICNVNHKPSNTKVDKXVEPKSCDKT |
| | | HTCPPCPAPELLGGPSVFLFPPKPKDTLMISRTPEVTCVVVD |
| | | VSHEDPEVKFNW |
| 617. | lightchain | TQSPDSLAVSLGERATINCKSSQSVFDSSSNKNYLGWYQQ |
| | | NPGQPPKLLIYWASNRESGVPDRFSGSGSGTDFTLTIDSLQ |
| | | AEDVAIYYCQQYHSTPHTFGQGTKLEIKAPSVFIFPPSDEQL |
| | | KSGTASVVCLLNNFYPREAKVQWKVDNALQSGNSQESVTE |
| | | QDSKDSTYSLSSTLTLSKADYEKHKVYACEVTHQGLSSPVT |
| | | KSFNRGEC*K |
| 618. | VH_nuc | agtgaaggtctcctgcaaggcttctggatacaccttcaacagatatgatatcaattggg |
| | | tgcgacaggccactggacaagggcttgagtgggtgggatggatgaacccgaaga |
| | | ccggcaacacaggctatgcacagaagttccagggcagagtcaccatgaccaggg |
| | | acacttccatgagtacagcctacatggagctgaacaacctgacatctgaggacacg |
| | | gccgtatattattgtgcgagaggggggacgc |
| 619. | VL_nuc | acccagtctccagactccctggctgtgtctctgggcgagagggccaccatcaactgc |
| | | aagtccagccagagtgtttttgacagctccagcaataagaactacttaggttggtacc |
| | | agcagaacccaggacagcctcctaagttgctcatttactgggcatctaaccgggaat |
| | | ctggggtccctgaccgattcagtggcagcgggtctgggacagatttcactctcaccat |
| | | cgacagcctgcaggctgaagatgtg |
| SEQ ID NO | | 21BU2U1 |
| 620. | CDR-H1 | GYIFSKFD |
| 621. | CDR-H2 | TNPKSGNA |
| 622. | CDR-H3 | ARGVDNRD |
| 623. | CDR-L1 | QSIFDSSSDTNY |
| 624. | CDR-L2 | WAS |
| 625. | CDR-L3 | HQYYRPPHT |
| 626. | VH | XVXLVQSGSEVKKPGASVKVSCQASGYIFSKFDINWVRQAP |
| | | GQGLEWMGWTNPKSGNAGYAPKFLGRVTMTTDTSTNTAY |
| | | MELSNLRSDDTAVYYCARGVDNRDWGQGTLVTVSS |
| 627. | VL | EXXXTQSPDSLAVSLGERATINCQSSQSIFDSSSDTNYLAW |
| | | YQQKTGQPPKLLIYWASARESGVPDRFSGGGSGTDFTLTIS |
| | | SLQAEDVAIYFCHQYYRPPHTFGQGTRLEIN |
| 628. | FR-H1 | XVXLVQSGSEVKKPGASVKVSCQAS |
| 629. | FR-H2 | INWVRQAPGQGLEWMGW |
| 630. | FR-H3 | GYAPKFLGRVTMTTDTSTNTAYMELSNLRSDDTAVYYC |
| 631. | FR-H4 | WGQGTLVTVSS |
| 632. | FR-L1 | EXXXTQSPDSLAVSLGERATINCQSS |
| 633. | FR-L2 | LAWYQQKTGQPPKLLIY |
| 634. | FR-L3 | ARESGVPDRFSGGGSGTDFTLTISSLQAEDVAIYFC |
| 635. | FR-L4 | FGQGTRLEIN |
| 636. | heavychain | XVXLVQSGSEVKKPGASVKVSCQASGYIFSKFDINWVRQAP |
| | | GQGLEWMGWTNPKSGNAGYAPKFLGRVTMTTDTSTNTAY |
| | | MELSNLRSDDTAVYYCARGVDNRDWGQGTLVTVSSKGPS |
| | | VFPLAPSSKSTSGGTAALGXLVKDYFPEPVTVSWNSGALTS |
| | | GVHTFPAVLQSSGLYSLSSVVTVPSSSLGTQTYICNVNHKP |
| | | SNTKVDKXVEPKSCDKTHTCPPCPAPELLGGPSVFLFPPKP |
| | | KDTLMISRTPEVT |
| 637. | lightchain | EXXXTQSPDSLAVSLGERATINCQSSQSIFDSSSDTNYLAW |
| | | YQQKTGQPPKLLIYWASARESGVPDRFSGGGSGTDFTLTIS |
| | | SLQAEDVAIYFCHQYYRPPHTFGQGTRLEINAPSVFIFPPSD |
| | | EQLKSGTASVVCLLNNFYPREAKVQWKVDNALQSGNSQES |
| | | VTEQDSKDSTYSLSSTLTLSKADYEKHKVYACEVTHQGLSS |
| | | PVTKSFNRGEC*K |
| 638. | VH_nuc | naggtgnnnctggtgcagtctgggagtgaggtgaagaagccyggggcctcagtga |
| | | aggtctcctgtcaggcctctggatacatcttcagtaaatttgatatcaactgggtgcgac |
| | | aggcccctggacaaggacttgagtggatgggatggacgaaccctaagagtggtaa |
| | | tgcagggtatgcaccgaaattcctgggcagagtcaccatgaccacggatacctcaa |
| | | caaacacagcctacatggagctgagcaac |
| 639. | VL_nuc | gaaatnnnnnngacgcagtctccagactccctggctgtgtctctgggcgagagggc |
| | | caccatcaactgccagtccagccagagtatttttgacagttccagcgataccaactac |
| | | ttagcttggtaccagcagaaaacaggacagcctcctaagttgctcatttactgggcat |
| | | ctgcccgggaatccggggtccctgaccgattcagtggcggcgggtctgggacagat |
| | | ttcactctcaccatcagcagcctgcag |
| SEQ ID NO | | 21BU2U2 |
| 640. | CDR-H1 | GYIFSGFD |
| 641. | CDR-H2 | MNPKSGRT |
| 642. | CDR-H3 | ARGVDNRD |
| 643. | CDR-L1 | QSVFDSSTNTNY |
| 644. | CDR-L2 | WAS |
| 645. | CDR-L3 | HQYHSTPHT |
| 646. | VH | XVXLVQSGAELKKPXASVKVSCRGSGYIFSGFDINWVRQAT |
| | | GQGLEWMGWMNPKSGRTGSAQKFQGRVTLTRNMSTNTA |
| | | YMELNSLMSEDTAVYYCARGVDNRDWGQGTLVTVSS |
| 647. | VL | DXXVTQSPDSLAVPLGERATINCKSSQSVFDSSTNTNYLAW |
| | | YQQKPGQPPKLLIYWASSRESGVPDRFSGSGSGTDFTLTIS |
| | | SLQTEDVAVYFCHQYHSTPHTFGQGTKLEIS |
| 648. | FR-H1 | XVXLVQSGAELKKPXASVKVSCRGS |
| 649. | FR-H2 | INWVRQATGQGLEWMGW |
| 650. | FR-H3 | GSAQKFQGRVTLTRNMSTNTAYMELNSLMSEDTAVYYC |
| 651. | FR-H4 | WGQGTLVTVSS |
| 652. | FR-L1 | DXXVTQSPDSLAVPLGERATINCKSS |
| 653. | FR-L2 | LAWYQQKPGQPPKLLIY |
| 654. | FR-L3 | SRESGVPDRFSGSGSGTDFTLTISSLQTEDVAVYFC |
| 655. | FR-L4 | FGQGTKLEIS |
| 656. | heavychain | XVXLVQSGAELKKPXASVKVSCRGSGYIFSGFDINWVRQAT |
| | | GQGLEWMGWMNPKSGRTGSAQKFQGRVTLTRNMSTNTA |
| | | YMELNSLMSEDTAVYYCARGVDNRDWGQGTLVTVSSKGP |
| | | SVFPLAPSSKSTSGGTAALGXLVKDYFPEPVTVSWNSGALT |
| | | SGVHTFPAVLQSSGLYSLSSVVTVPSSSLGTQTYICNVNHK |
| | | PSNTKVDKXVEPKSCDKTHTCPPCPAPELLGGPSVFLFPPK |
| | | PKDTLMISRTPEVT |
| 657. | lightchain | DXXVTQSPDSLAVPLGERATINCKSSQSVFDSSTNTNYLAW |
| | | YQQKPGQPPKLLIYWASSRESGVPDRFSGSGSGTDFTLTIS |
| | | SLQTEDVAVYFCHQYHSTPHTFGQGTKLEISAPSVFIFPPSD |
| | | EQLKSGTASVVCLLNNFYPREAKVQWKVDNALQSGNSQES |
| | | VTEQDSKDSTYSLSSTLTLSKADYEKHKVYACEVTHQGLSS |
| | | PVTKSFNRGEC*K |
| 658. | VH_nuc | gangtgcnnctggtgcagtctggggctgagttgaagaagcctsgggcctcagtgaa |
| | | ggtctcctgtaggggctctggatacatcttcagcggatttgatatcaattgggtgcgaca |
| | | ggccactggacaagggcttgagtggatggggtggatgaaccctaagagtggtagg |
| | | acaggctctgcacagaagttccagggcagagtcaccttgaccagaaatatgtccac |
| | | gaacacagcctacatggagctgaacagc |
| 659. | VL_nuc | gacatngnngtgacccagtcaccagactccctggctgtgcctctgggcgagagggc |
| | | caccatcaactgcaagtccagccagagtgtttttgacagctccaccaatacgaattac |
| | | ttagcttggtaccagcagaaaccaggacagcctcctaagctgctcatttattgggcat |
| | | cttcccgggaatccggggtccctgaccgattcagtggcagcgggtctgggacagatt |
| | | tcactctcaccatcagcagcctgcag |
| SEQ ID NO | | 21BU2U3 |
| 660. | CDR-H1 | DFTFSFYA |
| 661. | CDR-H2 | ISNNGNSQ |
| 662. | CDR-H3 | AKTLDYSEHQFYFGLDV |
| 663. | CDR-L1 | QTISSKY |
| 664. | CDR-L2 | GAS |
| 665. | CDR-L3 | QHYSNSPPYT |
| 666. | VH | QVXXVESGGGVVQPGGSLRLSCAGSDFTFSFYAIHWVRRT |
| | | PGEGLEWLTVISNNGNSQSYSDSVKGRFTVSRDNSKDTLYL |
| | | QMNNVRTDDTAVYYCAKTLDYSEHQFYFGLDVWGQGTTVI |
| | | VSS |
| 667. | VL | RNXETQSPGTLSLSPGERATLSCRTSQTISSKYLAWYQHKP |
| | | GQPPRLLLYGASRRATGVPDRFSGSGSGTDFTLTISRLEPE |
| | | DFAVYYCQHYSNSPPYTFGQGTKLDVK |
| 668. | FR-H1 | QVXXVESGGGVVQPGGSLRLSCAGS |
| 669. | FR-H2 | IHWVRRTPGEGLEWLTV |
| 670. | FR-H3 | SYSDSVKGRFTVSRDNSKDTLYLQMNNVRTDDTAVYYC |
| 671. | FR-H4 | WGQGTTVIVSS |
| 672. | FR-L1 | RNXETQSPGTLSLSPGERATLSCRTS |
| 673. | FR-L2 | LAWYQHKPGQPPRLLLY |
| 674. | FR-L3 | RRATGVPDRFSGSGSGTDFTLTISRLEPEDFAVYYC |
| 675. | FR-L4 | FGQGTKLDVK |
| 676. | heavychain | QVXXVESGGGVVQPGGSLRLSCAGSDFTFSFYAIHWVRRT |
| | | PGEGLEWLTVISNNGNSQSYSDSVKGRFTVSRDNSKDTLYL |
| | | QMNNVRTDDTAVYYCAKTLDYSEHQFYFGLDVWGQGTTVI |
| | | VSSKGPSVFPLAPSSKSTSGGTAALGXLVKDYFPEPVTVSW |
| | | NSGALTSGVHTFPAVLQSSGLYSLSSVVTVPSSSLGTQTYIC |
| | | NVNHKPSNTKVDKXVEPKSCDKTHTCPPCPAPELLGGPSVF |
| | | LFPPKPKDTL |
| 677. | lightchain | RNXETQSPGTLSLSPGERATLSCRTSQTISSKYLAWYQHKP |
| | | GQPPRLLLYGASRRATGVPDRFSGSGSGTDFTLTISRLEPE |
| | | DFAVYYCQHYSNSPPYTFGQGTKLDVKAPSVFIFPPSDEQL |
| | | KSGTASVVCLLNNFYPREAKVQWKVDNALQSGNSQESVTE |
| | | QDSKDSTYSLSSTLTLSKADYEKHKVYACEVTHQGLSSPVT |
| | | KSFNRGEC*K |
| 678. | VH_nuc | caggtgnccnnggtggagtctgggggaggcgtggtccagccgggggggtccctgc |
| | | gactctcctgtgcaggctctgattttacttttagtttttacgccatacactgggtccgccgg |
| | | actccaggtgaggggctggagtggctcacagttatctcgaataatggtaatagtcaat |
| | | cctattcagactccgtgaagggccgattcaccgtctccagagacaattccaaggata |
| | | cgttgtatctgcaaatgaacaat |
| 679. | VL_nuc | cgaaatngngagacgcagtctccaggcaccctgtctttgtctccaggggaaagagc |
| | | caccctctcctgcaggaccagtcagactataagtagtaaatatttagcctggtaccag |
| | | cataagcctggccagcctcccaggctcctcctctatggtgcatccaggagggccact |
| | | ggcgtcccagacaggttcagtggcagtgggtctgggacagacttcactctcaccatc |
| | | agcagactggagcctgaagattttgcg |
| SEQ ID NO | | 13FU1P1A3 |
| 680. | CDR-H1 | GFTFSIFG |
| 681. | CDR-H2 | ISGTGEIT |
| 682. | CDR-H3 | AKERTKYQLAYPFDY |
| 683. | CDR-L1 | QDITTY |
| 684. | CDR-L2 | DAS |
| 685. | CDR-L3 | QQYENFPRT |
| 686. | VH | VQLVESGGGLVQPGGSLRLSCTASGFTFSIFGMSWVRQAP |
| | | GKGLEWVSSISGTGEITKYTDSVKGRFTISRDNSKTTVYLQM |
| | | KSLRAEDTAFYFCAKERTKYQLAYPFDYWGQGTLVTVSS |
| 687. | VL | TQSPSSLSASVGDRVTITCQASQDITTYLSWYQQIPGKAPKL |
| | | LISDASFLQAGVPSRFSGSGSGTDFTFTITNLQPEDVATYYC |
| | | QQYENFPRTFGGGTKVEIR |
| 688. | FR-H1 | VQLVESGGGLVQPGGSLRLSCTAS |
| 689. | FR-H2 | MSWVRQAPGKGLEWVSS |
| 690. | FR-H3 | KYTDSVKGRFTISRDNSKTTVYLQMKSLRAEDTAFYFC |
| 691. | FR-H4 | WGQGTLVTVSS |
| 692. | FR-L1 | TQSPSSLSASVGDRVTITCQAS |
| 693. | FR-L2 | LSWYQQIPGKAPKLLIS |
| 694. | FR-L3 | FLQAGVPSRFSGSGSGTDFTFTITNLQPEDVATYYC |
| 695. | FR-L4 | FGGGTKVEIR |
| 696. | heavychain | VQLVESGGGLVQPGGSLRLSCTASGFTFSIFGMSWVRQAP |
| | | GKGLEWVSSISGTGEITKYTDSVKGRFTISRDNSKTTVYLQM |
| | | KSLRAEDTAFYFCAKERTKYQLAYPFDYWGQGTLVTVSSK |
| | | GPSVFPLAPSSKSTSGGTAALGXLVKDYFPEPVTVSWNSGA |
| | | LTSGVHTFPAVLQSSGLYSLSSVVTVPSSSLGTQTYICNVNH |
| | | KPSNTKVDKXVEPKSCDKTHTCPPCPAPELLGGPSVFLFPP |
| | | KPKDTLMIS |
| 697. | lightchain | TQSPSSLSASVGDRVTITCQASQDITTYLSWYQQIPGKAPKL |
| | | LISDASFLQAGVPSRFSGSGSGTDFTFTITNLQPEDVATYYC |
| | | QQYENFPRTFGGGTKVEIRAPSVFIFPPSDEQLKSGTASVV |
| | | CLLNNFYPREAKVQWKVDNALQSGNSQESVTEQDSKDSTY |
| | | SLSSTLTLSKADYEKHKVYACEVTHQGLSSPVTKSFNRGEC |
| | | *K |
| 698. | VH_nuc | gtgcagctggtggagtctgggggaggcttggtacagcctggggggtccctgagactc |
| | | tcgtgtactgcctctggcttcacttttagcatctttggcatgagttgggtccgccaggctcc |
| | | agggaaggggctggagtgggtctcaagtatcagtggtactggtgaaattacgaaat |
| | | acacagactccgtgaagggccggttcaccatctccagagacaattccaagactacc |
| | | gtgtatctgcagatgaagagcctg |
| 699. | VL_nuc | acccagtctccatcctccctgtctgcttctgttggagacagagtcaccatcacttgccag |
| | | gcgagtcaagacattaccacttatttgagttggtatcagcagataccagggaaagcc |
| | | cctaaactcctgatctccgatgcatcctttttgcaagcaggggtcccttcaagattcagt |
| | | ggaagtgggtctgggacagattttactttcaccatcaccaacctgcagcctgaggatg |
| | | ttgcaacatactactgtcaa |
| SEQ ID NO | | 13FU1P1A4 |
| 700. | CDR-H1 | GFTFNRFA |
| 701. | CDR-H2 | ISGTGAVT |
| 702. | CDR-H3 | AKDRTPVTNYYGMDV |
| 703. | CDR-L1 | QTIDNY |
| 704. | CDR-L2 | AAS |
| 705. | CDR-L3 | QQTSSTPYT |
| 706. | VH | VQLVESGGTLGQPGGSLRLSCTASGFTFNRFAINWVRQAP |
| | | GKGLEWVAAISGTGAVTYYADSVEGRFSISRENSNNTVFLE |
| | | MNNLRGEDTAVYFCAKDRTPVTNYYGMDVWGQGTTVTVS |
| | | S |
| 707. | VL | TQSPSSLSASVGDRVIITORGSQTIDNYLNWYQQKPGKAPR |
| | | LLISAASSLQGGVPSRFSGSGYGTDFTLTISSLOPEDFATYY |
| | | CQQTSSTPYTFGQGTKVEIK |
| 708. | FR-H1 | VQLVESGGTLGQPGGSLRLSCTAS |
| 709. | FR-H2 | INWVRQAPGKGLEWVAA |
| 710. | FR-H3 | YYADSVEGRFSISRENSNNTVFLEMNNLRGEDTAVYFC |
| 711. | FR-H4 | WGQGTTVTVSS |
| 712. | FR-L1 | TQSPSSLSASVGDRVIITORGS |
| 713. | FR-L2 | LNWYQQKPGKAPRLLIS |
| 714. | FR-L3 | SLQGGVPSRFSGSGYGTDFTLTISSLQPEDFATYYC |
| 715. | FR-L4 | FGQGTKVEIK |
| 716. | heavychain | VQLVESGGTLGQPGGSLRLSCTASGFTFNRFAINWVRQAP |
| | | GKGLEWVAAISGTGAVTYYADSVEGRFSISRENSNNTVFLE |
| | | MNNLRGEDTAVYFCAKDRTPVTNYYGMDVWGQGTTVTVS |
| | | SKGPSVFPLAPSSKSTSGGTAALGXLVKDYFPEPVTVSWNS |
| | | GALTSGVHTFPAVLQSSGLYSLSSVVTVPSSSLGTQTYICNV |
| | | NHKPSNTKVDKXVEPKSCDKTHTCPPCPAPELLGGPSVFLF |
| | | PPKPKDTLMIS |
| 717. | lightchain | TQSPSSLSASVGDRVIITCRGSQTIDNYLNWYQQKPGKAPR |
| | | LLISAASSLQGGVPSRFSGSGYGTDFTLTISSLOPEDFATYY |
| | | CQQTSSTPYTFGQGTKVEIKAPSVFIFPPSDEQLKSGTASVV |
| | | CLLNNFYPREAKVQWKVDNALQSGNSQESVTEQDSKDSTY |
| | | SLSSTLTLSKADYEKHKVYACEVTHQGLSSPVTKSFNRGEC |
| | | *K |
| 718. | VH_nuc | gtgcagctggtggagtctgggggaaccctgggacagccgggggggtccctgagac |
| | | tctcctgtacagcctctggattcacctttaatagatttgccatcaactgggtccgccagg |
| | | ctccagggaaggggctggaatgggtcgccgctattagtggcactggtgctgtcacat |
| | | actacgcagactccgtggagggtcggttctccatctccagagagaattccaacaaca |
| | | cggtttttctggaaatgaacaacctg |
| 719. | VL_nuc | agacccagtctccatcgtccctgtctgcatctgtgggagacagagtcatcatcacttgc |
| | | cggggaagtcagaccattgacaattatttaaattggtatcagcagaaacctgggaaa |
| | | gcccccaggctccttatctctgcggcatccagcttgcaagggggggtcccttcaaggt |
| | | tcagtggcagtggatatgggacggatttcaccctcaccatcagcagtctgcaacctg |
| | | aagattttgcaacttactactgtc |
| SEQ ID NO | | 13FU1P1A7 |
| 720. | CDR-H1 | QTIDNY |
| 721. | CDR-H2 | GAF |
| 722. | CDR-H3 | QQTYSTPYT |
| 723. | CDR-L1 | LVESGGNLGQPGGSLRLSCAASGFPFEKFAINWVRQAPGK |
| | | GLEWVSAISGTGAVTYYADSVEGRFSISRDNSKNTVFLEMN |
| | | SLRVEDTAVYFCAKDRTPVTNYYGMDVWGQGTTVTVSS |
| 724. | CDR-L2 | QSPASLSASVGDRVTITCRGSQTIDNYLNWYQHKPGKAPKL |
| | | LIYGAFSLQGGVPSRFSGSGYGTDFTLTISSLOPEDFATYYC |
| | | QQTYSTPYTFGQGTKLETN |
| 725. | CDR-L3 | LVESGGNLGQPGGSLRLSCAAS |
| 726. | VH | INWVRQAPGKGLEWVSA |
| 727. | VL | YYADSVEGRFSISRDNSKNTVFLEMNSLRVEDTAVYFC |
| 728. | FR-H1 | WGQGTTVTVSS |
| 729. | FR-H2 | QSPASLSASVGDRVTITCRGS |
| 730. | FR-H3 | LNWYQHKPGKAPKLLIY |
| 731. | FR-H4 | SLQGGVPSRFSGSGYGTDFTLTISSLOPEDFATYYC |
| 732. | FR-L1 | FGQGTKLETN |
| 733. | FR-L2 | LVESGGNLGQPGGSLRLSCAASGFPFEKFAINWVRQAPGK |
| | | GLEWVSAISGTGAVTYYADSVEGRFSISRDNSKNTVFLEMN |
| | | SLRVEDTAVYFCAKDRTPVTNYYGMDVWGQGTTVTVSSKG |
| | | PSVFPLAPSSKSTSGGTAALGXLVKDYFPEPVTVSWNSGAL |
| | | TSGVHTFPAVLQSSGLYSLSSVVTVPSSSLGTQTYICNVNHK |
| | | PSNTKVDKXVEPKSCDKTHTCPPCPAPELLGGPSVFLFPPK |
| | | PKDTLMISRT |
| 734. | FR-L3 | QSPASLSASVGDRVTITCRGSQTIDNYLNWYQHKPGKAPKL |
| | | LIYGAFSLQGGVPSRFSGSGYGTDFTLTISSLOPEDFATYYC |
| | | QQTYSTPYTFGQGTKLETNAPSVFIFPPSDEQLKSGTASVV |
| | | CLLNNFYPREAKVQWKVDNALQSGNSQESVTEQDSKDSTY |
| | | SLSSTLTLSKADYEKHKVYACEVTHQGLSSPVTKSFNRGEC |
| | | *K |
| 735. | FR-L4 | agctggtggagtctgggggaaacttgggacagccgggggggtccctgagactgtcc |
| | | tgtgcagcctctggattcccctttgagaagtttgccatcaactgggtccgccaggctcc |
| | | agggaaggggctggagtgggtctcggctattagtggtactggtgctgtcacatactac |
| | | gcagactccgtggagggccggttctccatctccagagacaattccaagaacacggt |
| | | gtttctggagatgaacagcctgagag |
| 736. | heavychain | cccagtcgccagcgtccctgtccgcatctgtaggagacagagtcaccatcacttgcc |
| | | ggggaagtcagaccattgacaattatttaaattggtatcagcacaaacctgggaaag |
| | | cccctaaactcctgatctatggggctttcagtttgcagggtggggtcccatcaaggttc |
| | | agtggcagtggatatgggacagatttcactctcaccatcagcagtctgcaacctgaa |
| | | gactttgcaacttactactgtcagc |
| 737. | lightchain | QTIDNY |
| 738. | VH_nuc | GAF |
| 739. | VL_nuc | QQTYSTPYT |
| SEQ ID NO | | 14FU2P1A11 |
| 740. | CDR-H1 | GFNFDDYT |
| 741. | CDR-H2 | IKWNSNNI |
| 742. | CDR-H3 | VKDNGFRGFHI |
| 743. | CDR-L1 | QSLLHRNGYNY |
| 744 | CDR-L2 | LGS |
| 745. | CDR-L3 | MQALESWT |
| 746. | VH | VXXVESGGGFVQPGGSLRLSCVASGFNFDDYTMHWVRQV |
| | | AGTGLEWVSSIKWNSNNIDYADSVKGRFTISRDNAKNSLFL |
| | | QMDSLRVEDTAFYYCVKDNGFRGFHIWGQGTMVTVSS |
| 747. | VL | TQSPLSLSVTPGESASISCRSSQSLLHRNGYNYLDWYLQKP |
| | | GQSPQLLIHLGSKRASGVPDRFSGSGSGTDFTLKISRVEAE |
| | | DVGVYYCMQALESWTFGQGTKVEIK |
| 748. | FR-H1 | VXXVESGGGFVQPGGSLRLSCVAS |
| 749. | FR-H2 | MHWVRQVAGTGLEWVSS |
| 750. | FR-H3 | DYADSVKGRFTISRDNAKNSLFLQMDSLRVEDTAFYYC |
| 751. | FR-H4 | WGQGTMVTVSS |
| 752. | FR-L1 | TQSPLSLSVTPGESASISCRSS |
| 753. | FR-L2 | LDWYLQKPGQSPQLLIH |
| 754. | FR-L3 | KRASGVPDRFSGSGSGTDFTLKISRVEAEDVGVYYC |
| 755. | FR-L4 | FGQGTKVEIK |
| 756. | heavychain | VXXVESGGGFVQPGGSLRLSCVASGFNFDDYTMHWVRQV |
| | | AGTGLEWVSSIKWNSNNIDYADSVKGRFTISRDNAKNSLFL |
| | | QMDSLRVEDTAFYYCVKDNGFRGFHIWGQGTMVTVSSKGP |
| | | SVFPLAPSSKSTSGGTAALGXLVKDYFPEPVTVSWNSGALT |
| | | SGVHTFPAVLQSSGLYSLSSVVTVPSSSLGTQTYICNVNHK |
| | | PSNTKVDKXVEPKSCDKTHTCPPCPAPELLGGPSVFLFPPK |
| | | PKDTLMISRTPE |
| 757. | lightchain | TQSPLSLSVTPGESASISCRSSQSLLHRNGYNYLDWYLQKP |
| | | GQSPQLLIHLGSKRASGVPDRFSGSGSGTDFTLKISRVEAE |
| | | DVGVYYCMQALESWTFGQGTKVEIKAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKS |
| | | FNRGEC*K |
| 758. | VH_nuc | aggtgncagnggtggagtctgggggaggcttcgtacagcctggcgggtccctgag |
| | | actctcctgtgtagcctctggattcaactttgatgattataccatgcactgggtccgaca |
| | | agttgcagggacgggcctggagtgggtctcaagtattaaatggaactctaataacat |
| | | agactatgcggactctgtgaagggccggttcaccatctccagagacaacgccaag |
| | | aattccctgtttctgcagatggatagtc |
| 759. | VL_nuc | gactcagtctccactctccctgtccgtcacccctggagagtcggcctccatctcctgca |
| | | ggtctagtcagagcctcctgcatcgtaatggatacaactatttggattggtacctgcag |
| | | aagccagggcagtctccacaactcctgatccatttgggttctaagcgggcctccggg |
| | | gtccctgacaggttcagtggcagtggatcaggcacagattttacactgaaaatcagc |
| | | agagtggaggctgaggatgttgg |
| SEQ ID NO | | 14FU2P1D6 |
| 760. | CDR-H1 | GDSISSYY |
| 761. | CDR-H2 | IFTSGST |
| 762. | CDR-H3 | AKHTRDFWSDDSRVHWYFNL |
| 763. | CDR-L1 | QSVSNNF |
| 764. | CDR-L2 | GAS |
| 765. | CDR-L3 | QQYDSSPYT |
| 766. | VH | ESGPGLVKPSETLSLTCTVSGDSISSYYWSWIRQPAGKGLE |
| | | WIGRIFTSGSTNINPSLKSRVTMSVDTSKNQFSLQLGSVTAA |
| | | DTAIYYCAKHTRDFWSDDSRVHWYFNLWGRGTLVTVSS |
| 767. | VL | QSPGTLSLSPGERATLSCRASQSVSNNFLAWYQQQPGQAP |
| | | SLLIYGASTRATGIPDRFSGSGSGTDFTLTISRLEPEDFAVYF |
| | | CQQYDSSPYTFGQGTKLEIK |
| 768. | FR-H1 | ESGPGLVKPSETLSLTCTVS |
| 769. | FR-H2 | WSWIRQPAGKGLEWIGR |
| 770. | FR-H3 | NINPSLKSRVTMSVDTSKNQFSLQLGSVTAADTAIYYC |
| 771. | FR-H4 | WGRGTLVTVSS |
| 772. | FR-L1 | QSPGTLSLSPGERATLSCRAS |
| 773. | FR-L2 | LAWYQQQPGQAPSLLIY |
| 774. | FR-L3 | TRATGIPDRFSGSGSGTDFTLTISRLEPEDFAVYFC |
| 775. | FR-L4 | FGQGTKLEIK |
| 776. | heavychain | ESGPGLVKPSETLSLTCTVSGDSISSYYWSWIRQPAGKGLE |
| | | WIGRIFTSGSTNINPSLKSRVTMSVDTSKNQFSLQLGSVTAA |
| | | DTAIYYCAKHTRDFWSDDSRVHWYFNLWGRGTLVTVSSKG |
| | | PSVFPLAPSSKSTSGGTAALGXLVKDYFPEPVTVSWNSGAL |
| | | TSGVHTFPAVLQSSGLYSLSSVVTVPSSSLGTQTYICNVNHK |
| | | PSNTKVDKXVEPKSCDKTHTCPPCPAPELLGGPSVFLFPPK |
| | | PKDTLMIS |
| 777. | lightchain | QSPGTLSLSPGERATLSCRASQSVSNNFLAWYQQQPGQAP |
| | | SLLIYGASTRATGIPDRFSGSGSGTDFTLTISRLEPEDFAVYF |
| | | CQQYDSSPYTFGQGTKLEIKAPSVFIFPPSDEQLKSGTASVV |
| | | CLLNNFYPREAKVQWKVDNALQSGNSQESVTEQDSKDSTY |
| | | SLSSTLTLSKADYEKHKVYACEVTHQGLSSPVTKSFNRGEC |
| | | *K |
| 778. | VH_nuc | ggagtcgggcccaggactggtgaagccttcggagaccctgtccctcacctgcactgt |
| | | ctctggtgactccatcagtagttattattggagctggatccggcagcccgccgggaag |
| | | ggactggagtggatcgggcgtatcttcaccagtgggagcaccaacatcaacccctc |
| | | cctcaagagtcgagtcaccatgtcagtagacacgtccaaaaatcagttctccctgca |
| | | gctgggttcggtgaccgccgcggacac |
| 779. | VL_nuc | cagtctccaggcaccctgtctttgtctccaggggaaagagccaccctctcctgcagg |
| | | gccagtcagagtgttagcaacaacttcttagcctggtaccagcagcaacctggccag |
| | | gctcccagtctcctcatctatggtgcttccactagggccactggcatcccagacaggtt |
| | | cagtggcagtgggtctgggacagacttcactctcaccatcagcagactggagcctg |
| | | aagattttgcagtgtatttctgtcag |
| SEQ ID NO | | 15FU1P1A3 |
| 780. | CDR-H1 | GYTFTTYA |
| 781. | CDR-H2 | INAGNGNP |
| 782. | CDR-H3 | ARDAAGTRGNWLDP |
| 783. | CDR-L1 | QSVLYSSNNKNY |
| 784. | CDR-L2 | WAS |
| 785. | CDR-L3 | QQYYSTPLT |
| 786. | VH | QSGAEVKKPGASVKISCKASGYTFTTYAMHWVRQAPGQRL |
| | | EWMGGINAGNGNPKYSEKFQDRVTITRDTSATTAYMELSSL |
| | | RSEDTAVYYCARDAAGTRGNWLDPWGQGTLVTVSS |
| 787. | VL | QSPDSLAVSLGERATINCKSSQSVLYSSNNKNYLAWYQQKP |
| | | GQPPKLLIYWASTRESGVPDRFSGSGSGTDFTLTISSLQAE |
| | | DVAVYYCQQYYSTPLTFGGGTKVEIK |
| 788. | FR-H1 | QSGAEVKKPGASVKISCKAS |
| 789. | FR-H2 | MHWVRQAPGQRLEWMGG |
| 790. | FR-H3 | KYSEKFQDRVTITRDTSATTAYMELSSLRSEDTAVYYC |
| 791. | FR-H4 | WGQGTLVTVSS |
| 792. | FR-L1 | QSPDSLAVSLGERATINCKSS |
| 793. | FR-L2 | LAWYQQKPGQPPKLLIY |
| 794. | FR-L3 | TRESGVPDRFSGSGSGTDFTLTISSLQAEDVAVYYC |
| 795. | FR-L4 | FGGGTKVEIK |
| 796. | heavychain | QSGAEVKKPGASVKISCKASGYTFTTYAMHWVRQAPGQRL |
| | | EWMGGINAGNGNPKYSEKFQDRVTITRDTSATTAYMELSSL |
| | | RSEDTAVYYCARDAAGTRGNWLDPWGQGTLVTVSSKGPS |
| | | VFPLAPSSKSTSGGTAALGXLVKDYFPEPVTVSWNSGALTS |
| | | GVHTFPAVLQSSGLYSLSSVVTVPSSSLGTQTYICNVNHKP |
| | | SNTKVDKXVEPKSCDKTHTCPPCPAPELLGGPSVFLFPPKP |
| | | KDTLMISRTPEV |
| 797. | lightchain | QSPDSLAVSLGERATINCKSSQSVLYSSNNKNYLAWYQQKP |
| | | GQPPKLLIYWASTRESGVPDRFSGSGSGTDFTLTISSLQAE |
| | | DVAVYYCQQYYSTPLTFGGGTKVEIKAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKS |
| | | FNRGEC*K |
| 798. | VH_nuc | gcagtccggggctgaggtgaagaagcctggggcctcagtgaagatttcctgcaagg |
| | | cttctggatacaccttcactacttatgctatgcattgggtgcgccaggcccccggacaa |
| | | aggcttgagtggatgggagggatcaacgctggcaatggtaatccaaaatattcaga |
| | | gaagttccaggacagagtcaccattaccagggacacatccgcgaccacagcctac |
| | | atggagctgagcagcctgagatctgagga |
| 799. | VL_nuc | ccagtctccagactccctggctgtgtctctgggcgagagggccaccatcaactgcaa |
| | | gtccagccagagtgttttatacagctccaacaataagaactacttagcttggtaccag |
| | | cagaaaccaggacagcctcctaagctgctcatttactgggcatctactegggaatcc |
| | | ggggtccctgaccgattcagtggcagcgggtctgggacagatttcactctcaccatca |
| | | gcagcctgcaggctgaagatgtggc |
| SEQ ID NO | | 15FU1P2A11 |
| 800. | CDR-H1 | GVSISTVNW |
| 801. | CDR-H2 | IFHSGSI |
| 802. | CDR-H3 | ARGTLVFHYGLDV |
| 803. | CDR-L1 | QSISNY |
| 804. | CDR-L2 | AAS |
| 805. | CDR-L3 | QQSYNTPPRT |
| 806. | VH | SGPGLVKPSGTLSLTCAVSGVSISTVNWWSWVRQTPGKGL |
| | | EWIGEIFHSGSINYNPSLKSRVTISLDKSKNQFSLKVTSVTAA |
| | | DTAVYFCARGTLVFHYGLDVWGQGTTVTVSS |
| 807. | VL | QSPSSLSASVGDRVTITCRASQSISNYLNWYQQKLGRAPKL |
| | | LIYAASSLQRGVPSRFSGSGSGTDFTLTISSLOPEDFATYYC |
| | | QQSYNTPPRTFGGGTKVEIK |
| 808. | FR-H1 | SGPGLVKPSGTLSLTCAVS |
| 809. | FR-H2 | WSWVRQTPGKGLEWIGE |
| 810. | FR-H3 | NYNPSLKSRVTISLDKSKNQFSLKVTSVTAADTAVYFC |
| 811. | FR-H4 | WGQGTTVTVSS |
| 812. | FR-L1 | QSPSSLSASVGDRVTITCRAS |
| 813. | FR-L2 | LNWYQQKLGRAPKLLIY |
| 814. | FR-L3 | SLQRGVPSRFSGSGSGTDFTLTISSLQPEDFATYYC |
| 815. | FR-L4 | FGGGTKVEIK |
| 816. | heavychain | SGPGLVKPSGTLSLTCAVSGVSISTVNWWSWVRQTPGKGL |
| | | EWIGEIFHSGSINYNPSLKSRVTISLDKSKNQFSLKVTSVTAA |
| | | DTAVYFCARGTLVFHYGLDVWGQGTTVTVSSKGPSVFPLA |
| | | PSSKSTSGGTAALGXLVKDYFPEPVTVSWNSGALTSGVHTF |
| | | PAVLQSSGLYSLSSVVTVPSSSLGTQTYICNVNHKPSNTKV |
| | | DKXVEPKSCDKTHTCPPCPAPELLGGPSVFLFPPKPKDTLMI |
| | | SRTPEVTC |
| 817. | lightchain | QSPSSLSASVGDRVTITCRASQSISNYLNWYQQKLGRAPKL |
| | | LIYAASSLQRGVPSRFSGSGSGTDFTLTISSLOPEDFATYYC |
| | | QQSYNTPPRTFGGGTKVEIKAPSVFIFPPSDEQLKSGTASVV |
| | | CLLNNFYPREAKVQWKVDNALQSGNSQESVTEQDSKDSTY |
| | | SLSSTLTLSKADYEKHKVYACEVTHQGLSSPVTKSFNRGEC |
| | | *K |
| 818. | VH_nuc | gtcgggcccaggactggtgaagccttcggggaccctgtccctcacctgcgctgtctct |
| | | ggtgtctccatcagtactgtaaactggtggagttgggtccgccagaccccagggaag |
| | | gggctggagtggattggggaaatctttcatagtgggagcatcaactacaacccgtcc |
| | | ctcaagagtcgagtcaccatatcacttgacaagtccaagaaccagttctccctgaag |
| | | gtgacctctgtgaccgccgcggacac |
| 819. | VL_nuc | ccagtctccatcctccctgtctgcatctgtaggagacagagtcaccatcacttgccgg |
| | | gcaagtcagagcatcagcaactatttaaattggtatcagcagaaattagggagagc |
| | | ccctaagctcctgatctatgctgcatccagtttgcaaagaggggtcccatcaaggttca |
| | | gtggcagtggatctgggacagatttcactctcaccatcagcagtctgcaacctgaag |
| | | attttgcgacttactactgtcagca |
| SEQ ID NO | | 15FU1P2B11 |
| 820. | CDR-H1 | GGSISSNSYY |
| 821. | CDR-H2 | IYYSGST |
| 822. | CDR-H3 | ARHVCFDDDYFDY |
| 823. | CDR-L1 | QSISNY |
| 824. | CDR-L2 | AAS |
| 825. | CDR-L3 | QHSYVTPYT |
| 826. | VH | VQLQESGPGLVKPSETLSLTCTVSGGSISSNSYYWGWIRQP |
| | | PGKGLEWIGSIYYSGSTYYNPSLKSRVTISVDTSKNQFSLKL |
| | | SSVTAADTAVYYCARHVCFDDDYFDYWGQGTLVTVSS |
| 827. | VL | QSPSSLSASVGDRVTITCRTSQSISNYLNWYQQKPGKAPKL |
| | | LIYAASSLQSGVPSRFSGSGSGTDFTLTISSLHPEDFATYFC |
| | | QHSYVTPYTFGQGTKLEIK |
| 828. | FR-H1 | VQLQESGPGLVKPSETLSLTCTVS |
| 829. | FR-H2 | WGWIRQPPGKGLEWIGS |
| 830. | FR-H3 | YYNPSLKSRVTISVDTSKNQFSLKLSSVTAADTAVYYC |
| 831. | FR-H4 | WGQGTLVTVSS |
| 832. | FR-L1 | QSPSSLSASVGDRVTITCRTS |
| 833. | FR-L2 | LNWYQQKPGKAPKLLIY |
| 834. | FR-L3 | SLQSGVPSRFSGSGSGTDFTLTISSLHPEDFATYFC |
| 835. | FR-L4 | FGQGTKLEIK |
| 836. | heavychain | VQLQESGPGLVKPSETLSLTCTVSGGSISSNSYYWGWIRQP |
| | | PGKGLEWIGSIYYSGSTYYNPSLKSRVTISVDTSKNQFSLKL |
| | | SSVTAADTAVYYCARHVCFDDDYFDYWGQGTLVTVSSKGP |
| | | SVFPLAPSSKSTSGGTAALGXLVKDYFPEPVTVSWNSGALT |
| | | SGVHTFPAVLQSSGLYSLSSVVTVPSSSLGTQTYICNVNHK |
| | | PSNTKVDKXVEPKSCDKTHTCPPCPAPELLGGPSVFLFPPK |
| | | PKDTLMISR |
| 837. | lightchain | QSPSSLSASVGDRVTITCRTSQSISNYLNWYQQKPGKAPKL |
| | | LIYAASSLQSGVPSRFSGSGSGTDFTLTISSLHPEDFATYFC |
| | | QHSYVTPYTFGQGTKLEIKAPSVFIFPPSDEQLKSGTASVVC |
| | | LLNNFYPREAKVQWKVDNALQSGNSQESVTEQDSKDSTYS |
| | | LSSTLTLSKADYEKHKVYACEVTHQGLSSPVTKSFNRGEC* |
| | | K |
| 838 | VH_nuc | ggtgcagctgcaggagtcgggcccaggactggtgaagccttcggagaccctgtccc |
| | | tcacctgcactgtctctggtggctccatcagcagtaatagttactactggggctggatcc |
| | | gccagcccccagggaaggggctggagtggattgggagtatctattatagtgggagc |
| | | acctactacaacccgtccctcaagagtcgagtcaccatatccgtagacacgtccaa |
| | | gaaccagttctccctgaagctgagctc |
| 839. | VL_nuc | ccagtctccatcctccctgtctgcatctgtaggagacagagtcaccatcacttgccgg |
| | | acaagtcagagcattagcaactatttaaattggtatcagcagaaaccagggaaagc |
| | | ccctaagctcctgatctatgctgcatccagtttgcaaagtggggtcccatcaaggttca |
| | | gtggcagtggatctgggacagatttcactctcaccatcagcagtctgcaccctgaag |
| | | attttgcaacttacttctgtcaaca |
| SEQ ID NO | | 15FU1P3A1 |
| 840. | CDR-H1 | GYTFTRYD |
| 841. | CDR-H2 | MNPKSGET |
| 842. | CDR-H3 | TRGVDSGV |
| 843. | CDR-L1 | QSVLDSSNNKKF |
| 844. | CDR-L2 | WAS |
| 845. | CDR-L3 | QQYYSPPHT |
| 846. | VH | LVQSGAEVKKPGASVKVSCKASGYTFTRYDINWVRQATGQ |
| | | GLEWMGWMNPKSGETGYAQKFHGRVIMSWNTSISTAYME |
| | | LSSLRSEDTAVYYCTRGVDSGVWGQGTTVTVSS |
| 847. | VL | TQSPDSLAVSLGERATINCKSSQSVLDSSNNKKFLAWFQQK |
| | | PGQRPKLLIYWASTRESGVPDRFTGSGSGTDFTLTISSLQA |
| | | EDVAVYYCQQYYSPPHTFGQGTKLEIK |
| 848. | FR-H1 | LVQSGAEVKKPGASVKVSCKAS |
| 849. | FR-H2 | INWVRQATGQGLEWMGW |
| 850. | FR-H3 | GYAQKFHGRVIMSWNTSISTAYMELSSLRSEDTAVYYC |
| 851. | FR-H4 | WGQGTTVTVSS |
| 852. | FR-L1 | TQSPDSLAVSLGERATINCKSS |
| 853. | FR-L2 | LAWFQQKPGQRPKLLIY |
| 854. | FR-L3 | TRESGVPDRFTGSGSGTDFTLTISSLQAEDVAVYYC |
| 855. | FR-L4 | FGQGTKLEIK |
| 856. | heavychain | LVQSGAEVKKPGASVKVSCKASGYTFTRYDINWVRQATGQ |
| | | GLEWMGWMNPKSGETGYAQKFHGRVIMSWNTSISTAYME |
| | | LSSLRSEDTAVYYCTRGVDSGVWGQGTTVTVSSKGPSVFP |
| | | LAPSSKSTSGGTAALGXLVKDYFPEPVTVSWNSGALTSGVH |
| | | TFPAVLQSSGLYSLSSVVTVPSSSLGTQTYICNVNHKPSNTK |
| | | VDKXVEPKSCDKTHTCPPCPAPELLGGPSVFLFPPKPKDTL |
| | | MISRTPEVTCVV |
| 857. | lightchain | TQSPDSLAVSLGERATINCKSSQSVLDSSNNKKFLAWFQQK |
| | | PGQRPKLLIYWASTRESGVPDRFTGSGSGTDFTLTISSLQA |
| | | EDVAVYYCQQYYSPPHTFGQGTKLEIKAPSVFIFPPSDEQLK |
| | | SGTASVVCLLNNFYPREAKVQWKVDNALQSGNSQESVTEQ |
| | | DSKDSTYSLSSTLTLSKADYEKHKVYACEVTHQGLSSPVTK |
| | | SFNRGEC*K |
| 858. | VH_nuc | ctggtgcagtctggggctgaggtgaagaagcctggggcctcagtgaaggtctcctgc |
| | | aaggcttctggatacaccttcaccagatatgatatcaattgggtgcgacaggccactg |
| | | gacaagggcttgagtggatgggatggatgaaccctaaaagtggtgagacagggta |
| | | tgcacagaagttccacggcagagtcatcatgagctggaacacctccattagtacag |
| | | cctacatggagctgagcagcctgagatct |
| 859. | VL_nuc | tgacccagtctccagactccctggctgtgtctctgggcgagagggccaccatcaact |
| | | gcaagtccagccagagtgttttagacagctccaacaataagaagttcttagcctggtt |
| | | ccagcagaaaccaggacagcgtcctaagttgctcatttactgggcatctacccggga |
| | | atccggggtccctgaccgattcactggcagcgggtctgggacagatttcactctcacc |
| | | atcagcagcctgcaggctgaagatg |
| SEQ ID NO | | 13FU1P2B10 |
| 860. | CDR-H1 | GGSISSYY |
| 861. | CDR-H2 | IFTSGST |
| 862. | CDR-H3 | ARDRRGLSYGTNWNWYFDL |
| 863. | CDR-L1 | QSISSN |
| 864. | CDR-L2 | AIS |
| 865. | CDR-L3 | QQSDNTPRT |
| 866. | VH | LQESGPGLVKPSETLSLTCIVSGGSISSYYWGWIRQPAGRG |
| | | LEWIGRIFTSGSTNYNPSLKSRVSMSVDTSRNQFSLTLTSVT |
| | | AADTAIYFCARDRRGLSYGTNWNWYFDLWGRGTLVTVSS |
| 867. | VL | XQSPSSLSASVGDRVTITCRASQSISSNLNWYQQKPGKAPK |
| | | LLIYAISTLQSGVPSRFTGSGSGTEFTLTISHLQPEDFATYYC |
| | | QQSDNTPRTFGQGTKVDVK |
| 868. | FR-H1 | LQESGPGLVKPSETLSLTCIVS |
| 869. | FR-H2 | WGWIRQPAGRGLEWIGR |
| 870. | FR-H3 | NYNPSLKSRVSMSVDTSRNQFSLTLTSVTAADTAIYFC |
| 871. | FR-H4 | WGRGTLVTVSS |
| 872. | FR-L1 | XQSPSSLSASVGDRVTITCRAS |
| 873. | FR-L2 | LNWYQQKPGKAPKLLIY |
| 874. | FR-L3 | TLQSGVPSRFTGSGSGTEFTLTISHLQPEDFATYYC |
| 875. | FR-L4 | FGQGTKVDVK |
| 876. | heavychain | LQESGPGLVKPSETLSLTCIVSGGSISSYYWGWIRQPAGRG |
| | | LEWIGRIFTSGSTNYNPSLKSRVSMSVDTSRNQFSLTLTSVT |
| | | AADTAIYFCARDRRGLSYGTNWNWYFDLWGRGTLVTVSSK |
| | | GPSVFPLAPSSKSTSGGTAALGXLVKDYFPEPVTVSWNSGA |
| | | LTSGVHTFPAVLQSSGLYSLSSVVTVPSSSLGTQTYICNVNH |
| | | KPSNTKVDKXVEPKSCDKTHTCPPCPAPELLGGPSVFLFPP |
| | | KPKDTLMI |
| 877. | lightchain | XQSPSSLSASVGDRVTITCRASQSISSNLNWYQQKPGKAPK |
| | | LLIYAISTLQSGVPSRFTGSGSGTEFTLTISHLQPEDFATYYC |
| | | QQSDNTPRTFGQGTKVDVKAPSVFIFPPSDEQLKSGTASVV |
| | | CLLNNFYPREAKVQWKVDNALQSGNSQESVTEQDSKDSTY |
| | | SLSSTLTLSKADYEKHKVYACEVTHQGLSSPVTKSFNRGEC |
| | | *K |
| 878. | VH_nuc | ctgcaggagtcgggcccaggactggtgaagccttcggagaccctgtccctcacctg |
| | | cattgtctctggtggctccatcagtagttattactggggctggatccggcagcccgccg |
| | | ggagggggctggaatggattgggcgtatctttaccagtgggagcaccaactataac |
| | | ccctccctcaagagtcgagtcagcatgtcagtagacacgtccaggaaccagttctcc |
| | | ctgacactgacctctgtgaccgccgcg |
| 879. | VL_nuc | ganccagtctccatcctccctgtctgcatctgtaggagacagagtcaccatcacttgc |
| | | cgggcaagtcagagcattagcagcaatttaaattggtatcagcagaaaccaggga |
| | | aagcccctaaactcctgatctatgccatatccactttgcaaagtggggtcccatcgcg |
| | | gttcactggaagtggatctgggacagaattcactctcaccatcagtcatctgcaacct |
| | | gaagattttgcaacgtactactgtca |
| SEQ ID NO | | 13FU1P2B12 |
| 880. | CDR-H1 | GFIFSDYS |
| 881. | CDR-H2 | IRFDSTTR |
| 882. | CDR-H3 | VKDSGLRTLSD |
| 883. | CDR-L1 | QSLLHSNGYNY |
| 884. | CDR-L2 | MAS |
| 885. | CDR-L3 | MQALQTWT |
| 886. | VH | RCPAVESGGGVVQPGGSVRLSCAASGFIFSDYSMHWVRQ |
| | | APGKGLEWVSFIRFDSTTRDYGDSVKGRFIISRDDSKNTLYL |
| | | QMTSLRPDDTAVYYCVKDSGLRTLSDWGPGTLVTVSS |
| 887. | VL | RCXXTQSPLSLPVTPGEPASISCRCSQSLLHSNGYNYLDWY |
| | | VQKPGQSPQLLIYMASKRASGVPDRFSGSGSGTDFTLKISR |
| | | VEAEDVGVYYCMQALQTWTFGPGTKVEIK |
| 888. | FR-H1 | RCPAVESGGGVVQPGGSVRLSCAAS |
| 889. | FR-H2 | MHWVRQAPGKGLEWVSF |
| 890. | FR-H3 | DYGDSVKGRFIISRDDSKNTLYLQMTSLRPDDTAVYYC |
| 891. | FR-H4 | WGPGTLVTVSS |
| 892. | FR-L1 | RCXXTQSPLSLPVTPGEPASISCRCS |
| 893. | FR-L2 | LDWYVQKPGQSPQLLIY |
| 894. | FR-L3 | KRASGVPDRFSGSGSGTDFTLKISRVEAEDVGVYYC |
| 895. | FR-L4 | FGPGTKVEIK |
| 896. | heavychain | RCPAVESGGGVVQPGGSVRLSCAASGFIFSDYSMHWVRQ |
| | | APGKGLEWVSFIRFDSTTRDYGDSVKGRFIISRDDSKNTLYL |
| | | QMTSLRPDDTAVYYCVKDSGLRTLSDWGPGTLVTVSSKGP |
| | | SVFPLAPSSKSTSGGTAALGXLVKDYFPEPVTVSWNSGALT |
| | | SGVHTFPAVLQSSGLYSLSSVVTVPSSSLGTQTYICNVNHK |
| | | PSNTKVDKXVEPKSCDKTHTCPPCPAPELLGGPSVFLFPPK |
| | | PKDTLMISRTP |
| 897. | lightchain | RCXXTQSPLSLPVTPGEPASISCRCSQSLLHSNGYNYLDWY |
| | | VQKPGQSPQLLIYMASKRASGVPDRFSGSGSGTDFTLKISR |
| | | VEAEDVGVYYCMQALQTWTFGPGTKVEIKAPSVFIFPPSDE |
| | | QLKSGTASVVCLLNNFYPREAKVQWKVDNALQSGNSQESV |
| | | TEQDSKDSTYSLSSTLTLSKADYEKHKVYACEVTHQGLSSP |
| | | VTKSFNRGEC*K |
| 898. | VH_nuc | aggtgcccagcggtggagtctgggggaggcgtggtccagcctggggggtccgtga |
| | | gactctcctgtgcagcgtctggattcatcttcagtgactattccatgcactgggtccgcc |
| | | aggctccaggcaaggggctggagtgggtgtcgtttataagatttgactcaaccacta |
| | | gagattatggagactccgtgaagggccgattcatcatctccagagacgattccaaga |
| | | acacgttgtatctccaaatgaccagc |
| 899. | VL_nuc | cgatgttgnnagactcagtctccactctccctgcccgtcacccctggagagccggcct |
| | | ccatctcctgcaggtgtagtcaaagcctcctgcatagtaatggatacaactatttggatt |
| | | ggtacgtgcagaagccagggcagtctccacaactgctgatctatatggcttctaagc |
| | | gggcctccggggtccctgacaggttcagtggcagtggatcaggcacagactttactc |
| | | tgaaaatcagcagagtggaggct |
| SEQ ID NO | | 27FU1P3A4 |
| 900. | CDR-H1 | GFPFDDFV |
| 901. | CDR-H2 | ISSRRTNT |
| 902. | CDR-H3 | AKGETRYLEWIIQSSGFDY |
| 903. | CDR-L1 | QTISTY |
| 904. | CDR-L2 | AAS |
| 905. | CDR-L3 | QQTYNVPPT |
| 906. | VH | ESGGGLVQPGGSLRLSCVASGFPFDDFVFAWVRQAPGGG |
| | | LEWVSAISSRRTNTFYADSVKGRFTISRDNTRKTVNLQMNS |
| | | LRGEDTAVYYCAKGETRYLEWIIQSSGFDYWGPGTLVAVSS |
| 907. | VL | QSPSSLSASVGERVTITCRANQTISTYLNWYQQKVGKAPKS |
| | | LIRAASSLESGVPSRFSGSGSGTDFTLTINSLQREDFATYYC |
| | | QQTYNVPPTFGQGTRVELK |
| 908. | FR-H1 | ESGGGLVQPGGSLRLSCVAS |
| 909. | FR-H2 | FAWVRQAPGGGLEWVSA |
| 910. | FR-H3 | FYADSVKGRFTISRDNTRKTVNLQMNSLRGEDTAVYYC |
| 911. | FR-H4 | WGPGTLVAVSS |
| 912. | FR-L1 | QSPSSLSASVGERVTITCRAN |
| 913. | FR-L2 | LNWYQQKVGKAPKSLIR |
| 914. | FR-L3 | SLESGVPSRFSGSGSGTDFTLTINSLQREDFATYYC |
| 915. | FR-L4 | FGQGTRVELK |
| 916. | heavychain | ESGGGLVQPGGSLRLSCVASGFPFDDFVFAWVRQAPGGG |
| | | LEWVSAISSRRTNTFYADSVKGRFTISRDNTRKTVNLQMNS |
| | | LRGEDTAVYYCAKGETRYLEWIIQSSGFDYWGPGTLVAVSS |
| | | KGPSVFPLAPSSKSTSGGTAALGXLVKDYFPEPVTVSWNSG |
| | | ALTSGVHTFPAVLQSSGLYSLSSVVTVPSSSLGTQTYICNVN |
| | | HKPSNTKVDKXVEPKSCDKTHTCPPCPAPELLGGPSVFLFP |
| | | PKPKDTLMIS |
| 917. | lightchain | QSPSSLSASVGERVTITCRANQTISTYLNWYQQKVGKAPKS |
| | | LIRAASSLESGVPSRFSGSGSGTDFTLTINSLQREDFATYYC |
| | | QQTYNVPPTFGQGTRVELKAPSVFIFPPSDEQLKSGTASVV |
| | | CLLNNFYPREAKVQWKVDNALQSGNSQESVTEQDSKDSTY |
| | | SLSSTLTLSKADYEKHKVYACEVTHQGLSSPVTKSFNRGEC |
| | | *K |
| 918. | VH_nuc | ggagtctgggggaggcttggtacaaccgggggggtccctaagactctcctgtgtcgc |
| | | ctctggattcccctttgacgactttgtcttcgcctgggtccgccaggctccaggggggg |
| | | gtctggagtgggtctcggctattagtagtcgtagaactaacacgttctacgcagactct |
| | | gtgaagggccgattcaccatctccagagacaataccaggaagacagtgaatctgc |
| | | aaatgaacagcctgagaggcgagga |
| 919. | VL_nuc | ccagtcgccgtcctccctgtctgcatctgtgggagaaagagtcaccatcacttgccgg |
| | | gccaatcagaccataagtacttatttaaattggtatcaacaaaaagtagggaaagcc |
| | | cctaagtccctgatccgtgctgcatccagtttagaaagtggggtcccatcaaggtttag |
| | | tggcagtggatctgggacagacttcactctcaccatcaacagtctgcaacgtgaaga |
| | | ttttgcaacttactactgtcaaca |
| SEQ ID NO | | 27FU1P3A10 |
| 920. | CDR-H1 | GDSIRSYY |
| 921. | CDR-H2 | IFTTGST |
| 922. | CDR-H3 | ARDQTLGRRMDV |
| 923. | CDR-L1 | QDISSW |
| 924. | CDR-L2 | TAS |
| 925. | CDR-L3 | QQGNSFPRT |
| 926. | VH | ESGPGLVKPSETLSLTCTVSGDSIRSYYWSWIRQPAGKGLE |
| | | WVGRIFTTGSTTYNPSLKSRVTMSVDMSKNQISLNLSSVTA |
| | | ADTAVYYCARDQTLGRRMDVWGQGTTVTVSS |
| 927. | VL | XQSPSSVSASVGDRVTITCRASQDISSWLAWYQQKPGKAP |
| | | KLLIYTASSLQSGVPSRFSGSGSGTDFTLTISSLOPEDFATY |
| | | YCQQGNSFPRTFGQGTKVEIK |
| 928. | FR-H1 | ESGPGLVKPSETLSLTCTVS |
| 929. | FR-H2 | WSWIRQPAGKGLEWVGR |
| 930. | FR-H3 | TYNPSLKSRVTMSVDMSKNQISLNLSSVTAADTAVYYC |
| 931. | FR-H4 | WGQGTTVTVSS |
| 932. | FR-L1 | XQSPSSVSASVGDRVTITCRAS |
| 933. | FR-L2 | LAWYQQKPGKAPKLLIY |
| 934. | FR-L3 | SLQSGVPSRFSGSGSGTDFTLTISSLQPEDFATYYC |
| 935. | FR-L4 | FGQGTKVEIK |
| 936. | heavychain | ESGPGLVKPSETLSLTCTVSGDSIRSYYWSWIRQPAGKGLE |
| | | WVGRIFTTGSTTYNPSLKSRVTMSVDMSKNQISLNLSSVTA |
| | | ADTAVYYCARDQTLGRRMDVWGQGTTVTVSSKGPSVFPLA |
| | | PSSKSTSGGTAALGXLVKDYFPEPVTVSWNSGALTSGVHTF |
| | | PAVLQSSGLYSLSSVVTVPSSSLGTQTYICNVNHKPSNTKV |
| | | DKXVEPKSCDKTHTCPPCPAPELLGGPSVFLFPPKPKDTLMI |
| | | SRTPEVTCV |
| 937. | lightchain | XQSPSSVSASVGDRVTITCRASQDISSWLAWYQQKPGKAP |
| | | KLLIYTASSLQSGVPSRFSGSGSGTDFTLTISSLOPEDFATY |
| | | YCQQGNSFPRTFGQGTKVEIKAPSVFIFPPSDEQLKSGTAS |
| | | VVCLLNNFYPREAKVQWKVDNALQSGNSQESVTEQDSKDS |
| | | TYSLSSTLTLSKADYEKHKVYACEVTHQGLSSPVTKSFNRG |
| | | EC*K |
| 938. | VH_nuc | aggagtcgggcccaggactggtgaagccttcggagaccctgtccctcacctgcact |
| | | gtctctggtgactccatcagaagttactactggagttggatccggcagcccgccggg |
| | | aagggactggagtgggttggacgtatctttaccactgggagtaccacctacaacccc |
| | | tccctcaagagtcgagtcaccatgtcagtggatatgtccaagaaccagatctccctga |
| | | acctgagctctgtgaccgccgcggaca |
| 939. | VL_nuc | ganccagtctccatcttctgtgtctgcatctgtcggagacagagtcaccatcacttgtcg |
| | | ggcgagtcaggatattagcagctggttagcctggtatcagcagaagccagggaaa |
| | | gcccctaaactcctgatctatactgcatccagtttgcaaagtggggtcccatcaaggtt |
| | | cagcggcagtggatctgggacagatttcactctcactatcagcagcctgcagcctga |
| | | agattttgcaacttactattgtca |
| SEQ ID NO | | 6BU4P2B1 |
| 940. | CDR-H1 | GFSFDDYA |
| 941. | CDR-H2 | ISWNSGTI |
| 942. | CDR-H3 | AKGTGWELVSALEN |
| 943. | CDR-L1 | QTISDY |
| 944. | CDR-L2 | AAS |
| 945. | CDR-L3 | QQSYSPPFT |
| 946. | VH | VESGGGLIQPGRSLRLSCAVSGFSFDDYAMYWVRQVPGKG |
| | | LEWVSGISWNSGTIEYADSVKGRFTISRDNAKKSLFLEMNSL |
| | | RSEDTAIYYCAKGTGWELVSALENWGQGTVVTVSS |
| 947. | VL | QSPSSLSASVGNKVTITCRASQTISDYLNWYQQKPGKAPKL |
| | | LIYAASSLQSAVPSRFAGSGSGTEFTLTISSLOPEDFATYYC |
| | | QQSYSPPFTFGPGTKVDIK |
| 948. | FR-H1 | VESGGGLIQPGRSLRLSCAVS |
| 949. | FR-H2 | MYWVRQVPGKGLEWVSG |
| 950. | FR-H3 | EYADSVKGRFTISRDNAKKSLFLEMNSLRSEDTAIYYC |
| 951. | FR-H4 | WGQGTVVTVSS |
| 952. | FR-L1 | QSPSSLSASVGNKVTITCRAS |
| 953. | FR-L2 | LNWYQQKPGKAPKLLIY |
| 954. | FR-L3 | SLQSAVPSRFAGSGSGTEFTLTISSLOPEDFATYYC |
| 955. | FR-L4 | FGPGTKVDIK |
| 956. | heavychain | VESGGGLIQPGRSLRLSCAVSGFSFDDYAMYWVRQVPGKG |
| | | LEWVSGISWNSGTIEYADSVKGRFTISRDNAKKSLFLEMNSL |
| | | RSEDTAIYYCAKGTGWELVSALENWGQGTVVTVSSKGPSV |
| | | FPLAPSSKSTSGGTAALGXLVKDYFPEPVTVSWNSGALTSG |
| | | VHTFPAVLQSSGLYSLSSVVTVPSSSLGTQTYICNVNHKPSN |
| | | TKVDKXVEPKSCDKTHTCPPCPAPELLGGPSVFLFPPKPKD |
| | | TLMISRTPE |
| 957. | lightchain | QSPSSLSASVGNKVTITCRASQTISDYLNWYQQKPGKAPKL |
| | | LIYAASSLQSAVPSRFAGSGSGTEFTLTISSLOPEDFATYYC |
| | | QQSYSPPFTFGPGTKVDIKAPSVFIFPPSDEQLKSGTASVVC |
| | | LLNNFYPREAKVQWKVDNALQSGNSQESVTEQDSKDSTYS |
| | | LSSTLTLSKADYEKHKVYACEVTHQGLSSPVTKSFNRGEC* |
| | | K |
| 958. | VH_nuc | gtggaatctgggggaggactgatacagcctggcaggtccctgagactctcctgtgca |
| | | gtctctggattcagttttgatgactatgccatgtactgggtccgacaagttccagggaag |
| | | ggcctggagtgggtctcaggcattagttggaatagtggaactatagagtatgcggact |
| | | ctgtgaagggccgattcaccatttccagagacaacgccaagaagtccctgtttctag |
| | | aaatgaacagtctgagaagcgag |
| 959. | VL_nuc | ccagtctccatcctccctgtctgcatctgtcggaaacaaagtcaccatcacttgccggg |
| | | caagtcagactattagcgactatttaaattggtatcagcaaaaaccagggaaagccc |
| | | ctaaactcctgatctatgccgcgtccagtttgcaaagtgcagtcccatcaaggttcgct |
| | | ggcagtggatctgggacagagttcaccctcaccatcagcagtctgcaacctgaaga |
| | | ttttgcaacttactactgtcaaca |
| SEQ ID NO | | 6BU4P2B11 |
| 960. | CDR-H1 | GFTFDDYA |
| 961. | CDR-H2 | IGWNSGSI |
| 962. | CDR-H3 | AKDIDSSSWWYFES |
| 963. | CDR-L1 | QSLTNNY |
| 964. | CDR-L2 | GAS |
| 965. | CDR-L3 | QKYGTSLT |
| 966. | VH | VESGGNLVQPGRSLRLSCAASGFTFDDYAMHWVRQSPGK |
| | | GLEWVSGIGWNSGSIEYADSVKGRFTISRDNAKNSLYLQMN |
| | | SLRLEDTAVYYCAKDIDSSSWWYFESWGQGTLVTVSS |
| 967. | VL | TQSPGTLSLSPGERVTLSCRASQSLTNNYLAWYQHKPGQA |
| | | PRLLIYGASTRATGIPDRFSGSGSGTDFTLTISRLEPEDLAVY |
| | | YCQKYGTSLTFGGGTKVEIK |
| 968. | FR-H1 | VESGGNLVQPGRSLRLSCAAS |
| 969. | FR-H2 | MHWVRQSPGKGLEWVSG |
| 970. | FR-H3 | EYADSVKGRFTISRDNAKNSLYLQMNSLRLEDTAVYYC |
| 971. | FR-H4 | WGQGTLVTVSS |
| 972. | FR-L1 | TQSPGTLSLSPGERVTLSCRAS |
| 973. | FR-L2 | LAWYQHKPGQAPRLLIY |
| 974. | FR-L3 | TRATGIPDRFSGSGSGTDFTLTISRLEPEDLAVYYC |
| 975. | FR-L4 | FGGGTKVEIK |
| 976. | heavychain | VESGGNLVQPGRSLRLSCAASGFTFDDYAMHWVRQSPGK |
| | | GLEWVSGIGWNSGSIEYADSVKGRFTISRDNAKNSLYLQMN |
| | | SLRLEDTAVYYCAKDIDSSSWWYFESWGQGTLVTVSSKGP |
| | | SVFPLAPSSKSTSGGTAALGXLVKDYFPEPVTVSWNSGALT |
| | | SGVHTFPAVLQSSGLYSLSSVVTVPSSSLGTQTYICNVNHK |
| | | PSNTKVDKXVEPKSCDKTHTCPPCPAPELLGGPSVFLFPPK |
| | | PKDTLMISRTPE |
| 977. | lightchain | TQSPGTLSLSPGERVTLSCRASQSLTNNYLAWYQHKPGQA |
| | | PRLLIYGASTRATGIPDRFSGSGSGTDFTLTISRLEPEDLAVY |
| | | YCQKYGTSLTFGGGTKVEIKAPSVFIFPPSDEQLKSGTASVV |
| | | CLLNNFYPREAKVQWKVDNALQSGNSQESVTEQDSKDSTY |
| | | SLSSTLTLSKADYEKHKVYACEVTHQGLSSPVTKSFNRGEC |
| | | *K |
| 978. | VH_nuc | ggtggagtctgggggaaacttggtacagcctggcaggtccctgagactctcctgtgc |
| | | agcctctggattcacctttgacgactatgccatgcactgggtccggcaaagtccaggg |
| | | aagggcctggagtgggtctcaggtattggttggaacagcggtagcatcgaatatgcg |
| | | gactctgtgaagggccgattcaccatctccagagacaacgccaagaactccctgtat |
| | | ttgcaaatgaacagtctgagacttga |
| 979. | VL_nuc | gacgcagtctccaggcaccctgtctttgtctccaggggaaagagtcaccctctcctgc |
| | | agggccagtcagagtctcaccaacaattacttagcctggtaccaacacaaacctgg |
| | | ccaggctcccaggctactcatctatggtgcatctacccgggccaccggcatcccaga |
| | | caggttcagtggcagtgggtctgggacagacttcactctcaccatcagcagactgga |
| | | gcctgaagatttggcagtatattactg |
| SEQ ID NO | | 6FU1P1A7 |
| 980. | CDR-H1 | GFPFEKFA |
| 981. | CDR-H2 | ISGTGAVT |
| 982. | CDR-H3 | AKDRTPVTNYYGMDV |
| 983. | CDR-L1 | QSISSY |
| 984. | CDR-L2 | AAS |
| 985. | CDR-L3 | QQSYSTPMAT |
| 986. | VH | QVQLVESGGNLGQPGGSLRLSCAASGFPFEKFAINWVRQA |
| | | PGKGLEWVSAISGTGAVTYYADSVEGRFSISRDNSKNTVFL |
| | | EMNSLRVEDTAVYFCAKDRTPVTNYYGMDVWGQGTTVTVS |
| | | S |
| 987. | VL | DIGXTQSPSSLSASVGDRVTITCRASQSISSYLNWYQQKPG |
| | | KAPKLLIYAASSLQSGVPSRFSGSGSGTDFTLTISSLQPEDF |
| | | ATYYCQQSYSTPMATFGGGTKVEIK |
| 988. | FR-H1 | QVQLVESGGNLGQPGGSLRLSCAAS |
| 989. | FR-H2 | INWVRQAPGKGLEWVSA |
| 990. | FR-H3 | YYADSVEGRFSISRDNSKNTVFLEMNSLRVEDTAVYFC |
| 991. | FR-H4 | WGQGTTVTVSS |
| 992. | FR-L1 | DIGXTQSPSSLSASVGDRVTITCRAS |
| 993. | FR-L2 | LNWYQQKPGKAPKLLIY |
| 994. | FR-L3 | SLQSGVPSRFSGSGSGTDFTLTISSLQPEDFATYYC |
| 995. | FR-L4 | FGGGTKVEIK |
| 996. | heavychain | QVQLVESGGNLGQPGGSLRLSCAASGFPFEKFAINWVRQA |
| | | PGKGLEWVSAISGTGAVTYYADSVEGRFSISRDNSKNTVFL |
| | | EMNSLRVEDTAVYFCAKDRTPVTNYYGMDVWGQGTTVTVS |
| | | SKGPSVFPLAPSSKSTSGGTAALGXLVKDYFPEPVTVSWNS |
| | | GALTSGVHTFPAVLQSSGLYSLSSVVTVPSSSLGTQTYICNV |
| | | NHKPSNTKVDKXVEPKSCDKTHTCPPCPAPELLGGPSVFLF |
| | | PPKPKDTLMI |
| 997. | lightchain | DIGXTQSPSSLSASVGDRVTITCRASQSISSYLNWYQQKPG |
| | | KAPKLLIYAASSLQSGVPSRFSGSGSGTDFTLTISSLQPEDF |
| | | ATYYCQQSYSTPMATFGGGTKVEIKAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKS |
| | | FNRGEC*K |
| 998. | VH_nuc | caggtgcagctggtggagtctgggggaaacttgggacagccgggggggtccctga |
| | | gactgtcctgtgcagcctctggattcccctttgagaagtttgccatcaactgggtccgcc |
| | | aggctccagggaaggggctggagtgggtctcggctattagtggtactggtgctgtca |
| | | catactacgcagactccgtggagggccggttctccatctccagagacaattccaaga |
| | | acacggtgtttctggagatgaacagc |
| 999. | VL_nuc | gacatcggnntgacccagtctccatcctccctgtctgcatctgtaggagacagagtca |
| | | ccatcacttgccgggcaagtcagagcattagcagctatttaaattggtatcagcagaa |
| | | accagggaaagcccctaagctcctgatctatgctgcatccagtttgcaaagtggggt |
| | | cccatcaaggttcagtggcagtggatctgggacagatttcactctcaccatcagcagt |
| | | ctgcaacctgaagattttgcaact |
| SEQ ID NO | | 6FU1P1B9 |
| 1000. | CDR-H1 | GGSFSGYS |
| 1001. | CDR-H2 | FNHSGNT |
| 1002. | CDR-H3 | ARERDCSGGGCYYPYSYYYGMDV |
| 1003. | CDR-L1 | QGISIY |
| 1004. | CDR-L2 | AAS |
| 1005. | CDR-L3 | QQFNSYPRV |
| 1006. | VH | VQLQQWGAGLLKPSETLSLTCAVYGGSFSGYSWSWIRQPP |
| | | GKGLEWIGEFNHSGNTNYTPSLKSRVTISVDMSKNHFSLKL |
| | | SSVTAADTAVYYCARERDCSGGGCYYPYSYYYGMDVWGQ |
| | | GTTVTVSS |
| 1007. | VL | XXTQSPSFLSASVGDRVTITCRASQGISIYLAWYQQNPGKA |
| | | PKLLIYAASTLQSGVPSRFSGSGSGTEFTLTISNLQPEDFAT |
| | | YYCQQFNSYPRVFGGGTKVEIK |
| 1008. | FR-H1 | VQLQQWGAGLLKPSETLSLTCAVY |
| 1009. | FR-H2 | WSWIRQPPGKGLEWIGE |
| 1010. | FR-H3 | NYTPSLKSRVTISVDMSKNHFSLKLSSVTAADTAVYYC |
| 1011. | FR-H4 | WGQGTTVTVSS |
| 1012. | FR-L1 | XXTQSPSFLSASVGDRVTITCRAS |
| 1013. | FR-L2 | LAWYQQNPGKAPKLLIY |
| 1014. | FR-L3 | TLQSGVPSRFSGSGSGTEFTLTISNLQPEDFATYYC |
| 1015. | FR-L4 | FGGGTKVEIK |
| 1016. | heavychain | VQLQQWGAGLLKPSETLSLTCAVYGGSFSGYSWSWIRQPP |
| | | GKGLEWIGEFNHSGNTNYTPSLKSRVTISVDMSKNHFSLKL |
| | | SSVTAADTAVYYCARERDCSGGGCYYPYSYYYGMDVWGQ |
| | | GTTVTVSSKGPSVFPLAPSSKSTSGGTAALGXLVKDYFPEP |
| | | VTVSWNSGALTSGVHTFPAVLQSSGLYSLSSVVTVPSSSLG |
| | | TQTYICNVNHKPSNTKVDKXVEPKSCDKTHTCPPCPAPELL |
| | | GGPSVFLFPPKP |
| 1017. | lightchain | XXTQSPSFLSASVGDRVTITCRASQGISIYLAWYQQNPGKA |
| | | PKLLIYAASTLQSGVPSRFSGSGSGTEFTLTISNLQPEDFAT |
| | | YYCQQFNSYPRVFGGGTKVEIKAPSVFIFPPSDEQLKSGTA |
| | | SVVCLLNNFYPREAKVQWKVDNALQSGNSQESVTEQDSKD |
| | | STYSLSSTLTLSKADYEKHKVYACEVTHQGLSSPVTKSFNR |
| | | GEC*K |
| 1018. | VH_nuc | aggtgcagctgcagcagtggggcgcaggactgttgaagccttcggagaccctgtcc |
| | | ctcacctgcgctgtttatggtgggtccttcagtggttactcctggagctggatccgccag |
| | | cccccagggaaggggctagagtggattggggaattcaatcatagtggaaacacca |
| | | actacaccccgtccctcaagagtcgagtcaccatatcagtagacatgtccaagaac |
| | | cacttctccctgaagctgagctctgtga |
| 1019. | VL_nuc | tnccngnacccagtctccatccttcctgtctgcatctgtaggagacagagtcaccatc |
| | | acttgccgggccagtcagggcattagcatttatttagcctggtatcagcaaaacccag |
| | | ggaaagcccctaagctcctgatctatgctgcatccactttgcaaagtggggtcccatc |
| | | aaggttcagcggcagtgggtctgggacagagttcactctcacaatcagcaacctgc |
| | | agcctgaagattttgcaacttatta |
| SEQ ID NO | | 11FUP1A2 |
| 1020. | CDR-H1 | GFTVEDYT |
| 1021. | CDR-H2 | ISWDGDRT |
| 1022. | CDR-H3 | VKDNGWRSFAY |
| 1023. | CDR-L1 | QSLLHSNGYNY |
| 1024. | CDR-L2 | LGS |
| 1025. | CDR-L3 | MQALQAFT |
| 1026. | VH | ESGGAVVQPGGSLRLSCAASGFTVEDYTIHWVRQVPGKGL |
| | | EWISLISWDGDRTAYTDSVKGRFTISRDNIKNSLYLLMNSLR |
| | | TEDTALYYCVKDNGWRSFAYWGQGILVTVST |
| 1027. | VL | TQSPLSLPVTPGEPASISCRSSQSLLHSNGYNYLDWYLQRP |
| | | GQSPQLLIYLGSKRASGVPDRFSGSGSGTDFTLKISRVEAE |
| | | DVGVYYCMQALQAFTFGPGTKVDIK |
| 1028. | FR-H1 | ESGGAVVQPGGSLRLSCAAS |
| 1029. | FR-H2 | IHWVRQVPGKGLEWISL |
| 1030. | FR-H3 | AYTDSVKGRFTISRDNIKNSLYLLMNSLRTEDTALYYC |
| 1031. | FR-H4 | WGQGILVTVST |
| 1032. | FR-L1 | TQSPLSLPVTPGEPASISCRSS |
| 1033. | FR-L2 | LDWYLQRPGQSPQLLIY |
| 1034. | FR-L3 | KRASGVPDRFSGSGSGTDFTLKISRVEAEDVGVYYC |
| 1035. | FR-L4 | FGPGTKVDIK |
| 1036. | heavychain | ESGGAVVQPGGSLRLSCAASGFTVEDYTIHWVRQVPGKGL |
| | | EWISLISWDGDRTAYTDSVKGRFTISRDNIKNSLYLLMNSLR |
| | | TEDTALYYCVKDNGWRSFAYWGQGILVTVSTKGPSVFPLAP |
| | | SSKSTSGGTAALGXLVKDYFPEPVTVSWNSGALTSGVHTFP |
| | | AVLQSSGLYSLSSVVTVPSSSLGTQTYICNVNHKPSNTKVD |
| | | KXVEPKSCDKTHTCPPCPAPELLGGPSVFLFPPKPKDTLMIS |
| | | RTPEVTCV |
| 1037. | lightchain | TQSPLSLPVTPGEPASISCRSSQSLLHSNGYNYLDWYLQRP |
| | | GQSPQLLIYLGSKRASGVPDRFSGSGSGTDFTLKISRVEAE |
| | | DVGVYYCMQALQAFTFGPGTKVDIKAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKS |
| | | FNRGEC*K |
| 1038. | VH_nuc | tggagtctgggggagccgtggttcagcctggggggtccctgagactctcctgtgcag |
| | | cctctggattcaccgttgaagattataccatccactgggtccgtcaggttccggggaa |
| | | gggtctggagtggatctctcttattagttgggatggtgatagaacagcgtatacagact |
| | | ctgtgaagggccgattcaccatctcccgagacaacatcaaaaactccctgtatctact |
| | | aatgaacagtctcagaactgagg |
| 1039. | VL_nuc | gactcagtctccactctccctgcccgtcacccctggagagccggcctccatctcctgc |
| | | aggtcaagtcagagtctcctgcatagtaatggatacaactatttggattggtacctgca |
| | | gaggccagggcagtctcctcaactcctgatctatttgggctctaagcgggcctccggg |
| | | gtccctgacaggttcagcggcagtggatcaggcacagattttacactgaaaatcagt |
| | | agagtggaggctgaggacgttgg |
| SEQ ID NO | | 18FU1P1A7 |
| 1040. | CDR-H1 | GFSFNXYA |
| 1041. | CDR-H2 | VSYSGETT |
| 1042. | CDR-H3 | AIGGLALYCSGGSCWH |
| 1043. | CDR-L1 | QGISSY |
| 1044. | CDR-L2 | AAS |
| 1045. | CDR-L3 | QQLNTYPLT |
| 1046. | VH | GXGXXXXGXSLXXSCAASGFSFNXYAMSWVRQAPGKGLD |
| | | WVATVSYSGETTHYAESVKGRFTISRDNSENTVSLQMNSLR |
| | | AEDSAAYYCAIGGLALYCSGGSCWHWGQGTLVTVSP |
| 1047. | VL | TQSPSFLSASVGDRVTITCRASQGISSYLAWYQQKPGKAPK |
| | | LLIYAASTLONGVPSRFSGGGSGTEFTLTISSLOPEDFATYY |
| | | CQQLNTYPLTFGGGTKVEIK |
| 1048. | FR-H1 | GXGXXXXGXSLXXSCAAS |
| 1049. | FR-H2 | MSWVRQAPGKGLDWVAT |
| 1050. | FR-H3 | HYAESVKGRFTISRDNSENTVSLQMNSLRAEDSAAYYC |
| 1051. | FR-H4 | WGQGTLVTVSP |
| 1052. | FR-L1 | TQSPSFLSASVGDRVTITCRAS |
| 1053. | FR-L2 | LAWYQQKPGKAPKLLIY |
| 1054. | FR-L3 | TLQNGVPSRFSGGGSGTEFTLTISSLOPEDFATYYC |
| 1055. | FR-L4 | FGGGTKVEIK |
| 1056. | heavychain | GXGXXXXGXSLXXSCAASGFSFNXYAMSWVRQAPGKGLD |
| | | WVATVSYSGETTHYAESVKGRFTISRDNSENTVSLQMNSLR |
| | | AEDSAAYYCAIGGLALYCSGGSCWHWGQGTLVTVSPKGPS |
| | | VFPLAPSSKSTSGGTAALGXLVKDYFPEPVTVSWNSGALTS |
| | | GVHTFPAVLQSSGLYSLSSVVTVPSSSLGTQTYICNVNHKP |
| | | SNTKVDKXVEPKSCDKTHTCPPCPAPELLGGPSVFLFPPKP |
| | | KDTLMISRTPEV |
| 1057. | lightchain | TQSPSFLSASVGDRVTITCRASQGISSYLAWYQQKPGKAPK |
| | | LLIYAASTLONGVPSRFSGGGSGTEFTLTISSLOPEDFATYY |
| | | CQQLNTYPLTFGGGTKVEIKAPSVFIFPPSDEQLKSGTASVV |
| | | CLLNNFYPREAKVQWKVDNALQSGNSQESVTEQDSKDSTY |
| | | SLSSTLTLSKADYEKHKVYACEVTHQGLSSPVTKSFNRGEC |
| | | *K |
| 1058. | VH_nuc | wgggkgaggmktsgkwcascmkgggkggtccctganastctcmtgtgcagcct |
| | | ctggattctcctttaacwactatgccatgagctgggtccgccaggctccagggaagg |
| | | ggctggactgggtcgcaacggttagttatagtggtgaaaccacacactacgcagaat |
| | | ccgtgaagggccggttcaccatctccagagacaattccgagaacacggtgtctctgc |
| | | agatgaacagcctgagagccgaggactcggc |
| 1059. | VL_nuc | acccagtctccatccttcctgtctgcatctgtgggagacagagtcaccatcacttgccg |
| | | ggccagtcagggcattagcagttatttagcctggtatcagcaaaaaccagggaaag |
| | | cccctaagctcctgatctatgctgcatccactttgcaaaatggggtcccatcaaggttc |
| | | agcggcggtggatctgggacagagttcactctcacaatcagcagcctgcagcctga |
| | | agattttgcaacttattactgtcaa |
| SEQ ID NO | | 23FUP1A8 |
| 1060. | CDR-H1 | GFTFDNYA |
| 1061. | CDR-H2 | ISRRSDNL |
| 1062. | CDR-H3 | VRDRETSLMFDFDY |
| 1063. | CDR-L1 | QSIDKY |
| 1064. | CDR-L2 | AAF |
| 1065. | CDR-L3 | QQTYSTRCS |
| 1066. | VH | VESGGGVVQPGRSLRLSCVASGFTFDNYAMHWVRQAPGK |
| | | GLEWVSGISRRSDNLDYADSVKGRFTISRDNAKNSLYLQMN |
| | | SLHQGXIXXXYCVRDRETSLMFDFDYWGQGSLVTVSS |
| 1067. | VL | QSPSSLSASVGDSVTITCRASQSIDKYLNWYQQRPGKAPKL |
| | | LIYAAFSLQSGVPSRFSGSGSGTDFTLTIGGLQPEDIATYYC |
| | | QQTYSTRCSFGQGTKLEIK |
| 1068. | FR-H1 | VESGGGVVQPGRSLRLSCVAS |
| 1069. | FR-H2 | MHWVRQAPGKGLEWVSG |
| 1070. | FR-H3 | DYADSVKGRFTISRDNAKNSLYLQMNSLHQGXIXXXYC |
| 1071. | FR-H4 | WGQGSLVTVSS |
| 1072. | FR-L1 | QSPSSLSASVGDSVTITCRAS |
| 1073. | FR-L2 | LNWYQQRPGKAPKLLIY |
| 1074. | FR-L3 | SLQSGVPSRFSGSGSGTDFTLTIGGLQPEDIATYYC |
| 1075. | FR-L4 | FGQGTKLEIK |
| 1076. | heavychain | VESGGGVVQPGRSLRLSCVASGFTFDNYAMHWVRQAPGK |
| | | GLEWVSGISRRSDNLDYADSVKGRFTISRDNAKNSLYLQMN |
| | | SLHQGXIXXXYCVRDRETSLMFDFDYWGQGSLVTVSSKGP |
| | | SVFPLAPSSKSTSGGTAALGXLVKDYFPEPVTVSWNSGALT |
| | | SGVHTFPAVLQSSGLYSLSSVVTVPSSSLGTQTYICNVNHK |
| | | PSNTKVDKXVEPKSCDKTHTCPPCPAPELLGGPSVFLFPPK |
| | | PKDTLMISRTPE |
| 1077. | lightchain | QSPSSLSASVGDSVTITCRASQSIDKYLNWYQQRPGKAPKL |
| | | LIYAAFSLQSGVPSRFSGSGSGTDFTLTIGGLQPEDIATYYC |
| | | QQTYSTRCSFGQGTKLEIKAPSVFIFPPSDEQLKSGTASVVC |
| | | LLNNFYPREAKVQWKVDNALQSGNSQESVTEQDSKDSTYS |
| | | LSSTLTLSKADYEKHKVYACEVTHQGLSSPVTKSFNRGEC* |
| | | K |
| 1078. | VH_nuc | ggtggaatctgggggaggcgtggtacagccgggcaggtccctgagactctcctgtgt |
| | | cgcctctggattcacctttgataattatgccatgcactgggtccggcaagctccaggga |
| | | agggcctggagtgggtctcaggtattagtcggagaagtgataaccttgactacgcgg |
| | | actctgtgaagggccgattcaccatctccagagacaatgccaagaactccctgtatct |
| | | gcaaatgaacagcctccaccaagg |
| 1079. | VL_nuc | ccagtctccatcctccctgtctgcatctgtaggagacagcgtcaccatcacttgccggg |
| | | caagtcagagcattgataagtatttaaattggtatcaacagagaccaggaaaagcc |
| | | cctaaactcctcatctatgctgcattcagtttacaaagtggagtcccttcaaggttcagt |
| | | ggcagtggatctgggacagatttcactctcaccatcggcggtctgcaacctgaagat |
| | | attgcaacttactactgtcagca |
| SEQ ID NO | | 23FUP1B8 |
| 1080. | CDR-H1 | GFIFDDYS |
| 1081. | CDR-H2 | IKWNGEST |
| 1082. | CDR-H3 | VKDGGLRYXQH |
| 1083. | CDR-L1 | QSLLHSNGYMY |
| 1084. | CDR-L2 | LGS |
| 1085. | CDR-L3 | MQSLKSFT |
| 1086. | VH | XXVESGGDVVQPGGSLRLSCAASGFIFDDYSMHWVRQGP |
| | | GKTLEWVSLIKWNGESTSYADSVKGRFTISRDNTKSALYLE |
| | | MSNLRPDDTAFYYCVKDGGLRYXQHWGRGTLVTVSS |
| 1087. | VL | TQSPLSLPVTPGEPASISCRSSQSLLHSNGYMYLDWYLQKP |
| | | GQSPRLLIYLGSNRASGVPDRFSGSGSGTDFTLKISRVEAE |
| | | DVGVYFCMQSLKSFTFGPGTKVDIK |
| 1088. | FR-H1 | XXVESGGDVVQPGGSLRLSCAAS |
| 1089. | FR-H2 | MHWVRQGPGKTLEWVSL |
| 1090. | FR-H3 | SYADSVKGRFTISRDNTKSALYLEMSNLRPDDTAFYYC |
| 1091. | FR-H4 | WGRGTLVTVSS |
| 1092. | FR-L1 | TQSPLSLPVTPGEPASISCRSS |
| 1093. | FR-L2 | LDWYLQKPGQSPRLLIY |
| 1094. | FR-L3 | NRASGVPDRFSGSGSGTDFTLKISRVEAEDVGVYFC |
| 1095. | FR-L4 | FGPGTKVDIK |
| 1096. | heavychain | XXVESGGDVVQPGGSLRLSCAASGFIFDDYSMHWVRQGP |
| | | GKTLEWVSLIKWNGESTSYADSVKGRFTISRDNTKSALYLE |
| | | MSNLRPDDTAFYYCVKDGGLRYXQHWGRGTLVTVSSKGP |
| | | SVFPLAPSSKSTSGGTAALGXLVKDYFPEPVTVSWNSGALT |
| | | SGVHTFPAVLQSSGLYSLSSVVTVPSSSLGTQTYICNVNHK |
| | | PSNTKVDKXVEPKSCDKTHTCPPCPAPELLGGPSVFLFPPK |
| | | PKDTLMISRTPEV |
| 1097. | lightchain | TQSPLSLPVTPGEPASISCRSSQSLLHSNGYMYLDWYLQKP |
| | | GQSPRLLIYLGSNRASGVPDRFSGSGSGTDFTLKISRVEAE |
| | | DVGVYFCMQSLKSFTFGPGTKVDIKAPSVFIFPPSDEQLKSG |
| | | TASVVCLLNNFYPREAKVQWKVDNALQSGNSQESVTEQDS |
| | | KDSTYSLSSTLTLSKADYEKHKVYACEVTHQGLSSPVTKSF |
| | | NRGEC*K |
| 1098. | VH_nuc | ccangnnngtggaatcggggggagacgtggttcagcctggggggtccctgagact |
| | | ctcctgtgcagcctctggattcatctttgatgattactccatgcactgggtccgtcaaggt |
| | | ccggggaagactctggagtgggtctctctcataaaatggaatggtgaaagcacttcct |
| | | acgcagactctgtgaagggtcgattcaccatctccagagacaacaccaaaagcgc |
| | | cctttatctggaaatgagcaatctga |
| 1099. | VL_nuc | agactcagtctccactctccctgcccgtcacccctggagagccggcctccatctcctg |
| | | caggtctagtcagagcctcctgcatagtaatggatacatgtatttggattggtacctgca |
| | | aaagccagggcagtctccacgcctcctgatctatttgggttctaatcgggcctccggg |
| | | gtccctgacaggttcagtggcagtggatcaggcacagattttacactgaaaatcagc |
| | | agagtggaggctgaggatgttg |
| SEQ ID NO | | 23FUP1C4 |
| 1100. | CDR-H1 | GFTFDDFT |
| 1101. | CDR-H2 | INWDGRRT |
| 1102. | CDR-H3 | VKDGGLRYFQY |
| 1103. | CDR-L1 | QSLVHRNGYNY |
| 1104. | CDR-L2 | LGS |
| 1105. | CDR-L3 | MQSLETFT |
| 1106. | VH | ARXVESGGVVVQPGGSLRLLCAASGFTFDDFTMHWVRQSP |
| | | GKGLEWVSLINWDGRRTEYADSVKGRFSISRDSSQNSLYL |
| | | QMNSLKTEDTALYYCVKDGGLRYFQYWGRGTLVTVSS |
| 1107. | VL | TQSPLSLPVTPGEPASISCRSSQSLVHRNGYNYLDWYLQKP |
| | | GQSPQLLIYLGSKRASGVPDRFSGSGSGTDFTLKISRVEAE |
| | | DVGVYYCMQSLETFTFGPGTKVEIK |
| 1108. | FR-H1 | ARXVESGGVVVQPGGSLRLLCAAS |
| 1109. | FR-H2 | MHWVRQSPGKGLEWVSL |
| 1110. | FR-H3 | EYADSVKGRFSISRDSSQNSLYLQMNSLKTEDTALYYC |
| 1111. | FR-H4 | WGRGTLVTVSS |
| 1112. | FR-L1 | TQSPLSLPVTPGEPASISCRSS |
| 1113. | FR-L2 | LDWYLQKPGQSPQLLIY |
| 1114. | FR-L3 | KRASGVPDRFSGSGSGTDFTLKISRVEAEDVGVYYC |
| 1115. | FR-L4 | FGPGTKVEIK |
| 1116. | heavychain | ARXVESGGVVVQPGGSLRLLCAASGFTFDDFTMHWVRQSP |
| | | GKGLEWVSLINWDGRRTEYADSVKGRFSISRDSSQNSLYL |
| | | QMNSLKTEDTALYYCVKDGGLRYFQYWGRGTLVTVSSKGP |
| | | SVFPLAPSSKSTSGGTAALGXLVKDYFPEPVTVSWNSGALT |
| | | SGVHTFPAVLQSSGLYSLSSVVTVPSSSLGTQTYICNVNHK |
| | | PSNTKVDKXVEPKSCDKTHTCPPCPAPELLGGPSVFLFPPK |
| | | PKDTLMISRTPE |
| 1117. | lightchain | TQSPLSLPVTPGEPASISCRSSQSLVHRNGYNYLDWYLQKP |
| | | GQSPQLLIYLGSKRASGVPDRFSGSGSGTDFTLKISRVEAE |
| | | DVGVYYCMQSLETFTFGPGTKVEIKAPSVFIFPPSDEQLKSG |
| | | TASVVCLLNNFYPREAKVQWKVDNALQSGNSQESVTEQDS |
| | | KDSTYSLSSTLTLSKADYEKHKVYACEVTHQGLSSPVTKSF |
| | | NRGEC*K |
| 1118. | VH_nuc | tgccaggnnngtggagtctgggggagtcgtggtacagccgggggggtccctgaga |
| | | ctcttgtgtgcagcctctggattcacctttgatgattttaccatgcactgggtccgacaaa |
| | | gtccggggaagggtctcgagtgggtctcccttattaattgggatggtcgcagaacag |
| | | aatatgcagactctgtgaagggccgattttccatctccagagacagcagccaaaact |
| | | ccctctatctacaaatgaacagtct |
| 1119. | VL_nuc | gactcagtctccgctctccctgcccgtcacccctggagagccggcctccatctcctgc |
| | | aggtctagtcagagcctcgtgcatagaaatggatacaactacttggattggtacctgc |
| | | agaagccagggcagtctccacagctcctgatctatttgggttctaagcgagcctccgg |
| | | ggtccctgacaggttcagtggcagtggatcaggcacagattttacactgaaaatcag |
| | | cagagtggaggctgaggatgttgg |
| SEQ ID NO | | 23FUP1C10 |
| 1120. | CDR-H1 | GYTLTRYD |
| 1121. | CDR-H2 | MNPKSGGS |
| 1122. | CDR-H3 | ARAVDLDN |
| 1123. | CDR-L1 | QSLLDTSNNNNY |
| 1124. | CDR-L2 | WAS |
| 1125. | CDR-L3 | LQYYSLPHT |
| 1126. | VH | VQXVQSGAEVKKPGASVKVSCKASGYTLTRYDINWVRQAT |
| | | GQGLEWMGWMNPKSGGSGSAQRFQGRVTMTWNNSISTA |
| | | YMELSTLRSDDTAVYYCARAVDLDNWGQGTLVIVSS |
| 1127. | VL | TQSPDSLPVSLGERATINCKSSQSLLDTSNNNNYLGWYQQK |
| | | PGQPPKLLFYWASARESGVPDRFSGGGSGTDFTLTISSLQA |
| | | EDVAVYYCLQYYSLPHTFGGGTKVEIK |
| 1128. | FR-H1 | VQXVQSGAEVKKPGASVKVSCKAS |
| 1129. | FR-H2 | INWVRQATGQGLEWMGW |
| 1130. | FR-H3 | GSAQRFQGRVTMTWNNSISTAYMELSTLRSDDTAVYYC |
| 1131. | FR-H4 | WGQGTLVIVSS |
| 1132. | FR-L1 | TQSPDSLPVSLGERATINCKSS |
| 1133. | FR-L2 | LGWYQQKPGQPPKLLFY |
| 1134. | FR-L3 | ARESGVPDRFSGGGSGTDFTLTISSLQAEDVAVYYC |
| 1135. | FR-L4 | FGGGTKVEIK |
| 1136. | heavychain | VQXVQSGAEVKKPGASVKVSCKASGYTLTRYDINWVRQAT |
| | | GQGLEWMGWMNPKSGGSGSAQRFQGRVTMTWNNSISTA |
| | | YMELSTLRSDDTAVYYCARAVDLDNWGQGTLVIVSSKGPSV |
| | | FPLAPSSKSTSGGTAALGXLVKDYFPEPVTVSWNSGALTSG |
| | | VHTFPAVLQSSGLYSLSSVVTVPSSSLGTQTYICNVNHKPSN |
| | | TKVDKXVEPKSCDKTHTCPPCPAPELLGGPSVFLFPPKPKD |
| | | TLMISRTPEVTC |
| 1137. | lightchain | TQSPDSLPVSLGERATINCKSSQSLLDTSNNNNYLGWYQQK |
| | | PGQPPKLLFYWASARESGVPDRFSGGGSGTDFTLTISSLQA |
| | | EDVAVYYCLQYYSLPHTFGGGTKVEIKAPSVFIFPPSDEQLK |
| | | SGTASVVCLLNNFYPREAKVQWKVDNALQSGNSQESVTEQ |
| | | DSKDSTYSLSSTLTLSKADYEKHKVYACEVTHQGLSSPVTK |
| | | SFNRGEC*K |
| 1138. | VH_nuc | gtgcaagnngtgcagtctggggctgaggtgaagaagcctggggcctcagtgaagg |
| | | tctcctgcaaggcttctggatacaccctcaccagatacgatatcaactgggtacgaca |
| | | ggccactggccaaggacttgagtggatgggatggatgaaccctaaaagtgggggc |
| | | agtggctctgcacagagattccagggcagagtcaccatgacctggaacaattccat |
| | | aagcacagcctacatggagctgagcaccctg |
| 1139. | VL_nuc | agacccagtctccagactccctgcctgtgtctctgggcgagagggccaccatcaact |
| | | gcaagtccagccagagtcttttagacacctccaacaacaacaactacttaggttggt |
| | | accagcagaaaccaggacagcctccaaaattgctcttttactgggcatctgcccggg |
| | | aatccggggtccctgaccgattcagtggcggcgggtctgggacagatttcactctcac |
| | | catcagcagcctgcaggctgaagatg |
| SEQ ID NO | | 23FUP1D6 |
| 1140. | CDR-H1 | GFTFDDYT |
| 1141. | CDR-H2 | IRYDGTRR |
| 1142. | CDR-H3 | VKDGGLRYFDY |
| 1143. | CDR-L1 | QSLLHSNGIHY |
| 1144. | CDR-L2 | LGS |
| 1145. | CDR-L3 | MQSLQTFT |
| 1146. | VH | GAXXVESGGDVVQPGGSLRLSCEASGFTFDDYTMHWVRQI |
| | | PGKSLEWLSLIRYDGTRREYADSVKGRFTISRDNSKNSLFLH |
| | | MNSLKTDDSGFYYCVKDGGLRYFDYWGQGALVTVSS |
| 1147. | VL | TQSPLSLSVTPGEPASISCRSSQSLLHSNGIHYLDWYLQKP |
| | | GQSPQLLIYLGSKRASGVPDRFSGSGSGTDFTLKISRVEAE |
| | | DVGVYYCMQSLQTFTFGPGTKVDIK |
| 1148. | FR-H1 | GAXXVESGGDVVQPGGSLRLSCEAS |
| 1149. | FR-H2 | MHWVRQIPGKSLEWLSL |
| 1150. | FR-H3 | EYADSVKGRFTISRDNSKNSLFLHMNSLKTDDSGFYYC |
| 1151. | FR-H4 | WGQGALVTVSS |
| 1152. | FR-L1 | TQSPLSLSVTPGEPASISCRSS |
| 1153. | FR-L2 | LDWYLQKPGQSPQLLIY |
| 1154. | FR-L3 | KRASGVPDRFSGSGSGTDFTLKISRVEAEDVGVYYC |
| 1155. | FR-L4 | FGPGTKVDIK |
| 1156. | heavychain | GAXXVESGGDVVQPGGSLRLSCEASGFTFDDYTMHWVRQI |
| | | PGKSLEWLSLIRYDGTRREYADSVKGRFTISRDNSKNSLFLH |
| | | MNSLKTDDSGFYYCVKDGGLRYFDYWGQGALVTVSSKGP |
| | | SVFPLAPSSKSTSGGTAALGXLVKDYFPEPVTVSWNSGALT |
| | | SGVHTFPAVLQSSGLYSLSSVVTVPSSSLGTQTYICNVNHK |
| | | PSNTKVDKXVEPKSCDKTHTCPPCPAPELLGGPSVFLFPPK |
| | | PKDTLMISRTP |
| 1157. | lightchain | TQSPLSLSVTPGEPASISCRSSQSLLHSNGIHYLDWYLQKP |
| | | GQSPQLLIYLGSKRASGVPDRFSGSGSGTDFTLKISRVEAE |
| | | DVGVYYCMQSLQTFTFGPGTKVDIKAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKS |
| | | FNRGEC*K |
| 1158. | VH_nuc | ggtgcccannnggtggagtctgggggagacgtggtgcagcctggggggtccctga |
| | | gactctcctgtgaagcctctggattcacctttgatgattatactatgcactgggtccgtca |
| | | aattccggggaagagtctggagtggctctctcttattcgttacgacgggactaggaga |
| | | gaatatgcagactccgtgaagggtcgattcaccatctccagagacaacagcaaaa |
| | | actccctgtttctgcacatgaacagt |
| 1159. | VL_nuc | agactcagtcaccactctccctgtccgtcacccctggagagccggcctccatctcctg |
| | | caggtcaagtcagagcctcctgcacagtaatggaatccactatttggattggtacctg |
| | | cagaagccagggcagtctccacagctcctgatctatttgggttctaaacgggcctccg |
| | | gggtccctgacaggttcagtggcagtggatcaggcacagattttacactgaaaatca |
| | | gcagagtggaggctgaggatgttg |
| SEQ ID NO | | 23FUP1D7 |
| 1160. | CDR-H1 | GFYIGGSS |
| 1161. | CDR-H2 | IRSFSHAFAT |
| 1162. | CDR-H3 | TRPFRGYDLSSDFYPD |
| 1163. | CDR-L1 | QTISGGS |
| 1164. | CDR-L2 | DAS |
| 1165. | CDR-L3 | QQYSESPQT |
| 1166. | VH | GASLKLSCAVSGFYIGGSSIHWVRQTPGRGLEWLGRIRSFS |
| | | HAFATAYTPSLRGRITISTNESQNTAFLVLTSLSRDDTAIYYC |
| | | TRPFRGYDLSSDFYPDWGQGTLVTVSS |
| 1167. | VL | TQSPGTLSLSPGEGATLSCRASQTISGGSLAWYQQRPGLA |
| | | PRLLMYDASTRATGIPKRFSGSGSGTDFTLTISRLEPEDFAV |
| | | YYCQQYSESPQTFGQGTKVEIK |
| 1168. | FR-H1 | GASLKLSCAVS |
| 1169. | FR-H2 | IHWVRQTPGRGLEWLGR |
| 1170. | FR-H3 | AYTPSLRGRITISTNESQNTAFLVLTSLSRDDTAIYYC |
| 1171. | FR-H4 | WGQGTLVTVSS |
| 1172. | FR-L1 | TQSPGTLSLSPGEGATLSCRAS |
| 1173. | FR-L2 | LAWYQQRPGLAPRLLMY |
| 1174. | FR-L3 | TRATGIPKRFSGSGSGTDFTLTISRLEPEDFAVYYC |
| 1175. | FR-L4 | FGQGTKVEIK |
| 1176. | heavychain | GASLKLSCAVSGFYIGGSSIHWVRQTPGRGLEWLGRIRSFS |
| | | HAFATAYTPSLRGRITISTNESQNTAFLVLTSLSRDDTAIYYC |
| | | TRPFRGYDLSSDFYPDWGQGTLVTVSSKGPSVFPLAPSSK |
| | | STSGGTAALGXLVKDYFPEPVTVSWNSGALTSGVHTFPAVL |
| | | QSSGLYSLSSVVTVPSSSLGTQTYICNVNHKPSNTKVDKXV |
| | | EPKSCDKTHTCPPCPAPELLGGPSVFLFPPKPKDTLMISRTP |
| | | EVTCVVV |
| 1177. | lightchain | TQSPGTLSLSPGEGATLSCRASQTISGGSLAWYQQRPGLA |
| | | PRLLMYDASTRATGIPKRFSGSGSGTDFTLTISRLEPEDFAV |
| | | YYCQQYSESPQTFGQGTKVEIKAPSVFIFPPSDEQLKSGTA |
| | | SVVCLLNNFYPREAKVQWKVDNALQSGNSQESVTEQDSKD |
| | | STYSLSSTLTLSKADYEKHKVYACEVTHQGLSSPVTKSFNR |
| | | GEC*K |
| 1178. | VH_nuc | ggtgcatccctcaaactctcctgtgcagtgtcgggattctacattggcggctcgtccata |
| | | cactgggtccgccagactcccgggagagggctggagtggcttggccgaataagaa |
| | | gcttttctcacgcttttgcgacggcctatactccgtcgctgaggggcaggatcaccattt |
| | | ccacaaatgagtcgcagaacacggcctttttggtgctgaccagcctgagtcgggac |
| | | gacacggccatttactactgtact |
| 1179. | VL_nuc | gacgcagtctccaggcaccctgtctttgtctccaggggaaggcgccaccctctcatgt |
| | | agggccagtcagactattagtggcggctctttagcctggtatcaacaaagacctggc |
| | | ctggcgcccaggctcctcatgtatgacgcgtccaccagggccactggcatcccaaa |
| | | gagattcagtggcagtgggtccgggacagacttcacgctcacaattagcagactgg |
| | | agcctgaagattttgcagtatattactg |
| SEQ ID NO | | 23FUP1D8 |
| 1180. | CDR-H1 | GFTLTTYA |
| 1181. | CDR-H2 | MSKDGSAT |
| 1182. | CDR-H3 | ARDTQDWPTLPHHFYGMDV |
| 1183. | CDR-L1 | QDIKNY |
| 1184. | CDR-L2 | DAS |
| 1185. | CDR-L3 | QQYDNLPLT |
| 1186. | VH | WXXVESGGGVVQPGRSLTISCEVSGFTLTTYAVHWVRQAP |
| | | GKGLEWVAVMSKDGSATYYAGSVRGRFTISRDISKKMMYL |
| | | EMNSLRGEDTGVYYCARDTQDWPTLPHHFYGMDVWGQG |
| | | TTVIVSS |
| 1187. | VL | DIVMTQSPSSLSASVGDRVTITCQASQDIKNYLNWYQQKPG |
| | | KPPKFLIYDASSLETGVPSRFSGSGSGTDFTLTINSLQPEDIA |
| | | TYYCQQYDNLPLTFGGGTKVEIK |
| 1188. | FR-H1 | WXXVESGGGVVQPGRSLTISCEVS |
| 1189. | FR-H2 | VHWVRQAPGKGLEWVAV |
| 1190. | FR-H3 | YYAGSVRGRFTISRDISKKMMYLEMNSLRGEDTGVYYC |
| 1191. | FR-H4 | WGQGTTVIVSS |
| 1192. | FR-L1 | DIVMTQSPSSLSASVGDRVTITCQAS |
| 1193. | FR-L2 | LNWYQQKPGKPPKFLIY |
| 1194. | FR-L3 | SLETGVPSRFSGSGSGTDFTLTINSLQPEDIATYYC |
| 1195. | FR-L4 | FGGGTKVEIK |
| 1196. | heavychain | WXXVESGGGVVQPGRSLTISCEVSGFTLTTYAVHWVRQAP |
| | | GKGLEWVAVMSKDGSATYYAGSVRGRFTISRDISKKMMYL |
| | | EMNSLRGEDTGVYYCARDTQDWPTLPHHFYGMDVWGQG |
| | | TTVIVSSKGPSVFPLAPSSKSTSGGTAALGXLVKDYFPEPVT |
| | | VSWNSGALTSGVHTFPAVLQSSGLYSLSSVVTVPSSSLGTQ |
| | | TYICNVNHKPSNTKVDKXVEPKSCDKTHTCPPCPAPELLGG |
| | | PSVFLFPPKPKDT |
| 1197. | lightchain | DIVMTQSPSSLSASVGDRVTITCQASQDIKNYLNWYQQKPG |
| | | KPPKFLIYDASSLETGVPSRFSGSGSGTDFTLTINSLQPEDIA |
| | | TYYCQQYDNLPLTFGGGTKVEIKAPSVFIFPPSDEQLKSGTA |
| | | SVVCLLNNFYPREAKVQWKVDNALQSGNSQESVTEQDSKD |
| | | STYSLSSTLTLSKADYEKHKVYACEVTHQGLSSPVTKSFNR |
| | | GEC*K |
| 1198. | VH_nuc | gtggnnnnnngtngagtctgggggaggcgtggtccagcctgggaggtccctgaca |
| | | atctcctgtgaagtctctggattcaccctcacaacttatgctgtccactgggtccgccag |
| | | gctccaggcaaggggctggagtgggtggcagttatgtcaaaagatggaagcgcga |
| | | catattatgcaggctccgtgaggggccgattcaccatctccagagacatttccaaga |
| | | aaatgatgtatttggaaatgaacagcct |
| 1199. | VL_nuc | gacatcgtgatgacccagtctccatcctccctgtctgcatctgtaggagacagagtca |
| | | ccatcacttgccaggcgagtcaagacattaagaactatttaaattggtatcagcagaa |
| | | accagggaaaccccctaagttcctcatctacgatgcatccagtttggaaacgggggt |
| | | cccatcaaggttcagtggaagtggatctgggacagattttactctcaccatcaacagc |
| | | ctgcagcctgaagatattgcaaca |
| SEQ ID NO | | 23FUP1D12 |
| 1200. | CDR-H1 | GFTFDDYT |
| 1201. | CDR-H2 | ISWKNHSL |
| 1202. | CDR-H3 | AKDNGFRSFDS |
| 1203. | CDR-L1 | QSLLHSNGYKY |
| 1204. | CDR-L2 | MGS |
| 1205. | CDR-L3 | MQALQTPWT |
| 1206. | VH | CXXXESGGGLVQPGRXLRLSCAASGFTFDDYTMHWVRQV |
| | | PGKGLEWVSGISWKNHSLGYADSVKGRFTISRDNAKNSLYL |
| | | QMNSLRSEDTALYYCAKDNGFRSFDSWGQGTLVTVSS |
| 1207. | VL | TQSPLSLAVTPGEPASISCRSSQSLLHSNGYKYLDWYLQKP |
| | | GQSPQLLIYMGSNRASGVPDRFSVSGSGTDFTLKISRVEAA |
| | | DVGVYYCMQALQTPWTFGQGTKVEIK |
| 1208. | FR-H1 | CXXXESGGGLVQPGRXLRLSCAAS |
| 1209. | FR-H2 | MHWVRQVPGKGLEWVSG |
| 1210. | FR-H3 | GYADSVKGRFTISRDNAKNSLYLQMNSLRSEDTALYYC |
| 1211. | FR-H4 | WGQGTLVTVSS |
| 1212. | FR-L1 | TQSPLSLAVTPGEPASISCRSS |
| 1213. | FR-L2 | LDWYLQKPGQSPQLLIY |
| 1214. | FR-L3 | NRASGVPDRFSVSGSGTDFTLKISRVEAADVGVYYC |
| 1215. | FR-L4 | FGQGTKVEIK |
| 1216. | heavychain | CXXXESGGGLVQPGRXLRLSCAASGFTFDDYTMHWVRQV |
| | | PGKGLEWVSGISWKNHSLGYADSVKGRFTISRDNAKNSLYL |
| | | QMNSLRSEDTALYYCAKDNGFRSFDSWGQGTLVTVSSKGP |
| | | SVFPLAPSSKSTSGGTAALGXLVKDYFPEPVTVSWNSGALT |
| | | SGVHTFPAVLQSSGLYSLSSVVTVPSSSLGTQTYICNVNHK |
| | | PSNTKVDKXVEPKSCDKTHTCPPCPAPELLGGPSVFLFPPK |
| | | PKDTLMISRTPE |
| 1217. | lightchain | TQSPLSLAVTPGEPASISCRSSQSLLHSNGYKYLDWYLQKP |
| | | GQSPQLLIYMGSNRASGVPDRFSVSGSGTDFTLKISRVEAA |
| | | DVGVYYCMQALQTPWTFGQGTKVEIKAPSVFIFPPSDEQLK |
| | | SGTASVVCLLNNFYPREAKVQWKVDNALQSGNSQESVTEQ |
| | | DSKDSTYSLSSTLTLSKADYEKHKVYACEVTHQGLSSPVTK |
| | | SFNRGEC*K |
| 1218. | VH_nuc | gtgccannnnnnggagtctgggggaggcttggttcagcctggcaggtscctgagac |
| | | tctcctgtgcagcctctggattcacctttgatgattataccatgcactgggtccggcaagt |
| | | tccagggaagggcctggagtgggtctcaggtattagttggaagaatcacagtctagg |
| | | ctatgcggactctgtgaagggccgattcaccatctccagagacaacgccaagaact |
| | | ccctatatctgcaaatgaacagtct |
| 1219. | VL_nuc | gactcagtctccactctccctggccgtcacccctggagagccggcctccatctcctgc |
| | | aggtctagtcagagcctcctgcatagtaatggatacaagtatttggattggtacctgca |
| | | gaagccagggcagtctccacagctcctgatctatatgggttctaaccgggcctccgg |
| | | ggtccctgacaggttcagtgtcagtgggtcaggcacagattttacactgaaaatcagc |
| | | agagtggaggctgcggatgttgg |
| SEQ ID NO | | 24BU7P1A10 |
| 1220. | CDR-H1 | GFVFDDYS |
| 1221. | CDR-H2 | ISWDGGRT |
| 1222. | CDR-H3 | VKDTGLRSFDS |
| 1223. | CDR-L1 | QSLMHTNGYIY |
| 1224. | CDR-L2 | LGS |
| 1225. | CDR-L3 | MQALQSWT |
| 1226. | VH | ESGGEVVQPGRSLRLSCAASGFVFDDYSMHWVRQVPGKG |
| | | LEWVALISWDGGRTSYGHSVKGRFTISRDNTKDSLFLQMNS |
| | | LRPEDTALYYCVKDTGLRSFDSWGQGILVTVSS |
| 1227. | VL | MXXXTQSPLSLPVTPGEPASISCRSSQSLMHTNGYIYLDWY |
| | | LQKPGQSPQLLIYLGSKRAPGVPDRFSGSGSGTDFTLKISR |
| | | VEAEDVGVYYCMQALQSWTFGQGTKVEIK |
| 1228. | FR-H1 | ESGGEVVQPGRSLRLSCAAS |
| 1229. | FR-H2 | MHWVRQVPGKGLEWVAL |
| 1230. | FR-H3 | SYGHSVKGRFTISRDNTKDSLFLQMNSLRPEDTALYYC |
| 1231. | FR-H4 | WGQGILVTVSS |
| 1232. | FR-L1 | MXXXTQSPLSLPVTPGEPASISCRSS |
| 1233. | FR-L2 | LDWYLQKPGQSPQLLIY |
| 1234. | FR-L3 | KRAPGVPDRFSGSGSGTDFTLKISRVEAEDVGVYYC |
| 1235. | FR-L4 | FGQGTKVEIK |
| 1236. | heavychain | ESGGEVVQPGRSLRLSCAASGFVFDDYSMHWVRQVPGKG |
| | | LEWVALISWDGGRTSYGHSVKGRFTISRDNTKDSLFLQMNS |
| | | LRPEDTALYYCVKDTGLRSFDSWGQGILVTVSSKGPSVFPL |
| | | APSSKSTSGGTAALGXLVKDYFPEPVTVSWNSGALTSGVHT |
| | | FPAVLQSSGLYSLSSVVTVPSSSLGTQTYICNVNHKPSNTKV |
| | | DKXVEPKSCDKTHTCPPCPAPELLGGPSVFLFPPKPKDTLMI |
| | | SRTPEVTCV |
| 1237. | lightchain | MXXXTQSPLSLPVTPGEPASISCRSSQSLMHTNGYIYLDWY |
| | | LQKPGQSPQLLIYLGSKRAPGVPDRFSGSGSGTDFTLKISR |
| | | VEAEDVGVYYCMQALQSWTFGQGTKVEIKAPSVFIFPPSDE |
| | | QLKSGTASVVCLLNNFYPREAKVQWKVDNALQSGNSQESV |
| | | TEQDSKDSTYSLSSTLTLSKADYEKHKVYACEVTHQGLSSP |
| | | VTKSFNRGEC*K |
| 1238. | VH_nuc | tggagtctggcggagaggtcgtacagccggggaggtccctgaggctgtcctgtgca |
| | | gcctctggattcgtctttgatgattattccatgcactgggtccgtcaagtcccggggaag |
| | | ggccttgagtgggtcgctctaattagttgggatggtggtcgcacatcctatggacactct |
| | | gtgaagggccgattcaccatctccagagacaacaccaaggactccctgtttctgcaa |
| | | atgaacagtctgagacccgagg |
| 1239. | VL_nuc | atgttngnnnagactcagtctccactctccctgcccgtcactcctggagagccggcct |
| | | ccatctcctgcaggtctagtcagagcctcatgcatactaatggatacatctatttggatt |
| | | ggtacctgcagaagcctgggcagtctccacagctcctgatctatctgggttctaagcg |
| | | ggcccccggggtccccgacaggttcagtggcagtggatcaggcacagattttacact |
| | | gaaaatcagcagagtggaggct |
| SEQ ID NO | | 24BU7P1B6 |
| 1240. | CDR-H1 | GFTFSHAS |
| 1241. | CDR-H2 | IRSKTDGGTT |
| 1242. | CDR-H3 | NTHFYCNRSMCYGDY |
| 1243. | CDR-L1 | QSVLYSSNNKNY |
| 1244. | CDR-L2 | WAS |
| 1245. | CDR-L3 | QQYYSGRT |
| 1246. | VH | ESGGGLVKPGGSLRLSCAASGFTFSHASMSWVRQAPGKG |
| | | LEWVGRIRSKTDGGTTDYAAPVKGRFTISRDDSKNTLYLQM |
| | | NSLKTEDTAVYYCNTHFYCNRSMCYGDYWGQGTLVTVSS |
| 1247. | VL | DIXXTQSPDSLAVSLGERATINCKSSQSVLYSSNNKNYLAW |
| | | YQQKPGQPPKLLIYWASTRESGVPDRFRGSGSGTDFSLTIS |
| | | SLQAEDVAVYYCQQYYSGRTFGPGTKVDFK |
| 1248. | FR-H1 | ESGGGLVKPGGSLRLSCAAS |
| 1249. | FR-H2 | MSWVRQAPGKGLEWVGR |
| 1250. | FR-H3 | DYAAPVKGRFTISRDDSKNTLYLQMNSLKTEDTAVYYC |
| 1251. | FR-H4 | WGQGTLVTVSS |
| 1252. | FR-L1 | DIXXTQSPDSLAVSLGERATINCKSS |
| 1253. | FR-L2 | LAWYQQKPGQPPKLLIY |
| 1254. | FR-L3 | TRESGVPDRFRGSGSGTDFSLTISSLQAEDVAVYYC |
| 1255. | FR-L4 | FGPGTKVDFK |
| 1256. | heavychain | ESGGGLVKPGGSLRLSCAASGFTFSHASMSWVRQAPGKG |
| | | LEWVGRIRSKTDGGTTDYAAPVKGRFTISRDDSKNTLYLQM |
| | | NSLKTEDTAVYYCNTHFYCNRSMCYGDYWGQGTLVTVSSK |
| | | GPSVFPLAPSSKSTSGGTAALGXLVKDYFPEPVTVSWNSGA |
| | | LTSGVHTFPAVLQSSGLYSLSSVVTVPSSSLGTQTYICNVNH |
| | | KPSNTKVDKXVEPKSCDKTHTCPPCPAPELLGGPSVFLFPP |
| | | KPKDTLMISRT |
| 1257. | lightchain | DIXXTQSPDSLAVSLGERATINCKSSQSVLYSSNNKNYLAW |
| | | YQQKPGQPPKLLIYWASTRESGVPDRFRGSGSGTDFSLTIS |
| | | SLQAEDVAVYYCQQYYSGRTFGPGTKVDFKAPSVFIFPPSD |
| | | EQLKSGTASVVCLLNNFYPREAKVQWKVDNALQSGNSQES |
| | | VTEQDSKDSTYSLSSTLTLSKADYEKHKVYACEVTHQGLSS |
| | | PVTKSFNRGEC*K |
| 1258. | VH_nuc | tngagtctgggggaggcttggtaaagcctggggggtcccttagactctcctgtgcagc |
| | | ctctggattcactttcagtcacgcctcgatgagctgggtccgccaggctccagggaag |
| | | gggctggagtgggttggccgtattagaagcaaaactgatggcgggacaacagact |
| | | acgctgcacccgtgaaaggcagattcaccatctcaagagatgattcaaaaaacac |
| | | actgtatctgcaaatgaacagcctgaaaa |
| 1259. | VL_nuc | gacatcgnnnagacccagtctccagactccctggctgtgtctctgggcgagagggc |
| | | caccatcaactgcaagtccagccagagtgttttatacagctccaataataagaattac |
| | | ttagcttggtaccagcagaaaccagggcagcctcctaagttgctcatttactgggcat |
| | | ctacccgggaatccggggtccctgaccgattcagaggcagcgggtctgggacaga |
| | | tttctctctcaccatcagcagcctgcag |
| SEQ ID NO | | 24BU7P1D1 |
| 1260. | CDR-H1 | GFTFEDYS |
| 1261. | CDR-H2 | ISWDGGRT |
| 1262. | CDR-H3 | VKDTGLRSFDS |
| 1263. | CDR-L1 | QSLVHSNGINY |
| 1264. | CDR-L2 | MGF |
| 1265. | CDR-L3 | MQALQSWT |
| 1266. | VH | VESGGVVVQRGGSLRLSCAGSGFTFEDYSMHWVRQSPGK |
| | | GLEWVSLISWDGGRTSYGDSVKGRFTISRDNSKKSLFLQMT |
| | | NLRPEDTALYYCVKDTGLRSFDSWGQGTLVTVSS |
| 1267. | VL | MXXXTQSPLSLPVTPGEPASISCRSSQSLVHSNGINYLDWYI |
| | | QKPGQSPQLLIYMGFKRAAGVPDRFSGSVSGTDFTLMISRV |
| | | EAEDVGVYYCMQALQSWTFGQGTRVEIK |
| 1268. | FR-H1 | VESGGVVVQRGGSLRLSCAGS |
| 1269. | FR-H2 | MHWVRQSPGKGLEWVSL |
| 1270. | FR-H3 | SYGDSVKGRFTISRDNSKKSLFLQMTNLRPEDTALYYC |
| 1271. | FR-H4 | WGQGTLVTVSS |
| 1272. | FR-L1 | MXXXTQSPLSLPVTPGEPASISCRSS |
| 1273. | FR-L2 | LDWYIQKPGQSPQLLIY |
| 1274. | FR-L3 | KRAAGVPDRFSGSVSGTDFTLMISRVEAEDVGVYYC |
| 1275. | FR-L4 | FGQGTRVEIK |
| 1276. | heavychain | VESGGVVVQRGGSLRLSCAGSGFTFEDYSMHWVRQSPGK |
| | | GLEWVSLISWDGGRTSYGDSVKGRFTISRDNSKKSLFLQMT |
| | | NLRPEDTALYYCVKDTGLRSFDSWGQGTLVTVSSKGPSVF |
| | | PLAPSSKSTSGGTAALGXLVKDYFPEPVTVSWNSGALTSGV |
| | | HTFPAVLQSSGLYSLSSVVTVPSSSLGTQTYICNVNHKPSNT |
| | | KVDKXVEPKSCDKTHTCPPCPAPELLGGPSVFLFPPKPKDT |
| | | LMISRTPEVTC |
| 1277. | lightchain | MXXXTQSPLSLPVTPGEPASISCRSSQSLVHSNGINYLDWYI |
| | | QKPGQSPQLLIYMGFKRAAGVPDRFSGSVSGTDFTLMISRV |
| | | EAEDVGVYYCMQALQSWTFGQGTRVEIKAPSVFIFPPSDEQ |
| | | LKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC*K |
| 1278. | VH_nuc | gtggagtctggcggagtcgtggtacagcggggggggtccctgagactctcctgtgca |
| | | ggctctggattcacctttgaggattattccatgcactgggtccgtcaatctccggggaa |
| | | gggtcttgagtgggtctctcttattagttgggatggtggccggacatcctatggagactc |
| | | tgtgaaggggcggttcaccatctccagagacaacagcaagaagtccctgtttctgca |
| | | aatgaccaatctgagacccgaa |
| 1279. | VL_nuc | atgttngnngngactcagtctcctctctccctgcccgtcacccctggagagccggcct |
| | | ccatctcctgtcggtctagtcagagcctcgtgcatagtaatggaatcaactatttggact |
| | | ggtacatccagaagccagggcagtctccacaactcctgatctatatgggttttaagcg |
| | | ggccgccggggtccctgacaggttcagtggcagtgtctcaggcacagattttacact |
| | | gatgatcagcagagtggaggct |
| SEQ ID NO | | 24BU7P1D4 |
| 1280. | CDR-H1 | GFIFDDYS |
| 1281. | CDR-H2 | ISWDGGRT |
| 1282. | CDR-H3 | VKDTGLRSFDS |
| 1283. | CDR-L1 | QSLVHSNGYTY |
| 1284. | CDR-L2 | MGS |
| 1285. | CDR-L3 | MQALQSWT |
| 1286. | VH | ESGGEVVQPGGSLRLSCVASGFIFDDYSMHWVRQVPGKGL |
| | | EWVSLISWDGGRTSYGDSVKGRFTISRDNNKKTLFLQMNSL |
| | | RPEDTALYYCVKDTGLRSFDSWGQGILVTVSS |
| 1287. | VL | DVXETQSPLSLPVTPGEPASISCRSSQSLVHSNGYTYLDWY |
| | | LQRPGQSPQLLIYMGSKRASGVPDRFSGSFSGTDFTLKISR |
| | | VEAEDVGVYYCMQALQSWTFGLGTKVEIK |
| 1288. | FR-H1 | ESGGEVVQPGGSLRLSCVAS |
| 1289. | FR-H2 | MHWVRQVPGKGLEWVSL |
| 1290. | FR-H3 | SYGDSVKGRFTISRDNNKKTLFLQMNSLRPEDTALYYC |
| 1291. | FR-H4 | WGQGILVTVSS |
| 1292. | FR-L1 | DVXETQSPLSLPVTPGEPASISCRSS |
| 1293. | FR-L2 | LDWYLQRPGQSPQLLIY |
| 1294. | FR-L3 | KRASGVPDRFSGSFSGTDFTLKISRVEAEDVGVYYC |
| 1295. | FR-L4 | FGLGTKVEIK |
| 1296. | heavychain | ESGGEVVQPGGSLRLSCVASGFIFDDYSMHWVRQVPGKGL |
| | | EWVSLISWDGGRTSYGDSVKGRFTISRDNNKKTLFLQMNSL |
| | | RPEDTALYYCVKDTGLRSFDSWGQGILVTVSSKGPSVFPLA |
| | | PSSKSTSGGTAALGXLVKDYFPEPVTVSWNSGALTSGVHTF |
| | | PAVLQSSGLYSLSSVVTVPSSSLGTQTYICNVNHKPSNTKV |
| | | DKXVEPKSCDKTHTCPPCPAPELLGGPSVFLFPPKPKDTLMI |
| | | SRTPEVTCV |
| 1297. | lightchain | DVXETQSPLSLPVTPGEPASISCRSSQSLVHSNGYTYLDWY |
| | | LQRPGQSPQLLIYMGSKRASGVPDRFSGSFSGTDFTLKISR |
| | | VEAEDVGVYYCMQALQSWTFGLGTKVEIKAPSVFIFPPSDE |
| | | QLKSGTASVVCLLNNFYPREAKVQWKVDNALQSGNSQESV |
| | | TEQDSKDSTYSLSSTLTLSKADYEKHKVYACEVTHQGLSSP |
| | | VTKSFNRGEC*K |
| 1298. | VH_nuc | ggagtctggcggagaggtcgtacagcctggggggtccctgagactctcttgtgtagc |
| | | ctctggattcatctttgatgattattccatgcactgggtccgtcaagtcccggggaaggg |
| | | tcttgagtgggtctctctaattagttgggatggtggtcgcacatcctatggagactctgtg |
| | | aagggtcgattcaccatctccagagacaacaacaagaagaccctgtttctgcaaat |
| | | gaacagtctgagacctgagga |
| 1299. | VL_nuc | gatgttgnngagactcagtctccactctccctgcccgtcacccctggagagccggcct |
| | | ccatctcctgcaggtctagtcagagcctcgttcatagtaatggatacacctatttggatt |
| | | ggtacctgcagaggccagggcagtctccgcaactcctgatctatatgggttctaagc |
| | | gggcctccggggtccctgacaggttcagtggcagtttctcaggcacagattttacact |
| | | gaaaatcagcagagtggaggct |
| SEQ ID NO | | 24BU7P1C10 |
| 1300. | CDR-H1 | GFTFDDYS |
| 1301. | CDR-H2 | ISWDGGRT |
| 1302. | CDR-H3 | VKDTGLRSFDS |
| 1303. | CDR-L1 | QSVFDDSSNKNY |
| 1304. | CDR-L2 | WAS |
| 1305. | CDR-L3 | HQYYSTPHS |
| 1306. | VH | ESGGVVVQPGGSLRLSCAASGFTFDDYSMHWVRQVPGKG |
| | | LEWVSLISWDGGRTSYGDSVKGRFTISRDNSKRSLFLQMTN |
| | | LRPEDTAFYYCVKDTGLRSFDSWGQGTLVTVSS |
| 1307. | VL | DXXXTQSPDSVAVSLGQRATINCESSQSVFDDSSNKNYLA |
| | | WYQHKPGQPPKLLIYWASSRESGVPDRFIGSGSGTDFTLTI |
| | | SSLQAADVAVYYCHQYYSTPHSFGQGTKVAIK |
| 1308. | FR-H1 | ESGGVVVQPGGSLRLSCAAS |
| 1309. | FR-H2 | MHWVRQVPGKGLEWVSL |
| 1310. | FR-H3 | SYGDSVKGRFTISRDNSKRSLFLQMTNLRPEDTAFYYC |
| 1311. | FR-H4 | WGQGTLVTVSS |
| 1312. | FR-L1 | DXXXTQSPDSVAVSLGQRATINCESS |
| 1313. | FR-L2 | LAWYQHKPGQPPKLLIY |
| 1314. | FR-L3 | SRESGVPDRFIGSGSGTDFTLTISSLQAADVAVYYC |
| 1315. | FR-L4 | FGQGTKVAIK |
| 1316. | heavychain | ESGGVVVQPGGSLRLSCAASGFTFDDYSMHWVRQVPGKG |
| | | LEWVSLISWDGGRTSYGDSVKGRFTISRDNSKRSLFLQMTN |
| | | LRPEDTAFYYCVKDTGLRSFDSWGQGTLVTVSSKGPSVFPL |
| | | APSSKSTSGGTAALGXLVKDYFPEPVTVSWNSGALTSGVHT |
| | | FPAVLQSSGLYSLSSVVTVPSSSLGTQTYICNVNHKPSNTKV |
| | | DKXVEPKSCDKTHTCPPCPAPELLGGPSVFLFPPKPKDTLMI |
| | | SRTPEVTCV |
| 1317. | lightchain | DXXXTQSPDSVAVSLGQRATINCESSQSVFDDSSNKNYLA |
| | | WYQHKPGQPPKLLIYWASSRESGVPDRFIGSGSGTDFTLTI |
| | | SSLQAADVAVYYCHQYYSTPHSFGQGTKVAIKAPSVFIFPPS |
| | | DEQLKSGTASVVCLLNNFYPREAKVQWKVDNALQSGNSQE |
| | | SVTEQDSKDSTYSLSSTLTLSKADYEKHKVYACEVTHQGLS |
| | | SPVTKSFNRGEC*K |
| 1318. | VH_nuc | tggagtctggcggagtcgtggtacagcctggggggtccctgagactctcctgtgcag |
| | | cctctggattcacctttgatgattattccatgcactgggtccgtcaagttccggggaagg |
| | | gtcttgagtgggtctctcttattagttgggatggtggccggacatcctatggagactcag |
| | | tgaagggtcgattcaccatctccagagacaacagcaagaggtccctgtttctgcaaa |
| | | tgaccaatctgagacctgaag |
| 1319. | VL_nuc | gacatnngnntgacccagtctccagactccgtggctgtgtctctgggccagagggcc |
| | | accatcaactgcgagtccagccagagtgttttcgatgactccagcaataagaactac |
| | | ttagcttggtatcaacacaaaccaggacagcctcctaaactactcatttactgggcatc |
| | | tagccgggaatccggggtccctgaccgattcattggcagcgggtctgggacagactt |
| | | cactctcaccatcagcagcctgcag |
| SEQ ID NO | | 24BU7P1D3 |
| 1320. | CDR-H1 | GFTFDDYS |
| 1321. | CDR-H2 | ISWDGGRT |
| 1322. | CDR-H3 | VKDTGLRSFDY |
| 1323. | CDR-L1 | QGLQHSNGYNY |
| 1324. | CDR-L2 | MGS |
| 1325. | CDR-L3 | MQALQSWT |
| 1326. | VH | ESGGVVVQPGGSLRLSCAASGFTFDDYSMHWVRQLPGKG |
| | | LEWVSLISWDGGRTDYADSVKGRFTISRDNSKSSLLLQMNS |
| | | LRLEDTALYYCVKDTGLRSFDYWGQGTLVTVSS |
| 1327. | VL | DVGETQSPLSLPVTPGEPASMSCRSSQGLQHSNGYNYVD |
| | | WYLQKPGQSPQLLIYMGSKRASGVPDRFSGSGSGTDFTLKI |
| | | SRVEAEDVGVYYCMQALQSWTFGQGTKVEIK |
| 1328. | FR-H1 | ESGGVVVQPGGSLRLSCAAS |
| 1329. | FR-H2 | MHWVRQLPGKGLEWVSL |
| 1330. | FR-H3 | DYADSVKGRFTISRDNSKSSLLLQMNSLRLEDTALYYC |
| 1331. | FR-H4 | WGQGTLVTVSS |
| 1332. | FR-L1 | DVGETQSPLSLPVTPGEPASMSCRSS |
| 1333. | FR-L2 | VDWYLQKPGQSPQLLIY |
| 1334. | FR-L3 | KRASGVPDRFSGSGSGTDFTLKISRVEAEDVGVYYC |
| 1335. | FR-L4 | FGQGTKVEIK |
| 1336. | heavychain | ESGGVVVQPGGSLRLSCAASGFTFDDYSMHWVRQLPGKG |
| | | LEWVSLISWDGGRTDYADSVKGRFTISRDNSKSSLLLQMNS |
| | | LRLEDTALYYCVKDTGLRSFDYWGQGTLVTVSSKGPSVFPL |
| | | APSSKSTSGGTAALGXLVKDYFPEPVTVSWNSGALTSGVHT |
| | | FPAVLQSSGLYSLSSVVTVPSSSLGTQTYICNVNHKPSNTKV |
| | | DKXVEPKSCDKTHTCPPCPAPELLGGPSVFLFPPKPKDTLMI |
| | | SRTPEVTCV |
| 1337. | lightchain | DVGETQSPLSLPVTPGEPASMSCRSSQGLQHSNGYNYVD |
| | | WYLQKPGQSPQLLIYMGSKRASGVPDRFSGSGSGTDFTLKI |
| | | SRVEAEDVGVYYCMQALQSWTFGQGTKVEIKAPSVFIFPPS |
| | | DEQLKSGTASVVCLLNNFYPREAKVQWKVDNALQSGNSQE |
| | | SVTEQDSKDSTYSLSSTLTLSKADYEKHKVYACEVTHQGLS |
| | | SPVTKSFNRGEC*K |
| 1338. | VH_nuc | ggagtctggcggagtcgtggtacagcctgggggatccctgagactctcctgtgcagc |
| | | ctctggattcacttttgatgattactccatgcactgggtccgtcaacttccggggaaagg |
| | | tctggagtgggtctctcttattagttgggatggaggtcgcacagactatgcagactctgt |
| | | gaagggtcgattcaccatctccagagacaatagcaagagctccctgcttctgcaaat |
| | | gaacagtctgagacttgaaga |
| 1339. | VL_nuc | gatgttggngagactcagtctccactctccctgcccgtcacccctggagagccggcct |
| | | ccatgtcctgcaggtctagtcagggcctccagcatagtaatggatacaactatgtgga |
| | | ttggtacctgcagaagccagggcagtctccacagctcctgatctatatgggctctaag |
| | | cgggcctccggggtccctgacaggttcagtggcagtggatcaggcacagattttaca |
| | | ctgaaaatcagcagagtggaggct |
| SEQ ID NO | | 24BU7P1D9 |
| 1340. | CDR-H1 | GFTFNYAV |
| 1341. | CDR-H2 | IDSESDGGTT |
| 1342. | CDR-H3 | NTHFYCTTTSCYGDH |
| 1343. | CDR-L1 | QSVLYSPKNKHY |
| 1344. | CDR-L2 | WAS |
| 1345. | CDR-L3 | QQYYSGRT |
| 1346. | VH | ESGGGLVKPGESLRLSCEGSGFTFNYAVMSWVRQAPGKG |
| | | LEWVGRIDSESDGGTTDYAAPVKGRFTISRDDSRNTLHLQM |
| | | NSLEIGDTAVYYCNTHFYCTTTSCYGDHWGQGTLVTVSS |
| 1347. | VL | TXXVTQSPEFLSLSLGERASINCKASQSVLYSPKNKHYLAW |
| | | YQQKPGQPPKLLMYWASTRESGVPDRFSGSGSGTDFTLTI |
| | | NTFQAEDVAVYYCQQYYSGRTFGPGTKVDIK |
| 1348. | FR-H1 | ESGGGLVKPGESLRLSCEGS |
| 1349. | FR-H2 | MSWVRQAPGKGLEWVGR |
| 1350. | FR-H3 | DYAAPVKGRFTISRDDSRNTLHLQMNSLEIGDTAVYYC |
| 1351. | FR-H4 | WGQGTLVTVSS |
| 1352. | FR-L1 | TXXVTQSPEFLSLSLGERASINCKAS |
| 1353. | FR-L2 | LAWYQQKPGQPPKLLMY |
| 1354. | FR-L3 | TRESGVPDRFSGSGSGTDFTLTINTFQAEDVAVYYC |
| 1355. | FR-L4 | FGPGTKVDIK |
| 1356. | heavychain | ESGGGLVKPGESLRLSCEGSGFTFNYAVMSWVRQAPGKG |
| | | LEWVGRIDSESDGGTTDYAAPVKGRFTISRDDSRNTLHLQM |
| | | NSLEIGDTAVYYCNTHFYCTTTSCYGDHWGQGTLVTVSSK |
| | | GPSVFPLAPSSKSTSGGTAALGXLVKDYFPEPVTVSWNSGA |
| | | LTSGVHTFPAVLQSSGLYSLSSVVTVPSSSLGTQTYICNVNH |
| | | KPSNTKVDKXVEPKSCDKTHTCPPCPAPELLGGPSVFLFPP |
| | | KPKDTLMISRT |
| 1357. | lightchain | TXXVTQSPEFLSLSLGERASINCKASQSVLYSPKNKHYLAW |
| | | YQQKPGQPPKLLMYWASTRESGVPDRFSGSGSGTDFTLTI |
| | | NTFQAEDVAVYYCQQYYSGRTFGPGTKVDIKAPSVFIFPPS |
| | | DEQLKSGTASVVCLLNNFYPREAKVQWKVDNALQSGNSQE |
| | | SVTEQDSKDSTYSLSSTLTLSKADYEKHKVYACEVTHQGLS |
| | | SPVTKSFNRGEC*K |
| 1358. | VH_nuc | gagtctgggggaggcttggtaaagcctggggagtcccttcgactctcctgtgaaggct |
| | | ctggattcactttcaattacgccgtgatgagctgggtccgccaggctccagggaagg |
| | | ggctggagtgggttggccgtattgatagcgaaagtgacggtgggacaacagactac |
| | | gctgcacccgtgaaaggcagattcaccatctcaagagacgattcaagaaacacgc |
| | | tgcatctgcagatgaacagcctggaaatc |
| 1359. | VL_nuc | acatnggnngtgacccagtctccagagttcctgagtctgtctctgggcgagagggcc |
| | | tccatcaactgcaaggccagccagagtgttttatacagccccaaaaataagcactac |
| | | ttggcttggtatcagcagaaacctggacagcctcctaagctgctcatgtattgggcttct |
| | | acccgggaatccggggtccctgaccgattcagtggcagcgggtctggtacagatttc |
| | | actctcaccatcaacaccttccag |
| SEQ ID NO | | 24BU7P1B1 |
| 1360. | CDR-H1 | GYSFTRFD |
| 1361. | CDR-H2 | MNPKSGHS |
| 1362. | CDR-H3 | ARGVDNRX |
| 1363. | CDR-L1 | QSVFDDSSNKNY |
| 1364. | CDR-L2 | WAS |
| 1365. | CDR-L3 | LQYYSTPHS |
| 1366. | VH | SGAEVKKPGASVKVSCKTSGYSFTRFDINWVRQATGQGLE |
| | | WMGWMNPKSGHSGPAQKFQGRITMTVNTSISTAYMELSSL |
| | | RFEDTAVYYCARGVDNRXWGQGTLITVSS |
| 1367. | VL | DIXXTQSPDSVAVSLGQRATINCESSQSVFDDSSNKNYLAW |
| | | YQHKPGQPPKLLIYWASSRESGVPDRFIGSGSGTDFTLTISS |
| | | LQAADVAVYYCLQYYSTPHSFGQGTKVAIN |
| 1368. | FR-H1 | SGAEVKKPGASVKVSCKTS |
| 1369. | FR-H2 | INWVRQATGQGLEWMGW |
| 1370. | FR-H3 | GPAQKFQGRITMTVNTSISTAYMELSSLRFEDTAVYYC |
| 1371. | FR-H4 | WGQGTLITVSS |
| 1372. | FR-L1 | DIXXTQSPDSVAVSLGQRATINCESS |
| 1373. | FR-L2 | LAWYQHKPGQPPKLLIY |
| 1374. | FR-L3 | SRESGVPDRFIGSGSGTDFTLTISSLQAADVAVYYC |
| 1375. | FR-L4 | FGQGTKVAIN |
| 1376. | heavychain | SGAEVKKPGASVKVSCKTSGYSFTRFDINWVRQATGQGLE |
| | | WMGWMNPKSGHSGPAQKFQGRITMTVNTSISTAYMELSSL |
| | | RFEDTAVYYCARGVDNRXWGQGTLITVSSKGPSVFPLAPSS |
| | | KSTSGGTAALGXLVKDYFPEPVTVSWNSGALTSGVHTFPAV |
| | | LQSSGLYSLSSVVTVPSSSLGTQTYICNVNHKPSNTKVDKX |
| | | VEPKSCDKTHTCPPCPAPELLGGPSVFLFPPKPKDTLMISRT |
| | | PEVTCVVVDV |
| 1377. | lightchain | DIXXTQSPDSVAVSLGQRATINCESSQSVFDDSSNKNYLAW |
| | | YQHKPGQPPKLLIYWASSRESGVPDRFIGSGSGTDFTLTISS |
| | | LQAADVAVYYCLQYYSTPHSFGQGTKVAINAPSVFIFPPSDE |
| | | QLKSGTASVVCLLNNFYPREAKVQWKVDNALQSGNSQESV |
| | | TEQDSKDSTYSLSSTLTLSKADYEKHKVYACEVTHQGLSSP |
| | | VTKSFNRGEC*K |
| 1378. | VH_nuc | agtctggggctgaggtgaagaagcctggggcctcagtgaaggtctcctgcaagactt |
| | | ctggatacagcttcacccgttttgatatcaactgggtgcgacaggccactggacaag |
| | | ggcttgagtggatgggatggatgaaccctaagagtggtcactcaggccctgcacag |
| | | aagttccagggcagaatcaccatgaccgttaacacctccataagtacagcctacatg |
| | | gagctgagcagcctgagatttgaggaca |
| 1379. | VL_nuc | gacatcgnnnnnacccagtctccagactccgtggctgtgtctctgggccagagggc |
| | | caccatcaactgcgagtccagccagagtgttttcgatgactccagcaataagaacta |
| | | cttagcttggtatcaacacaaaccaggacagcctcctaaactactcatttactgggcat |
| | | ctagccgggaatccggggtccctgaccgattcattggcagcgggtctgggacagac |
| | | ttcactctcaccatcagcagcctgcag |
| SEQ ID NO | | 24BU7P1C2 |
| 1380. | CDR-H1 | GFTFDDYS |
| 1381. | CDR-H2 | ISWDGART |
| 1382. | CDR-H3 | VKDTGLRSFDS |
| 1383. | CDR-L1 | QSLLHSNGYNY |
| 1384. | CDR-L2 | MGS |
| 1385. | CDR-L3 | MQALQTWT |
| 1386. | VH | VESGGVVVQPGGSLRLSCAASGFTFDDYSMHWVRQVPGK |
| | | GLEWVSLISWDGARTSYGDSVKGRFTISRDNSKRSLFLQMT |
| | | NLRPEDTAFYYCVKDTGLRSFDSWGQGTLVTVSS |
| 1387. | VL | DVXETQSPLSLPVTPGEPASISCRSSQSLLHSNGYNYLDWY |
| | | LQKPGQSPQLLIYMGSKRASGVPDRFSGSVSGTDFTLKISR |
| | | VEAEDVGVYYCMQALQTWTFGQGTKVEIK |
| 1388. | FR-H1 | VESGGVVVQPGGSLRLSCAAS |
| 1389. | FR-H2 | MHWVRQVPGKGLEWVSL |
| 1390. | FR-H3 | SYGDSVKGRFTISRDNSKRSLFLQMTNLRPEDTAFYYC |
| 1391. | FR-H4 | WGQGTLVTVSS |
| 1392. | FR-L1 | DVXETQSPLSLPVTPGEPASISCRSS |
| 1393. | FR-L2 | LDWYLQKPGQSPQLLIY |
| 1394. | FR-L3 | KRASGVPDRFSGSVSGTDFTLKISRVEAEDVGVYYC |
| 1395. | FR-L4 | FGQGTKVEIK |
| 1396. | heavychain | VESGGVVVQPGGSLRLSCAASGFTFDDYSMHWVRQVPGK |
| | | GLEWVSLISWDGARTSYGDSVKGRFTISRDNSKRSLFLQMT |
| | | NLRPEDTAFYYCVKDTGLRSFDSWGQGTLVTVSSKGPSVF |
| | | PLAPSSKSTSGGTAALGXLVKDYFPEPVTVSWNSGALTSGV |
| | | HTFPAVLQSSGLYSLSSVVTVPSSSLGTQTYICNVNHKPSNT |
| | | KVDKXVEPKSCDKTHTCPPCPAPELLGGPSVFLFPPKPKDT |
| | | LMISRTPEVTC |
| 1397. | lightchain | DVXETQSPLSLPVTPGEPASISCRSSQSLLHSNGYNYLDWY |
| | | LQKPGQSPQLLIYMGSKRASGVPDRFSGSVSGTDFTLKISR |
| | | VEAEDVGVYYCMQALQTWTFGQGTKVEIKAPSVFIFPPSDE |
| | | QLKSGTASVVCLLNNFYPREAKVQWKVDNALQSGNSQESV |
| | | TEQDSKDSTYSLSSTLTLSKADYEKHKVYACEVTHQGLSSP |
| | | VTKSFNRGEC*K |
| 1398. | VH_nuc | gtggagtctggcggagtcgtggtacagcctggggggtccctgagactctcctgtgca |
| | | gcctctggattcacctttgatgattattccatgcactgggtccgtcaagttccggggaag |
| | | ggtcttgagtgggtctctcttattagttgggatggtgcccggacatcctatggagactctg |
| | | tgaagggtcgattcaccatctccagagacaacagcaagaggtccctgtttctgcaaa |
| | | tgaccaatctgagacctgaa |
| 1399. | VL_nuc | gatgttnnngagactcagtctccactctccctgcccgtcacccctggagagccggcct |
| | | ccatctcctgcaggtctagtcagagcctcctgcatagtaatggatacaactatttggatt |
| | | ggtacctccagaagccagggcagtctccacagctcctgatctatatgggttctaagcg |
| | | ggcctccggggtccctgacaggttcagtggcagtgtctcaggcacagattttacactg |
| | | aaaatcagcagagtggaggct |
| SEQ ID NO | | 105BU7P1A11 |
| 1400. | CDR-H1 | GFTFGDYT |
| 1401. | CDR-H2 | IKWNGGGI |
| 1402. | CDR-H3 | AKDIGLRSLDS |
| 1403. | CDR-L1 | QSLLHSNGYTY |
| 1404. | CDR-L2 | MGS |
| 1405. | CDR-L3 | MQALQGWT |
| 1406. | VH | CXXVESGGGLVQPGRSLRLSCTASGFTFGDYTMHWVRQA |
| | | PGKGLEWVSNIKWNGGGIGYADSXKGRFTISRDNAGNSLYL |
| | | QMNSLRPEDTAFYYCAKDIGLRSLDSWGQGTLVIVSS |
| 1407. | VL | DVVMTQSPLSLPVTPGEPASISCRSSQSLLHSNGYTYLDWY |
| | | LQKPGQSPQLLIYMGSKRASGVPDRFSGSFSDTDFTLTISR |
| | | VEAEDVGVYYCMQALQGWTFGQGTKVEIK |
| 1408. | FR-H1 | CXXVESGGGLVQPGRSLRLSCTAS |
| 1409. | FR-H2 | MHWVRQAPGKGLEWVSN |
| 1410. | FR-H3 | GYADSXKGRFTISRDNAGNSLYLQMNSLRPEDTAFYYC |
| 1411. | FR-H4 | WGQGTLVIVSS |
| 1412. | FR-L1 | DVVMTQSPLSLPVTPGEPASISCRSS |
| 1413. | FR-L2 | LDWYLQKPGQSPQLLIY |
| 1414. | FR-L3 | KRASGVPDRFSGSFSDTDFTLTISRVEAEDVGVYYC |
| 1415. | FR-L4 | FGQGTKVEIK |
| 1416. | heavychain | CXXVESGGGLVQPGRSLRLSCTASGFTFGDYTMHWVRQA |
| | | PGKGLEWVSNIKWNGGGIGYADSXKGRFTISRDNAGNSLYL |
| | | QMNSLRPEDTAFYYCAKDIGLRSLDSWGQGTLVIVSSKGPS |
| | | VFPLAPSSKSTSGGTAALGXLVKDYFPEPVTVSWNSGALTS |
| | | GVHTFPAVLQSSGLYSLSSVVTVPSSSLGTQTYICNVNHKP |
| | | SNTKVDKXVEPKSCDKTHTCPPCPAPELLGGPSVFLFPPKP |
| | | KDTLMISRTPE |
| 1417. | lightchain | DVVMTQSPLSLPVTPGEPASISCRSSQSLLHSNGYTYLDWY |
| | | LQKPGQSPQLLIYMGSKRASGVPDRFSGSFSDTDFTLTISR |
| | | VEAEDVGVYYCMQALQGWTFGQGTKVEIKAPSVFIFPPSDE |
| | | QLKSGTASVVCLLNNFYPREAKVQWKVDNALQSGNSQESV |
| | | TEQDSKDSTYSLSSTLTLSKADYEKHKVYACEVTHQGLSSP |
| | | VTKSFNRGEC*K |
| 1418. | VH_nuc | gtgcnannnngtggaatctgggggaggcttggtacagcctggcaggtccctgaga |
| | | ctctcctgtacagcctctggtttcacctttggtgattataccatgcattgggtccggcaag |
| | | ctccagggaagggcctggagtgggtctcaaatattaagtggaatggtggtggcatag |
| | | gctatgcggactctgntaagggccgattcaccatctccagagacaacgccgggaac |
| | | tccctctatctacaaatgaacagtct |
| 1419. | VL_nuc | gatgttgtgatgactcagtctccactctccctgcccgtcacccctggagagccggcctc |
| | | catctcctgcaggtctagtcagagcctcctgcatagtaatggatacacctatttggattg |
| | | gtacctgcagaagccagggcagtctccacagctcctgatctatatgggttctaagcg |
| | | ggcctccggggtccctgacaggttcagtggcagtttctcagacacagatttcacactg |
| | | acaatcagcagagtggaggct |
| SEQ ID NO | | 105BU7P1C3 |
| 1420. | CDR-H1 | GFTFNNYA |
| 1421. | CDR-H2 | ISASGGSR |
| 1422. | CDR-H3 | AKDRVEDGNYEKDWHFDL |
| 1423. | CDR-L1 | PIVSGNY |
| 1424. | CDR-L2 | DAS |
| 1425. | CDR-L3 | QQYAKSPVT |
| 1426. | VH | AXXVESGGGLGQPGGSLRLSCPASGFTFNNYAMSWIRQAP |
| | | GKGLEWVSAISASGGSRFYADSVRGRFTISRDNAKNTLYLQ |
| | | LNSLRAEDTAVYFCAKDRVEDGNYEKDWHFDLWGRGTLVT |
| | | VSS |
| 1427. | VL | EIVXTQSPGTLSLSPGERATLSCRASPIVSGNYLAWYQQRP |
| | | GQSPRLLIYDASSRASGVPDRFSGGGSATDFTLTISRLEPED |
| | | FAVYYCQQYAKSPVTFGGGTKVEIK |
| 1428. | FR-H1 | AXXVESGGGLGQPGGSLRLSCPAS |
| 1429. | FR-H2 | MSWIRQAPGKGLEWVSA |
| 1430. | FR-H3 | FYADSVRGRFTISRDNAKNTLYLQLNSLRAEDTAVYFC |
| 1431. | FR-H4 | WGRGTLVTVSS |
| 1432. | FR-L1 | EIVXTQSPGTLSLSPGERATLSCRAS |
| 1433. | FR-L2 | LAWYQQRPGQSPRLLIY |
| 1434. | FR-L3 | SRASGVPDRFSGGGSATDFTLTISRLEPEDFAVYYC |
| 1435. | FR-L4 | FGGGTKVEIK |
| 1436. | heavychain | AXXVESGGGLGQPGGSLRLSCPASGFTFNNYAMSWIRQAP |
| | | GKGLEWVSAISASGGSRFYADSVRGRFTISRDNAKNTLYLQ |
| | | LNSLRAEDTAVYFCAKDRVEDGNYEKDWHFDLWGRGTLVT |
| | | VSSKGPSVFPLAPSSKSTSGGTAALGXLVKDYFPEPVTVSW |
| | | NSGALTSGVHTFPAVLQSSGLYSLSSVVTVPSSSLGTQTYIC |
| | | NVNHKPSNTKVDKXVEPKSCDKTHTCPPCPAPELLGGPSVF |
| | | LFPPKPKDTL |
| 1437. | lightchain | EIVXTQSPGTLSLSPGERATLSCRASPIVSGNYLAWYQQRP |
| | | GQSPRLLIYDASSRASGVPDRFSGGGSATDFTLTISRLEPED |
| | | FAVYYCQQYAKSPVTFGGGTKVEIKAPSVFIFPPSDEQLKSG |
| | | TASVVCLLNNFYPREAKVQWKVDNALQSGNSQESVTEQDS |
| | | KDSTYSLSSTLTLSKADYEKHKVYACEVTHQGLSSPVTKSF |
| | | NRGEC*K |
| 1438. | VH_nuc | gtgccaannnngtggagtcggggggaggcttggggcagccgggggggtccctga |
| | | gactctcctgtccagcctctggattcacctttaacaactatgccatgagttggatccgcc |
| | | aggctccagggaaggggctggagtgggtctcagctattagcgccagtggcggtagc |
| | | agattctacgcagactccgtgaggggccgattcaccatctccagagacaatgccaa |
| | | gaacaccctatatctccaactgaacagcc |
| 1439. | VL_nuc | gaaattgtnnngacgcagtctccaggcaccctgtctttgtctccaggggagagagcc |
| | | accctctcctgcagggccagtccgattgttagcggcaattacttagcctggtatcagca |
| | | gagacctggccagtctccccggctcctcatctatgatgcatccagcagggccagtgg |
| | | cgtcccagacaggttcagtggcggtgggtctgcgacagacttcactctcaccatcag |
| | | cagactggagcctgaagactttgca |
| SEQ ID NO | | 105BU7P1D6 |
| 1440. | CDR-H1 | GYTFSSYD |
| 1441. | CDR-H2 | MNPKNGYS |
| 1442. | CDR-H3 | VRVRTISQGDSWYFDL |
| 1443. | CDR-L1 | QGLTVS |
| 1444. | CDR-L2 | AAS |
| 1445. | CDR-L3 | MHTLQPPYT |
| 1446. | VH | VQSGPEVKKPGASVKVSCKASGYTFSSYDFNWVRQAPGQ |
| | | GLEWMGWMNPKNGYSGFAQNFRGRISMTRNSSITTAYMEL |
| | | TSLTSDDTAVYYCVRVRTISQGDSWYFDLWGRGTLVTVSS |
| 1447. | VL | DMATTQSPSSVSASEGDRAVISCRASQGLTVSLARNHPETG |
| | | EAPNLLISAASSLRGTVAAPXSGSGSGTDFTLKITRVEAEDV |
| | | GVYYCMHTLQPPYTFGRGTKLEIK |
| 1448. | FR-H1 | VQSGPEVKKPGASVKVSCKAS |
| 1449. | FR-H2 | FNWVRQAPGQGLEWMGW |
| 1450. | FR-H3 | GFAQNFRGRISMTRNSSITTAYMELTSLTSDDTAVYYC |
| 1451. | FR-H4 | WGRGTLVTVSS |
| 1452. | FR-L1 | DMATTQSPSSVSASEGDRAVISCRAS |
| 1453. | FR-L2 | LARNHPETGEAPNLLIS |
| 1454. | FR-L3 | SLRGTVAAPXSGSGSGTDFTLKITRVEAEDVGVYYC |
| 1455. | FR-L4 | FGRGTKLEIK |
| 1456. | heavychain | VQSGPEVKKPGASVKVSCKASGYTFSSYDFNWVRQAPGQ |
| | | GLEWMGWMNPKNGYSGFAQNFRGRISMTRNSSITTAYMEL |
| | | TSLTSDDTAVYYCVRVRTISQGDSWYFDLWGRGTLVTVSSK |
| | | GPSVFPLAPSSKSTSGGTAALGXLVKDYFPEPVTVSWNSGA |
| | | LTSGVHTFPAVLQSSGLYSLSSVVTVPSSSLGTQTYICNVNH |
| | | KPSNTKVDKXVEPKSCDKTHTCPPCPAPELLGGPSVFLFPP |
| | | KPKDTLMISRT |
| 1457. | lightchain | DMATTQSPSSVSASEGDRAVISCRASQGLTVSLARNHPETG |
| | | EAPNLLISAASSLRGTVAAPXSGSGSGTDFTLKITRVEAEDV |
| | | GVYYCMHTLQPPYTFGRGTKLEIKAPSVFIFPPSDEQLKSGT |
| | | ASVVCLLNNFYPREAKVQWKVDNALQSGNSQESVTEQDSK |
| | | DSTYSLSSTLTLSKADYEKHKVYACEVTHQGLSSPVTKSFN |
| | | RGEC*K |
| 1458. | VH_nuc | ggtgcagtctgggcctgaggtgaagaagcctggggcctcagtgaaggtctcctgca |
| | | aggcttctggatataccttcagtagttatgacttcaactgggtgcgacaggcccctgga |
| | | caagggcttgagtggatgggatggatgaaccctaaaaatggttactcaggcttcgca |
| | | cagaatttccggggccggatatccatgaccaggaactcctccataaccacagccta |
| | | catggagctgaccagcctgacatctga |
| 1459. | VL_nuc | gacatggcgacgacccagtctccatcttccgtgtctgcatctgaaggggacagagcc |
| | | gtcatctcttgtcgggcgagtcagggtcttaccgtgtcgttagccaggaatcacccgg |
| | | agacaggggaagcccctaacctcctaatctctgctgcatcgagtttgcgaggaactg |
| | | tggctgcaccatncagtggcagtggatcaggcacagattttacactgaaaatcacca |
| | | gagtggaggctgaggatgttggggtt |
| SEQ ID NO | | 105BU7P1D7 |
| 1460. | CDR-H1 | GYTFSNYD |
| 1461. | CDR-H2 | MNPKNGYS |
| 1462. | CDR-H3 | VRVRTISKGDSWYFDL |
| 1463. | CDR-L1 | QSLLHNNGYNY |
| 1464. | CDR-L2 | LVS |
| 1465. | CDR-L3 | MHTLQPPYT |
| 1466. | VH | QSGPEVKKPGASVKVSCKSSGYTFSNYDFNWVRQAPGQG |
| | | LEWMGWMNPKNGYSGFAQNFRGRISMTRNSSITTAYMELT |
| | | SLTSDDTALYYCVRVRTISKGDSWYFDLWGRGTLVTVSS |
| 1467. | VL | RCXXTQSQVSLPVTPGEPASISCRSSQSLLHNNGYNYVDW |
| | | YLQKPGQSPQLLIYLVSNPVSIFRVCIXGSGSGTDFTLKITRV |
| | | EAEDVGVYYCMHTLQPPYTFGQGTKLXIK |
| 1468. | FR-H1 | QSGPEVKKPGASVKVSCKSS |
| 1469. | FR-H2 | FNWVRQAPGQGLEWMGW |
| 1470. | FR-H3 | GFAQNFRGRISMTRNSSITTAYMELTSLTSDDTALYYC |
| 1471. | FR-H4 | WGRGTLVTVSS |
| 1472. | FR-L1 | RCXXTQSQVSLPVTPGEPASISCRSS |
| 1473. | FR-L2 | VDWYLQKPGQSPQLLIY |
| 1474. | FR-L3 | NPVSIFRVCIXGSGSGTDFTLKITRVEAEDVGVYYC |
| 1475. | FR-L4 | FGQGTKLXIK |
| 1476. | heavychain | QSGPEVKKPGASVKVSCKSSGYTFSNYDFNWVRQAPGQG |
| | | LEWMGWMNPKNGYSGFAQNFRGRISMTRNSSITTAYMELT |
| | | SLTSDDTALYYCVRVRTISKGDSWYFDLWGRGTLVTVSSKG |
| | | PSVFPLAPSSKSTSGGTAALGXLVKDYFPEPVTVSWNSGAL |
| | | TSGVHTFPAVLQSSGLYSLSSVVTVPSSSLGTQTYICNVNHK |
| | | PSNTKVDKXVEPKSCDKTHTCPPCPAPELLGGPSVFLFPPK |
| | | PKDTLMISRTP |
| 1477. | lightchain | RCXXTQSQVSLPVTPGEPASISCRSSQSLLHNNGYNYVDW |
| | | YLQKPGQSPQLLIYLVSNPVSIFRVCIXGSGSGTDFTLKITRV |
| | | EAEDVGVYYCMHTLQPPYTFGQGTKLXIKAPSVFIFPPSDE |
| | | QLKSGTASVVCLLNNFYPREAKVQWKVDNALQSGNSQESV |
| | | TEQDSKDSTYSLSSTLTLSKADYEKHKVYACEVTHQGLSSP |
| | | VTKSFNRGEC*K |
| 1478. | VH_nuc | gcagtctgggcctgaggtgaagaagcctggggcctcagtgaaggtctcctgcaagt |
| | | cttctggatataccttcagtaattatgacttcaactgggtgcgacaggcccctggacaa |
| | | gggcttgagtggatgggatggatgaaccctaaaaatggctactcaggcttcgcaca |
| | | gaatttccggggccggatatccatgaccaggaactcctccataaccacagcctacat |
| | | ggagctgaccagcctgacatctgatga |
| 1479. | VL_nuc | cgatgttgnnagactcagtctcaagtctccctgcccgtcacccctggagagccggcc |
| | | tccatctcctgcaggtctagtcagagcctcctgcataacaatggatacaactatgtgg |
| | | attggtacctgcagaagccagggcagtctccacaactcctgatctatttggtttcgaac |
| | | ccagtctccatcttccgtgtctgcatcngtggcagtggatcaggcacagattttacactg |
| | | aagatcaccagagtggaggct |
| SEQ ID NO | | 105BU7P1D8 |
| 1480. | CDR-H1 | GFTFNNYA |
| 1481. | CDR-H2 | ISGRTGSA |
| 1482. | CDR-H3 | ARDGQEGGNYAAEYFQN |
| 1483. | CDR-L1 | QSISKW |
| 1484. | CDR-L2 | KAS |
| 1485. | CDR-L3 | QHFNTELLT |
| 1486. | VH | VESGGGLAQPGGSLTLSCAASGFTFNNYAMSWVRQAPGK |
| | | GLEWVSGISGRTGSAHYADAVRGRFTISRDNAKNTLYLQMS |
| | | SLRADDTATYYCARDGQEGGNYAAEYFQNWGQGTLVTVS |
| | | S |
| 1487. | VL | RHXXTQSPSTLAASVGDRVTITCRASQSISKWLAWYQQKPG |
| | | KAPKLLIYKASRLQSGAPSRFSGSGSGTEFTLTINSLQPDDF |
| | | ATYYCQHFNTELLTFGGGTKVEIK |
| 1488. | FR-H1 | VESGGGLAQPGGSLTLSCAAS |
| 1489. | FR-H2 | MSWVRQAPGKGLEWVSG |
| 1490. | FR-H3 | HYADAVRGRFTISRDNAKNTLYLQMSSLRADDTATYYC |
| 1491. | FR-H4 | WGQGTLVTVSS |
| 1492. | FR-L1 | RHXXTQSPSTLAASVGDRVTITCRAS |
| 1493. | FR-L2 | LAWYQQKPGKAPKLLIY |
| 1494. | FR-L3 | RLQSGAPSRFSGSGSGTEFTLTINSLQPDDFATYYC |
| 1495. | FR-L4 | FGGGTKVEIK |
| 1496. | heavychain | VESGGGLAQPGGSLTLSCAASGFTFNNYAMSWVRQAPGK |
| | | GLEWVSGISGRTGSAHYADAVRGRFTISRDNAKNTLYLQMS |
| | | SLRADDTATYYCARDGQEGGNYAAEYFQNWGQGTLVTVS |
| | | SKGPSVFPLAPSSKSTSGGTAALGXLVKDYFPEPVTVSWNS |
| | | GALTSGVHTFPAVLQSSGLYSLSSVVTVPSSSLGTQTYICNV |
| | | NHKPSNTKVDKXVEPKSCDKTHTCPPCPAPELLGGPSVFLF |
| | | PPKPKDTLMISR |
| 1497. | lightchain | RHXXTQSPSTLAASVGDRVTITCRASQSISKWLAWYQQKPG |
| | | KAPKLLIYKASRLQSGAPSRFSGSGSGTEFTLTINSLQPDDF |
| | | ATYYCQHFNTELLTFGGGTKVEIKAPSVFIFPPSDEQLKSGT |
| | | ASVVCLLNNFYPREAKVQWKVDNALQSGNSQESVTEQDSK |
| | | DSTYSLSSTLTLSKADYEKHKVYACEVTHQGLSSPVTKSFN |
| | | RGEC*K |
| 1498. | VH_nuc | gtngagtcggggggaggcctcgcacagcctgggggatccctgacactctcctgtgc |
| | | agcgtctgggttcacattcaacaattatgccatgagctgggtccgccaggctccagg |
| | | gaaggggctggagtgggtctcaggaataagtgggagaactggtagcgcacactac |
| | | gcagacgccgtgaggggccggttcaccatctccagagacaatgccaagaacaca |
| | | ctgtatctccaaatgagcagcctgagagccgat |
| 1499 | VL_nuc | cgacatcnnnagacccagtctccttccaccctggctgcatctgtaggagacagagtc |
| | | accatcacttgccgggccagtcagagtattagtaagtggttggcctggtatcagcaga |
| | | aaccagggaaagcccctaagctgctgatctataaggcatctcgtttacaaagtgggg |
| | | ccccatcaaggttcagcggcagtggatctgggacagagttcactctcaccataaaca |
| | | gcctgcagcctgatgattttgcaact |
| SEQ ID NO | | 105BU7P1D12 |
| 1500. | CDR-H1 | GFTFRSYA |
| 1501. | CDR-H2 | ISGSGGST |
| 1502. | CDR-H3 | AKDGQTGGNYAAEYFQH |
| 1503. | CDR-L1 | QSVSKW |
| 1504. | CDR-L2 | KAS |
| 1505. | CDR-L3 | QHFNTEQLT |
| 1506. | VH | ESGGGLVQPGGSLRLSCAASGFTFRSYAMSWVRQAPGKG |
| | | LEWVSGISGSGGSTHYADSVEGRFAVSRDNSKNTVYLEMN |
| | | SLRAEDTAVYYCAKDGQTGGNYAAEYFQHWGQGSLVTVS |
| | | S |
| 1507. | VL | RHRXTQSPSTLSASVGDRVTITCRASQSVSKWLAWYQQKA |
| | | GKAPRLLIYKASTLDSGVPSRFSGSGSGTEFTLTISSLQPDD |
| | | FATYYCQHENTEQLTFGGGTKVEIK |
| 1508. | FR-H1 | ESGGGLVQPGGSLRLSCAAS |
| 1509. | FR-H2 | MSWVRQAPGKGLEWVSG |
| 1510. | FR-H3 | HYADSVEGRFAVSRDNSKNTVYLEMNSLRAEDTAVYYC |
| 1511. | FR-H4 | WGQGSLVTVSS |
| 1512. | FR-L1 | RHRXTQSPSTLSASVGDRVTITCRAS |
| 1513. | FR-L2 | LAWYQQKAGKAPRLLIY |
| 1514. | FR-L3 | TLDSGVPSRFSGSGSGTEFTLTISSLQPDDFATYYC |
| 1515. | FR-L4 | FGGGTKVEIK |
| 1516. | heavychain | ESGGGLVQPGGSLRLSCAASGFTFRSYAMSWVRQAPGKG |
| | | LEWVSGISGSGGSTHYADSVEGRFAVSRDNSKNTVYLEMN |
| | | SLRAEDTAVYYCAKDGQTGGNYAAEYFQHWGQGSLVTVS |
| | | SKGPSVFPLAPSSKSTSGGTAALGXLVKDYFPEPVTVSWNS |
| | | GALTSGVHTFPAVLQSSGLYSLSSVVTVPSSSLGTQTYICNV |
| | | NHKPSNTKVDKXVEPKSCDKTHTCPPCPAPELLGGPSVFLF |
| | | PPKPKDTLMISRT |
| 1517. | lightchain | RHRXTQSPSTLSASVGDRVTITCRASQSVSKWLAWYQQKA |
| | | GKAPRLLIYKASTLDSGVPSRFSGSGSGTEFTLTISSLQPDD |
| | | FATYYCQHFNTEQLTFGGGTKVEIKAPSVFIFPPSDEQLKSG |
| | | TASVVCLLNNFYPREAKVQWKVDNALQSGNSQESVTEQDS |
| | | KDSTYSLSSTLTLSKADYEKHKVYACEVTHQGLSSPVTKSF |
| | | NRGEC*K |
| 1518. | VH_nuc | gagtctgggggaggcttggtgcagcctggggggtccctgagactctcgtgtgcagcc |
| | | tctggattcacctttagaagttatgccatgagttgggtccgacaggctccagggaagg |
| | | gactggagtgggtctctggtattagtggtagtggcggcagcacacactatgcagactc |
| | | cgtggagggccggttcgccgtctccagagacaattccaagaacacggtgtatctgg |
| | | aaatgaacagcctgagagccgaggac |
| 1519. | VL_nuc | cgacatcgnnngacccagtctccttccaccctgtctgcatctgtgggagacagagtc |
| | | accatcacttgccgggccagtcagagtgttagtaagtggttggcctggtatcagcaga |
| | | aagcagggaaagcccctaggctcctgatctacaaggcgtccactttagatagtggg |
| | | gtcccatcaaggttcagcggcagtggatctgggacagaattcactcttaccatcagc |
| | | agcctgcagcctgatgattttgcaact |
| SEQ ID NO | | 111BU7P1A12 |
| 1520. | CDR-H1 | GFTFXSXA |
| 1521. | CDR-H2 | IXXXGGXT |
| 1522. | CDR-H3 | XXXXXXXGXXXXXTX |
| 1523. | CDR-L1 | QPISSTY |
| 1524. | CDR-L2 | ATS |
| 1525. | CDR-L3 | QHYSNSPPYT |
| 1526. | VH | GXLRXSCAASGFTFXSXAMXWVRQXPGKGLEWVXXIXXXG |
| | | GXTXYXXXVKGRFXISRDXSKXTLYLQMNSLXXEDTAXYXC |
| | | XXXXXXXGXXXXXTXWGQGTXVTVSS |
| 1527. | VL | EIXXTQSPGTLSLSLGEGATLSCRASQPISSTYLTWYQQRP |
| | | GQAPRLLIYATSTRATDIPDRFSGSGSGTDFTLTISRLETEDF |
| | | AVYYCQHYSNSPPYTFGQGTKLEIK |
| 1528. | FR-H1 | GXLRXSCAAS |
| 1529. | FR-H2 | MXWVRQXPGKGLEWVXX |
| 1530. | FR-H3 | XYXXXVKGRFXISRDXSKXTLYLQMNSLXXEDTAXYXC |
| 1531. | FR-H4 | WGQGTXVTVSS |
| 1532. | FR-L1 | EIXXTQSPGTLSLSLGEGATLSCRAS |
| 1533. | FR-L2 | LTWYQQRPGQAPRLLIY |
| 1534. | FR-L3 | TRATDIPDRFSGSGSGTDFTLTISRLETEDFAVYYC |
| 1535. | FR-L4 | FGQGTKLEIK |
| 1536. | heavychain | GXLRXSCAASGFTFXSXAMXWVRQXPGKGLEWVXXIXXXG |
| | | GXTXYXXXVKGRFXISRDXSKXTLYLQMNSLXXEDTAXYXC |
| | | XXXXXXXGXXXXXTXWGQGTXVTVSSKGPSVFPLAPSSKS |
| | | TSGGTAALGXLVKDYFPEPVTVSWNSGALTSGVHTFPAVLQ |
| | | SSGLYSLSSVVTVPSSSLGTQTYICNVNHKPSNTKVDKXVE |
| | | PKSCDKTHTCPPCPAPELLGGPSVFLFPPKPKDTLMISRTPE |
| | | VTCVVVDVSH |
| 1537. | lightchain | EIXXTQSPGTLSLSLGEGATLSCRASQPISSTYLTWYQQRP |
| | | GQAPRLLIYATSTRATDIPDRFSGSGSGTDFTLTISRLETEDF |
| | | AVYYCQHYSNSPPYTFGQGTKLEIKAPSVFIFPPSDEQLKSG |
| | | TASVVCLLNNFYPREAKVQWKVDNALQSGNSQESVTEQDS |
| | | KDSTYSLSSTLTLSKADYEKHKVYACEVTHQGLSSPVTKSF |
| | | NRGEC*K |
| 1538. | VH_nuc | ggggncctgagantctcctgtgcagcctctggattcacctttnncagywakgccatg |
| | | asctgggtccgccagrctccagggaagggrctggagtgggtyksmsstattarwrg |
| | | nrrtggtggkasmacakactacrcwgmmyccgtgaarggcmgrttcaycatytc |
| | | magagayrattcmaarracacgytgtatctrcaratgaayagyctgararccgagg |
| | | acacrgccstmtattwctgtrcsanaakaysa |
| 1539. | VL_nuc | gaaattgnnntgacgcagtctccaggcaccctgtctttgtctctaggtgaaggagcca |
| | | ccctctcctgcagggccagtcagcctattagtagcacctacttaacctggtaccagca |
| | | gagacctggccaggctccccggctcctcatctacgctacgtccaccagggccactg |
| | | acatcccagacaggttcagtggcagtggctctgggacagacttcactctcaccatca |
| | | gtagactggagactgaagattttgca |
| SEQ ID NO | | 111BU7P1D2 |
| 1540. | CDR-H1 | GFPFDSHA |
| 1541. | CDR-H2 | ISNNGASA |
| 1542. | CDR-H3 | ARTQYYDSRGFYFSLDA |
| 1543. | CDR-L1 | QPISSTY |
| 1544. | CDR-L2 | ATS |
| 1545. | CDR-L3 | QHYSNSPPYT |
| 1546. | VH | ESGGGVVQPGRSVRLSCVGSGFPFDSHAIHWVRQAPGWG |
| | | LEWVAVISNNGASAHYTDSVKGRFTVSRDNSKHTVYLLLNS |
| | | LTKEDTAVYYCARTQYYDSRGFYFSLDAWGQGTLVTVSS |
| 1547. | VL | EIXXTQSPGTLSLSLGEGATLSCRASQPISSTYLTWYQQRP |
| | | GQAPRLLIYATSTRATDIPDRFSGSGSGTDFTLTISRLETEDF |
| | | AVYYCQHYSNSPPYTFGQGTKLEIK |
| 1548. | FR-H1 | ESGGGVVQPGRSVRLSCVGS |
| 1549. | FR-H2 | IHWVRQAPGWGLEWVAV |
| 1550. | FR-H3 | HYTDSVKGRFTVSRDNSKHTVYLLLNSLTKEDTAVYYC |
| 1551. | FR-H4 | WGQGTLVTVSS |
| 1552. | FR-L1 | EIXXTQSPGTLSLSLGEGATLSCRAS |
| 1553. | FR-L2 | LTWYQQRPGQAPRLLIY |
| 1554. | FR-L3 | TRATDIPDRFSGSGSGTDFTLTISRLETEDFAVYYC |
| 1555. | FR-L4 | FGQGTKLEIK |
| 1556. | heavychain | ESGGGVVQPGRSVRLSCVGSGFPFDSHAIHWVRQAPGWG |
| | | LEWVAVISNNGASAHYTDSVKGRFTVSRDNSKHTVYLLLNS |
| | | LTKEDTAVYYCARTQYYDSRGFYFSLDAWGQGTLVTVSSK |
| | | GPSVFPLAPSSKSTSGGTAALGXLVKDYFPEPVTVSWNSGA |
| | | LTSGVHTFPAVLQSSGLYSLSSVVTVPSSSLGTQTYICNVNH |
| | | KPSNTKVDKXVEPKSCDKTHTCPPCPAPELLGGPSVFLFPP |
| | | KPKDTLMISRT |
| 1557. | lightchain | EIXXTQSPGTLSLSLGEGATLSCRASQPISSTYLTWYQQRP |
| | | GQAPRLLIYATSTRATDIPDRFSGSGSGTDFTLTISRLETEDF |
| | | AVYYCQHYSNSPPYTFGQGTKLEIKAPSVFIFPPSDEQLKSG |
| | | TASVVCLLNNFYPREAKVQWKVDNALQSGNSQESVTEQDS |
| | | KDSTYSLSSTLTLSKADYEKHKVYACEVTHQGLSSPVTKSF |
| | | NRGEC*K |
| 1558. | VH_nuc | gagtctgggggaggcgttgtccagcctgggaggtccgtgagactctcctgtgtaggct |
| | | ctggattcccattcgatagtcatgccatacactgggtccgccaggctccaggctgggg |
| | | cctggagtgggtggcagttatctcaaacaatggcgccagtgcacattacacagactc |
| | | cgtgaagggccgattcaccgtctccagagacaattccaagcacaccgtctatctactt |
| | | ctgaacagtctgacaaaagaagac |
| 1559. | VL_nuc | gaaattgnnntgacgcagtctccaggcaccctgtctttgtctctaggtgaaggagcca |
| | | ccctctcctgcagggccagtcagcctattagtagcacctacttaacctggtaccagca |
| | | gagacctggccaggctccccggctcctcatctacgctacgtccaccagggccactg |
| | | acatcccagacaggttcagtggcagtggctctgggacagacttcactctcaccatca |
| | | gtagactggagactgaagattttgca |
| SEQ ID NO | | 111BU7P1D5 |
| 1560. | CDR-H1 | GFTFSTFA |
| 1561. | CDR-H2 | ISDNGNRK |
| 1562. | CDR-H3 | AKTRDYDSRGYYFGLDH |
| 1563. | CDR-L1 | QSVISNY |
| 1564. | CDR-L2 | AAS |
| 1565. | CDR-L3 | QHYGNSPPYT |
| 1566. | VH | CXLVESGGGVVQPGRSLRLSCAASGFTFSTFALHWVRQAP |
| | | GKGLEWMAVISDNGNRKDYADSVKGRFTISRDNSENTLYLE |
| | | MNSLRPEDTXVYYCAKTRDYDSRGYYFGLDHWGQGTLVTV |
| | | SS |
| 1567. | VL | EIGXTQSPGTLSLSPGERATLSCRASQSVISNYLAWYQHKP |
| | | GQAPRLLIYAASSRATDIPDRFSGSGSGTDFILTISRLEPEDF |
| | | AVYYCQHYGNSPPYTFGQGTKVEIK |
| 1568. | FR-H1 | CXLVESGGGVVQPGRSLRLSCAAS |
| 1569. | FR-H2 | LHWVRQAPGKGLEWMAV |
| 1570. | FR-H3 | DYADSVKGRFTISRDNSENTLYLEMNSLRPEDTXVYYC |
| 1571. | FR-H4 | WGQGTLVTVSS |
| 1572. | FR-L1 | EIGXTQSPGTLSLSPGERATLSCRAS |
| 1573. | FR-L2 | LAWYQHKPGQAPRLLIY |
| 1574. | FR-L3 | SRATDIPDRFSGSGSGTDFILTISRLEPEDFAVYYC |
| 1575. | FR-L4 | FGQGTKVEIK |
| 1576. | heavychain | CXLVESGGGVVQPGRSLRLSCAASGFTFSTFALHWVRQAP |
| | | GKGLEWMAVISDNGNRKDYADSVKGRFTISRDNSENTLYLE |
| | | MNSLRPEDTXVYYCAKTRDYDSRGYYFGLDHWGQGTLVTV |
| | | SSKGPSVFPLAPSSKSTSGGTAALGXLVKDYFPEPVTVSWN |
| | | SGALTSGVHTFPAVLQSSGLYSLSSVVTVPSSSLGTQTYICN |
| | | VNHKPSNTKVDKXVEPKSCDKTHTCPPCPAPELLGGPSVFL |
| | | FPPKPKDTLM |
| 1577. | lightchain | EIGXTQSPGTLSLSPGERATLSCRASQSVISNYLAWYQHKP |
| | | GQAPRLLIYAASSRATDIPDRFSGSGSGTDFILTISRLEPEDF |
| | | AVYYCQHYGNSPPYTFGQGTKVEIKAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKS |
| | | FNRGEC*K |
| 1578. | VH_nuc | ggtgccnnctggtggagtctgggggaggcgtggtccagcctgggaggtccctgaga |
| | | ctctcctgtgcagcctctggattcacatttagtacctttgctctacattgggtccgccagg |
| | | ctccaggcaaggggctggagtggatggccgttatatcagataatgggaatagaaaa |
| | | gactacgcagactccgtgaagggccgattcaccatctcccgagacaactccgaga |
| | | acacactgtatctggaaatgaacagcc |
| 1579. | VL_nuc | gaaataggnnnnacgcagtctccaggcaccctgtctttgtctccaggggaaagagc |
| | | caccctctcctgcagggccagtcagagtgttatcagcaactacttagcctggtatcag |
| | | cacaaacctggccaggctcccaggctcctcatctatgctgcctccagcagggccact |
| | | gacatcccagacaggttcagtggcagtgggtctgggacagacttcattctcaccatc |
| | | agcagactggagcctgaagattttgcg |
| SEQ ID NO | | 22BU7P1C8 |
| 1580. | CDR-H1 | GFTFSYYA |
| 1581. | CDR-H2 | ISNDESHR |
| 1582. | CDR-H3 | AKTLDYSNYGYYFGLDV |
| 1583. | CDR-L1 | QSLSTNF |
| 1584. | CDR-L2 | AAS |
| 1585. | CDR-L3 | QRYGDSPPYT |
| 1586. | VH | VESGGGVVHPGKSLRLSCAASGFTFSYYAMHWVRQAPGK |
| | | GLEWVAIISNDESHRTYADSVRGRFTISRDNSNNILFLQMNN |
| | | VRVEDTAVYYCAKTLDYSNYGYYFGLDVWGQGTTVTVSS |
| 1587. | VL | ATLSCRSSQSLSTNFLAWYQQKPGQTPRLLIYAASNRATGIP |
| | | DRFSGSGSGTDFTLTISRLEPEDFAVYYCQRYGDSPPYTXG |
| 1588. | FR-H1 | VESGGGVVHPGKSLRLSCAAS |
| 1589. | FR-H2 | MHWVRQAPGKGLEWVAI |
| 1590. | FR-H3 | TYADSVRGRFTISRDNSNNILFLQMNNVRVEDTAVYYC |
| 1591. | FR-H4 | WGQGTTVTVSS |
| 1592. | FR-L1 | ATLSCRSS |
| 1593. | FR-L2 | LAWYQQKPGQTPRLLIY |
| 1594. | FR-L3 | NRATGIPDRFSGSGSGTDFTLTISRLEPEDFAVYYC |
| 1595. | FR-L4 | XG |
| 1596. | heavychain | VESGGGVVHPGKSLRLSCAASGFTFSYYAMHWVRQAPGK |
| | | GLEWVAIISNDESHRTYADSVRGRFTISRDNSNNILFLQMNN |
| | | VRVEDTAVYYCAKTLDYSNYGYYFGLDVWGQGTTVTVSSK |
| | | GPSVFPLAPSSKSTSGGTAALGXLVKDYFPEPVTVSWNSGA |
| | | LTSGVHTFPAVLQSSGLYSLSSVVTVPSSSLGTQTYICNVNH |
| | | KPSNTKVDKXVEPKSCDKTHTCPPCPAPELLGGPSVFLFPP |
| | | KPKDTLMISR |
| 1597. | lightchain | ATLSCRSSQSLSTNFLAWYQQKPGQTPRLLIYAASNRATGIP |
| | | DRFSGSGSGTDFTLTISRLEPEDFAVYYCQRYGDSPPYTXG |
| | | APSVFIFPPSDEQLKSGTASVVCLLNNFYPREAKVQWKVDN |
| | | ALQSGNSQESVTEQDSKDSTYSLSSTLTLSKADYEKHKVYA |
| | | CEVTHQGLSSPVTKSFNRGEC*K |
| 1598. | VH_nuc | gtngagtctgggggcggcgttgtccaccctgggaagtccctgagactctcctgtgca |
| | | gcctctggattcacattctcttactatgcaatgcattgggtccgccaggctccaggcaa |
| | | gggactggagtgggtggcaattatatcaaatgatgaaagtcatagaacctacgcag |
| | | actccgtgaggggccggttcaccatttccagagacaattccaacaacatcttatttctg |
| | | caaatgaacaacgtaagagtcgag |
| 1599. | VL_nuc | gccaccctctcctgcaggtccagtcagagtctcagcaccaatttcttagcctggtacca |
| | | gcagaaacctggccagactcccaggctcctcatctacgctgcatccaacagggcca |
| | | ctggcatcccagacaggttcagtggcagtgggtctgggacagacttcactctcaccat |
| | | cagccggctggagcctgaagattttgcagtatattactgtcagcgctatggtgattcac |
| | | ctccgtacacttncggcc |
| SEQ ID NO | | 22BU7P1D12 |
| 1600. | CDR-H1 | GFTFNFFA |
| 1601. | CDR-H2 | ISDNGGHK |
| 1602. | CDR-H3 | AKTLDYSNYGYYFGLDA |
| 1603. | CDR-L1 | QSVTSNY |
| 1604. | CDR-L2 | GAS |
| 1605. | CDR-L3 | QRYGNSPPYT |
| 1606. | VH | VXXVESGGGVAQPGRSLTLSCATSGFTFNFFAMHWVRQAP |
| | | GKGLEWVAVISDNGGHKSHAESLQGRFTISRDNSRSTLFLE |
| | | MNSLRAEDTAVYYCAKTLDYSNYGYYFGLDAWGPGTTVVV |
| | | SS |
| 1607. | VL | ATLSCRASQSVTSNYLAWYQQKPGQAPRLLIYGASNRATGI |
| | | PDRFSGSGSGTDFTLTISGLEPEDFAVYYCQRYGNSPPYTF |
| | | GPXT |
| 1608. | FR-H1 | VXXVESGGGVAQPGRSLTLSCATS |
| 1609. | FR-H2 | MHWVRQAPGKGLEWVAV |
| 1610. | FR-H3 | SHAESLQGRFTISRDNSRSTLFLEMNSLRAEDTAVYYC |
| 1611. | FR-H4 | WGPGTTVVVSS |
| 1612. | FR-L1 | ATLSCRAS |
| 1613. | FR-L2 | LAWYQQKPGQAPRLLIY |
| 1614. | FR-L3 | NRATGIPDRFSGSGSGTDFTLTISGLEPEDFAVYYC |
| 1615. | FR-L4 | FGPXT |
| 1616. | heavychain | VXXVESGGGVAQPGRSLTLSCATSGFTFNFFAMHWVRQAP |
| | | GKGLEWVAVISDNGGHKSHAESLQGRFTISRDNSRSTLFLE |
| | | MNSLRAEDTAVYYCAKTLDYSNYGYYFGLDAWGPGTTVVV |
| | | SSKGPSVFPLAPSSKSTSGGTAALGXLVKDYFPEPVTVSWN |
| | | SGALTSGVHTFPAVLQSSGLYSLSSVVTVPSSSLGTQTYICN |
| | | VNHKPSNTKVDKXVEPKSCDKTHTCPPCPAPELLGGPSVFL |
| | | FPPKPKDTLM |
| 1617. | lightchain | ATLSCRASQSVTSNYLAWYQQKPGQAPRLLIYGASNRATGI |
| | | PDRFSGSGSGTDFTLTISGLEPEDFAVYYCQRYGNSPPYTF |
| | | GPXTAPSVFIFPPSDEQLKSGTASVVCLLNNFYPREAKVQW |
| | | KVDNALQSGNSQESVTEQDSKDSTYSLSSTLTLSKADYEKH |
| | | KVYACEVTHQGLSSPVTKSFNRGEC*K |
| 1618. | VH_nuc | gtgcnannggtggagtctgggggaggcgtggcccagcctgggaggtcactgacgc |
| | | tctcctgtgcgacctctggattcaccttcaatttctttgcaatgcactgggtccgccaggc |
| | | tccaggcaaggggctggagtgggtggcagttatctcagataatggcggtcacaaat |
| | | cccatgcagagtccctacagggccgattcaccatttccagagacaattccaggagc |
| | | acgttgtttctggagatgaacagtctc |
| 1619. | VL_nuc | gccaccctctcctgcagggccagtcagagtgttaccagcaactacttagcctggtac |
| | | cagcagaaacctggccaggctcccaggctcctcatctatggtgcatccaacagggc |
| | | cactggcatcccagacaggttcagtggcagtgggtctgggacagacttcactctcac |
| | | catcagcggactggaacctgaagattttgcagtgtattactgtcagcgatatggtaact |
| | | cacctccgtacacnttcggccctgng |
| SEQ ID NO | | 29BU7P1A2 |
| 1620. | CDR-H1 | GFTLSTYG |
| 1621. | CDR-H2 | SPNRGSST |
| 1622. | CDR-H3 | ARQTKYDFSRGYYKPYSWFDP |
| 1623. | CDR-L1 | QSISTY |
| 1624. | CDR-L2 | AAS |
| 1625. | CDR-L3 | QQNYNMWT |
| 1626. | VH | QVQLVESGGGSVQPGGSLRLSCVGSGFTLSTYGMTWVRQ |
| | | APRKGLEWVSLSPNRGSSTYYADSVKGRFTISRDNSKNTLY |
| | | LQMNSLRVEDTAVYYCARQTKYDFSRGYYKPYSWFDPWG |
| | | QGTLVTVSS |
| 1627. | VL | ASVGDTVTISCRASQSISTYLNWYQQKPGKVPKLLIYAASSL |
| | | ESGVPSRFSGSGSGTDFTLTISSLQSEDFAIYYCQQNYNMW |
| | | TFGQGTKVX |
| 1628. | FR-H1 | QVQLVESGGGSVQPGGSLRLSCVGS |
| 1629. | FR-H2 | MTWVRQAPRKGLEWVSL |
| 1630. | FR-H3 | YYADSVKGRFTISRDNSKNTLYLQMNSLRVEDTAVYYC |
| 1631. | FR-H4 | WGQGTLVTVSS |
| 1632. | FR-L1 | ASVGDTVTISCRAS |
| 1633. | FR-L2 | LNWYQQKPGKVPKLLIY |
| 1634. | FR-L3 | SLESGVPSRFSGSGSGTDFTLTISSLQSEDFAIYYC |
| 1635. | FR-L4 | FGQGTKVX |
| 1636. | heavychain | QVQLVESGGGSVQPGGSLRLSCVGSGFTLSTYGMTWVRQ |
| | | APRKGLEWVSLSPNRGSSTYYADSVKGRFTISRDNSKNTLY |
| | | LQMNSLRVEDTAVYYCARQTKYDFSRGYYKPYSWFDPWG |
| | | QGTLVTVSSKGPSVFPLAPSSKSTSGGTAALGXLVKDYFPE |
| | | PVTVSWNSGALTSGVHTFPAVLQSSGLYSLSSVVTVPSSSL |
| | | GTQTYICNVNHKPSNTKVDKXVEPKSCDKTHTCPPCPAPEL |
| | | LGGPSVFLFPPKP |
| 1637. | lightchain | ASVGDTVTISCRASQSISTYLNWYQQKPGKVPKLLIYAASSL |
| | | ESGVPSRFSGSGSGTDFTLTISSLQSEDFAIYYCQQNYNMW |
| | | TFGQGTKVXAPSVFIFPPSDEQLKSGTASVVCLLNNFYPRE |
| | | AKVQWKVDNALQSGNSQESVTEQDSKDSTYSLSSTLTLSK |
| | | ADYEKHKVYACEVTHQGLSSPVTKSFNRGEC*K |
| 1638. | VH_nuc | caggtgcagctggtggagtctgggggaggctcagtccagcctggggggtccctgag |
| | | actctcctgtgtaggctcgggattcacgcttagcacctatggcatgacgtgggtccgcc |
| | | aggctccacggaaggggctggagtgggtctcacttagtcccaatcgtggaagttcca |
| | | catactacgcggactccgtgaagggccggttcaccatctccagagacaattccaag |
| | | aacacactgtatctgcaaatgaatagc |
| 1639. | VL_nuc | tgcatctgtaggagacacagtcaccatctcttgccgggcaagtcagagcattagcac |
| | | ctatttaaattggtatcaacagaaaccagggaaagtccctaaactcctaatctatgctg |
| | | catccagtttggaaagtggggtcccatcaaggttcagtggcagtggatctgggacag |
| | | atttcactctcaccatcagcagtctgcagtctgaagattttgcaatttactactgtcaaca |
| | | gaattacaatatgtggacgtt |
| SEQ ID NO | | 29BU7P1C11 |
| 1640. | CDR-H1 | GDSISSYY |
| 1641. | CDR-H2 | IFTSGST |
| 1642. | CDR-H3 | ARDRRGLTPSGTWRRWFDP |
| 1643. | CDR-L1 | QNIGSH |
| 1644. | CDR-L2 | STS |
| 1645. | CDR-L3 | QQSNSNTPT |
| 1646. | VH | QESGPGLVKPSETLSLTCSVSGDSISSYYWSWIRQPVGKRP |
| | | EWIGRIFTSGSTNYNPSLTSRVTMSVDTPKNQFSLHLTSVTA |
| | | ADTAVYYCARDRRGLTPSGTWRRWFDPWGQGILVTISS |
| 1647. | VL | LSASVGDXITITCRASQNIGSHLNWYQQKTGTAPKLLIYSTS |
| | | NLQDGVPSRFSGSGSGTDFTLTISSLLPEDFTIYYCQQSNSN |
| | | TPTFGGXTKVE |
| 1648. | FR-H1 | QESGPGLVKPSETLSLTCSVS |
| 1649. | FR-H2 | WSWIRQPVGKRPEWIGR |
| 1650. | FR-H3 | NYNPSLTSRVTMSVDTPKNQFSLHLTSVTAADTAVYYC |
| 1651. | FR-H4 | WGQGILVTISS |
| 1652. | FR-L1 | LSASVGDXITITCRAS |
| 1653. | FR-L2 | LNWYQQKTGTAPKLLIY |
| 1654. | FR-L3 | NLQDGVPSRFSGSGSGTDFTLTISSLLPEDFTIYYC |
| 1655. | FR-L4 | FGGXTKVE |
| 1656. | heavychain | QESGPGLVKPSETLSLTCSVSGDSISSYYWSWIRQPVGKRP |
| | | EWIGRIFTSGSTNYNPSLTSRVTMSVDTPKNQFSLHLTSVTA |
| | | ADTAVYYCARDRRGLTPSGTWRRWFDPWGQGILVTISSKG |
| | | PSVFPLAPSSKSTSGGTAALGXLVKDYFPEPVTVSWNSGAL |
| | | TSGVHTFPAVLQSSGLYSLSSVVTVPSSSLGTQTYICNVNHK |
| | | PSNTKVDKXVEPKSCDKTHTCPPCPAPELLGGPSVFLFPPK |
| | | PKDTLMIS |
| 1657. | lightchain | LSASVGDXITITCRASQNIGSHLNWYQQKTGTAPKLLIYSTS |
| | | NLQDGVPSRFSGSGSGTDFTLTISSLLPEDFTIYYCQQSNSN |
| | | TPTFGGXTKVEAPSVFIFPPSDEQLKSGTASVVCLLNNFYPR |
| | | EAKVQWKVDNALQSGNSQESVTEQDSKDSTYSLSSTLTLS |
| | | KADYEKHKVYACEVTHQGLSSPVTKSFNRGEC*K |
| 1658. | VH_nuc | gcaggagtcgggcccaggactagtgaagccttcggagaccctgtccctcacttgca |
| | | gtgtctctggtgactccatcagtagttactattggagttggatccgccagcccgtcggg |
| | | aagcgaccggagtggattggccgtatttttaccagtggcagtaccaattataacccct |
| | | ccctaacgagtcgagtcactatgtcagtggacacgcccaagaaccagttctccctgc |
| | | acctgacctctgtgaccgccgcgga |
| 1659. | VL_nuc | ctgtctgcatctgtgggagacngaatcaccatcacttgccgggcaagtcagaatattg |
| | | gcagccatttaaattggtatcagcagaaaacagggacagcccctaagctcctgatct |
| | | attctacatccaatttgcaagatggggtcccatcaaggttcagtggcagtggatctggg |
| | | acagatttcactctcaccatcagcagtctgctacctgaagattttacaatttactactgtc |
| | | aacagagtaacagtaatact |
| SEQ ID NO | | 29BU7P1D1 |
| 1660. | CDR-H1 | GYTFSRYA |
| 1661. | CDR-H2 | INGGDGNT |
| 1662. | CDR-H3 | ARGRAYTYGRLSLSYAMDV |
| 1663. | CDR-L1 | QSLLHSNGYNY |
| 1664. | CDR-L2 | LGS |
| 1665. | CDR-L3 | MQGLQTPL |
| 1666. | VH | GAEVKKPGASVRVFCKASGYTFSRYAIHWVRRAPGQRLEW |
| | | MGRINGGDGNTDSSQKFQGRVTFTRDTSASTAYMELRSLR |
| | | SEDSAVFYCARGRAYTYGRLSLSYAMDVWGQGTTVTVSS |
| 1667. | VL | CRSSQSLLHSNGYNYLDWYLQKPGQPPQLLIYLGSNRASG |
| | | VPDRFSXXGSGTXFTLXISRVEAEDVGVYYCMQGLQTPLXG |
| | | PGTR |
| 1668. | FR-H1 | GAEVKKPGASVRVFCKAS |
| 1669. | FR-H2 | IHWVRRAPGQRLEWMGR |
| 1670. | FR-H3 | DSSQKFQGRVTFTRDTSASTAYMELRSLRSEDSAVFYC |
| 1671. | FR-H4 | WGQGTTVTVSS |
| 1672. | FR-L1 | CRSS |
| 1673. | FR-L2 | LDWYLQKPGQPPQLLIY |
| 1674. | FR-L3 | NRASGVPDRFSXXGSGTXFTLXISRVEAEDVGVYYC |
| 1675. | FR-L4 | XGPGTR |
| 1676. | heavychain | GAEVKKPGASVRVFCKASGYTFSRYAIHWVRRAPGQRLEW |
| | | MGRINGGDGNTDSSQKFQGRVTFTRDTSASTAYMELRSLR |
| | | SEDSAVFYCARGRAYTYGRLSLSYAMDVWGQGTTVTVSSK |
| | | GPSVFPLAPSSKSTSGGTAALGXLVKDYFPEPVTVSWNSGA |
| | | LTSGVHTFPAVLQSSGLYSLSSVVTVPSSSLGTQTYICNVNH |
| | | KPSNTKVDKXVEPKSCDKTHTCPPCPAPELLGGPSVFLFPP |
| | | KPKDTLMISRT |
| 1677. | lightchain | CRSSQSLLHSNGYNYLDWYLQKPGQPPQLLIYLGSNRASG |
| | | VPDRFSXXGSGTXFTLXISRVEAEDVGVYYCMQGLQTPLXG |
| | | PGTRAPSVFIFPPSDEQLKSGTASVVCLLNNFYPREAKVQW |
| | | KVDNALQSGNSQESVTEQDSKDSTYSLSSTLTLSKADYEKH |
| | | KVYACEVTHQGLSSPVTKSFNRGEC*K |
| 1678. | VH_nuc | ggggctgaggtgaagaagcctggggcctcagtgagggttttctgcaaggcctctgg |
| | | atacaccttcagtaggtatgcaatacattgggtgcgccgggcacccggacaaaggc |
| | | ttgagtggatgggaaggatcaacggtggagatggcaacacagactcttcacagaa |
| | | gttccagggcagagtcacctttaccagggacacatccgcgagcacagcctatatgg |
| | | agctgaggagcctcagatctgaagactcggct |
| 1679. | VL_nuc | ctgcaggtctagtcagagcctcctgcatagtaatggatacaactatttggattggtacct |
| | | gcagaagccagggcagcctccacagctcctgatctatttgggttctaatcgggcctcc |
| | | ggggtccctgacaggttcagtkgyrgtggatcaggyacwgaktttacactgawaatc |
| | | agcagagtggaggctgaggatgttggggtttattactgcatgcaaggtctacaaactc |
| | | ccctttncggccctggnaccag |
| SEQ ID NO | | 29BU7P1D3 |
| 1680. | CDR-H1 | GYTFTSKG |
| 1681. | CDR-H2 | ISAYDGNT |
| 1682. | CDR-H3 | ATSGSPRAGRDSTVAFDY |
| 1683. | CDR-L1 | QSVISSY |
| 1684. | CDR-L2 | GAS |
| 1685. | CDR-L3 | HQYGSSPGT |
| 1686. | VH | VQSGAEVKKPGASVKVSCRASGYTFTSKGITWVRQAPGQG |
| | | LEWMGWISAYDGNTNYAQKFQGRVTMTTDTSTRTAYMELR |
| | | SLRSDDTAVYHCATSGSPRAGRDSTVAFDYWGQGTLVTVS |
| | | P |
| 1687. | VL | PGXRATLSCRASQSVISSYLAWYQQKPGQAPRLLIYGASSR |
| | | VTGIPDRFSGSQSGTDFTFTISRLEPEDFAVYYCHQYGSSP |
| | | GTFGQXTK |
| 1688. | FR-H1 | VQSGAEVKKPGASVKVSCRAS |
| 1689. | FR-H2 | ITWVRQAPGQGLEWMGW |
| 1690. | FR-H3 | NYAQKFQGRVTMTTDTSTRTAYMELRSLRSDDTAVYHC |
| 1691. | FR-H4 | WGQGTLVTVSP |
| 1692. | FR-L1 | PGXRATLSCRAS |
| 1693. | FR-L2 | LAWYQQKPGQAPRLLIY |
| 1694. | FR-L3 | SRVTGIPDRFSGSQSGTDFTFTISRLEPEDFAVYYC |
| 1695. | FR-L4 | FGQXTK |
| 1696. | heavychain | VQSGAEVKKPGASVKVSCRASGYTFTSKGITWVRQAPGQG |
| | | LEWMGWISAYDGNTNYAQKFQGRVTMTTDTSTRTAYMELR |
| | | SLRSDDTAVYHCATSGSPRAGRDSTVAFDYWGQGTLVTVS |
| | | PKGPSVFPLAPSSKSTSGGTAALGXLVKDYFPEPVTVSWNS |
| | | GALTSGVHTFPAVLQSSGLYSLSSVVTVPSSSLGTQTYICNV |
| | | NHKPSNTKVDKXVEPKSCDKTHTCPPCPAPELLGGPSVFLF |
| | | PPKPKDTLMIS |
| 1697. | lightchain | PGXRATLSCRASQSVISSYLAWYQQKPGQAPRLLIYGASSR |
| | | VTGIPDRFSGSQSGTDFTFTISRLEPEDFAVYYCHQYGSSP |
| | | GTFGQXTKAPSVFIFPPSDEQLKSGTASVVCLLNNFYPREA |
| | | KVQWKVDNALQSGNSQESVTEQDSKDSTYSLSSTLTLSKA |
| | | DYEKHKVYACEVTHQGLSSPVTKSFNRGEC*K |
| 1698. | VH_nuc | gtgcagtctggagctgaggtgaagaagccgggggcctcagtgaaggtctcctgcag |
| | | ggcttctggttacacctttaccagcaagggtatcacctgggtgcgacaggcccctgga |
| | | caagggcttgagtggatgggatggatcagcgcttacgatggtaacacaaactatgc |
| | | acagaaattccagggtagagtcaccatgaccacagacacatccacgaggactgcc |
| | | tacatggagctgaggagcctgagatctgac |
| 1699. | VL_nuc | ctccrgggnnnagagccaccctctcctgcagggccagtcagagtgttatcagcagct |
| | | acttagcctggtaccagcagaagcctggccaggctcccaggctcctcatctatggtg |
| | | catccagcagggtcactggcatcccagacaggttcagtggcagtcagtctgggaca |
| | | gacttcactttcaccatcagcagactggagcctgaagattttgcagtgtattactgtcac |
| | | cagtatggtagctcacctgggacgt |
| SEQ ID NO | | 29BU7P1D12 |
| 1700. | CDR-H1 | GFTFSPYS |
| 1701. | CDR-H2 | ISASGDKR |
| 1702. | CDR-H3 | ARDQLENFESGGYYWPLAFDV |
| 1703. | CDR-L1 | EDIGTY |
| 1704. | CDR-L2 | VAS |
| 1705. | CDR-L3 | QQLNSYPLT |
| 1706. | VH | RCKLVESGGGLVQPGGSLRLSCVASGFTFSPYSMNWVRQ |
| | | APGKGLEWISYISASGDKRDSADSVKGRFIISRDNSQNSLYL |
| | | QLNSLRVDDTAVYYCARDQLENFESGGYYWPLAFDVWGQ |
| | | GTTVTVSS |
| 1707. | VL | SVGXXLTITCRASEDIGTYLAWYQQKPGTAPKLLIYVASTLQ |
| | | TGVPSRFSGSGSRTEFTLTINSLQPEDIATYYCQQLNSYPLT |
| | | FGGXT |
| 1708. | FR-H1 | RCKLVESGGGLVQPGGSLRLSCVAS |
| 1709. | FR-H2 | MNWVRQAPGKGLEWISY |
| 1710. | FR-H3 | DSADSVKGRFIISRDNSQNSLYLQLNSLRVDDTAVYYC |
| 1711. | FR-H4 | WGQGTTVTVSS |
| 1712. | FR-L1 | SVGXXLTITCRAS |
| 1713. | FR-L2 | LAWYQQKPGTAPKLLIY |
| 1714. | FR-L3 | TLQTGVPSRFSGSGSRTEFTLTINSLQPEDIATYYC |
| 1715. | FR-L4 | FGGXT |
| 1716. | heavychain | RCKLVESGGGLVQPGGSLRLSCVASGFTFSPYSMNWVRQ |
| | | APGKGLEWISYISASGDKRDSADSVKGRFIISRDNSQNSLYL |
| | | QLNSLRVDDTAVYYCARDQLENFESGGYYWPLAFDVWGQ |
| | | GTTVTVSSKGPSVFPLAPSSKSTSGGTAALGXLVKDYFPEP |
| | | VTVSWNSGALTSGVHTFPAVLQSSGLYSLSSVVTVPSSSLG |
| | | TQTYICNVNHKPSNTKVDKXVEPKSCDKTHTCPPCPAPELL |
| | | GGPSVFLFPPKP |
| 1717. | lightchain | SVGXXLTITCRASEDIGTYLAWYQQKPGTAPKLLIYVASTLQ |
| | | TGVPSRFSGSGSRTEFTLTINSLQPEDIATYYCQQLNSYPLT |
| | | FGGXTAPSVFIFPPSDEQLKSGTASVVCLLNNFYPREAKVQ |
| | | WKVDNALQSGNSQESVTEQDSKDSTYSLSSTLTLSKADYE |
| | | KHKVYACEVTHQGLSSPVTKSFNRGEC*K |
| 1718. | VH_nuc | aggtgcaagctggtggagtctgggggaggcttggtccagccgggggggtccctga |
| | | gactctcctgtgtagcctctggattcaccttcagtccctacagtatgaactgggtccgcc |
| | | aggctccagggaagggactggaatggatttcatacattagtgctagtggagataaaa |
| | | gagactctgcagactctgtgaagggccgattcatcatctccagagacaactcccaga |
| | | actcactgtacctccaactcaacagt |
| 1719. | VL_nuc | atctgtagggracannctcaccatcacttgccgggccagtgaagacattggcacttat |
| | | ttagcctggtatcaacaaaaaccagggacagcccctaaactcctgatctatgttgcat |
| | | ccactttgcaaactggggtcccatcaaggttcagcggcagtggatctaggacagaat |
| | | tcactctcacaatcaacagcctgcagcctgaagatattgcgacttattactgtcaacaa |
| | | cttaatagttacccgctcacttt |
| SEQ ID NO | | 33BU7P1A11 |
| 1720. | CDR-H1 | GFMFSRFV |
| 1721. | CDR-H2 | MSYDEKNK |
| 1722. | CDR-H3 | VRGAYESSGHSFDH |
| 1723. | CDR-L1 | QSVGVH |
| 1724. | CDR-L2 | HTS |
| 1725. | CDR-L3 | QHRSTWPPAWT |
| 1726. | VH | VESGGGVVQPGKSLRLSCAASGFMFSRFVLHWVRQAPGK |
| | | GLEWVAVMSYDEKNKDYADSVKGRFTISRDNSKNTLDLQM |
| | | TTVTPEDTAVYYCVRGAYESSGHSFDHWGQGTLVTVSS |
| 1727. | VL | PGXGATLSCRASQSVGVHLAWYQQRPGQAPRLLLWHTSN |
| | | RATDIPARFSGSGSGTDFTLTISSLEPEDFAIYYCQHRSTWP |
| | | PAWTFGQXTK |
| 1728. | FR-H1 | VESGGGVVQPGKSLRLSCAAS |
| 1729. | FR-H2 | LHWVRQAPGKGLEWVAV |
| 1730. | FR-H3 | DYADSVKGRFTISRDNSKNTLDLQMTTVTPEDTAVYYC |
| 1731. | FR-H4 | WGQGTLVTVSS |
| 1732. | FR-L1 | PGXGATLSCRAS |
| 1733. | FR-L2 | LAWYQQRPGQAPRLLLW |
| 1734. | FR-L3 | NRATDIPARFSGSGSGTDFTLTISSLEPEDFAIYYC |
| 1735. | FR-L4 | FGQXTK |
| 1736. | heavychain | VESGGGVVQPGKSLRLSCAASGFMFSRFVLHWVRQAPGK |
| | | GLEWVAVMSYDEKNKDYADSVKGRFTISRDNSKNTLDLQM |
| | | TTVTPEDTAVYYCVRGAYESSGHSFDHWGQGTLVTVSSKG |
| | | PSVFPLAPSSKSTSGGTAALGXLVKDYFPEPVTVSWNSGAL |
| | | TSGVHTFPAVLQSSGLYSLSSVVTVPSSSLGTQTYICNVNHK |
| | | PSNTKVDKXVEPKSCDKTHTCPPCPAPELLGGPSVFLFPPK |
| | | PKDTLMISRTPE |
| 1737. | lightchain | PGXGATLSCRASQSVGVHLAWYQQRPGQAPRLLLWHTSN |
| | | RATDIPARFSGSGSGTDFTLTISSLEPEDFAIYYCQHRSTWP |
| | | PAWTFGQXTKAPSVFIFPPSDEQLKSGTASVVCLLNNFYPR |
| | | EAKVQWKVDNALQSGNSQESVTEQDSKDSTYSLSSTLTLS |
| | | KADYEKHKVYACEVTHQGLSSPVTKSFNRGEC*K |
| 1738. | VH_nuc | tggtggagtctgggggaggcgtggtccagcctgggaagtccctgcgactctcctgtg |
| | | cagcctctggattcatgttcagtaggtttgttctgcactgggtccgccaggccccgggc |
| | | aagggcctagagtgggtggcagttatgtcatatgatgaaaagaataaagactacgc |
| | | agactccgtgaagggccgattcaccatctccagagacaattccaagaacacgctgg |
| | | atctgcagatgactacagtgacacctg |
| 1739. | VL_nuc | ctccngggnnaggagccactctctcctgcagggccagtcagagtgttggcgtccact |
| | | tagcctggtaccaacagagacctggccaggctcccaggctcctcttgtggcatacat |
| | | ccaacagggccactgacatcccagccaggttcagcggcagtgggtctgggacaga |
| | | cttcactctcaccatcagcagccttgagcctgaagattttgcaatttattactgtcagcac |
| | | cgtagcacctggcctccggcgtgga |
| SEQ ID NO | | 33BU7P1B2 |
| 1740. | CDR-H1 | QFRFHRYA |
| 1741. | CDR-H2 | ISDVGRNE |
| 1742. | CDR-H3 | ARTMDYDRHNNYFGLDV |
| 1743. | CDR-L1 | HSVSSNF |
| 1744. | CDR-L2 | GSS |
| 1745. | CDR-L3 | QHYGDSPPYT |
| 1746. | VH | XLVESGGSVVQPGRSLRLSCGGSQFRFHRYALHWVRQVP |
| | | GKGLEWLAVISDVGRNEHYADSVKGRFTISRDNSQNMFYL |
| | | QMNSLRAEDTAVYFCARTMDYDRHNNYFGLDVWGQGTTVI |
| | | VSS |
| 1747. | VL | GXRVTLSCRTSHSVSSNFLAWYQQRPGQAPRLLIYGSSIRA |
| | | AGIPDRISGSGSGTDFTLTISRLEPEDFAVYFCQHYGDSPPY |
| | | TXGQXTK |
| 1748. | FR-H1 | XLVESGGSVVQPGRSLRLSCGGS |
| 1749. | FR-H2 | LHWVRQVPGKGLEWLAV |
| 1750. | FR-H3 | HYADSVKGRFTISRDNSQNMFYLQMNSLRAEDTAVYFC |
| 1751. | FR-H4 | WGQGTTVIVSS |
| 1752. | FR-L1 | GXRVTLSCRTS |
| 1753. | FR-L2 | LAWYQQRPGQAPRLLIY |
| 1754. | FR-L3 | IRAAGIPDRISGSGSGTDFTLTISRLEPEDFAVYFC |
| 1755. | FR-L4 | XGQXTK |
| 1756. | heavychain | XLVESGGSVVQPGRSLRLSCGGSQFRFHRYALHWVRQVP |
| | | GKGLEWLAVISDVGRNEHYADSVKGRFTISRDNSQNMFYL |
| | | QMNSLRAEDTAVYFCARTMDYDRHNNYFGLDVWGQGTTVI |
| | | VSSKGPSVFPLAPSSKSTSGGTAALGXLVKDYFPEPVTVSW |
| | | NSGALTSGVHTFPAVLQSSGLYSLSSVVTVPSSSLGTQTYIC |
| | | NVNHKPSNTKVDKXVEPKSCDKTHTCPPCPAPELLGGPSVF |
| | | LFPPKPKDTLMI |
| 1757. | lightchain | GXRVTLSCRTSHSVSSNFLAWYQQRPGQAPRLLIYGSSIRA |
| | | AGIPDRISGSGSGTDFTLTISRLEPEDFAVYFCQHYGDSPPY |
| | | TXGQXTKAPSVFIFPPSDEQLKSGTASVVCLLNNFYPREAKV |
| | | QWKVDNALQSGNSQESVTEQDSKDSTYSLSSTLTLSKADY |
| | | EKHKVYACEVTHQGLSSPVTKSFNRGEC*K |
| 1758. | VH_nuc | canctggtggagtctgggggaagcgtagtccagcctgggaggtccctgagactctc |
| | | ctgtggaggctctcaattcaggttccatagatacgctctacactgggtccgccaggttc |
| | | ctgggaaggggctggagtggttggcagtcatctcagatgttggcaggaatgagcact |
| | | atgcagactccgtgaagggccgcttcacgatctccagagacaactcccagaatatgt |
| | | tctatctccaaatgaacagcctgaga |
| 1759. | VL_nuc | caggggnncgggtcaccctctcttgcaggacaagtcacagtgtcagcagcaacttct |
| | | tagcctggtaccagcagagacctggccaggctcccaggctcctcatttacggttcatc |
| | | catcagggccgctggcatcccagacaggatcagtggcagtgggtctgggactgact |
| | | tcactctcaccatcagtagactggagcctgaagattttgcagtgtatttttgtcaacacta |
| | | tggtgactcacctccgtacactt |
| SEQ ID NO | | 33BU7P1D11 |
| 1760. | CDR-H1 | GFDFGDDG |
| 1761. | CDR-H2 | INWNGNKR |
| 1762. | CDR-H3 | ARVNGRWLQLEN |
| 1763. | CDR-L1 | QRIDSY |
| 1764. | CDR-L2 | AAS |
| 1765. | CDR-L3 | QQSYSTPRT |
| 1766. | VH | ESGGGVVRPGESLRLSCEVSGFDFGDDGMTWVRQGPGKG |
| | | LEWVSGINWNGNKRGYADSVKGRFTISRDNTKNSLYLQMT |
| | | SLRAEDTAFYYCARVNGRWLQLENWGQGILVTVSS |
| 1767. | VL | ASIGDRVTITCQASQRIDSYLNWYQQKPGKAPKLLIYAASRL |
| | | QSGVPSRFSGRESGTDFTLTISSLQSEDFATYYCQQSYSTP |
| | | RTFGQXT |
| 1768. | FR-H1 | ESGGGVVRPGESLRLSCEVS |
| 1769. | FR-H2 | MTWVRQGPGKGLEWVSG |
| 1770. | FR-H3 | GYADSVKGRFTISRDNTKNSLYLQMTSLRAEDTAFYYC |
| 1771. | FR-H4 | WGQGILVTVSS |
| 1772. | FR-L1 | ASIGDRVTITCQAS |
| 1773. | FR-L2 | LNWYQQKPGKAPKLLIY |
| 1774. | FR-L3 | RLQSGVPSRFSGRESGTDFTLTISSLQSEDFATYYC |
| 1775. | FR-L4 | FGQXT |
| 1776. | heavychain | ESGGGVVRPGESLRLSCEVSGFDFGDDGMTWVRQGPGKG |
| | | LEWVSGINWNGNKRGYADSVKGRFTISRDNTKNSLYLQMT |
| | | SLRAEDTAFYYCARVNGRWLQLENWGQGILVTVSSKGPSV |
| | | FPLAPSSKSTSGGTAALGXLVKDYFPEPVTVSWNSGALTSG |
| | | VHTFPAVLQSSGLYSLSSVVTVPSSSLGTQTYICNVNHKPSN |
| | | TKVDKXVEPKSCDKTHTCPPCPAPELLGGPSVFLFPPKPKD |
| | | TLMISRTPEVTC |
| 1777. | lightchain | ASIGDRVTITCQASQRIDSYLNWYQQKPGKAPKLLIYAASRL |
| | | QSGVPSRFSGRESGTDFTLTISSLQSEDFATYYCQQSYSTP |
| | | RTFGQXTAPSVFIFPPSDEQLKSGTASVVCLLNNFYPREAKV |
| | | QWKVDNALQSGNSQESVTEQDSKDSTYSLSSTLTLSKADY |
| | | EKHKVYACEVTHQGLSSPVTKSFNRGEC*K |
| 1778. | VH_nuc | gagtctgggggaggtgtggtacggcctggggagtccctgagactgtcctgtgaagtc |
| | | tctgggtttgactttggtgatgatggcatgacctgggtccgccaaggtccagggaagg |
| | | ggctggaatgggtctctggaattaattggaatggaaataagagaggttatgcagact |
| | | ctgtgaagggccgattcaccatctctagagacaacaccaagaactccctctatctac |
| | | aaatgaccagtctcagagccgaggac |
| 1779. | VL_nuc | gcatctataggagacagagtcaccatcacttgccaggcaagtcagaggattgacag |
| | | ttatttaaattggtatcagcagaaaccagggaaagcccctaagctcctgatctatgctg |
| | | catcccgcttgcaaagtggggtcccatcgaggttcagtggccgtgaatctgggacag |
| | | atttcactctcaccatcagcagtctccaatctgaagattttgcaacttactactgtcaaca |
| | | gagttacagtacccctcgaacg |
| SEQ ID NO | | 89BU7P1A12 |
| 1780. | CDR-H1 | GGSISRYY |
| 1781. | CDR-H2 | IYYSDTP |
| 1782. | CDR-H3 | ARHNEPYGGNSDDYFDL |
| 1783. | CDR-L1 | QTISDY |
| 1784. | CDR-L2 | GAS |
| 1785. | CDR-L3 | QQSYSIPLT |
| 1786. | VH | QESGPGLVKPSETLSLTCSVSGGSISRYYWSWIRQPPGQG |
| | | LEWIAGIYYSDTPKYNPSLESRVTLSVDTSKNQFSLKLTSMT |
| | | AADTAVYYCARHNEPYGGNSDDYFDLWGQGTLVTVSS |
| 1787. | VL | QSPSSLSAFVGDRVTITCRASQTISDYLNWYQHKPGKGPILL |
| | | IYGASRLESGVPSRFTGSGSGTDFTLTIDSLEAEDFATYYCQ |
| | | QSYSIPLTFGGGTKVEMK |
| 1788. | FR-H1 | QESGPGLVKPSETLSLTCSVS |
| 1789. | FR-H2 | WSWIRQPPGQGLEWIAG |
| 1790. | FR-H3 | KYNPSLESRVTLSVDTSKNQFSLKLTSMTAADTAVYYC |
| 1791. | FR-H4 | WGQGTLVTVSS |
| 1792. | FR-L1 | QSPSSLSAFVGDRVTITCRAS |
| 1793. | FR-L2 | LNWYQHKPGKGPILLIY |
| 1794. | FR-L3 | RLESGVPSRFTGSGSGTDFTLTIDSLEAEDFATYYC |
| 1795. | FR-L4 | FGGGTKVEMK |
| 1796. | heavychain | QESGPGLVKPSETLSLTCSVSGGSISRYYWSWIRQPPGQG |
| | | LEWIAGIYYSDTPKYNPSLESRVTLSVDTSKNQFSLKLTSMT |
| | | AADTAVYYCARHNEPYGGNSDDYFDLWGQGTLVTVSSKGP |
| | | SVFPLAPSSKSTSGGTAALGXLVKDYFPEPVTVSWNSGALT |
| | | SGVHTFPAVLQSSGLYSLSSVVTVPSSSLGTQTYICNVNHK |
| | | PSNTKVDKXVEPKSCDKTHTCPPCPAPELLGGPSVFLFPPK |
| | | PKDTLMISRT |
| 1797. | lightchain | QSPSSLSAFVGDRVTITCRASQTISDYLNWYQHKPGKGPILL |
| | | IYGASRLESGVPSRFTGSGSGTDFTLTIDSLEAEDFATYYCQ |
| | | QSYSIPLTFGGGTKVEMKAPSVFIFPPSDEQLKSGTASVVCL |
| | | LNNFYPREAKVQWKVDNALQSGNSQESVTEQDSKDSTYSL |
| | | SSTLTLSKADYEKHKVYACEVTHQGLSSPVTKSFNRGEC*K |
| 1798. | VH_nuc | caggagtcgggcccaggactggtgaagccctcggaaaccctgtccctcacctgca |
| | | gtgtctctggtggctccatcagtcgttactactggagctggatccggcagcccccagg |
| | | gcaggggctggagtggattgccggtatctattacagtgacacccccaagtacaacc |
| | | cctccctcgagagtcgagtcaccctatcagtagacacgtccaagaaccagttttccct |
| | | gaagctgacctctatgaccgccgcagac |
| 1799. | VL_nuc | ccagtctccatcctccctgtctgcatttgtaggagacagagtcaccattacttgccggg |
| | | caagtcagaccataagcgactatttaaattggtatcaacataaaccaggaaaaggc |
| | | cccatcctcctcatctatggcgcatcccgtttggaaagtggggtcccatcaaggttcac |
| | | tggcagcggatctgggacagatttcactctcaccatcgacagtctggaagctgaaga |
| | | ttttgcaacttattactgtcaaca |
| SEQ ID NO | | 89BU7P1B10 |
| 1800. | CDR-H1 | GGSIGRHY |
| 1801. | CDR-H2 | IYDSGST |
| 1802. | CDR-H3 | ARHNAPYGGNSDDYFEF |
| 1803. | CDR-L1 | ETISDY |
| 1804. | CDR-L2 | AAS |
| 1805. | CDR-L3 | QQSYSVPLT |
| 1806. | VH | QESGPGLVKPSETLSLTCTVSGGSIGRHYWSWIRQPPGKG |
| | | LEWIAYIYDSGSTKYNPSLESRVTISEDTSQNQFSLKLTSVTA |
| | | ADTAIYYCARHNAPYGGNSDDYFEFWGPGTLVTVSS |
| 1807. | VL | TQSPSSLSASVGDRVTITCRANETISDYLNWYQGKPGTAPK |
| | | PLIYAASSLQSGVPSRFSGSGSETYFTLTISSLOPEDFATYY |
| | | CQQSYSVPLTFGGGTKVESK |
| 1808. | FR-H1 | QESGPGLVKPSETLSLTCTVS |
| 1809. | FR-H2 | WSWIRQPPGKGLEWIAY |
| 1810. | FR-H3 | KYNPSLESRVTISEDTSQNQFSLKLTSVTAADTAIYYC |
| 1811. | FR-H4 | WGPGTLVTVSS |
| 1812. | FR-L1 | TQSPSSLSASVGDRVTITCRAN |
| 1813. | FR-L2 | LNWYQGKPGTAPKPLIY |
| 1814. | FR-L3 | SLQSGVPSRFSGSGSETYFTLTISSLOPEDFATYYC |
| 1815. | FR-L4 | FGGGTKVESK |
| 1816. | heavychain | QESGPGLVKPSETLSLTCTVSGGSIGRHYWSWIRQPPGKG |
| | | LEWIAYIYDSGSTKYNPSLESRVTISEDTSQNQFSLKLTSVTA |
| | | ADTAIYYCARHNAPYGGNSDDYFEFWGPGTLVTVSSKGPS |
| | | VFPLAPSSKSTSGGTAALGXLVKDYFPEPVTVSWNSGALTS |
| | | GVHTFPAVLQSSGLYSLSSVVTVPSSSLGTQTYICNVNHKP |
| | | SNTKVDKXVEPKSCDKTHTCPPCPAPELLGGPSVFLFPPKP |
| | | KDTLMISRT |
| 1817. | lightchain | TQSPSSLSASVGDRVTITCRANETISDYLNWYQGKPGTAPK |
| | | PLIYAASSLQSGVPSRFSGSGSETYFTLTISSLOPEDFATYY |
| | | CQQSYSVPLTFGGGTKVESKAPSVFIFPPSDEQLKSGTASV |
| | | VCLLNNFYPREAKVQWKVDNALQSGNSQESVTEQDSKDST |
| | | YSLSSTLTLSKADYEKHKVYACEVTHQGLSSPVTKSFNRGE |
| | | C*K |
| 1818. | VH_nuc | caggagtcgggcccaggcctggtgaagccttcggagaccctgtccctcacctgcac |
| | | tgtctctggtggctccatcggtcgtcactactggagctggatccggcagcccccaggg |
| | | aaggggctggagtggattgcatatatctatgacagtgggagcaccaagtacaaccc |
| | | ctccctcgagagtcgagtcaccatttcagaagacacgtcccagaaccagttctccct |
| | | gaagctgacctctgtgaccgccgcagac |
| 1819. | VL_nuc | gacccagtctccatcctccctgtctgcatctgtaggagacagagtcaccatcacttgcc |
| | | gggcaaatgagaccattagcgactatttaaattggtatcagggcaaaccagggaca |
| | | gcccctaaacccctgatctatgctgcatccagtttgcaaagtggggtcccatcaaggtt |
| | | cagtggcagtggatctgagacatatttcactctcaccatcagcagtctgcaacctgag |
| | | gattttgcaacttactactgtca |
| SEQ ID NO | | 93BU7P1B12 |
| 1820. | CDR-H1 | GYPFTRFD |
| 1821. | CDR-H2 | MNPKSGNT |
| 1822. | CDR-H3 | AKGVESSF |
| 1823. | CDR-L1 | QSLLDTSNNKNY |
| 1824. | CDR-L2 | WAS |
| 1825. | CDR-L3 | QQYYETPYI |
| 1826. | VH | VQSGAEVKKPGASVKVSCKASGYPFTRFDINWVRQAPGQG |
| | | LEWVGWMNPKSGNTGHALKFQGRVAMTRNTSISTAYMELN |
| | | SLTSEDTATYFCAKGVESSFWGPGTTVIVSS |
| 1827. | VL | GERATINCKSSQSLLDTSNNKNYLGWYQQKRGQPPKLLIYW |
| | | ASNRESGVPDRFSGSGSGTEFTLTINSLQAEDVAVYYCQQY |
| | | YETPYIXG |
| 1828. | FR-H1 | VQSGAEVKKPGASVKVSCKAS |
| 1829. | FR-H2 | INWVRQAPGQGLEWVGW |
| 1830. | FR-H3 | GHALKFQGRVAMTRNTSISTAYMELNSLTSEDTATYFC |
| 1831. | FR-H4 | WGPGTTVIVSS |
| 1832. | FR-L1 | GERATINCKSS |
| 1833. | FR-L2 | LGWYQQKRGQPPKLLIY |
| 1834. | FR-L3 | NRESGVPDRFSGSGSGTEFTLTINSLQAEDVAVYYC |
| 1835. | FR-L4 | XG |
| 1836. | heavychain | VQSGAEVKKPGASVKVSCKASGYPFTRFDINWVRQAPGQG |
| | | LEWVGWMNPKSGNTGHALKFQGRVAMTRNTSISTAYMELN |
| | | SLTSEDTATYFCAKGVESSFWGPGTTVIVSSKGPSVFPLAP |
| | | SSKSTSGGTAALGXLVKDYFPEPVTVSWNSGALTSGVHTFP |
| | | AVLQSSGLYSLSSVVTVPSSSLGTQTYICNVNHKPSNTKVD |
| | | KXVEPKSCDKTHTCPPCPAPELLGGPSVFLFPPKPKDTLMIS |
| | | RTPEVTCVVV |
| 1837. | lightchain | GERATINCKSSQSLLDTSNNKNYLGWYQQKRGQPPKLLIYW |
| | | ASNRESGVPDRFSGSGSGTEFTLTINSLQAEDVAVYYCQQY |
| | | YETPYIXGAPSVFIFPPSDEQLKSGTASVVCLLNNFYPREAK |
| | | VQWKVDNALQSGNSQESVTEQDSKDSTYSLSSTLTLSKAD |
| | | YEKHKVYACEVTHQGLSSPVTKSFNRGEC*K |
| 1838. | VH_nuc | gtgcaatctggggctgaggtgaagaagcctggggcctcagtgaaggtctcctgcaa |
| | | ggcttctggataccccttcaccagatttgatataaactgggtgcgacaggcccctgga |
| | | caaggacttgagtgggtggggtggatgaatcctaagagtggcaatacaggccatgc |
| | | actgaagttccagggcagagtcgccatgaccaggaacacctccataagcacagcc |
| | | tacatggagctgaacagcctgacatctgaa |
| 1839. | VL_nuc | gggcgagagggccactatcaactgcaagtccagtcagagtcttttggacacctcca |
| | | acaataagaattacttaggttggtaccagcagaaaaggggacagcctcctaaactg |
| | | ctcatttactgggcttccaaccgggaatccggggtccctgaccgattcagtggcagcg |
| | | ggtctgggacagagttcactctcaccatcaacagcctgcaggctgaagatgtggca |
| | | gtttattactgtcagcagtattatgagac |
| SEQ ID NO | | 13FU1P1B4 |
| 1840. | CDR-H1 | GGSISSYY |
| 1841. | CDR-H2 | IFTSGST |
| 1842. | CDR-H3 | VRDRRGLLYSNIWYWSFDL |
| 1843. | CDR-L1 | QSITNY |
| 1844. | CDR-L2 | AAS |
| 1845. | CDR-L3 | QQSYSTPWT |
| 1846. | VH | QVQLXXXGPGXVKPSETLSLTCSVSGGSISSYYWGWIRQPP |
| | | GKGLEWIGRIFTSGSTNYNPSLESRVTMSVDMSKNQFSLSL |
| | | SSVTAADTAVYYCVRDRRGLLYSNIWYWSFDLWGRGTLVT |
| | | VSS |
| 1847. | VL | QSPSSLSASAGDRVTITCRASQSITNYLNWYQQKPMRAPKL |
| | | LIYAASTLQSGVPSRFSGSGSGTDFTLTISSLOPEDFATYYC |
| | | QQSYSTPWTFGQGTKVEIR |
| 1848. | FR-H1 | QVQLXXXGPGXVKPSETLSLTCSVS |
| 1849. | FR-H2 | WGWIRQPPGKGLEWIGR |
| 1850. | FR-H3 | NYNPSLESRVTMSVDMSKNQFSLSLSSVTAADTAVYYC |
| 1851. | FR-H4 | WGRGTLVTVSS |
| 1852. | FR-L1 | QSPSSLSASAGDRVTITCRAS |
| 1853. | FR-L2 | LNWYQQKPMRAPKLLIY |
| 1854. | FR-L3 | TLQSGVPSRFSGSGSGTDFTLTISSLOPEDFATYYC |
| 1855. | FR-L4 | FGQGTKVEIR |
| 1856. | heavychain | QVQLXXXGPGXVKPSETLSLTCSVSGGSISSYYWGWIRQPP |
| | | GKGLEWIGRIFTSGSTNYNPSLESRVTMSVDMSKNQFSLSL |
| | | SSVTAADTAVYYCVRDRRGLLYSNIWYWSFDLWGRGTLVT |
| | | VSSKGPSVFPLAPSSKSTSGGTAALGXLVKDYFPEPVTVSW |
| | | NSGALTSGVHTFPAVLQSSGLYSLSSVVTVPSSSLGTQTYIC |
| | | NVNHKPSNTKVDKXVEPKSCDKTHTCPPCPAPELLGGPSVF |
| | | LFPPKPKDT |
| 1857. | lightchain | QSPSSLSASAGDRVTITCRASQSITNYLNWYQQKPMRAPKL |
| | | LIYAASTLQSGVPSRFSGSGSGTDFTLTISSLOPEDFATYYC |
| | | QQSYSTPWTFGQGTKVEIRAPSVFIFPPSDEQLKSGTASVV |
| | | CLLNNFYPREAKVQWKVDNALQSGNSQESVTEQDSKDSTY |
| | | SLSSTLTLSKADYEKHKVYACEVTHQGLSSPVTKSFNRGEC |
| | | *K |
| 1858. | VH_nuc | caggtgcagctgcannnntngggcccaggantggtgaagccttcggagaccctgt |
| | | ccctcacctgcagtgtctctggtggctccatcagtagttactactggggctggatccgg |
| | | cagccccccgggaagggactggagtggattgggcgtatttttaccagtgggagcac |
| | | caactataacccctccctcgagagtcgcgtcaccatgtcagtagacatgtccaagaa |
| | | ccagttctccctgagcctgagctctgtg |
| 1859. | VL_nuc | ccagtctccatcctccctgtctgcatctgcaggagacagagtcaccatcacttgccgg |
| | | gcaagtcagagcattactaactatttaaattggtatcaacagaaaccaatgagagcc |
| | | cctaagctcctgatctatgctgcatccactttgcaaagtggggtcccatcaaggttcagt |
| | | ggcagtggctctgggacagatttcactctcaccatcagcagtctgcaacctgaagatt |
| | | ttgcaacttactactgtcaaca |
| SEQ ID NO | | 15FU1P3A6 |
| 1860. | CDR-H1 | GVYFSDWA |
| 1861. | CDR-H2 | ISGRGANI |
| 1862. | CDR-H3 | AKTPLLTRAFDV |
| 1863. | CDR-L1 | QSVNTW |
| 1864. | CDR-L2 | KVS |
| 1865. | CDR-L3 | QQYNIDSRYS |
| 1866. | VH | LVESGGGSVPPGGSLRLSCVASGVYFSDWAMNWVRQAPG |
| | | KGLEWISSISGRGANIYYAESVRGRFTTSRDNSQNTVFLDLT |
| | | DLTVEDTALYFCAKTPLLTRAFDVWGQGTAVTVSA |
| 1867. | VL | DIVMTQSPSTLSASVGDRVTITCRASQSVNTWLAWYQQKP |
| | | GKAPRLLIYKVSTLESGVPSRFSGSGSGTEFTLTISSLQPDD |
| | | FATYYCQQYNIDSRYSFGPGTKVEIK |
| 1868. | FR-H1 | LVESGGGSVPPGGSLRLSCVAS |
| 1869. | FR-H2 | MNWVRQAPGKGLEWISS |
| 1870. | FR-H3 | YYAESVRGRFTTSRDNSQNTVFLDLTDLTVEDTALYFC |
| 1871. | FR-H4 | WGQGTAVTVSA |
| 1872. | FR-L1 | DIVMTQSPSTLSASVGDRVTITCRAS |
| 1873. | FR-L2 | LAWYQQKPGKAPRLLIY |
| 1874. | FR-L3 | TLESGVPSRFSGSGSGTEFTLTISSLQPDDFATYYC |
| 1875. | FR-L4 | FGPGTKVEIK |
| 1876. | heavychain | LVESGGGSVPPGGSLRLSCVASGVYFSDWAMNWVRQAPG |
| | | KGLEWISSISGRGANIYYAESVRGRFTTSRDNSQNTVFLDLT |
| | | DLTVEDTALYFCAKTPLLTRAFDVWGQGTAVTVSAKGPSVF |
| | | PLAPSSKSTSGGTAALGXLVKDYFPEPVTVSWNSGALTSGV |
| | | HTFPAVLQSSGLYSLSSVVTVPSSSLGTQTYICNVNHKPSNT |
| | | KVDKXVEPKSCDKTHTCPPCPAPELLGGPSVFLFPPKPKDT |
| | | LMISRTPEV |
| 1877. | lightchain | DIVMTQSPSTLSASVGDRVTITCRASQSVNTWLAWYQQKP |
| | | GKAPRLLIYKVSTLESGVPSRFSGSGSGTEFTLTISSLQPDD |
| | | FATYYCQQYNIDSRYSFGPGTKVEIKAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKS |
| | | FNRGEC*K |
| 1878. | VH_nuc | ctggtggagtctgggggaggctcggttccgccgggggggtctttaagactctcctgtgt |
| | | ggcctctggagtttactttagcgactgggccatgaattgggtccgccaggctccaggg |
| | | aaggggctggagtggatctcaagtatcagcggccgcggcgctaacatatactacgc |
| | | cgagtccgtgaggggccgcttcaccacatccagagacaactcccagaacaccgtg |
| | | tttctggatttgaccgacctgacagtc |
| 1879. | VL_nuc | gacatcgtgatgacccagtctccttccaccctgtctgcatctgtcggagacagagtca |
| | | ccatcacctgccgggccagtcagtctgttaatacgtggttggcctggtatcagcagaa |
| | | accagggaaagcccctaggctcctgatctataaggtgtctactttagaaagcggagt |
| | | cccatcaaggttcagcggcagtgggtctgggacagagttcactctcaccatcagcag |
| | | cctgcagcctgatgattttgcaact |

| SEQ ID NO | Synthetic Peptides |
|---|---|
| 1880. | DPYS |
| 1881. | DPYSZS |
| 1882. | QSQLER |
| 1883. | KRELRNL |
| 1884. | KRELRNLPQ |
| 1885. | RQQEQQ |
| 1886. | QGRQQEQQF |
| 1887. | CEALQQ |

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

### P3:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 1);
(b) an CDR-H2 comprising the amino acid sequence of (SEQ ID NO: 2);
(c) an CDR-H3 comprising the amino acid sequence of (SEQ ID NO: 3);
(d) an CDR-L1 comprising the amino acid sequence of (SEQ ID NO: 4);
(e) an CDR-L2 comprising the amino acid sequence of (SEQ ID NO: 5); and
(f) an CDR-L3 comprising the amino acid sequence of (SEQ ID NO: 6).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 7;
(b) a light chain variable (VL) domain comprising an amino acid sequence having at least 90%, at least 95%, or at least 99% identity to the amino acid sequence of SEQ ID NO: 8; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 9);
(b) an FR-H2 comprising the amino acid sequence of (SEQ ID NO: 10);
(c) an FR-H3 comprising the amino acid sequence of (SEQ ID NO: 11); and
(d) an FR-H4 comprising the amino acid sequence of (SEQ ID NO: 12).
4. The antibody of any one of paragraphs 1-3, wherein the VH domain comprises the amino acid sequence of SEQ ID NO: 7.
5. The antibody of any one of paragraphs 1-4, further comprising the following VL domain FRs:
(a) an FR-L1 comprising the amino acid sequence of (SEQ ID NO: 13);
(b) an FR-L2 comprising the amino acid sequence of (SEQ ID NO: 14);
(c) an FR-L3 comprising the amino acid sequence of (SEQ ID NO: 15); and
(d) an FR-L4 comprising the amino acid sequence of (SEQ ID NO: 16).
6. The antibody of any one of paragraphs 1-5, wherein the VL domain comprises the amino acid sequence of SEQ ID NO: 8.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 7 and
(b) a VL domain comprising an amino acid sequence having at least 90%, at least 95%, or at least 99% sequence identity to the amino acid sequence of SEQ ID NO: 8.
8. The antibody of paragraph 7, wherein the antibody comprises a VH domain comprising the amino acid sequence of SEQ ID NO: 7 and a VL domain comprising the amino acid sequence of SEQ ID NO: 8.
9. The antibody of any one of paragraphs 1-8, wherein the antibody comprises (a) a heavy chain comprising the amino acid sequence of SEQ ID NO: 17 and (b) a light chain comprising the amino acid sequence of SEQ ID NO: 18.
10. The antibody of any one of paragraphs 1-9, wherein the antibody binds Ara h 2 with a K_{D} of about 50 nM or less.
11. The method of paragraph 10, wherein the K_{D} is measured by a surface plasmon resonance assay.
12. The antibody of paragraph 11, wherein the antibody binds Ara h 2 with a K_{D} of between about 0.1 pM and about 40 nM.
13. The antibody of paragraph 12, wherein the antibody binds Ara h 2 with a K_{D} of between about 120 pM and about 20 nM.
14. The antibody of paragraph 13, wherein the antibody binds Ara h 2 with a K_{D} of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)₂ fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
   (a) providing a sample from a subject;
   (b) contacting the sample with a mixture comprising:
      (i) the antibody of any one of paragraphs 1-30 and
      (ii) Ara h 2, or a fragment thereof; and
   (c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### P6:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 21);
(b) an CDR-H2 comprising the amino acid sequence of (SEQ ID NO: 22);
(c) an CDR-H3 comprising the amino acid sequence of (SEQ ID NO: 23);
(d) an CDR-L1 comprising the amino acid sequence of (SEQ ID NO: 24);
(e) an CDR-L2 comprising the amino acid sequence of (SEQ ID NO: 25); and
(f) an CDR-L3 comprising the amino acid sequence of (SEQ ID NO: 26).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 27;
(b) a light chain variable (VL) domain comprising an amino acid sequence having at least 90%, at least 95%, or at least 99% identity to the amino acid sequence of SEQ ID NO: 28; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 29);
(b) an FR-H2 comprising the amino acid sequence of (SEQ ID NO: 30);
(c) an FR-H3 comprising the amino acid sequence of (SEQ ID NO: 31); and
(d) an FR-H4 comprising the amino acid sequence of (SEQ ID NO: 32).
4. The antibody of any one of paragraphs 1-3, wherein the VH domain comprises the amino acid sequence of SEQ ID NO: 27.
5. The antibody of any one of paragraphs 1-4, further comprising the following VL domain FRs:
(a) an FR-L1 comprising the amino acid sequence of (SEQ ID NO: 33);
(b) an FR-L2 comprising the amino acid sequence of (SEQ ID NO: 34);
(c) an FR-L3 comprising the amino acid sequence of (SEQ ID NO: 35); and
(d) an FR-L4 comprising the amino acid sequence of (SEQ ID NO: 36).
6. The antibody of any one of paragraphs 1-5, wherein the VL domain comprises the amino acid sequence of SEQ ID NO: 28.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 27 and
(b) a VL domain comprising an amino acid sequence having at least 90%, at least 95%, or at least 99% sequence identity to the amino acid sequence of SEQ ID NO: 28.
8. The antibody of paragraph 7, wherein the antibody comprises a VH domain comprising the amino acid sequence of SEQ ID NO: 27 and a VL domain comprising the amino acid sequence of SEQ ID NO: 28.
9. The antibody of any one of paragraphs 1-8, wherein the antibody comprises (a) a heavy chain comprising the amino acid sequence of SEQ ID NO: 37 and (b) a light chain comprising the amino acid sequence of SEQ ID NO: 38.
10. The antibody of any one of paragraphs 1-9, wherein the antibody binds Ara h 2 with a KD of about 50 nM or less.
11. The method of paragraph 10, wherein the KD is measured by a surface plasmon resonance assay.
12. The antibody of paragraph 11, wherein the antibody binds Ara h 2 with a KD of between about 0.1 pM and about 40 nM.
13. The antibody of paragraph 12, wherein the antibody binds Ara h 2 with a KD of between about 120 pM and about 20 nM.
14. The antibody of paragraph 13, wherein the antibody binds Ara h 2 with a KD of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)2 fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### P7:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 41);
(b) an CDR-H2 comprising the amino acid sequence of (SEQ ID NO: 42);
(c) an CDR-H3 comprising the amino acid sequence of (SEQ ID NO: 43);
(d) an CDR-L1 comprising the amino acid sequence of (SEQ ID NO: 44);
(e) an CDR-L2 comprising the amino acid sequence of (SEQ ID NO: 45); and
(f) an CDR-L3 comprising the amino acid sequence of (SEQ ID NO: 46).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 47;
(b) a light chain variable (VL) domain comprising an amino acid sequence having at least 90%, at least 95%, or at least 99% identity to the amino acid sequence of SEQ ID NO: 48; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 49);
(b) an FR-H2 comprising the amino acid sequence of (SEQ ID NO: 50);
(c) an FR-H3 comprising the amino acid sequence of (SEQ ID NO: 51); and
(d) an FR-H4 comprising the amino acid sequence of (SEQ ID NO: 52).
4. The antibody of any one of paragraphs 1-3, wherein the VH domain comprises the amino acid sequence of SEQ ID NO: 47.
5. The antibody of any one of paragraphs 1-4, further comprising the following VL domain FRs:
(a) an FR-L1 comprising the amino acid sequence of (SEQ ID NO: 53);
(b) an FR-L2 comprising the amino acid sequence of (SEQ ID NO: 54);
(c) an FR-L3 comprising the amino acid sequence of (SEQ ID NO: 55); and
(d) an FR-L4 comprising the amino acid sequence of (SEQ ID NO: 56).
6. The antibody of any one of paragraphs 1-5, wherein the VL domain comprises the amino acid sequence of SEQ ID NO: 48.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 47 and
(b) a VL domain comprising an amino acid sequence having at least 90%, at least 95%, or at least 99% sequence identity to the amino acid sequence of SEQ ID NO: 48.
8. The antibody of paragraph 7, wherein the antibody comprises a VH domain comprising the amino acid sequence of SEQ ID NO: 47 and a VL domain comprising the amino acid sequence of SEQ ID NO: 48.
9. The antibody of any one of paragraphs 1-8, wherein the antibody comprises (a) a heavy chain comprising the amino acid sequence of SEQ ID NO: 57 and (b) a light chain comprising the amino acid sequence of SEQ ID NO: 58.
10. The antibody of any one of paragraphs 1-9, wherein the antibody binds Ara h 2 with a KD of about 50 nM or less.
11. The method of paragraph 10, wherein the KD is measured by a surface plasmon resonance assay.
12. The antibody of paragraph 11, wherein the antibody binds Ara h 2 with a KD of between about 0.1 pM and about 40 nM.
13. The antibody of paragraph 12, wherein the antibody binds Ara h 2 with a KD of between about 120 pM and about 20 nM.
14. The antibody of paragraph 13, wherein the antibody binds Ara h 2 with a KD of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)2 fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### P8:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 61);
(b) an CDR-H2 comprising the amino acid sequence of (SEQ ID NO: 62);
(c) an CDR-H3 comprising the amino acid sequence of (SEQ ID NO: 63);
(d) an CDR-L1 comprising the amino acid sequence of (SEQ ID NO: 64);
(e) an CDR-L2 comprising the amino acid sequence of (SEQ ID NO: 65); and
(f) an CDR-L3 comprising the amino acid sequence of (SEQ ID NO: 66).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 67;
(b) a light chain variable (VL) domain comprising an amino acid sequence having at least 90%, at least 95%, or at least 99% identity to the amino acid sequence of SEQ ID NO: 68; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 69);
(b) an FR-H2 comprising the amino acid sequence of (SEQ ID NO: 70);
(c) an FR-H3 comprising the amino acid sequence of (SEQ ID NO: 71); and
(d) an FR-H4 comprising the amino acid sequence of (SEQ ID NO: 72).
4. The antibody of any one of paragraphs 1-3, wherein the VH domain comprises the amino acid sequence of SEQ ID NO: 67.
5. The antibody of any one of paragraphs 1-4, further comprising the following VL domain FRs:
(a) an FR-L1 comprising the amino acid sequence of (SEQ ID NO: 73);
(b) an FR-L2 comprising the amino acid sequence of (SEQ ID NO: 74);
(c) an FR-L3 comprising the amino acid sequence of (SEQ ID NO: 75); and
(d) an FR-L4 comprising the amino acid sequence of (SEQ ID NO: 76).
6. The antibody of any one of paragraphs 1-5, wherein the VL domain comprises the amino acid sequence of SEQ ID NO: 68.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 67 and
(b) a VL domain comprising an amino acid sequence having at least 90%, at least 95%, or at least 99% sequence identity to the amino acid sequence of SEQ ID NO: 68.
8. The antibody of paragraph 7, wherein the antibody comprises a VH domain comprising the amino acid sequence of SEQ ID NO: 67 and a VL domain comprising the amino acid sequence of SEQ ID NO: 68.
9. The antibody of any one of paragraphs 1-8, wherein the antibody comprises (a) a heavy chain comprising the amino acid sequence of SEQ ID NO: 77 and (b) a light chain comprising the amino acid sequence of SEQ ID NO: 78.
10. The antibody of any one of paragraphs 1-9, wherein the antibody binds Ara h 2 with a KD of about 50 nM or less.
11. The method of paragraph 10, wherein the KD is measured by a surface plasmon resonance assay.
12. The antibody of paragraph 11, wherein the antibody binds Ara h 2 with a KD of between about 0.1 pM and about 40 nM.
13. The antibody of paragraph 12, wherein the antibody binds Ara h 2 with a KD of between about 120 pM and about 20 nM.
14. The antibody of paragraph 13, wherein the antibody binds Ara h 2 with a KD of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)2 fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### P10:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 81);
(b) an CDR-H2 comprising the amino acid sequence of (SEQ ID NO: 82);
(c) an CDR-H3 comprising the amino acid sequence of (SEQ ID NO: 83);
(d) an CDR-L1 comprising the amino acid sequence of (SEQ ID NO: 84);
(e) an CDR-L2 comprising the amino acid sequence of (SEQ ID NO: 85); and
(f) an CDR-L3 comprising the amino acid sequence of (SEQ ID NO: 86).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 87;
(b) a light chain variable (VL) domain comprising an amino acid sequence having at least 90%, at least 95%, or at least 99% identity to the amino acid sequence of SEQ ID NO: 88; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 89);
(b) an FR-H2 comprising the amino acid sequence of (SEQ ID NO: 90);
(c) an FR-H3 comprising the amino acid sequence of (SEQ ID NO: 91); and
(d) an FR-H4 comprising the amino acid sequence of (SEQ ID NO:92).
4. The antibody of any one of paragraphs 1-3, wherein the VH domain comprises the amino acid sequence of SEQ ID NO: 87.
5. The antibody of any one of paragraphs 1-4, further comprising the following VL domain FRs:
(a) an FR-L1 comprising the amino acid sequence of (SEQ ID NO: 93);
(b) an FR-L2 comprising the amino acid sequence of (SEQ ID NO: 94);
(c) an FR-L3 comprising the amino acid sequence of (SEQ ID NO: 95); and
(d) an FR-L4 comprising the amino acid sequence of (SEQ ID NO: 96).
6. The antibody of any one of paragraphs 1-5, wherein the VL domain comprises the amino acid sequence of SEQ ID NO: 88.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 87 and
(b) a VL domain comprising an amino acid sequence having at least 90%, at least 95%, or at least 99% sequence identity to the amino acid sequence of SEQ ID NO: 88.
8. The antibody of paragraph 7, wherein the antibody comprises a VH domain comprising the amino acid sequence of SEQ ID NO: 87 and a VL domain comprising the amino acid sequence of SEQ ID NO: 88.
9. The antibody of any one of paragraphs 1-8, wherein the antibody comprises (a) a heavy chain comprising the amino acid sequence of SEQ ID NO: 97 and (b) a light chain comprising the amino acid sequence of SEQ ID NO: 98.
10. The antibody of any one of paragraphs 1-9, wherein the antibody binds Ara h 2 with a KD of about 50 nM or less.
11. The method of paragraph 10, wherein the KD is measured by a surface plasmon resonance assay.
12. The antibody of paragraph 11, wherein the antibody binds Ara h 2 with a KD of between about 0.1 pM and about 40 nM.
13. The antibody of paragraph 12, wherein the antibody binds Ara h 2 with a KD of between about 120 pM and about 20 nM.
14. The antibody of paragraph 13, wherein the antibody binds Ara h 2 with a KD of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)2 fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### P11:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 101);
(b) an CDR-H2 comprising the amino acid sequence of (SEQ ID NO: 102);
(c) an CDR-H3 comprising the amino acid sequence of (SEQ ID NO: 103);
(d) an CDR-L1 comprising the amino acid sequence of (SEQ ID NO: 104);
(e) an CDR-L2 comprising the amino acid sequence of (SEQ ID NO: 105); and
(f) an CDR-L3 comprising the amino acid sequence of (SEQ ID NO: 106).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 107;
(b) a light chain variable (VL) domain comprising an amino acid sequence having at least 90%, at least 95%, or at least 99% identity to the amino acid sequence of SEQ ID NO: 108; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 109);
(b) an FR-H2 comprising the amino acid sequence of (SEQ ID NO: 110);
(c) an FR-H3 comprising the amino acid sequence of (SEQ ID NO: 111); and
(d) an FR-H4 comprising the amino acid sequence of (SEQ ID NO: 112).
4. The antibody of any one of paragraphs 1-3, wherein the VH domain comprises the amino acid sequence of SEQ ID NO: 107.
5. The antibody of any one of paragraphs 1-4, further comprising the following VL domain FRs:
(a) an FR-L1 comprising the amino acid sequence of (SEQ ID NO: 113);
(b) an FR-L2 comprising the amino acid sequence of (SEQ ID NO: 114);
(c) an FR-L3 comprising the amino acid sequence of (SEQ ID NO: 115); and
(d) an FR-L4 comprising the amino acid sequence of (SEQ ID NO: 116).
6. The antibody of any one of paragraphs 1-5, wherein the VL domain comprises the amino acid sequence of SEQ ID NO: 108.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 107 and
(b) a VL domain comprising an amino acid sequence having at least 90%, at least 95%, or at least 99% sequence identity to the amino acid sequence of SEQ ID NO: 108.
8. The antibody of paragraph 7, wherein the antibody comprises a VH domain comprising the amino acid sequence of SEQ ID NO: 107 and a VL domain comprising the amino acid sequence of SEQ ID NO: 108.
9. The antibody of any one of paragraphs 1-8, wherein the antibody comprises (a) a heavy chain comprising the amino acid sequence of SEQ ID NO: 117 and (b) a light chain comprising the amino acid sequence of SEQ ID NO: 118.
10. The antibody of any one of paragraphs 1-9, wherein the antibody binds Ara h 2 with a KD of about 50 nM or less.
11. The method of paragraph 10, wherein the KD is measured by a surface plasmon resonance assay.
12. The antibody of paragraph 11, wherein the antibody binds Ara h 2 with a KD of between about 0.1 pM and about 40 nM.
13. The antibody of paragraph 12, wherein the antibody binds Ara h 2 with a KD of between about 120 pM and about 20 nM.
14. The antibody of paragraph 13, wherein the antibody binds Ara h 2 with a KD of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)2 fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### P13:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 121);
(b) an CDR-H2 comprising the amino acid sequence of (SEQ ID NO: 122);
(c) an CDR-H3 comprising the amino acid sequence of (SEQ ID NO: 123);
(d) an CDR-L1 comprising the amino acid sequence of (SEQ ID NO: 124);
(e) an CDR-L2 comprising the amino acid sequence of (SEQ ID NO: 125); and
(f) an CDR-L3 comprising the amino acid sequence of (SEQ ID NO: 126).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 127;
(b) a light chain variable (VL) domain comprising an amino acid sequence having at least 90%, at least 95%, or at least 99% identity to the amino acid sequence of SEQ ID NO: 128; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 129);
(b) an FR-H2 comprising the amino acid sequence of (SEQ ID NO: 130);
(c) an FR-H3 comprising the amino acid sequence of (SEQ ID NO: 131); and
(d) an FR-H4 comprising the amino acid sequence of (SEQ ID NO: 132).
4. The antibody of any one of paragraphs 1-3, wherein the VH domain comprises the amino acid sequence of SEQ ID NO: 127.
5. The antibody of any one of paragraphs 1-4, further comprising the following VL domain FRs:
(a) an FR-L1 comprising the amino acid sequence of (SEQ ID NO: 133);
(b) an FR-L2 comprising the amino acid sequence of (SEQ ID NO: 134);
(c) an FR-L3 comprising the amino acid sequence of (SEQ ID NO: 135); and
(d) an FR-L4 comprising the amino acid sequence of (SEQ ID NO: 136).
6. The antibody of any one of paragraphs 1-5, wherein the VL domain comprises the amino acid sequence of SEQ ID NO: 128.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 127 and
(b) a VL domain comprising an amino acid sequence having at least 90%, at least 95%, or at least 99% sequence identity to the amino acid sequence of SEQ ID NO: 128.
8. The antibody of paragraph 7, wherein the antibody comprises a VH domain comprising the amino acid sequence of SEQ ID NO: 127 and a VL domain comprising the amino acid sequence of SEQ ID NO: 128.
9. The antibody of any one of paragraphs 1-8, wherein the antibody comprises (a) a heavy chain comprising the amino acid sequence of SEQ ID NO: 137 and (b) a light chain comprising the amino acid sequence of SEQ ID NO: 138.
10. The antibody of any one of paragraphs 1-9, wherein the antibody binds Ara h 2 with a KD of about 50 nM or less.
11. The method of paragraph 10, wherein the KD is measured by a surface plasmon resonance assay.
12. The antibody of paragraph 11, wherein the antibody binds Ara h 2 with a KD of between about 0.1 pM and about 40 nM.
13. The antibody of paragraph 12, wherein the antibody binds Ara h 2 with a KD of between about 120 pM and about 20 nM.
14. The antibody of paragraph 13, wherein the antibody binds Ara h 2 with a KD of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)2 fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### P14:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 141);
(b) an CDR-H2 comprising the amino acid sequence of (SEQ ID NO: 142);
(c) an CDR-H3 comprising the amino acid sequence of (SEQ ID NO: 143);
(d) an CDR-L1 comprising the amino acid sequence of (SEQ ID NO: 144);
(e) an CDR-L2 comprising the amino acid sequence of (SEQ ID NO: 145); and
(f) an CDR-L3 comprising the amino acid sequence of (SEQ ID NO: 146).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 147;
(b) a light chain variable (VL) domain comprising an amino acid sequence having at least 90%, at least 95%, or at least 99% identity to the amino acid sequence of SEQ ID NO: 148; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 149);
(b) an FR-H2 comprising the amino acid sequence of (SEQ ID NO: 150);
(c) an FR-H3 comprising the amino acid sequence of (SEQ ID NO: 151); and
(d) an FR-H4 comprising the amino acid sequence of (SEQ ID NO: 152).
4. The antibody of any one of paragraphs 1-3, wherein the VH domain comprises the amino acid sequence of SEQ ID NO: 147.
5. The antibody of any one of paragraphs 1-4, further comprising the following VL domain FRs:
(a) an FR-L1 comprising the amino acid sequence of (SEQ ID NO: 153);
(b) an FR-L2 comprising the amino acid sequence of (SEQ ID NO: 154);
(c) an FR-L3 comprising the amino acid sequence of (SEQ ID NO: 155); and
(d) an FR-L4 comprising the amino acid sequence of (SEQ ID NO: 156).
6. The antibody of any one of paragraphs 1-5, wherein the VL domain comprises the amino acid sequence of SEQ ID NO: 148.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 147 and
(b) a VL domain comprising an amino acid sequence having at least 90%, at least 95%, or at least 99% sequence identity to the amino acid sequence of SEQ ID NO: 148.
8. The antibody of paragraph 7, wherein the antibody comprises a VH domain comprising the amino acid sequence of SEQ ID NO: 147 and a VL domain comprising the amino acid sequence of SEQ ID NO: 148.
9. The antibody of any one of paragraphs 1-8, wherein the antibody comprises (a) a heavy chain comprising the amino acid sequence of SEQ ID NO: 157 and (b) a light chain comprising the amino acid sequence of SEQ ID NO: 158.
10. The antibody of any one of paragraphs 1-9, wherein the antibody binds Ara h 2 with a KD of about 50 nM or less.
11. The method of paragraph 10, wherein the KD is measured by a surface plasmon resonance assay.
12. The antibody of paragraph 11, wherein the antibody binds Ara h 2 with a KD of between about 0.1 pM and about 40 nM.
13. The antibody of paragraph 12, wherein the antibody binds Ara h 2 with a KD of between about 120 pM and about 20 nM.
14. The antibody of paragraph 13, wherein the antibody binds Ara h 2 with a KD of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)2 fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### P16:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 161);
(b) an CDR-H2 comprising the amino acid sequence of (SEQ ID NO: 162);
(c) an CDR-H3 comprising the amino acid sequence of (SEQ ID NO: 163);
(d) an CDR-L1 comprising the amino acid sequence of (SEQ ID NO: 164);
(e) an CDR-L2 comprising the amino acid sequence of (SEQ ID NO: 165); and
(f) an CDR-L3 comprising the amino acid sequence of (SEQ ID NO: 166).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 167;
(b) a light chain variable (VL) domain comprising an amino acid sequence having at least 90%, at least 95%, or at least 99% identity to the amino acid sequence of SEQ ID NO: 168; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 169);
(b) an FR-H2 comprising the amino acid sequence of (SEQ ID NO: 170);
(c) an FR-H3 comprising the amino acid sequence of (SEQ ID NO: 171); and
(d) an FR-H4 comprising the amino acid sequence of (SEQ ID NO: 172).
4. The antibody of any one of paragraphs 1-3, wherein the VH domain comprises the amino acid sequence of SEQ ID NO: 167.
5. The antibody of any one of paragraphs 1-4, further comprising the following VL domain FRs:
(a) an FR-L1 comprising the amino acid sequence of (SEQ ID NO: 173);
(b) an FR-L2 comprising the amino acid sequence of (SEQ ID NO: 174);
(c) an FR-L3 comprising the amino acid sequence of (SEQ ID NO: 175); and
(d) an FR-L4 comprising the amino acid sequence of (SEQ ID NO: 176).
6. The antibody of any one of paragraphs 1-5, wherein the VL domain comprises the amino acid sequence of SEQ ID NO: 168.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 167 and
(b) a VL domain comprising an amino acid sequence having at least 90%, at least 95%, or at least 99% sequence identity to the amino acid sequence of SEQ ID NO: 168.
8. The antibody of paragraph 7, wherein the antibody comprises a VH domain comprising the amino acid sequence of SEQ ID NO: 167 and a VL domain comprising the amino acid sequence of SEQ ID NO: 168.
9. The antibody of any one of paragraphs 1-8, wherein the antibody comprises (a) a heavy chain comprising the amino acid sequence of SEQ ID NO: 177 and (b) a light chain comprising the amino acid sequence of SEQ ID NO: 178.
10. The antibody of any one of paragraphs 1-9, wherein the antibody binds Ara h 2 with a KD of about 50 nM or less.
11. The method of paragraph 10, wherein the KD is measured by a surface plasmon resonance assay.
12. The antibody of paragraph 11, wherein the antibody binds Ara h 2 with a KD of between about 0.1 pM and about 40 nM.
13. The antibody of paragraph 12, wherein the antibody binds Ara h 2 with a KD of between about 120 pM and about 20 nM.
14. The antibody of paragraph 13, wherein the antibody binds Ara h 2 with a KD of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)2 fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### P17:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 181);
(b) an CDR-H2 comprising the amino acid sequence of (SEQ ID NO: 182);
(c) an CDR-H3 comprising the amino acid sequence of (SEQ ID NO: 183);
(d) an CDR-L1 comprising the amino acid sequence of (SEQ ID NO: 184);
(e) an CDR-L2 comprising the amino acid sequence of (SEQ ID NO: 185); and
(f) an CDR-L3 comprising the amino acid sequence of (SEQ ID NO: 186).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 187;
(b) a light chain variable (VL) domain comprising an amino acid sequence having at least 90%, at least 95%, or at least 99% identity to the amino acid sequence of SEQ ID NO: 188; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 189);
(b) an FR-H2 comprising the amino acid sequence of (SEQ ID NO: 190);
(c) an FR-H3 comprising the amino acid sequence of (SEQ ID NO: 191); and
(d) an FR-H4 comprising the amino acid sequence of (SEQ ID NO: 192).
4. The antibody of any one of paragraphs 1-3, wherein the VH domain comprises the amino acid sequence of SEQ ID NO: 187.
5. The antibody of any one of paragraphs 1-4, further comprising the following VL domain FRs:
(a) an FR-L1 comprising the amino acid sequence of (SEQ ID NO: 193);
(b) an FR-L2 comprising the amino acid sequence of (SEQ ID NO: 194);
(c) an FR-L3 comprising the amino acid sequence of (SEQ ID NO: 195); and
(d) an FR-L4 comprising the amino acid sequence of (SEQ ID NO: 196).
6. The antibody of any one of paragraphs 1-5, wherein the VL domain comprises the amino acid sequence of SEQ ID NO: 188.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 187 and
(b) a VL domain comprising an amino acid sequence having at least 90%, at least 95%, or at least 99% sequence identity to the amino acid sequence of SEQ ID NO: 188.
8. The antibody of paragraph 7, wherein the antibody comprises a VH domain comprising the amino acid sequence of SEQ ID NO: 187 and a VL domain comprising the amino acid sequence of SEQ ID NO: 188.
9. The antibody of any one of paragraphs 1-8, wherein the antibody comprises (a) a heavy chain comprising the amino acid sequence of SEQ ID NO: 197 and (b) a light chain comprising the amino acid sequence of SEQ ID NO: 198.
10. The antibody of any one of paragraphs 1-9, wherein the antibody binds Ara h 2 with a KD of about 50 nM or less.
11. The method of paragraph 10, wherein the KD is measured by a surface plasmon resonance assay.
12. The antibody of paragraph 11, wherein the antibody binds Ara h 2 with a KD of between about 0.1 pM and about 40 nM.
13. The antibody of paragraph 12, wherein the antibody binds Ara h 2 with a KD of between about 120 pM and about 20 nM.
14. The antibody of paragraph 13, wherein the antibody binds Ara h 2 with a KD of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)2 fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### P19:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 201);
(b) an CDR-H2 comprising the amino acid sequence of (SEQ ID NO: 202);
(c) an CDR-H3 comprising the amino acid sequence of (SEQ ID NO: 203);
(d) an CDR-L1 comprising the amino acid sequence of (SEQ ID NO: 204);
(e) an CDR-L2 comprising the amino acid sequence of (SEQ ID NO: 205); and
(f) an CDR-L3 comprising the amino acid sequence of (SEQ ID NO: 206).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 207;
(b) a light chain variable (VL) domain comprising an amino acid sequence having at least 90%, at least 95%, or at least 99% identity to the amino acid sequence of SEQ ID NO: 208; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 209);
(b) an FR-H2 comprising the amino acid sequence of (SEQ ID NO: 210);
(c) an FR-H3 comprising the amino acid sequence of (SEQ ID NO: 211); and
(d) an FR-H4 comprising the amino acid sequence of (SEQ ID NO: 212).
4. The antibody of any one of paragraphs 1-3, wherein the VH domain comprises the amino acid sequence of SEQ ID NO: 207.
5. The antibody of any one of paragraphs 1-4, further comprising the following VL domain FRs:
(a) an FR-L1 comprising the amino acid sequence of (SEQ ID NO: 213);
(b) an FR-L2 comprising the amino acid sequence of (SEQ ID NO: 214);
(c) an FR-L3 comprising the amino acid sequence of (SEQ ID NO: 215); and
(d) an FR-L4 comprising the amino acid sequence of (SEQ ID NO: 216).
6. The antibody of any one of paragraphs 1-5, wherein the VL domain comprises the amino acid sequence of SEQ ID NO: 208.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 207 and
(b) a VL domain comprising an amino acid sequence having at least 90%, at least 95%, or at least 99% sequence identity to the amino acid sequence of SEQ ID NO: 208.
8. The antibody of paragraph 7, wherein the antibody comprises a VH domain comprising the amino acid sequence of SEQ ID NO: 207 and a VL domain comprising the amino acid sequence of SEQ ID NO: 208.
9. The antibody of any one of paragraphs 1-8, wherein the antibody comprises (a) a heavy chain comprising the amino acid sequence of SEQ ID NO: 217 and (b) a light chain comprising the amino acid sequence of SEQ ID NO: 218.
10. The antibody of any one of paragraphs 1-9, wherein the antibody binds Ara h 2 with a KD of about 50 nM or less.
11. The method of paragraph 10, wherein the KD is measured by a surface plasmon resonance assay.
12. The antibody of paragraph 11, wherein the antibody binds Ara h 2 with a KD of between about 0.1 pM and about 40 nM.
13. The antibody of paragraph 12, wherein the antibody binds Ara h 2 with a KD of between about 120 pM and about 20 nM.
14. The antibody of paragraph 13, wherein the antibody binds Ara h 2 with a KD of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)2 fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### P21:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 221);
(b) an CDR-H2 comprising the amino acid sequence of (SEQ ID NO: 222);
(c) an CDR-H3 comprising the amino acid sequence of (SEQ ID NO: 223);
(d) an CDR-L1 comprising the amino acid sequence of (SEQ ID NO: 224);
(e) an CDR-L2 comprising the amino acid sequence of (SEQ ID NO: 225); and
(f) an CDR-L3 comprising the amino acid sequence of (SEQ ID NO: 226).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 227;
(b) a light chain variable (VL) domain comprising an amino acid sequence having at least 90%, at least 95%, or at least 99% identity to the amino acid sequence of SEQ ID NO: 228; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 229);
(b) an FR-H2 comprising the amino acid sequence of (SEQ ID NO: 230);
(c) an FR-H3 comprising the amino acid sequence of (SEQ ID NO: 231); and
(d) an FR-H4 comprising the amino acid sequence of (SEQ ID NO: 232).
4. The antibody of any one of paragraphs 1-3, wherein the VH domain comprises the amino acid sequence of SEQ ID NO: 227.
5. The antibody of any one of paragraphs 1-4, further comprising the following VL domain FRs:
(a) an FR-L1 comprising the amino acid sequence of (SEQ ID NO: 233);
(b) an FR-L2 comprising the amino acid sequence of (SEQ ID NO: 234);
(c) an FR-L3 comprising the amino acid sequence of (SEQ ID NO: 235); and
(d) an FR-L4 comprising the amino acid sequence of (SEQ ID NO: 236).
6. The antibody of any one of paragraphs 1-5, wherein the VL domain comprises the amino acid sequence of SEQ ID NO: 228.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 227 and
(b) a VL domain comprising an amino acid sequence having at least 90%, at least 95%, or at least 99% sequence identity to the amino acid sequence of SEQ ID NO: 228.
8. The antibody of paragraph 7, wherein the antibody comprises a VH domain comprising the amino acid sequence of SEQ ID NO: 227 and a VL domain comprising the amino acid sequence of SEQ ID NO: 228.
9. The antibody of any one of paragraphs 1-8, wherein the antibody comprises (a) a heavy chain comprising the amino acid sequence of SEQ ID NO: 237 and (b) a light chain comprising the amino acid sequence of SEQ ID NO: 238.
10. The antibody of any one of paragraphs 1-9, wherein the antibody binds Ara h 2 with a KD of about 50 nM or less.
11. The method of paragraph 10, wherein the KD is measured by a surface plasmon resonance assay.
12. The antibody of paragraph 11, wherein the antibody binds Ara h 2 with a KD of between about 0.1 pM and about 40 nM.
13. The antibody of paragraph 12, wherein the antibody binds Ara h 2 with a KD of between about 120 pM and about 20 nM.
14. The antibody of paragraph 13, wherein the antibody binds Ara h 2 with a KD of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)2 fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### P22:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 241);
(b) an CDR-H2 comprising the amino acid sequence of (SEQ ID NO: 242);
(c) an CDR-H3 comprising the amino acid sequence of (SEQ ID NO: 243);
(d) an CDR-L1 comprising the amino acid sequence of (SEQ ID NO: 244);
(e) an CDR-L2 comprising the amino acid sequence of (SEQ ID NO: 245); and
(f) an CDR-L3 comprising the amino acid sequence of (SEQ ID NO: 246).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 247;
(b) a light chain variable (VL) domain comprising an amino acid sequence having at least 90%, at least 95%, or at least 99% identity to the amino acid sequence of SEQ ID NO: 248; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 249);
(b) an FR-H2 comprising the amino acid sequence of (SEQ ID NO: 250);
(c) an FR-H3 comprising the amino acid sequence of (SEQ ID NO: 251); and
(d) an FR-H4 comprising the amino acid sequence of (SEQ ID NO: 252).
4. The antibody of any one of paragraphs 1-3, wherein the VH domain comprises the amino acid sequence of SEQ ID NO: 247.
5. The antibody of any one of paragraphs 1-4, further comprising the following VL domain FRs:
(a) an FR-L1 comprising the amino acid sequence of (SEQ ID NO: 253);
(b) an FR-L2 comprising the amino acid sequence of (SEQ ID NO: 254);
(c) an FR-L3 comprising the amino acid sequence of (SEQ ID NO: 255); and
(d) an FR-L4 comprising the amino acid sequence of (SEQ ID NO: 256).
6. The antibody of any one of paragraphs 1-5, wherein the VL domain comprises the amino acid sequence of SEQ ID NO: 248.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 247 and
(b) a VL domain comprising an amino acid sequence having at least 90%, at least 95%, or at least 99% sequence identity to the amino acid sequence of SEQ ID NO: 248.
8. The antibody of paragraph 7, wherein the antibody comprises a VH domain comprising the amino acid sequence of SEQ ID NO: 247 and a VL domain comprising the amino acid sequence of SEQ ID NO: 248.
9. The antibody of any one of paragraphs 1-8, wherein the antibody comprises (a) a heavy chain comprising the amino acid sequence of SEQ ID NO: 257 and (b) a light chain comprising the amino acid sequence of SEQ ID NO: 258.
10. The antibody of any one of paragraphs 1-9, wherein the antibody binds Ara h 2 with a KD of about 50 nM or less.
11. The method of paragraph 10, wherein the KD is measured by a surface plasmon resonance assay.
12. The antibody of paragraph 11, wherein the antibody binds Ara h 2 with a KD of between about 0.1 pM and about 40 nM.
13. The antibody of paragraph 12, wherein the antibody binds Ara h 2 with a KD of between about 120 pM and about 20 nM.
14. The antibody of paragraph 13, wherein the antibody binds Ara h 2 with a KD of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)2 fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### P22:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 261);
(b) an CDR-H2 comprising the amino acid sequence of (SEQ ID NO: 262);
(c) an CDR-H3 comprising the amino acid sequence of (SEQ ID NO: 263);
(d) an CDR-L1 comprising the amino acid sequence of (SEQ ID NO: 264);
(e) an CDR-L2 comprising the amino acid sequence of (SEQ ID NO: 265); and
(f) an CDR-L3 comprising the amino acid sequence of (SEQ ID NO: 266).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 267;
(b) a light chain variable (VL) domain comprising an amino acid sequence having at least 90%, at least 95%, or at least 99% identity to the amino acid sequence of SEQ ID NO: 268; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 269);
(b) an FR-H2 comprising the amino acid sequence of (SEQ ID NO: 270);
(c) an FR-H3 comprising the amino acid sequence of (SEQ ID NO: 271); and
(d) an FR-H4 comprising the amino acid sequence of (SEQ ID NO: 272).
4. The antibody of any one of paragraphs 1-3, wherein the VH domain comprises the amino acid sequence of SEQ ID NO: 267.
5. The antibody of any one of paragraphs 1-4, further comprising the following VL domain FRs:
(a) an FR-L1 comprising the amino acid sequence of (SEQ ID NO: 273);
(b) an FR-L2 comprising the amino acid sequence of (SEQ ID NO: 274);
(c) an FR-L3 comprising the amino acid sequence of (SEQ ID NO: 275); and
(d) an FR-L4 comprising the amino acid sequence of (SEQ ID NO: 276).
6. The antibody of any one of paragraphs 1-5, wherein the VL domain comprises the amino acid sequence of SEQ ID NO: 268.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 267 and
(b) a VL domain comprising an amino acid sequence having at least 90%, at least 95%, or at least 99% sequence identity to the amino acid sequence of SEQ ID NO: 268.
8. The antibody of paragraph 7, wherein the antibody comprises a VH domain comprising the amino acid sequence of SEQ ID NO: 267 and a VL domain comprising the amino acid sequence of SEQ ID NO: 268.
9. The antibody of any one of paragraphs 1-8, wherein the antibody comprises (a) a heavy chain comprising the amino acid sequence of SEQ ID NO: 277 and (b) a light chain comprising the amino acid sequence of SEQ ID NO: 278.
10. The antibody of any one of paragraphs 1-9, wherein the antibody binds Ara h 2 with a KD of about 50 nM or less.
11. The method of paragraph 10, wherein the KD is measured by a surface plasmon resonance assay.
12. The antibody of paragraph 11, wherein the antibody binds Ara h 2 with a KD of between about 0.1 pM and about 40 nM.
13. The antibody of paragraph 12, wherein the antibody binds Ara h 2 with a KD of between about 120 pM and about 20 nM.
14. The antibody of paragraph 13, wherein the antibody binds Ara h 2 with a KD of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)2 fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### P30:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 281);
(b) an CDR-H2 comprising the amino acid sequence of (SEQ ID NO: 282);
(c) an CDR-H3 comprising the amino acid sequence of (SEQ ID NO: 283);
(d) an CDR-L1 comprising the amino acid sequence of (SEQ ID NO: 284);
(e) an CDR-L2 comprising the amino acid sequence of (SEQ ID NO: 285); and
(f) an CDR-L3 comprising the amino acid sequence of (SEQ ID NO: 286).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 287;
(b) a light chain variable (VL) domain comprising an amino acid sequence having at least 90%, at least 95%, or at least 99% identity to the amino acid sequence of SEQ ID NO: 288; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 289);
(b) an FR-H2 comprising the amino acid sequence of (SEQ ID NO: 290);
(c) an FR-H3 comprising the amino acid sequence of (SEQ ID NO: 291); and
(d) an FR-H4 comprising the amino acid sequence of (SEQ ID NO: 292).
4. The antibody of any one of paragraphs 1-3, wherein the VH domain comprises the amino acid sequence of SEQ ID NO: 287.
5. The antibody of any one of paragraphs 1-4, further comprising the following VL domain FRs:
(a) an FR-L1 comprising the amino acid sequence of (SEQ ID NO: 293);
(b) an FR-L2 comprising the amino acid sequence of (SEQ ID NO: 294);
(c) an FR-L3 comprising the amino acid sequence of (SEQ ID NO: 295); and
(d) an FR-L4 comprising the amino acid sequence of (SEQ ID NO: 296).
6. The antibody of any one of paragraphs 1-5, wherein the VL domain comprises the amino acid sequence of SEQ ID NO: 288.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 287 and
(b) a VL domain comprising an amino acid sequence having at least 90%, at least 95%, or at least 99% sequence identity to the amino acid sequence of SEQ ID NO: 288.
8. The antibody of paragraph 7, wherein the antibody comprises a VH domain comprising the amino acid sequence of SEQ ID NO: 287 and a VL domain comprising the amino acid sequence of SEQ ID NO: 288.
9. The antibody of any one of paragraphs 1-8, wherein the antibody comprises (a) a heavy chain comprising the amino acid sequence of SEQ ID NO: 297 and (b) a light chain comprising the amino acid sequence of SEQ ID NO: 298.
10. The antibody of any one of paragraphs 1-9, wherein the antibody binds Ara h 2 with a KD of about 50 nM or less.
11. The method of paragraph 10, wherein the KD is measured by a surface plasmon resonance assay.
12. The antibody of paragraph 11, wherein the antibody binds Ara h 2 with a KD of between about 0.1 pM and about 40 nM.
13. The antibody of paragraph 12, wherein the antibody binds Ara h 2 with a KD of between about 120 pM and about 20 nM.
14. The antibody of paragraph 13, wherein the antibody binds Ara h 2 with a KD of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)2 fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.
1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 301);
(b) an CDR-H2 comprising the amino acid sequence of (SEQ ID NO: 302);
(c) an CDR-H3 comprising the amino acid sequence of (SEQ ID NO: 303);
(d) an CDR-L1 comprising the amino acid sequence of (SEQ ID NO: 304);
(e) an CDR-L2 comprising the amino acid sequence of (SEQ ID NO: 305); and
(f) an CDR-L3 comprising the amino acid sequence of (SEQ ID NO: 306).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 307;
(b) a light chain variable (VL) domain comprising an amino acid sequence having at least 90%, at least 95%, or at least 99% identity to the amino acid sequence of SEQ ID NO: 308; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 309);
(b) an FR-H2 comprising the amino acid sequence of (SEQ ID NO: 310);
(c) an FR-H3 comprising the amino acid sequence of (SEQ ID NO: 311); and
(d) an FR-H4 comprising the amino acid sequence of (SEQ ID NO: 312).
4. The antibody of any one of paragraphs 1-3, wherein the VH domain comprises the amino acid sequence of SEQ ID NO: 307.
5. The antibody of any one of paragraphs 1-4, further comprising the following VL domain FRs:
(a) an FR-L1 comprising the amino acid sequence of (SEQ ID NO: 313);
(b) an FR-L2 comprising the amino acid sequence of (SEQ ID NO: 314);
(c) an FR-L3 comprising the amino acid sequence of (SEQ ID NO: 315); and
(d) an FR-L4 comprising the amino acid sequence of (SEQ ID NO: 316).
6. The antibody of any one of paragraphs 1-5, wherein the VL domain comprises the amino acid sequence of SEQ ID NO: 308.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 307 and
(b) a VL domain comprising an amino acid sequence having at least 90%, at least 95%, or at least 99% sequence identity to the amino acid sequence of SEQ ID NO: 308.
8. The antibody of paragraph 7, wherein the antibody comprises a VH domain comprising the amino acid sequence of SEQ ID NO: 307 and a VL domain comprising the amino acid sequence of SEQ ID NO: 308.
9. The antibody of any one of paragraphs 1-8, wherein the antibody comprises (a) a heavy chain comprising the amino acid sequence of SEQ ID NO: 317 and (b) a light chain comprising the amino acid sequence of SEQ ID NO: 318.
10. The antibody of any one of paragraphs 1-9, wherein the antibody binds Ara h 2 with a KD of about 50 nM or less.
11. The method of paragraph 10, wherein the KD is measured by a surface plasmon resonance assay.
12. The antibody of paragraph 11, wherein the antibody binds Ara h 2 with a KD of between about 0.1 pM and about 40 nM.
13. The antibody of paragraph 12, wherein the antibody binds Ara h 2 with a KD of between about 120 pM and about 20 nM.
14. The antibody of paragraph 13, wherein the antibody binds Ara h 2 with a KD of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)2 fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.
1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 321);
(b) an CDR-H2 comprising the amino acid sequence of (SEQ ID NO: 322);
(c) an CDR-H3 comprising the amino acid sequence of (SEQ ID NO: 323);
(d) an CDR-L1 comprising the amino acid sequence of (SEQ ID NO: 324);
(e) an CDR-L2 comprising the amino acid sequence of (SEQ ID NO: 325); and
(f) an CDR-L3 comprising the amino acid sequence of (SEQ ID NO: 326).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 327;
(b) a light chain variable (VL) domain comprising an amino acid sequence having at least 90%, at least 95%, or at least 99% identity to the amino acid sequence of SEQ ID NO: 328; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 329);
(b) an FR-H2 comprising the amino acid sequence of (SEQ ID NO: 330);
(c) an FR-H3 comprising the amino acid sequence of (SEQ ID NO: 331); and
(d) an FR-H4 comprising the amino acid sequence of (SEQ ID NO: 332).
4. The antibody of any one of paragraphs 1-3, wherein the VH domain comprises the amino acid sequence of SEQ ID NO: 327.
5. The antibody of any one of paragraphs 1-4, further comprising the following VL domain FRs:
(a) an FR-L1 comprising the amino acid sequence of (SEQ ID NO: 333);
(b) an FR-L2 comprising the amino acid sequence of (SEQ ID NO: 334);
(c) an FR-L3 comprising the amino acid sequence of (SEQ ID NO: 335); and
(d) an FR-L4 comprising the amino acid sequence of (SEQ ID NO: 336).
6. The antibody of any one of paragraphs 1-5, wherein the VL domain comprises the amino acid sequence of SEQ ID NO: 328.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 327 and
(b) a VL domain comprising an amino acid sequence having at least 90%, at least 95%, or at least 99% sequence identity to the amino acid sequence of SEQ ID NO: 328.
8. The antibody of paragraph 7, wherein the antibody comprises a VH domain comprising the amino acid sequence of SEQ ID NO: 327 and a VL domain comprising the amino acid sequence of SEQ ID NO: 328.
9. The antibody of any one of paragraphs 1-8, wherein the antibody comprises (a) a heavy chain comprising the amino acid sequence of SEQ ID NO: 337 and (b) a light chain comprising the amino acid sequence of SEQ ID NO: 338.
10. The antibody of any one of paragraphs 1-9, wherein the antibody binds Ara h 2 with a KD of about 50 nM or less.
11. The method of paragraph 10, wherein the KD is measured by a surface plasmon resonance assay.
12. The antibody of paragraph 11, wherein the antibody binds Ara h 2 with a KD of between about 0.1 pM and about 40 nM.
13. The antibody of paragraph 12, wherein the antibody binds Ara h 2 with a KD of between about 120 pM and about 20 nM.
14. The antibody of paragraph 13, wherein the antibody binds Ara h 2 with a KD of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)2 fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### P34:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 341);
(b) an CDR-H2 comprising the amino acid sequence of (SEQ ID NO: 342);
(c) an CDR-H3 comprising the amino acid sequence of (SEQ ID NO: 343);
(d) an CDR-L1 comprising the amino acid sequence of (SEQ ID NO: 344);
(e) an CDR-L2 comprising the amino acid sequence of (SEQ ID NO: 345); and
(f) an CDR-L3 comprising the amino acid sequence of (SEQ ID NO: 346).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 347;
(b) a light chain variable (VL) domain comprising an amino acid sequence having at least 90%, at least 95%, or at least 99% identity to the amino acid sequence of SEQ ID NO: 348; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 349);
(b) an FR-H2 comprising the amino acid sequence of (SEQ ID NO: 350);
(c) an FR-H3 comprising the amino acid sequence of (SEQ ID NO: 351); and
(d) an FR-H4 comprising the amino acid sequence of (SEQ ID NO: 352).
4. The antibody of any one of paragraphs 1-3, wherein the VH domain comprises the amino acid sequence of SEQ ID NO: 347.
5. The antibody of any one of paragraphs 1-4, further comprising the following VL domain FRs:
(a) an FR-L1 comprising the amino acid sequence of (SEQ ID NO: 353);
(b) an FR-L2 comprising the amino acid sequence of (SEQ ID NO: 354);
(c) an FR-L3 comprising the amino acid sequence of (SEQ ID NO: 355); and
(d) an FR-L4 comprising the amino acid sequence of (SEQ ID NO: 356).
6. The antibody of any one of paragraphs 1-5, wherein the VL domain comprises the amino acid sequence of SEQ ID NO: 348.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 347 and
(b) a VL domain comprising an amino acid sequence having at least 90%, at least 95%, or at least 99% sequence identity to the amino acid sequence of SEQ ID NO: 348.
8. The antibody of paragraph 7, wherein the antibody comprises a VH domain comprising the amino acid sequence of SEQ ID NO: 347 and a VL domain comprising the amino acid sequence of SEQ ID NO: 348.
9. The antibody of any one of paragraphs 1-8, wherein the antibody comprises (a) a heavy chain comprising the amino acid sequence of SEQ ID NO: 357 and (b) a light chain comprising the amino acid sequence of SEQ ID NO: 358.
10. The antibody of any one of paragraphs 1-9, wherein the antibody binds Ara h 2 with a KD of about 50 nM or less.
11. The method of paragraph 10, wherein the KD is measured by a surface plasmon resonance assay.
12. The antibody of paragraph 11, wherein the antibody binds Ara h 2 with a KD of between about 0.1 pM and about 40 nM.
13. The antibody of paragraph 12, wherein the antibody binds Ara h 2 with a KD of between about 120 pM and about 20 nM.
14. The antibody of paragraph 13, wherein the antibody binds Ara h 2 with a KD of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)2 fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### P39:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 361);
(b) an CDR-H2 comprising the amino acid sequence of (SEQ ID NO: 362);
(c) an CDR-H3 comprising the amino acid sequence of (SEQ ID NO: 363);
(d) an CDR-L1 comprising the amino acid sequence of (SEQ ID NO: 364);
(e) an CDR-L2 comprising the amino acid sequence of (SEQ ID NO: 365); and
(f) an CDR-L3 comprising the amino acid sequence of (SEQ ID NO: 366).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 367;
(b) a light chain variable (VL) domain comprising an amino acid sequence having at least 90%, at least 95%, or at least 99% identity to the amino acid sequence of SEQ ID NO: 368; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 369);
(b) an FR-H2 comprising the amino acid sequence of (SEQ ID NO: 370);
(c) an FR-H3 comprising the amino acid sequence of (SEQ ID NO: 371); and
(d) an FR-H4 comprising the amino acid sequence of (SEQ ID NO: 372).
4. The antibody of any one of paragraphs 1-3, wherein the VH domain comprises the amino acid sequence of SEQ ID NO: 367.
5. The antibody of any one of paragraphs 1-4, further comprising the following VL domain FRs:
(a) an FR-L1 comprising the amino acid sequence of (SEQ ID NO: 373);
(b) an FR-L2 comprising the amino acid sequence of (SEQ ID NO: 374);
(c) an FR-L3 comprising the amino acid sequence of (SEQ ID NO: 375); and
(d) an FR-L4 comprising the amino acid sequence of (SEQ ID NO: 376).
6. The antibody of any one of paragraphs 1-5, wherein the VL domain comprises the amino acid sequence of SEQ ID NO: 368.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 367 and
(b) a VL domain comprising an amino acid sequence having at least 90%, at least 95%, or at least 99% sequence identity to the amino acid sequence of SEQ ID NO: 368.
8. The antibody of paragraph 7, wherein the antibody comprises a VH domain comprising the amino acid sequence of SEQ ID NO: 367 and a VL domain comprising the amino acid sequence of SEQ ID NO: 368.
9. The antibody of any one of paragraphs 1-8, wherein the antibody comprises (a) a heavy chain comprising the amino acid sequence of SEQ ID NO: 377 and (b) a light chain comprising the amino acid sequence of SEQ ID NO: 378.
10. The antibody of any one of paragraphs 1-9, wherein the antibody binds Ara h 2 with a KD of about 50 nM or less.
11. The method of paragraph 10, wherein the KD is measured by a surface plasmon resonance assay.
12. The antibody of paragraph 11, wherein the antibody binds Ara h 2 with a KD of between about 0.1 pM and about 40 nM.
13. The antibody of paragraph 12, wherein the antibody binds Ara h 2 with a KD of between about 120 pM and about 20 nM.
14. The antibody of paragraph 13, wherein the antibody binds Ara h 2 with a KD of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)2 fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### S1:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 381);
(b) an CDR-H2 comprising the amino acid sequence of (SEQ ID NO: 382);
(c) an CDR-H3 comprising the amino acid sequence of (SEQ ID NO: 383);
(d) an CDR-L1 comprising the amino acid sequence of (SEQ ID NO: 384);
(e) an CDR-L2 comprising the amino acid sequence of (SEQ ID NO: 385); and
(f) an CDR-L3 comprising the amino acid sequence of (SEQ ID NO: 386).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 387;
(b) a light chain variable (VL) domain comprising an amino acid sequence having at least 90%, at least 95%, or at least 99% identity to the amino acid sequence of SEQ ID NO: 388; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 389);
(b) an FR-H2 comprising the amino acid sequence of (SEQ ID NO: 390);
(c) an FR-H3 comprising the amino acid sequence of (SEQ ID NO: 391); and
(d) an FR-H4 comprising the amino acid sequence of (SEQ ID NO: 392).
4. The antibody of any one of paragraphs 1-3, wherein the VH domain comprises the amino acid sequence of SEQ ID NO: 387.
5. The antibody of any one of paragraphs 1-4, further comprising the following VL domain FRs:
(a) an FR-L1 comprising the amino acid sequence of (SEQ ID NO: 393);
(b) an FR-L2 comprising the amino acid sequence of (SEQ ID NO: 394);
(c) an FR-L3 comprising the amino acid sequence of (SEQ ID NO: 395); and
(d) an FR-L4 comprising the amino acid sequence of (SEQ ID NO: 396).
6. The antibody of any one of paragraphs 1-5, wherein the VL domain comprises the amino acid sequence of SEQ ID NO: 388.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 387 and
(b) a VL domain comprising an amino acid sequence having at least 90%, at least 95%, or at least 99% sequence identity to the amino acid sequence of SEQ ID NO: 388.
8. The antibody of paragraph 7, wherein the antibody comprises a VH domain comprising the amino acid sequence of SEQ ID NO: 387 and a VL domain comprising the amino acid sequence of SEQ ID NO: 388.
9. The antibody of any one of paragraphs 1-8, wherein the antibody comprises (a) a heavy chain comprising the amino acid sequence of SEQ ID NO: 397 and (b) a light chain comprising the amino acid sequence of SEQ ID NO: 398.
10. The antibody of any one of paragraphs 1-9, wherein the antibody binds Ara h 2 with a KD of about 50 nM or less.
11. The method of paragraph 10, wherein the KD is measured by a surface plasmon resonance assay.
12. The antibody of paragraph 11, wherein the antibody binds Ara h 2 with a KD of between about 0.1 pM and about 40 nM.
13. The antibody of paragraph 12, wherein the antibody binds Ara h 2 with a KD of between about 120 pM and about 20 nM.
14. The antibody of paragraph 13, wherein the antibody binds Ara h 2 with a KD of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)2 fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### S4:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 401);
(b) an CDR-H2 comprising the amino acid sequence of (SEQ ID NO: 402);
(c) an CDR-H3 comprising the amino acid sequence of (SEQ ID NO: 403);
(d) an CDR-L1 comprising the amino acid sequence of (SEQ ID NO: 404);
(e) an CDR-L2 comprising the amino acid sequence of (SEQ ID NO: 405); and
(f) an CDR-L3 comprising the amino acid sequence of (SEQ ID NO: 406).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 407;
(b) a light chain variable (VL) domain comprising an amino acid sequence having at least 90%, at least 95%, or at least 99% identity to the amino acid sequence of SEQ ID NO: 408; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 409);
(b) an FR-H2 comprising the amino acid sequence of (SEQ ID NO: 410);
(c) an FR-H3 comprising the amino acid sequence of (SEQ ID NO: 411); and
(d) an FR-H4 comprising the amino acid sequence of (SEQ ID NO: 412).
4. The antibody of any one of paragraphs 1-3, wherein the VH domain comprises the amino acid sequence of SEQ ID NO: 407.
5. The antibody of any one of paragraphs 1-4, further comprising the following VL domain FRs:
(a) an FR-L1 comprising the amino acid sequence of (SEQ ID NO: 413);
(b) an FR-L2 comprising the amino acid sequence of (SEQ ID NO: 414);
(c) an FR-L3 comprising the amino acid sequence of (SEQ ID NO: 415); and
(d) an FR-L4 comprising the amino acid sequence of (SEQ ID NO: 416).
6. The antibody of any one of paragraphs 1-5, wherein the VL domain comprises the amino acid sequence of SEQ ID NO: 408.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 407 and
(b) a VL domain comprising an amino acid sequence having at least 90%, at least 95%, or at least 99% sequence identity to the amino acid sequence of SEQ ID NO: 408.
8. The antibody of paragraph 7, wherein the antibody comprises a VH domain comprising the amino acid sequence of SEQ ID NO: 407 and a VL domain comprising the amino acid sequence of SEQ ID NO: 408.
9. The antibody of any one of paragraphs 1-8, wherein the antibody comprises (a) a heavy chain comprising the amino acid sequence of SEQ ID NO: 417 and (b) a light chain comprising the amino acid sequence of SEQ ID NO: 418.
10. The antibody of any one of paragraphs 1-9, wherein the antibody binds Ara h 2 with a KD of about 50 nM or less.
11. The method of paragraph 10, wherein the KD is measured by a surface plasmon resonance assay.
12. The antibody of paragraph 11, wherein the antibody binds Ara h 2 with a KD of between about 0.1 pM and about 40 nM.
13. The antibody of paragraph 12, wherein the antibody binds Ara h 2 with a KD of between about 120 pM and about 20 nM.
14. The antibody of paragraph 13, wherein the antibody binds Ara h 2 with a KD of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)2 fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### 24B7D4:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 421);
(b) an CDR-H2 comprising the amino acid sequence of (SEQ ID NO: 422);
(c) an CDR-H3 comprising the amino acid sequence of (SEQ ID NO: 423);
(d) an CDR-L1 comprising the amino acid sequence of (SEQ ID NO: 424);
(e) an CDR-L2 comprising the amino acid sequence of (SEQ ID NO: 425); and
(f) an CDR-L3 comprising the amino acid sequence of (SEQ ID NO: 426).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 427;
(b) a light chain variable (VL) domain comprising an amino acid sequence having at least 90%, at least 95%, or at least 99% identity to the amino acid sequence of SEQ ID NO: 428; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 429);
(b) an FR-H2 comprising the amino acid sequence of (SEQ ID NO: 430);
(c) an FR-H3 comprising the amino acid sequence of (SEQ ID NO: 431); and
(d) an FR-H4 comprising the amino acid sequence of (SEQ ID NO: 432).
4. The antibody of any one of paragraphs 1-3, wherein the VH domain comprises the amino acid sequence of SEQ ID NO: 427.
5. The antibody of any one of paragraphs 1-4, further comprising the following VL domain FRs:
(a) an FR-L1 comprising the amino acid sequence of (SEQ ID NO: 433);
(b) an FR-L2 comprising the amino acid sequence of (SEQ ID NO: 434);
(c) an FR-L3 comprising the amino acid sequence of (SEQ ID NO: 435); and
(d) an FR-L4 comprising the amino acid sequence of (SEQ ID NO: 436).
6. The antibody of any one of paragraphs 1-5, wherein the VL domain comprises the amino acid sequence of SEQ ID NO: 428.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 427 and
(b) a VL domain comprising an amino acid sequence having at least 90%, at least 95%, or at least 99% sequence identity to the amino acid sequence of SEQ ID NO: 428.
8. The antibody of paragraph 7, wherein the antibody comprises a VH domain comprising the amino acid sequence of SEQ ID NO: 427 and a VL domain comprising the amino acid sequence of SEQ ID NO: 428.
9. The antibody of any one of paragraphs 1-8, wherein the antibody comprises (a) a heavy chain comprising the amino acid sequence of SEQ ID NO: 437 and (b) a light chain comprising the amino acid sequence of SEQ ID NO: 438.
10. The antibody of any one of paragraphs 1-9, wherein the antibody binds Ara h 2 with a KD of about 50 nM or less.
11. The method of paragraph 10, wherein the KD is measured by a surface plasmon resonance assay.
12. The antibody of paragraph 11, wherein the antibody binds Ara h 2 with a KD of between about 0.1 pM and about 40 nM.
13. The antibody of paragraph 12, wherein the antibody binds Ara h 2 with a KD of between about 120 pM and about 20 nM.
14. The antibody of paragraph 13, wherein the antibody binds Ara h 2 with a KD of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)2 fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### T1:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 442);
(b) an CDR-H2 comprising the amino acid sequence of (SEQ ID NO: 443);
(c) an CDR-H3 comprising the amino acid sequence of (SEQ ID NO: 444);
(d) an CDR-L1 comprising the amino acid sequence of (SEQ ID NO: 445);
(e) an CDR-L2 comprising the amino acid sequence of (SEQ ID NO: 446); and
(f) an CDR-L3 comprising the amino acid sequence of (SEQ ID NO: 447).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 448;
(b) a light chain variable (VL) domain comprising an amino acid sequence having at least 90%, at least 95%, or at least 99% identity to the amino acid sequence of SEQ ID NO: 449; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 450);
(b) an FR-H2 comprising the amino acid sequence of (SEQ ID NO: 451);
(c) an FR-H3 comprising the amino acid sequence of (SEQ ID NO: 452); and
(d) an FR-H4 comprising the amino acid sequence of (SEQ ID NO: 453).
4. The antibody of any one of paragraphs 1-3, wherein the VH domain comprises the amino acid sequence of SEQ ID NO: 448.
5. The antibody of any one of paragraphs 1-4, further comprising the following VL domain FRs:
(a) an FR-L1 comprising the amino acid sequence of (SEQ ID NO: 454);
(b) an FR-L2 comprising the amino acid sequence of (SEQ ID NO: 455);
(c) an FR-L3 comprising the amino acid sequence of (SEQ ID NO: 456); and
(d) an FR-L4 comprising the amino acid sequence of (SEQ ID NO: 457).
6. The antibody of any one of paragraphs 1-5, wherein the VL domain comprises the amino acid sequence of SEQ ID NO: 449.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 448 and
(b) a VL domain comprising an amino acid sequence having at least 90%, at least 95%, or at least 99% sequence identity to the amino acid sequence of SEQ ID NO: 449.
8. The antibody of paragraph 7, wherein the antibody comprises a VH domain comprising the amino acid sequence of SEQ ID NO: 448 and a VL domain comprising the amino acid sequence of SEQ ID NO: 449.
9. The antibody of any one of paragraphs 1-8, wherein the antibody comprises (a) a heavy chain comprising the amino acid sequence of SEQ ID NO: 458 and (b) a light chain comprising the amino acid sequence of SEQ ID NO: 459.
10. The antibody of any one of paragraphs 1-9, wherein the antibody binds Ara h 2 with a KD of about 50 nM or less.
11. The method of paragraph 10, wherein the KD is measured by a surface plasmon resonance assay.
12. The antibody of paragraph 11, wherein the antibody binds Ara h 2 with a KD of between about 0.1 pM and about 40 nM.
13. The antibody of paragraph 12, wherein the antibody binds Ara h 2 with a KD of between about 120 pM and about 20 nM.
14. The antibody of paragraph 13, wherein the antibody binds Ara h 2 with a KD of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)2 fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.
1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 462);
(b) an CDR-H2 comprising the amino acid sequence of (SEQ ID NO: 463);
(c) an CDR-H3 comprising the amino acid sequence of (SEQ ID NO: 464);
(d) an CDR-L1 comprising the amino acid sequence of (SEQ ID NO: 465);
(e) an CDR-L2 comprising the amino acid sequence of (SEQ ID NO: 466); and
(f) an CDR-L3 comprising the amino acid sequence of (SEQ ID NO: 467).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 468;
(b) a light chain variable (VL) domain comprising an amino acid sequence having at least 90%, at least 95%, or at least 99% identity to the amino acid sequence of SEQ ID NO: 469; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 470);
(b) an FR-H2 comprising the amino acid sequence of (SEQ ID NO: 471);
(c) an FR-H3 comprising the amino acid sequence of (SEQ ID NO: 472); and
(d) an FR-H4 comprising the amino acid sequence of (SEQ ID NO: 473).
4. The antibody of any one of paragraphs 1-3, wherein the VH domain comprises the amino acid sequence of SEQ ID NO: 468.
5. The antibody of any one of paragraphs 1-4, further comprising the following VL domain FRs:
(a) an FR-L1 comprising the amino acid sequence of (SEQ ID NO: 474);
(b) an FR-L2 comprising the amino acid sequence of (SEQ ID NO: 475);
(c) an FR-L3 comprising the amino acid sequence of (SEQ ID NO: 476); and
(d) an FR-L4 comprising the amino acid sequence of (SEQ ID NO: 477).
6. The antibody of any one of paragraphs 1-5, wherein the VL domain comprises the amino acid sequence of SEQ ID NO: 469.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 468 and
(b) a VL domain comprising an amino acid sequence having at least 90%, at least 95%, or at least 99% sequence identity to the amino acid sequence of SEQ ID NO: 469.
8. The antibody of paragraph 7, wherein the antibody comprises a VH domain comprising the amino acid sequence of SEQ ID NO: 468 and a VL domain comprising the amino acid sequence of SEQ ID NO: 469.
9. The antibody of any one of paragraphs 1-8, wherein the antibody comprises (a) a heavy chain comprising the amino acid sequence of SEQ ID NO: 478 and (b) a light chain comprising the amino acid sequence of SEQ ID NO: 479.
10. The antibody of any one of paragraphs 1-9, wherein the antibody binds Ara h 2 with a KD of about 50 nM or less.
11. The method of paragraph 10, wherein the KD is measured by a surface plasmon resonance assay.
12. The antibody of paragraph 11, wherein the antibody binds Ara h 2 with a KD of between about 0.1 pM and about 40 nM.
13. The antibody of paragraph 12, wherein the antibody binds Ara h 2 with a KD of between about 120 pM and about 20 nM.
14. The antibody of paragraph 13, wherein the antibody binds Ara h 2 with a KD of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)2 fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### T4:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 482);
(b) an CDR-H2 comprising the amino acid sequence of (SEQ ID NO: 483);
(c) an CDR-H3 comprising the amino acid sequence of (SEQ ID NO: 484);
(d) an CDR-L1 comprising the amino acid sequence of (SEQ ID NO: 485);
(e) an CDR-L2 comprising the amino acid sequence of (SEQ ID NO: 486); and
(f) an CDR-L3 comprising the amino acid sequence of (SEQ ID NO: 487).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 488;
(b) a light chain variable (VL) domain comprising an amino acid sequence having at least 90%, at least 95%, or at least 99% identity to the amino acid sequence of SEQ ID NO: 489; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 490);
(b) an FR-H2 comprising the amino acid sequence of (SEQ ID NO: 491);
(c) an FR-H3 comprising the amino acid sequence of (SEQ ID NO: 492); and
(d) an FR-H4 comprising the amino acid sequence of (SEQ ID NO: 493).
4. The antibody of any one of paragraphs 1-3, wherein the VH domain comprises the amino acid sequence of SEQ ID NO: 488.
5. The antibody of any one of paragraphs 1-4, further comprising the following VL domain FRs:
(a) an FR-L1 comprising the amino acid sequence of (SEQ ID NO: 494);
(b) an FR-L2 comprising the amino acid sequence of (SEQ ID NO: 495);
(c) an FR-L3 comprising the amino acid sequence of (SEQ ID NO: 496); and
(d) an FR-L4 comprising the amino acid sequence of (SEQ ID NO: 497).
6. The antibody of any one of paragraphs 1-5, wherein the VL domain comprises the amino acid sequence of SEQ ID NO: 489.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 488 and
(b) a VL domain comprising an amino acid sequence having at least 90%, at least 95%, or at least 99% sequence identity to the amino acid sequence of SEQ ID NO: 489.
8. The antibody of paragraph 7, wherein the antibody comprises a VH domain comprising the amino acid sequence of SEQ ID NO: 488 and a VL domain comprising the amino acid sequence of SEQ ID NO: 489.
9. The antibody of any one of paragraphs 1-8, wherein the antibody comprises (a) a heavy chain comprising the amino acid sequence of SEQ ID NO: 498 and (b) a light chain comprising the amino acid sequence of SEQ ID NO: 499.
10. The antibody of any one of paragraphs 1-9, wherein the antibody binds Ara h 2 with a KD of about 50 nM or less.
11. The method of paragraph 10, wherein the KD is measured by a surface plasmon resonance assay.
12. The antibody of paragraph 11, wherein the antibody binds Ara h 2 with a KD of between about 0.1 pM and about 40 nM.
13. The antibody of paragraph 12, wherein the antibody binds Ara h 2 with a KD of between about 120 pM and about 20 nM.
14. The antibody of paragraph 13, wherein the antibody binds Ara h 2 with a KD of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)2 fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### T5:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 502);
(b) an CDR-H2 comprising the amino acid sequence of (SEQ ID NO: 503);
(c) an CDR-H3 comprising the amino acid sequence of (SEQ ID NO: 504);
(d) an CDR-L1 comprising the amino acid sequence of (SEQ ID NO: 505);
(e) an CDR-L2 comprising the amino acid sequence of (SEQ ID NO: 506); and
(f) an CDR-L3 comprising the amino acid sequence of (SEQ ID NO: 507).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 508;
(b) a light chain variable (VL) domain comprising an amino acid sequence having at least 90%, at least 95%, or at least 99% identity to the amino acid sequence of SEQ ID NO: 509; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 510);
(b) an FR-H2 comprising the amino acid sequence of (SEQ ID NO: 511);
(c) an FR-H3 comprising the amino acid sequence of (SEQ ID NO: 512); and
(d) an FR-H4 comprising the amino acid sequence of (SEQ ID NO: 513).
4. The antibody of any one of paragraphs 1-3, wherein the VH domain comprises the amino acid sequence of SEQ ID NO: 508.
5. The antibody of any one of paragraphs 1-4, further comprising the following VL domain FRs:
(a) an FR-L1 comprising the amino acid sequence of (SEQ ID NO: 514);
(b) an FR-L2 comprising the amino acid sequence of (SEQ ID NO: 515);
(c) an FR-L3 comprising the amino acid sequence of (SEQ ID NO: 516); and
(d) an FR-L4 comprising the amino acid sequence of (SEQ ID NO: 517).
6. The antibody of any one of paragraphs 1-5, wherein the VL domain comprises the amino acid sequence of SEQ ID NO: 509.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 508 and
(b) a VL domain comprising an amino acid sequence having at least 90%, at least 95%, or at least 99% sequence identity to the amino acid sequence of SEQ ID NO: 509.
8. The antibody of paragraph 7, wherein the antibody comprises a VH domain comprising the amino acid sequence of SEQ ID NO: 508 and a VL domain comprising the amino acid sequence of SEQ ID NO: 509.
9. The antibody of any one of paragraphs 1-8, wherein the antibody comprises (a) a heavy chain comprising the amino acid sequence of SEQ ID NO: 518 and (b) a light chain comprising the amino acid sequence of SEQ ID NO: 519.
10. The antibody of any one of paragraphs 1-9, wherein the antibody binds Ara h 2 with a KD of about 50 nM or less.
11. The method of paragraph 10, wherein the KD is measured by a surface plasmon resonance assay.
12. The antibody of paragraph 11, wherein the antibody binds Ara h 2 with a KD of between about 0.1 pM and about 40 nM.
13. The antibody of paragraph 12, wherein the antibody binds Ara h 2 with a KD of between about 120 pM and about 20 nM.
14. The antibody of paragraph 13, wherein the antibody binds Ara h 2 with a KD of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)2 fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### 13FU1P1A4:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 700);
(b) an CDR-H2 comprising the amino acid sequence of (SEQ ID NO: 701);
(c) an CDR-H3 comprising the amino acid sequence of (SEQ ID NO: 702);
(d) an CDR-L1 comprising the amino acid sequence of (SEQ ID NO: 703);
(e) an CDR-L2 comprising the amino acid sequence of (SEQ ID NO: 704); and
(f) an CDR-L3 comprising the amino acid sequence of (SEQ ID NO: 705).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 706;
(b) a light chain variable (VL) domain comprising an amino acid sequence having at least 90%, at least 95%, or at least 99% identity to the amino acid sequence of SEQ ID NO: 707; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 708);
(b) an FR-H2 comprising the amino acid sequence of (SEQ ID NO: 709);
(c) an FR-H3 comprising the amino acid sequence of (SEQ ID NO: 710); and
(d) an FR-H4 comprising the amino acid sequence of (SEQ ID NO: 711).
4. The antibody of any one of paragraphs 1-3, wherein the VH domain comprises the amino acid sequence of SEQ ID NO: 706.
5. The antibody of any one of paragraphs 1-4, further comprising the following VL domain FRs:
(a) an FR-L1 comprising the amino acid sequence of (SEQ ID NO: 712);
(b) an FR-L2 comprising the amino acid sequence of (SEQ ID NO: 713);
(c) an FR-L3 comprising the amino acid sequence of (SEQ ID NO: 714); and
(d) an FR-L4 comprising the amino acid sequence of (SEQ ID NO: 715).
6. The antibody of any one of paragraphs 1-5, wherein the VL domain comprises the amino acid sequence of SEQ ID NO: 707.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 706 and
(b) a VL domain comprising an amino acid sequence having at least 90%, at least 95%, or at least 99% sequence identity to the amino acid sequence of SEQ ID NO: 707.
8. The antibody of paragraph 7, wherein the antibody comprises a VH domain comprising the amino acid sequence of SEQ ID NO: 706 and a VL domain comprising the amino acid sequence of SEQ ID NO: 707.
9. The antibody of any one of paragraphs 1-8, wherein the antibody comprises (a) a heavy chain comprising the amino acid sequence of SEQ ID NO: 716 and (b) a light chain comprising the amino acid sequence of SEQ ID NO: 717.
10. The antibody of any one of paragraphs 1-9, wherein the antibody binds Ara h 2 with a K_{D} of about 50 nM or less.
11. The method of paragraph 10, wherein the K_{D} is measured by a surface plasmon resonance assay.
12. The antibody of paragraph 11, wherein the antibody binds Ara h 2 with a K_{D} of between about 0.1 pM and about 40 nM.
13. The antibody of paragraph 12, wherein the antibody binds Ara h 2 with a K_{D} of between about 120 pM and about 20 nM.
14. The antibody of paragraph 13, wherein the antibody binds Ara h 2 with a K_{D} of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)₂ fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### 13FU1P1B4:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 1840);
(b) an CDR-H2 comprising the amino acid sequence of (SEQ ID NO: 1841);
(c) an CDR-H3 comprising the amino acid sequence of (SEQ ID NO: 1842);
(d) an CDR-L1 comprising the amino acid sequence of (SEQ ID NO: 1843);
(e) an CDR-L2 comprising the amino acid sequence of (SEQ ID NO: 1844); and
(f) an CDR-L3 comprising the amino acid sequence of (SEQ ID NO: 1845).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 1846;
(b) a light chain variable (VL) domain comprising an amino acid sequence having at least 90%, at least 95%, or at least 99% identity to the amino acid sequence of SEQ ID NO: 1847; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 1848);
(b) an FR-H2 comprising the amino acid sequence of (SEQ ID NO: 1849);
(c) an FR-H3 comprising the amino acid sequence of (SEQ ID NO: 1850); and
(d) an FR-H4 comprising the amino acid sequence of (SEQ ID NO: 1851).
4. The antibody of any one of paragraphs 1-3, wherein the VH domain comprises the amino acid sequence of SEQ ID NO: 1846.
5. The antibody of any one of paragraphs 1-4, further comprising the following VL domain FRs:
(a) an FR-L1 comprising the amino acid sequence of (SEQ ID NO: 1852);
(b) an FR-L2 comprising the amino acid sequence of (SEQ ID NO: 1853);
(c) an FR-L3 comprising the amino acid sequence of (SEQ ID NO: 1854); and
(d) an FR-L4 comprising the amino acid sequence of (SEQ ID NO: 1855).
6. The antibody of any one of paragraphs 1-5, wherein the VL domain comprises the amino acid sequence of SEQ ID NO: 1847.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 1846 and
(b) a VL domain comprising an amino acid sequence having at least 90%, at least 95%, or at least 99% sequence identity to the amino acid sequence of SEQ ID NO: 1847.
8. The antibody of paragraph 7, wherein the antibody comprises a VH domain comprising the amino acid sequence of SEQ ID NO: 1846 and a VL domain comprising the amino acid sequence of SEQ ID NO: 1847.
9. The antibody of any one of paragraphs 1-8, wherein the antibody comprises (a) a heavy chain comprising the amino acid sequence of SEQ ID NO: 1856 and (b) a light chain comprising the amino acid sequence of SEQ ID NO: 1857.
10. The antibody of any one of paragraphs 1-9, wherein the antibody binds Ara h 2 with a K_{D} of about 50 nM or less.
11. The method of paragraph 10, wherein the K_{D} is measured by a surface plasmon resonance assay.
12. The antibody of paragraph 11, wherein the antibody binds Ara h 2 with a K_{D} of between about 0.1 pM and about 40 nM.
13. The antibody of paragraph 12, wherein the antibody binds Ara h 2 with a K_{D} of between about 120 pM and about 20 nM.
14. The antibody of paragraph 13, wherein the antibody binds Ara h 2 with a K_{D} of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)2 fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### 15FU1P3A1:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 840);
(b) an CDR-H2 comprising the amino acid sequence of (SEQ ID NO: 841);
(c) an CDR-H3 comprising the amino acid sequence of (SEQ ID NO: 842);
(d) an CDR-L1 comprising the amino acid sequence of (SEQ ID NO: 843);
(e) an CDR-L2 comprising the amino acid sequence of (SEQ ID NO: 844); and
(f) an CDR-L3 comprising the amino acid sequence of (SEQ ID NO: 845).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 846;
(b) a light chain variable (VL) domain comprising an amino acid sequence having at least 90%, at least 95%, or at least 99% identity to the amino acid sequence of SEQ ID NO: 847; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 848);
(b) an FR-H2 comprising the amino acid sequence of (SEQ ID NO: 849);
(c) an FR-H3 comprising the amino acid sequence of (SEQ ID NO: 850); and
(d) an FR-H4 comprising the amino acid sequence of (SEQ ID NO: 851).
4. The antibody of any one of paragraphs 1-3, wherein the VH domain comprises the amino acid sequence of SEQ ID NO: 846.
5. The antibody of any one of paragraphs 1-4, further comprising the following VL domain FRs:
(a) an FR-L1 comprising the amino acid sequence of (SEQ ID NO: 852);
(b) an FR-L2 comprising the amino acid sequence of (SEQ ID NO: 853);
(c) an FR-L3 comprising the amino acid sequence of (SEQ ID NO: 854); and
(d) an FR-L4 comprising the amino acid sequence of (SEQ ID NO: 855).
6. The antibody of any one of paragraphs 1-5, wherein the VL domain comprises the amino acid sequence of SEQ ID NO: 847.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 846 and
(b) a VL domain comprising an amino acid sequence having at least 90%, at least 95%, or at least 99% sequence identity to the amino acid sequence of SEQ ID NO: 847.
8. The antibody of paragraph 7, wherein the antibody comprises a VH domain comprising the amino acid sequence of SEQ ID NO: 846 and a VL domain comprising the amino acid sequence of SEQ ID NO: 847.
9. The antibody of any one of paragraphs 1-8, wherein the antibody comprises (a) a heavy chain comprising the amino acid sequence of SEQ ID NO: 856 and (b) a light chain comprising the amino acid sequence of SEQ ID NO: 857.
10. The antibody of any one of paragraphs 1-9, wherein the antibody binds Ara h 2 with a K_{D} of about 50 nM or less.
11. The method of paragraph 10, wherein the K_{D} is measured by a surface plasmon resonance assay.
12. The antibody of paragraph 11, wherein the antibody binds Ara h 2 with a K_{D} of between about 0.1 pM and about 40 nM.
13. The antibody of paragraph 12, wherein the antibody binds Ara h 2 with a K_{D} of between about 120 pM and about 20 nM.
14. The antibody of paragraph 13, wherein the antibody binds Ara h 2 with a K_{D} of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)2 fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### 13FU1P2B10:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 860);
(b) an CDR-H2 comprising the amino acid sequence of (SEQ ID NO: 861);
(c) an CDR-H3 comprising the amino acid sequence of (SEQ ID NO: 862);
(d) an CDR-L1 comprising the amino acid sequence of (SEQ ID NO: 863);
(e) an CDR-L2 comprising the amino acid sequence of (SEQ ID NO: 864); and
(f) an CDR-L3 comprising the amino acid sequence of (SEQ ID NO: 865).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 866;
(b) a light chain variable (VL) domain comprising an amino acid sequence having at least 90%, at least 95%, or at least 99% identity to the amino acid sequence of SEQ ID NO: 867; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 868);
(b) an FR-H2 comprising the amino acid sequence of (SEQ ID NO: 869);
(c) an FR-H3 comprising the amino acid sequence of (SEQ ID NO: 870); and
(d) an FR-H4 comprising the amino acid sequence of (SEQ ID NO: 871).
4. The antibody of any one of paragraphs 1-3, wherein the VH domain comprises the amino acid sequence of SEQ ID NO: 866.
5. The antibody of any one of paragraphs 1-4, further comprising the following VL domain FRs:
(a) an FR-L1 comprising the amino acid sequence of (SEQ ID NO: 872);
(b) an FR-L2 comprising the amino acid sequence of (SEQ ID NO: 873);
(c) an FR-L3 comprising the amino acid sequence of (SEQ ID NO: 874); and
(d) an FR-L4 comprising the amino acid sequence of (SEQ ID NO: 875).
6. The antibody of any one of paragraphs 1-5, wherein the VL domain comprises the amino acid sequence of SEQ ID NO: 867.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 866 and
(b) a VL domain comprising an amino acid sequence having at least 90%, at least 95%, or at least 99% sequence identity to the amino acid sequence of SEQ ID NO: 867.
8. The antibody of paragraph 7, wherein the antibody comprises a VH domain comprising the amino acid sequence of SEQ ID NO: 866 and a VL domain comprising the amino acid sequence of SEQ ID NO: 867.
9. The antibody of any one of paragraphs 1-8, wherein the antibody comprises (a) a heavy chain comprising the amino acid sequence of SEQ ID NO: 876 and (b) a light chain comprising the amino acid sequence of SEQ ID NO: 877.
10. The antibody of any one of paragraphs 1-9, wherein the antibody binds Ara h 2 with a K_{D} of about 50 nM or less.
11. The method of paragraph 10, wherein the K_{D} is measured by a surface plasmon resonance assay.
12. The antibody of paragraph 11, wherein the antibody binds Ara h 2 with a K_{D} of between about 0.1 pM and about 40 nM.
13. The antibody of paragraph 12, wherein the antibody binds Ara h 2 with a K_{D} of between about 120 pM and about 20 nM.
14. The antibody of paragraph 13, wherein the antibody binds Ara h 2 with a K_{D} of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)₂ fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### 13FU1P2B12:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 880);
(b) an CDR-H2 comprising the amino acid sequence of (SEQ ID NO: 881);
(c) an CDR-H3 comprising the amino acid sequence of (SEQ ID NO: 882);
(d) an CDR-L1 comprising the amino acid sequence of (SEQ ID NO: 883);
(e) an CDR-L2 comprising the amino acid sequence of (SEQ ID NO: 884); and
(f) an CDR-L3 comprising the amino acid sequence of (SEQ ID NO: 885).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 886;
(b) a light chain variable (VL) domain comprising an amino acid sequence having at least 90%, at least 95%, or at least 99% identity to the amino acid sequence of SEQ ID NO: 887; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 888);
(b) an FR-H2 comprising the amino acid sequence of (SEQ ID NO: 889);
(c) an FR-H3 comprising the amino acid sequence of (SEQ ID NO: 890); and
(d) an FR-H4 comprising the amino acid sequence of (SEQ ID NO: 891).
4. The antibody of any one of paragraphs 1-3, wherein the VH domain comprises the amino acid sequence of SEQ ID NO: 886.
5. The antibody of any one of paragraphs 1-4, further comprising the following VL domain FRs:
(a) an FR-L1 comprising the amino acid sequence of (SEQ ID NO: 892);
(b) an FR-L2 comprising the amino acid sequence of (SEQ ID NO: 893);
(c) an FR-L3 comprising the amino acid sequence of (SEQ ID NO: 894); and
(d) an FR-L4 comprising the amino acid sequence of (SEQ ID NO: 895).
6. The antibody of any one of paragraphs 1-5, wherein the VL domain comprises the amino acid sequence of SEQ ID NO: 887.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 886 and
(b) a VL domain comprising an amino acid sequence having at least 90%, at least 95%, or at least 99% sequence identity to the amino acid sequence of SEQ ID NO: 887.
8. The antibody of paragraph 7, wherein the antibody comprises a VH domain comprising the amino acid sequence of SEQ ID NO: 886 and a VL domain comprising the amino acid sequence of SEQ ID NO: 887.
9. The antibody of any one of paragraphs 1-8, wherein the antibody comprises (a) a heavy chain comprising the amino acid sequence of SEQ ID NO: 896 and (b) a light chain comprising the amino acid sequence of SEQ ID NO: 897.
10. The antibody of any one of paragraphs 1-9, wherein the antibody binds Ara h 2 with a K_{D} of about 50 nM or less.
11. The method of paragraph 10, wherein the K_{D} is measured by a surface plasmon resonance assay.
12. The antibody of paragraph 11, wherein the antibody binds Ara h 2 with a K_{D} of between about 0.1 pM and about 40 nM.
13. The antibody of paragraph 12, wherein the antibody binds Ara h 2 with a K_{D} of between about 120 pM and about 20 nM.
14. The antibody of paragraph 13, wherein the antibody binds Ara h 2 with a K_{D} of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)2 fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### 6BU4P2B1:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 940);
(b) an CDR-H2 comprising the amino acid sequence of (SEQ ID NO: 941);
(c) an CDR-H3 comprising the amino acid sequence of (SEQ ID NO: 942);
(d) an CDR-L1 comprising the amino acid sequence of (SEQ ID NO: 943);
(e) an CDR-L2 comprising the amino acid sequence of (SEQ ID NO: 944); and
(f) an CDR-L3 comprising the amino acid sequence of (SEQ ID NO: 945).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 946;
(b) a light chain variable (VL) domain comprising an amino acid sequence having at least 90%, at least 95%, or at least 99% identity to the amino acid sequence of SEQ ID NO: 947; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 948);
(b) an FR-H2 comprising the amino acid sequence of (SEQ ID NO: 949);
(c) an FR-H3 comprising the amino acid sequence of (SEQ ID NO: 950); and
(d) an FR-H4 comprising the amino acid sequence of (SEQ ID NO: 951).
4. The antibody of any one of paragraphs 1-3, wherein the VH domain comprises the amino acid sequence of SEQ ID NO: 946.
5. The antibody of any one of paragraphs 1-4, further comprising the following VL domain FRs:
(a) an FR-L1 comprising the amino acid sequence of (SEQ ID NO: 952);
(b) an FR-L2 comprising the amino acid sequence of (SEQ ID NO: 953);
(c) an FR-L3 comprising the amino acid sequence of (SEQ ID NO: 954); and
(d) an FR-L4 comprising the amino acid sequence of (SEQ ID NO: 955).
6. The antibody of any one of paragraphs 1-5, wherein the VL domain comprises the amino acid sequence of SEQ ID NO: 947.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 946 and
(b) a VL domain comprising an amino acid sequence having at least 90%, at least 95%, or at least 99% sequence identity to the amino acid sequence of SEQ ID NO: 947.
8. The antibody of paragraph 7, wherein the antibody comprises a VH domain comprising the amino acid sequence of SEQ ID NO: 946 and a VL domain comprising the amino acid sequence of SEQ ID NO: 947.
9. The antibody of any one of paragraphs 1-8, wherein the antibody comprises (a) a heavy chain comprising the amino acid sequence of SEQ ID NO: 956 and (b) a light chain comprising the amino acid sequence of SEQ ID NO: 957.
10. The antibody of any one of paragraphs 1-9, wherein the antibody binds Ara h 2 with a K_{D} of about 50 nM or less.
11. The method of paragraph 10, wherein the K_{D} is measured by a surface plasmon resonance assay.
12. The antibody of paragraph 11, wherein the antibody binds Ara h 2 with a K_{D} of between about 0.1 pM and about 40 nM.
13. The antibody of paragraph 12, wherein the antibody binds Ara h 2 with a K_{D} of between about 120 pM and about 20 nM.
14. The antibody of paragraph 13, wherein the antibody binds Ara h 2 with a K_{D} of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)2 fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### 105BU7P1D6:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 1440);
(b) an CDR-H2 comprising the amino acid sequence of (SEQ ID NO: 1441);
(c) an CDR-H3 comprising the amino acid sequence of (SEQ ID NO: 1442);
(d) an CDR-L1 comprising the amino acid sequence of (SEQ ID NO: 1443);
(e) an CDR-L2 comprising the amino acid sequence of (SEQ ID NO: 1444); and
(f) an CDR-L3 comprising the amino acid sequence of (SEQ ID NO: 1445).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 1446;
(b) a light chain variable (VL) domain comprising an amino acid sequence having at least 90%, at least 95%, or at least 99% identity to the amino acid sequence of SEQ ID NO: 1447; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 1448);
(b) an FR-H2 comprising the amino acid sequence of (SEQ ID NO: 1449);
(c) an FR-H3 comprising the amino acid sequence of (SEQ ID NO: 1450); and
(d) an FR-H4 comprising the amino acid sequence of (SEQ ID NO: 1451).
4. The antibody of any one of paragraphs 1-3, wherein the VH domain comprises the amino acid sequence of SEQ ID NO: 1446.
5. The antibody of any one of paragraphs 1-4, further comprising the following VL domain FRs:
(a) an FR-L1 comprising the amino acid sequence of (SEQ ID NO: 1452);
(b) an FR-L2 comprising the amino acid sequence of (SEQ ID NO: 1453);
(c) an FR-L3 comprising the amino acid sequence of (SEQ ID NO: 1454); and
(d) an FR-L4 comprising the amino acid sequence of (SEQ ID NO: 1455).
6. The antibody of any one of paragraphs 1-5, wherein the VL domain comprises the amino acid sequence of SEQ ID NO: 1447.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 1446 and
(b) a VL domain comprising an amino acid sequence having at least 90%, at least 95%, or at least 99% sequence identity to the amino acid sequence of SEQ ID NO: 1447.
8. The antibody of paragraph 7, wherein the antibody comprises a VH domain comprising the amino acid sequence of SEQ ID NO: 1446 and a VL domain comprising the amino acid sequence of SEQ ID NO: 1447.
9. The antibody of any one of paragraphs 1-8, wherein the antibody comprises (a) a heavy chain comprising the amino acid sequence of SEQ ID NO: 1456 and (b) a light chain comprising the amino acid sequence of SEQ ID NO: 1457.
10. The antibody of any one of paragraphs 1-9, wherein the antibody binds Ara h 2 with a K_{D} of about 50 nM or less.
11. The method of paragraph 10, wherein the K_{D} is measured by a surface plasmon resonance assay.
12. The antibody of paragraph 11, wherein the antibody binds Ara h 2 with a K_{D} of between about 0.1 pM and about 40 nM.
13. The antibody of paragraph 12, wherein the antibody binds Ara h 2 with a K_{D} of between about 120 pM and about 20 nM.
14. The antibody of paragraph 13, wherein the antibody binds Ara h 2 with a K_{D} of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)2 fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### 105BU7P1D8:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 1480);
(b) an CDR-H2 comprising the amino acid sequence of (SEQ ID NO: 1481);
(c) an CDR-H3 comprising the amino acid sequence of (SEQ ID NO: 1482);
(d) an CDR-L1 comprising the amino acid sequence of (SEQ ID NO: 1483);
(e) an CDR-L2 comprising the amino acid sequence of (SEQ ID NO: 1484); and
(f) an CDR-L3 comprising the amino acid sequence of (SEQ ID NO: 1485).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 1486;
(b) a light chain variable (VL) domain comprising an amino acid sequence having at least 90%, at least 95%, or at least 99% identity to the amino acid sequence of SEQ ID NO: 1487; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 1488);
(b) an FR-H2 comprising the amino acid sequence of (SEQ ID NO: 1489);
(c) an FR-H3 comprising the amino acid sequence of (SEQ ID NO: 1490); and
(d) an FR-H4 comprising the amino acid sequence of (SEQ ID NO: 1491).
4. The antibody of any one of paragraphs 1-3, wherein the VH domain comprises the amino acid sequence of SEQ ID NO: 1486.
5. The antibody of any one of paragraphs 1-4, further comprising the following VL domain FRs:
(a) an FR-L1 comprising the amino acid sequence of (SEQ ID NO: 1452);
(b) an FR-L2 comprising the amino acid sequence of (SEQ ID NO: 1453);
(c) an FR-L3 comprising the amino acid sequence of (SEQ ID NO: 1454); and
(d) an FR-L4 comprising the amino acid sequence of (SEQ ID NO: 1455).
6. The antibody of any one of paragraphs 1-5, wherein the VL domain comprises the amino acid sequence of SEQ ID NO: 1487.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 1486 and
(b) a VL domain comprising an amino acid sequence having at least 90%, at least 95%, or at least 99% sequence identity to the amino acid sequence of SEQ ID NO: 1487.
8. The antibody of paragraph 7, wherein the antibody comprises a VH domain comprising the amino acid sequence of SEQ ID NO: 1486 and a VL domain comprising the amino acid sequence of SEQ ID NO: 1487.
9. The antibody of any one of paragraphs 1-8, wherein the antibody comprises (a) a heavy chain comprising the amino acid sequence of SEQ ID NO: 1496 and (b) a light chain comprising the amino acid sequence of SEQ ID NO: 1497.
10. The antibody of any one of paragraphs 1-9, wherein the antibody binds Ara h 2 with a K_{D} of about 50 nM or less.
11. The method of paragraph 10, wherein the K_{D} is measured by a surface plasmon resonance assay.
12. The antibody of paragraph 11, wherein the antibody binds Ara h 2 with a K_{D} of between about 0.1 pM and about 40 nM.
13. The antibody of paragraph 12, wherein the antibody binds Ara h 2 with a K_{D} of between about 120 pM and about 20 nM.
14. The antibody of paragraph 13, wherein the antibody binds Ara h 2 with a K_{D} of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)₂ fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### 111BU7A12:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 1520);
(b) an CDR-H2 comprising the amino acid sequence of (SEQ ID NO: 1521);
(c) an CDR-H3 comprising the amino acid sequence of (SEQ ID NO: 1522);
(d) an CDR-L1 comprising the amino acid sequence of (SEQ ID NO: 1523);
(e) an CDR-L2 comprising the amino acid sequence of (SEQ ID NO: 1524); and
(f) an CDR-L3 comprising the amino acid sequence of (SEQ ID NO: 1525).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 1526;
(b) a light chain variable (VL) domain comprising an amino acid sequence having at least 90%, at least 95%, or at least 99% identity to the amino acid sequence of SEQ ID NO: 1527; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 1528);
(b) an FR-H2 comprising the amino acid sequence of (SEQ ID NO: 1529);
(c) an FR-H3 comprising the amino acid sequence of (SEQ ID NO: 1530); and
(d) an FR-H4 comprising the amino acid sequence of (SEQ ID NO: 1531).
4. The antibody of any one of paragraphs 1-3, wherein the VH domain comprises the amino acid sequence of SEQ ID NO: 1526.
5. The antibody of any one of paragraphs 1-4, further comprising the following VL domain FRs:
(a) an FR-L1 comprising the amino acid sequence of (SEQ ID NO: 1532);
(b) an FR-L2 comprising the amino acid sequence of (SEQ ID NO: 1533);
(c) an FR-L3 comprising the amino acid sequence of (SEQ ID NO: 1534); and
(d) an FR-L4 comprising the amino acid sequence of (SEQ ID NO: 1535).
6. The antibody of any one of paragraphs 1-5, wherein the VL domain comprises the amino acid sequence of SEQ ID NO: 1527.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 1526 and
(b) a VL domain comprising an amino acid sequence having at least 90%, at least 95%, or at least 99% sequence identity to the amino acid sequence of SEQ ID NO: 1527.
8. The antibody of paragraph 7, wherein the antibody comprises a VH domain comprising the amino acid sequence of SEQ ID NO: 1526 and a VL domain comprising the amino acid sequence of SEQ ID NO: 1527.
9. The antibody of any one of paragraphs 1-8, wherein the antibody comprises (a) a heavy chain comprising the amino acid sequence of SEQ ID NO: 1536 and (b) a light chain comprising the amino acid sequence of SEQ ID NO: 1537.
10. The antibody of any one of paragraphs 1-9, wherein the antibody binds Ara h 2 with a K_{D} of about 50 nM or less.
11. The method of paragraph 10, wherein the K_{D} is measured by a surface plasmon resonance assay.
12. The antibody of paragraph 11, wherein the antibody binds Ara h 2 with a K_{D} of between about 0.1 pM and about 40 nM.
13. The antibody of paragraph 12, wherein the antibody binds Ara h 2 with a K_{D} of between about 120 pM and about 20 nM.
14. The antibody of paragraph 13, wherein the antibody binds Ara h 2 with a K_{D} of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)₂ fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### 111BU7P1D2:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 1540);
(b) an CDR-H2 comprising the amino acid sequence of (SEQ ID NO: 1541);
(c) an CDR-H3 comprising the amino acid sequence of (SEQ ID NO: 1542);
(d) an CDR-L1 comprising the amino acid sequence of (SEQ ID NO: 1543);
(e) an CDR-L2 comprising the amino acid sequence of (SEQ ID NO: 1544); and
(f) an CDR-L3 comprising the amino acid sequence of (SEQ ID NO: 1545).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 1546;
(b) a light chain variable (VL) domain comprising an amino acid sequence having at least 90%, at least 95%, or at least 99% identity to the amino acid sequence of SEQ ID NO: 1547; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 1548);
(b) an FR-H2 comprising the amino acid sequence of (SEQ ID NO: 1549);
(c) an FR-H3 comprising the amino acid sequence of (SEQ ID NO: 1550); and
(d) an FR-H4 comprising the amino acid sequence of (SEQ ID NO: 1551).
4. The antibody of any one of paragraphs 1-3, wherein the VH domain comprises the amino acid sequence of SEQ ID NO: 1546.
5. The antibody of any one of paragraphs 1-4, further comprising the following VL domain FRs:
(a) an FR-L1 comprising the amino acid sequence of (SEQ ID NO: 1552);
(b) an FR-L2 comprising the amino acid sequence of (SEQ ID NO: 1553);
(c) an FR-L3 comprising the amino acid sequence of (SEQ ID NO: 1554); and
(d) an FR-L4 comprising the amino acid sequence of (SEQ ID NO: 1555).
6. The antibody of any one of paragraphs 1-5, wherein the VL domain comprises the amino acid sequence of SEQ ID NO: 1547.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 1546 and
(b) a VL domain comprising an amino acid sequence having at least 90%, at least 95%, or at least 99% sequence identity to the amino acid sequence of SEQ ID NO: 1547.
8. The antibody of paragraph 7, wherein the antibody comprises a VH domain comprising the amino acid sequence of SEQ ID NO: 1546 and a VL domain comprising the amino acid sequence of SEQ ID NO: 1547.
9. The antibody of any one of paragraphs 1-8, wherein the antibody comprises (a) a heavy chain comprising the amino acid sequence of SEQ ID NO: 1556 and (b) a light chain comprising the amino acid sequence of SEQ ID NO: 1557.
10. The antibody of any one of paragraphs 1-9, wherein the antibody binds Ara h 2 with a K_{D} of about 50 nM or less.
11. The method of paragraph 10, wherein the K_{D} is measured by a surface plasmon resonance assay.
12. The antibody of paragraph 11, wherein the antibody binds Ara h 2 with a K_{D} of between about 0.1 pM and about 40 nM.
13. The antibody of paragraph 12, wherein the antibody binds Ara h 2 with a K_{D} of between about 120 pM and about 20 nM.
14. The antibody of paragraph 13, wherein the antibody binds Ara h 2 with a K_{D} of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)2 fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### 111BU7P1D5:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 1560);
(b) an CDR-H2 comprising the amino acid sequence of (SEQ ID NO: 1561);
(c) an CDR-H3 comprising the amino acid sequence of (SEQ ID NO: 1562);
(d) an CDR-L1 comprising the amino acid sequence of (SEQ ID NO: 1563);
(e) an CDR-L2 comprising the amino acid sequence of (SEQ ID NO: 1564); and
(f) an CDR-L3 comprising the amino acid sequence of (SEQ ID NO: 1565).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 1566;
(b) a light chain variable (VL) domain comprising an amino acid sequence having at least 90%, at least 95%, or at least 99% identity to the amino acid sequence of SEQ ID NO: 1567; or (c) a VH domain as in (a) and a VL domain as in (b).
3. The antibody of paragraph 1 or 2, further comprising the following VH domain framework regions (FRs):
(a) an FR-H1 comprising the amino acid sequence of (SEQ ID NO: 1568);
(b) an FR-H2 comprising the amino acid sequence of (SEQ ID NO: 1569);
(c) an FR-H3 comprising the amino acid sequence of (SEQ ID NO: 1570); and
(d) an FR-H4 comprising the amino acid sequence of (SEQ ID NO: 1571).
4. The antibody of any one of paragraphs 1-3, wherein the VH domain comprises the amino acid sequence of SEQ ID NO: 1566.
5. The antibody of any one of paragraphs 1-4, further comprising the following VL domain FRs:
(a) an FR-L1 comprising the amino acid sequence of (SEQ ID NO: 1572);
(b) an FR-L2 comprising the amino acid sequence of (SEQ ID NO: 1573);
(c) an FR-L3 comprising the amino acid sequence of (SEQ ID NO: 1574); and
(d) an FR-L4 comprising the amino acid sequence of (SEQ ID NO: 1575).
6. The antibody of any one of paragraphs 1-5, wherein the VL domain comprises the amino acid sequence of SEQ ID NO: 1567.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 1566 and
(b) a VL domain comprising an amino acid sequence having at least 90%, at least 95%, or at least 99% sequence identity to the amino acid sequence of SEQ ID NO: 1567.
8. The antibody of paragraph 7, wherein the antibody comprises a VH domain comprising the amino acid sequence of SEQ ID NO: 1566 and a VL domain comprising the amino acid sequence of SEQ ID NO: 1567.
9. The antibody of any one of paragraphs 1-8, wherein the antibody comprises (a) a heavy chain comprising the amino acid sequence of SEQ ID NO: 1576 and (b) a light chain comprising the amino acid sequence of SEQ ID NO: 1577.
10. The antibody of any one of paragraphs 1-9, wherein the antibody binds Ara h 2 with a K_{D} of about 50 nM or less.
11. The method of paragraph 10, wherein the K_{D} is measured by a surface plasmon resonance assay.
12. The antibody of paragraph 11, wherein the antibody binds Ara h 2 with a K_{D} of between about 0.1 pM and about 40 nM.
13. The antibody of paragraph 12, wherein the antibody binds Ara h 2 with a K_{D} of between about 120 pM and about 20 nM.
14. The antibody of paragraph 13, wherein the antibody binds Ara h 2 with a K_{D} of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)₂ fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### 29BU7P1D1:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of (SEQ ID NO: 1660);
(b) an CDR-H2 comprising the amino acid sequence of (SEQ ID NO: 1661);
(c) an CDR-H3 comprising the amino acid sequence of (SEQ ID NO: 1562);
(d) an CDR-L1 comprising the amino acid sequence of (SEQ ID NO: 1663);
(e) an CDR-L2 comprising the amino acid sequence of (SEQ ID NO: 1664); and
(f) an CDR-L3 comprising the amino acid sequence of (SEQ ID NO: 1665).
2. The antibody of paragraph 1, wherein the antibody comprises
(a) a heavy chain variable (VH) domain comprising an amino sequence having at least 90%, at least 95%, or at least 99% sequence identity to the amino acid sequence of SEQ ID NO: 1666;
(b) a light chain variable (VL) domain comprising an amino acid sequence having at least 90%, at least 95%, or at least 99% identity to the amino acid sequence of SEQ ID NO: 1667; or (c) a VH domain as in
(a) and a VL domain as in (b).
3. The antibody of paragraph 1 or 2, further comprising the following VH domain framework regions (FRs):
(a) an FR-H1 comprising the amino acid sequence of (SEQ ID NO: 1668);
(b) an FR-H2 comprising the amino acid sequence of (SEQ ID NO: 1669);
(c) an FR-H3 comprising the amino acid sequence of (SEQ ID NO: 1670); and
(d) an FR-H4 comprising the amino acid sequence of (SEQ ID NO: 1671).
4. The antibody of any one of paragraphs 1-3, wherein the VH domain comprises the amino acid sequence of SEQ ID NO: 1666.
5. The antibody of any one of paragraphs 1-4, further comprising the following VL domain FRs:
(a) an FR-L1 comprising the amino acid sequence of (SEQ ID NO: 1672);
(b) an FR-L2 comprising the amino acid sequence of (SEQ ID NO: 1673);
(c) an FR-L3 comprising the amino acid sequence of (SEQ ID NO: 1674); and
(d) an FR-L4 comprising the amino acid sequence of (SEQ ID NO: 1675).
6. The antibody of any one of paragraphs 1-5, wherein the VL domain comprises the amino acid sequence of SEQ ID NO: 1667.
7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 1666 and
(b) a VL domain comprising an amino acid sequence having at least 90%, at least 95%, or at least 99% sequence identity to the amino acid sequence of SEQ ID NO: 1667.
8. The antibody of paragraph 7, wherein the antibody comprises a VH domain comprising the amino acid sequence of SEQ ID NO: 1566 and a VL domain comprising the amino acid sequence of SEQ ID NO: 1667.
9. The antibody of any one of paragraphs 1-8, wherein the antibody comprises (a) a heavy chain comprising the amino acid sequence of SEQ ID NO: 1676 and (b) a light chain comprising the amino acid sequence of SEQ ID NO: 1677.
10. The antibody of any one of paragraphs 1-9, wherein the antibody binds Ara h 2 with a K_{D} of about 50 nM or less.
11. The method of paragraph 10, wherein the K_{D} is measured by a surface plasmon resonance assay.
12. The antibody of paragraph 11, wherein the antibody binds Ara h 2 with a K_{D} of between about 0.1 pM and about 40 nM.
13. The antibody of paragraph 12, wherein the antibody binds Ara h 2 with a K_{D} of between about 120 pM and about 20 nM.
14. The antibody of paragraph 13, wherein the antibody binds Ara h 2 with a K_{D} of between about 120 pM and about 10 nM.
15. The antibody of any one of paragraphs 1-14, wherein the antibody is capable neutralizing Ara h 2
16. An antibody that binds to the same epitope as the antibody of any one of paragraph 1-15.
17. An antibody that competes for binding to Ara h 2 with, or cross-blocks or is cross-blocked by, the antibody of any one of paragraphs 1-16.
18. The antibody of paragraph 16 or 17, wherein whether the antibody binds to the same epitope or competes for binding to Ara h 2 is determined by an epitope binning assay.
19. The antibody of any one of paragraphs 1-18, wherein the antibody is monoclonal.
20. The antibody of any one of paragraphs 1-19, wherein the antibody is humanized.
21. The antibody of any one of paragraphs 1-20, wherein the antibody is an antibody fragment that binds Ara h 2.
22. The antibody of paragraph 21, wherein the antibody fragment is selected from the group consisting of Fab, Fab′-SH, Fv, scFv, and (Fab′)₂ fragments.
23. The antibody of any one of paragraphs 1-22, wherein the antibody is a full-length antibody.
24. The antibody of paragraph 23, wherein the antibody is an IgG antibody.
25. The antibody of paragraph 24, wherein the IgG antibody is an IgG1 antibody.
26. The antibody of paragraph 25, wherein the IgG antibody is an IgG4 antibody.
27. The antibody of paragraph 26, wherein the IgG4 antibody comprises an S228P mutation (EU numbering).
28. The antibody of any one of paragraphs 1-27, wherein the antibody is a monospecific antibody.
29. The antibody of any one of paragraphs 1-28, wherein the antibody is a multispecific antibody.
30. The antibody of paragraph 29, wherein the antibody is a bispecific antibody.
31. A polynucleotide encoding an isolated antibody of any one of paragraphs 1-30.
32. A vector comprising the polynucleotide of paragraph 31.
33. A host cell comprising the vector of paragraph 32.
34. A method of producing the antibody of any one of paragraphs 1-33, the method comprising culturing a host cell that comprises the vector of paragraph 32 and recovering the antibody.
35. The method of paragraph 34, wherein the host cell is prokaryotic.
36. The method of paragraph 35, wherein the host cell is Escherichia coli.
37. The method of paragraph 34, wherein the host cell is eukaryotic.
38. The method of paragraph 37, wherein the host cell is a 293 cell, a CHO cell, a yeast cell, or a plant cell.
39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of paragraphs 1-30.
40. The method of paragraph 39, further comprising administering an effective amount of a second therapeutic agent useful for diminishing an allergic reaction an allergic reaction against, a nut or a portion thereof or an extract thereof, or to Ara h 2 protein.
41. A method for detecting the presence of Ara h 2 neutralizing antibodies in a biological sample, comprising:
(a) providing a sample from a subject;
(b) contacting the sample with a mixture comprising:
   (i) the antibody of any one of paragraphs 1-30 and
   (ii) Ara h 2, or a fragment thereof; and
(c) detecting the binding of antibodies present in the biological sample to Ara h 2, or a fragment thereof.
42. The method of paragraph 41, where antibody binding is detected using tandem bio-layer interferometry.
43. The method of paragraphs 41 or 42, where the antibody from step (b) is bound to a solid support.
44. The method of paragraphs 41 or 42, where Ara h 2, or a fragment thereof, is bound to a solid support.
45. The method of paragraph 41, where antibody binding is detected using an enzyme-linked immunosorbent assay.

### Compositions and Methods

1. A pharmaceutical composition comprising a therapeutically effective amount of one or more isolated antibodies, or antigen-binding fragments thereof, as is described in Table 8, together with one or more pharmaceutically acceptable excipients.
2. A method for treating a patient who demonstrates a sensitivity to a peanut allergen, an allergic reaction against a peanut allergen, or to Ara h 2 protein, or for treating at least one symptom or complication associated with a sensitivity to, or allergic reaction against a peanut allergen, an allergic reaction against a peanut allergen, or to Ara h 2 protein, comprising administering an effective amount of one or more isolated human monoclonal antibodies or antigen-binding fragments thereof that bind specifically to Ara h 2, according to paragraphs described above to a patient in need thereof, wherein the sensitivity to, or an allergic reaction against, to a peanut allergen, an allergic reaction against a peanut allergen, or to Ara h 2 protein, is lessened in severity and/or duration, or at least one symptom or complication associated with the sensitivity to, or allergic reaction against, to a peanut allergen, an allergic reaction against a peanut allergen, or to Ara h 2 protein is ameliorated, or that the frequency and/or duration of, or the severity of the sensitivity to or allergic reaction against, to a peanut allergen, an allergic reaction against a peanut allergen, or to Ara h 2 protein, is reduced following administration of one or more of the isolated human monoclonal antibodies or fragments thereof that bind specifically to Ara h 2.
3. The method of paragraph 2, further comprising administering an effective amount of a second therapeutic agent useful for diminishing an allergic reaction to a peanut allergen, an allergic reaction against a peanut allergen, or to Ara h 2 protein.
4. The method of paragraph 3, wherein the second therapeutic agent is selected from the group consisting of a corticosteroid, a bronchial dilator, an antihistamine, epinephrine, or a decongestant.
5. The method of paragraph 2, wherein the treatment results in a reduction in allergic rhinitis, allergic conjunctivitis, allergic asthma, or an anaphylactic response following exposure, direct or indirect, of the patient to a peanut allergen, an allergic reaction against a peanut allergen, or to Ara h 2 protein.

Other embodiments are within the claims.

What is claimed is:

1. A combination of anti-Ara h 2 antibodies for use in determining a treatment response of an individual with a peanut allergy to peanut exposure, wherein the combination comprises three or more anti-Ara h 2 antibodies that bind the following Ara h 2 epitope bins:
(a) a first epitope bin comprising epitopes of one or more of anti-Ara h 2 antibodies P34, T1, T3, P33, P17, P21, P3, P13, P10, P11, P14, P19, P28, P30, P39, U1, 13FU1P1A4, 13FU1P1B4, 14FU2P1A11, 15FU1P1A3, 13FU1P2B12, 11FUP1A2, 18FU1P1A7, 23FUP1A8, 23FUP1B8, 23FUP1C4, 23FUP1D6, 23FUP1D12, 24BU7P1A10, 24BU7P1B6, 24BU7P1D1, 24BU7P1C10, 24BU7P1D9, 24BU7P1C2, 105BU7P1A11, 105BU7P1C3, 105BU7P1D7, 105BU7P1 D12, and 33BU7P1 D11, wherein P34 comprises the following complementarity determining regions (CDRs): a CDR-H1 comprising the amino acid sequence of GFTFDDYT (SEQ ID NO:341), a CDR-H2 comprising the amino acid sequence of IRWDGSRT (SEQ ID NO:342), a CDR-H3 comprising the amino acid sequence of VKDGGLRYFDS (SEQ ID NO:343), a CDR-L1 comprising the amino acid sequence of QSLLHSNGIHY (SEQ ID NO:344), a CDR-L2 comprising the amino acid sequence of LGS (SEQ ID NO:345), and a CDR-L3 comprising the amino acid sequence of MQSLQTFT (SEQ ID NO:346);
(b) a second epitope bin comprising epitopes of one or more of anti-Ara h 2 antibodies P31, T4, T5, S4, 14FU2P1 D6, 15FU1P3A6, 13FU1P2B10, and 27FU1P3A10, wherein P31 comprises the following CDRs: a CDR-H1 comprising the amino acid sequence of GDPFTSYY (SEQ ID NO:301), a CDR-H2 comprising the amino acid sequence of IFTTGST (SEQ ID NO:302), a CDR-H3 comprising the amino acid sequence of ARVRRYCSGGRCYPYFYMDV (SEQ ID NO:303), a CDR-L1 comprising the amino acid sequence of ESISSW (SEQ ID NO:304), a CDR-L2 comprising the amino acid sequence of EAS (SEQ ID NO:305), and a CDR-L3 comprising the amino acid sequence of QHYNSDSLT (SEQ ID NO:306);
(c) a third epitope bin comprising an epitope of anti-Ara h 2 antibody S1, 27FU1P3A4, 6BU4P2B1, and 89BU7P1B10, wherein S1 comprises the following CDRs: a CDR-H1 comprising the amino acid sequence of GFSFSDSY (SEQ ID NO:381), a CDR-H2 comprising the amino acid sequence of ISGSGEII (SEQ ID NO:382), a CDR-H3 comprising the amino acid sequence of ARPSDYFETSEELD (SEQ ID NO:383), a CDR-L1 comprising the amino acid sequence of QSISTY (SEQ ID NO:384), a CDR-L2 comprising the amino acid sequence of AAS (SEQ ID NO:385), and a CDR-L3 comprising the amino acid sequence of HQSYSAPRT (SEQ ID NO:386); and, optionally,
(d) a fourth epitope bin comprising epitopes of one or more of anti-Ara h 2 antibodies P7, P6, 111BU7P1A12, 111BU7P1D2, 111BU7P1D5, 24BU7P1D3, and 24BU7P1B1, wherein P7 comprises the following CDRs: a CDR-H1 comprising the amino acid sequence of GFTFTRYA (SEQ ID NO:41), a CDR-H2 comprising the amino acid sequence of ISHDGGTK (SEQ ID NO:42), a CDR-H3 comprising the amino acid sequence of AKTCSSPSCYDTAYYFDY (SEQ ID NO:43), a CDR-L1 comprising the amino acid sequence of QSLGNY (SEQ ID NO:44), a CDR-L2 comprising the amino acid sequence of DAS (SEQ ID NO:45), and a CDR-L3 comprising the amino acid sequence of QQRSQFMWT (SEQ ID NO:46).

2. The combination of claim 1, wherein the epitopes of the one or more anti-Ara h 2 antibodies of the fourth epitope bin comprise the amino acid sequence of DPYS (SEQ ID NO:1880) or DPYSZS (SEQ ID NO:1881).

3. The combination of claim 1 or 2, wherein the combination further comprises one or more anti-Ara h 2 antibodies that bind:
(e) a fifth epitope bin comprising an epitopes of one or more anti-Ara h 2 antibodies 24B7D4, T6, 15FU1P3A1, 23FUP1C10, 23FUP1D8, and 24BU7P1D4.

4. The combination of claim 3, wherein the epitope of the one or more anti-Ara h 2 antibodies of the fifth epitope bin comprises the amino acid sequence of QSQLER (SEQ ID NO:1882).

5. The combination of any one of claims 1-4, wherein the combination further comprises one or more anti-Ara h 2 antibodies that bind:
(f) a sixth epitope bin comprising epitopes of one or more of anti-Ara h 2 antibodies P8, P16, and P22.

6. The combination of claim 5, wherein the epitopes of the one or more anti-Ara h 2 antibodies of the sixth epitope bin comprise the amino acid sequence of KRELRNL (SEQ ID NO:1883).

7. The combination of any one of claims 1-6, wherein the combination further comprises one or more anti-Ara h 2 antibodies that bind:
(g) a seventh epitope bin comprising epitopes of one or more anti-Ara h 2 antibodies 105BU7P1D6 and 105BU7P1D8.

8. The combination of claim 7, wherein the epitopes of the one or more anti-Ara h 2 antibodies of the seventh epitope bin comprise the amino acid sequence of RQQEQQ (SEQ ID NO:1885).

9. The combination of any one of claims 1-8, wherein the combination further comprises one or more anti-Ara h 2 antibodies that bind:
(h) an eighth epitope bin comprising an epitope of anti-Ara h 2 antibody 29BU7P1 D1.

10. The combination of claim 9, wherein epitopes of the one or more anti-Ara h 2 antibodies of the seventh epitope bin comprise the amino acid sequence of CEALQQ (SEQ ID NO:1887).

11. A combination of anti-Ara h 2 antibodies for use in determining a treatment response of an individual with a peanut allergy to peanut exposure, wherein the combination comprises anti-Ara h 2 antibodies:
(a) P34, P33, or P17;
(b) P31;
(c) S1; and
(d) P7.

12. The combination of claim 11, wherein the combination comprises anti-Ara h 2 antibodies:
(a) P34;
(b) P31;
(c) S1; and
(d) P7.

13. A method for assessing a treatment response of an individual with a peanut allergy to peanut exposure, the method comprising measuring anti-Ara h 2 antibodies in a sample from a subject using a competitive assay comprising one or more anti-Ara h 2 antibodies.

14. The method of claim 13, wherein the one or more anti-Ara h 2 antibodies comprise a combination of one or more anti-Ara h 2 antibodies that bind the following Ara h 2 epitope bins:
(a) a first epitope bin comprising epitopes of one or more of anti-Ara h 2 antibodies P34, T1, T3, P33, P17, P21, P3, P13, P10, P11, P14, P19, P28, P30, P39, U1, 13FU1P1A4, 13FU1P1B4, 14FU2P1A11, 15FU1P1A3, 13FU1P2B12, 11FUP1A2, 18FU1P1A7, 23FUP1A8, 23FUP1B8, 23FUP1C4, 23FUP1D6, 23FUP1D12, 24BU7P1A10, 24BU7P1B6, 24BU7P1D1, 24BU7P1C10, 24BU7P1D9, 24BU7P1C2, 105BU7P1A11, 105BU7P1C3, 105BU7P1D7, 105BU7P1D12, and 33BU7P1D11, wherein P34 comprises the following CDRs: a CDR-H1 comprising the amino acid sequence of GFTFDDYT (SEQ ID NO:341), a CDR-H2 comprising the amino acid sequence of IRWDGSRT (SEQ ID NO:342), a CDR-H3 comprising the amino acid sequence of VKDGGLRYFDS (SEQ ID NO:343), a CDR-L1 comprising the amino acid sequence of QSLLHSNGIHY (SEQ ID NO:344), a CDR-L2 comprising the amino acid sequence of LGS (SEQ ID NO:345), and a CDR-L3 comprising the amino acid sequence of MQSLQTFT (SEQ ID NO:346);
(b) a second epitope bin comprising epitopes of one or more of anti-Ara h 2 antibodies P31, T4, T5, S4, 14FU2P1 D6, 15FU1P3A6, 13FU1P2B10, and 27FU1P3A10, wherein P31 comprises the following CDRs: a CDR-H1 comprising the amino acid sequence of GDPFTSYY (SEQ ID NO:301), a CDR-H2 comprising the amino acid sequence of IFTTGST (SEQ ID NO:302), a CDR-H3 comprising the amino acid sequence of ARVRRYCSGGRCYPYFYMDV (SEQ ID NO:303), a CDR-L1 comprising the amino acid sequence of ESISSW (SEQ ID NO:304), a CDR-L2 comprising the amino acid sequence of EAS (SEQ ID NO:305), and a CDR-L3 comprising the amino acid sequence of QHYNSDSLT (SEQ ID NO:306);
(c) a third epitope bin comprising an epitope of anti-Ara h 2 antibody S1, 27FU1P3A4, 6BU4P2B1, and 89BU7P1B10, wherein S1 comprises the following CDRs: a CDR-H1 comprising the amino acid sequence of GFSFSDSY (SEQ ID NO:381), a CDR-H2 comprising the amino acid sequence of ISGSGEII (SEQ ID NO:382), a CDR-H3 comprising the amino acid sequence of ARPSDYFETSEELD (SEQ ID NO:383), a CDR-L1 comprising the amino acid sequence of QSISTY (SEQ ID NO:384), a CDR-L2 comprising the amino acid sequence of AAS (SEQ ID NO:385), and a CDR-L3 comprising the amino acid sequence of HQSYSAPRT (SEQ ID NO:386); and, optionally,
(d) a fourth epitope bin comprising epitopes of one or more of anti-Ara h 2 antibodies P7, P6, 111BU7P1A12, 111BU7P1D2, 111BU7P1D5, 24BU7P1D3, and 24BU7P1B1, wherein P7 comprises the following CDRs: a CDR-H1 comprising the amino acid sequence of GFTFTRYA (SEQ ID NO:41), a CDR-H2 comprising the amino acid sequence of ISHDGGTK (SEQ ID NO:42), a CDR-H3 comprising the amino acid sequence of AKTCSSPSCYDTAYYFDY (SEQ ID NO:43), a CDR-L1 comprising the amino acid sequence of QSLGNY (SEQ ID NO:44), a CDR-L2 comprising the amino acid sequence of DAS (SEQ ID NO:45), and a CDR-L3 comprising the amino acid sequence of QQRSQFMWT (SEQ ID NO:46).

15. The method of claim 14, wherein the epitopes of the one or more anti-Ara h 2 antibodies of the fourth epitope bin comprise the amino acid sequence of DPYS (SEQ ID NO:1880) or DPYSZS (SEQ ID NO:1881).

16. The method of claim 14 or 15, wherein the combination further comprises one or more anti-Ara h 2 antibodies that bind:
(e) a fifth epitope bin comprising epitopes of one or more anti-Ara h 2 antibodies 24B7D4, T6, 15FU1P3A1, 23FUP1C10, 23FUP1D8, and 24BU7P1D4.

17. The method of claim 16, wherein the epitope of the one or more anti-Ara h 2 antibodies of the fifth epitope bin comprises the amino acid sequence of QSQLER (SEQ ID NO:1882).

18. The method of any one of claims 14-17, wherein the combination further comprises one or more anti-Ara h 2 antibodies that bind:
(f) a sixth epitope bin comprising epitopes of one or more of anti-Ara h 2 antibodies P8, P16, and P22.

19. The method of claim 18, wherein the epitopes of the one or more anti-Ara h 2 antibodies of the sixth epitope bin comprise the amino acid sequence of KRELRNL (SEQ ID NO:1883).

20. The method of any one of claims 14-19, wherein the combination further comprises one or more anti-Ara h 2 antibodies that bind:
(g) a seventh epitope bin comprising epitopes of one or more anti-Ara h 2 antibodies 105BU7P1D6, and 105BU7P1D8.

21. The combination of claim 20, wherein the epitopes of the one or more anti-Ara h 2 antibodies of the seventh epitope bin comprise the amino acid sequence of RQQEQQ (SEQ ID NO:1885).

22. The combination of any one of claims 14-21, wherein the combination further comprises one or more anti-Ara h 2 antibodies that bind:
(h) an eighth epitope bin comprising an epitope of anti-Ara h 2 antibody 29BU7P1 D1.

23. The combination of claim 22, wherein epitopes of the one or more anti-Ara h 2 antibodies of the seventh epitope bin comprise the amino acid sequence of CEALQQ (SEQ ID NO:1887).

24. The method of any one of claims 14-23, wherein the combination comprises anti-Ara h 2 antibodies:
(a) P34, P33, or P17;
(b) P31;
(c) S1; and
(d) P7.

25. The method of claim 24, wherein the combination comprises anti-Ara h 2 antibodies:
(a) P34;
(b) P31;
(c) S1; and
(d) P7.

26. The method of any one of claims 13-25, wherein the competitive assay comprises bio-layer interferometry (BLI).

27. The method of any one of claims 13-26, wherein the sample is a plasma sample.

28. A kit comprising one or more anti-Ara h 2 antibodies and instructions for use in determining the presence or level of Ara h 2 antibodies in a sample.

29. The kit of claim 28, wherein the one or more anti-Ara h 2 antibodies comprise a combination of one or more anti-Ara h 2 antibodies that bind the following Ara h 2 epitope bins:
(a) a first epitope bin comprising epitopes of one or more of anti-Ara h 2 antibodies P34, T1, T3, P33, P17, P21, P3, P13, P10, P11, P14, P19, P28, P30, P39, U1, 13FU1P1A4, 13FU1P1B4, 14FU2P1A11, 15FU1P1A3, 13FU1P2B12, 11FUP1A2, 18FU1P1A7, 23FUP1A8, 23FUP1B8, 23FUP1C4, 23FUP1D6, 23FUP1 D12, 24BU7P1A10, 24BU7P1B6, 24BU7P1D1, 24BU7P1C10, 24BU7P1D9, 24BU7P1C2, 105BU7P1A11, 105BU7P1C3, 105BU7P1 D7, 105BU7P1 D12, and 33BU7P1 D11, wherein P34 comprises the following CDRs: a CDR-H1 comprising the amino acid sequence of GFTFDDYT (SEQ ID NO:341), a CDR-H2 comprising the amino acid sequence of IRWDGSRT (SEQ ID NO:342), a CDR-H3 comprising the amino acid sequence of VKDGGLRYFDS (SEQ ID NO:343), a CDR-L1 comprising the amino acid sequence of QSLLHSNGIHY (SEQ ID NO:344), a CDR-L2 comprising the amino acid sequence of LGS (SEQ ID NO:345), and a CDR-L3 comprising the amino acid sequence of MQSLQTFT (SEQ ID NO:346);
(b) a second epitope bin comprising epitopes of one or more of anti-Ara h 2 antibodies P31, T4, T5, S4, 14FU2P1 D6, 15FU1P3A6, 13FU1P2B10, and 27FU1P3A10, wherein P31 comprises the following CDRs: a CDR-H1 comprising the amino acid sequence of GDPFTSYY (SEQ ID NO:301), a CDR-H2 comprising the amino acid sequence of IFTTGST (SEQ ID NO:302), a CDR-H3 comprising the amino acid sequence of ARVRRYCSGGRCYPYFYMDV (SEQ ID NO:303), a CDR-L1 comprising the amino acid sequence of ESISSW (SEQ ID NO:304), a CDR-L2 comprising the amino acid sequence of EAS (SEQ ID NO:305), and a CDR-L3 comprising the amino acid sequence of QHYNSDSLT (SEQ ID NO:306);
(c) a third epitope bin comprising an epitope of anti-Ara h 2 antibody S1, 27FU1P3A4, 6BU4P2B1, and 89BU7P1B10, wherein S1 comprises the following CDRs: a CDR-H1 comprising the amino acid sequence of GFSFSDSY (SEQ ID NO:381), a CDR-H2 comprising the amino acid sequence of ISGSGEII (SEQ ID NO:382), a CDR-H3 comprising the amino acid sequence of ARPSDYFETSEELD (SEQ ID NO:383), a CDR-L1 comprising the amino acid sequence of QSISTY (SEQ ID NO:384), a CDR-L2 comprising the amino acid sequence of AAS (SEQ ID NO:385), and a CDR-L3 comprising the amino acid sequence of HQSYSAPRT (SEQ ID NO:386); and
(d) a fourth epitope bin comprising epitopes of one or more of anti-Ara h 2 antibodies P7, P6, 111BU7P1A12, 111BU7P1D2, 111BU7P1D5, 24BU7P1D3, and 24BU7P1B1, wherein P7 comprises the following CDRs: a CDR-H1 comprising the amino acid sequence of GFTFTRYA (SEQ ID NO:41), a CDR-H2 comprising the amino acid sequence of ISHDGGTK (SEQ ID NO:42), a CDR-H3 comprising the amino acid sequence of AKTCSSPSCYDTAYYFDY (SEQ ID NO:43), a CDR-L1 comprising the amino acid sequence of QSLGNY (SEQ ID NO:44), a CDR-L2 comprising the amino acid sequence of DAS (SEQ ID NO:45), and a CDR-L3 comprising the amino acid sequence of QQRSQFMWT (SEQ ID NO:46).

30. The kit of claim 29, wherein the epitopes of the one or more anti-Ara h 2 antibodies of the fourth epitope bin comprise the amino acid sequence of DPYS (SEQ ID NO:1880) or DPYSZS (SEQ ID NO:1881).

31. The kit of claim 29 or 30, wherein the combination further comprises one or more anti-Ara h 2 antibodies that bind:
(e) a fifth epitope bin comprising epitopes of one or more anti-Ara h 2 antibodies 24B7D4, T6, 15FU1P3A1, 23FUP1C10, 23FUP1D8, and 24BU7P1D4.

32. The kit of claim 31, wherein the epitope of the one or more anti-Ara h 2 antibodies of the fifth epitope bin comprises the amino acid sequence of QSQLER (SEQ ID NO:1882).

33. The kit of any one of claims 29-32, wherein the combination further comprises one or more anti Ara h 2 antibodies that bind:
(f) a sixth epitope bin comprising epitopes of one or more of anti-Ara h 2 antibodies P8, P16, and P22.

34. The kit of claim 33, wherein the epitopes of the one or more anti-Ara h 2 antibodies of the sixth epitope bin comprise the amino acid sequence of KRELRNL (SEQ ID NO:1883).

35. The kit of any one of claims 29-34, wherein the combination further comprises one or more anti-Ara h 2 antibodies that bind:
(g) a seventh epitope bin comprising epitopes of one or more anti-Ara h 2 antibodies 105BU7P1D6, and 105BU7P1D8.

36. The kit of claim 35, wherein the epitopes of the one or more anti-Ara h 2 antibodies of the seventh epitope bin comprise the amino acid sequence of RQQEQQ (SEQ ID NO:1885).

37. The kit of any one of claims 29-36, wherein the combination further comprises one or more anti-Ara h 2 antibodies that bind:
(h) an eighth epitope bin comprising an epitope of anti-Ara h 2 antibody 29BU7P1 D1.

38. The kit of claim 37, wherein epitopes of the one or more anti-Ara h 2 antibodies of the seventh epitope bin comprise the amino acid sequence of CEALQQ (SEQ ID NO:1887).

39. The kit of any one of claims 29-38, wherein the combination comprises anti-Ara h 2 antibodies:
(a) P34, P33, or P17;
(b) P31;
(c) S1; and
(d) P7.

40. The kit of claim 39, wherein the combination comprises anti-Ara h 2 antibodies:
(a) P34;
(b) P31;
(c) S1; and
(d) P7.
